SEQUENCE LISTING (684 sequences)

SEQ ID NO: 1 - RNA, 17 nt, Homo sapiens
  source 1..17: mol_type = transcribed RNA
ttggaggcgtgggtttt

SEQ ID NO: 2 - RNA, 21 nt, Homo sapiens
  source 1..21: mol_type = transcribed RNA
cggcggggacggcgattggtc

SEQ ID NO: 3 - RNA, 18 nt, Homo sapiens
  source 1..18: mol_type = transcribed RNA
ccagaggtggggactgag

SEQ ID NO: 4 - RNA, 23 nt, Homo sapiens
  source 1..23: mol_type = transcribed RNA
ggaggcgcaggctcggaaaggcg

SEQ ID NO: 5 - RNA, 23 nt, Homo sapiens
  source 1..23: mol_type = transcribed RNA
agaggctttgtgcggatacgggg

SEQ ID NO: 6 - RNA, 24 nt, Homo sapiens
  source 1..24: mol_type = transcribed RNA
tgggcgaggggtgggctctcagag

SEQ ID NO: 7 - RNA, 22 nt, Homo sapiens
  source 1..22: mol_type = transcribed RNA
ctcctggggcccgcactctcgc

SEQ ID NO: 8 - RNA, 22 nt, Homo sapiens
  source 1..22: mol_type = transcribed RNA
actgggtaggtggggctccagg

SEQ ID NO: 9 - RNA, 22 nt, Homo sapiens
  source 1..22: mol_type = transcribed RNA
tggggagcggcccccgggtggg

SEQ ID NO: 10 - RNA, 22 nt, Homo sapiens
  source 1..22: mol_type = transcribed RNA
agacacatttggagagggaccc

SEQ ID NO: 11 - RNA, 22 nt, Homo sapiens
  source 1..22: mol_type = transcribed RNA
gtgggtgctggtgggagccgtg

SEQ ID NO: 12 - RNA, 23 nt, Homo sapiens
  source 1..23: mol_type = transcribed RNA
tgagtggggctcccgggacggcg

SEQ ID NO: 13 - RNA, 23 nt, Homo sapiens
  source 1..23: mol_type = transcribed RNA
tcaataggaaagaggtgggacct

SEQ ID NO: 14 - RNA, 23 nt, Homo sapiens
  source 1..23: mol_type = transcribed RNA
tgagcaccacacaggccgggcgc

SEQ ID NO: 15 - RNA, 23 nt, Homo sapiens
  source 1..23: mol_type = transcribed RNA
tcgaggactggtggaagggcctt

SEQ ID NO: 16 - RNA, 22 nt, Homo sapiens
  source 1..22: mol_type = transcribed RNA
gggtggggatttgttgcattac

SEQ ID NO: 17 - RNA, 17 nt, Homo sapiens
  source 1..17: mol_type = transcribed RNA
ccccgccaccgccttgg

SEQ ID NO: 18 - RNA, 20 nt, Homo sapiens
  source 1..20: mol_type = transcribed RNA
ggctccttggtctaggggta

SEQ ID NO: 19 - RNA, 20 nt, Homo sapiens
  source 1..20: mol_type = transcribed RNA
gcggaaggcggagcggcgga

SEQ ID NO: 20 - RNA, 22 nt, Homo sapiens
  source 1..22: mol_type = transcribed RNA
tggtggaggaagagggcagctc

SEQ ID NO: 21 - RNA, 19 nt, Homo sapiens
  source 1..19: mol_type = transcribed RNA
agcagggctggggattgca

SEQ ID NO: 22 - RNA, 22 nt, Homo sapiens
  source 1..22: mol_type = transcribed RNA
tggcggcggtagttatgggctt

SEQ ID NO: 23 - RNA, 22 nt, Homo sapiens
  source 1..22: mol_type = transcribed RNA
tcgggcctggggttgggggagc

SEQ ID NO: 24 - RNA, 20 nt, Homo sapiens
  source 1..20: mol_type = transcribed RNA
taggatgggggtgagaggtg

SEQ ID NO: 25 - RNA, 21 nt, Homo sapiens
  source 1..21: mol_type = transcribed RNA
accactgcactccagcctgag

SEQ ID NO: 26 - RNA, 21 nt, Homo sapiens
  source 1..21: mol_type = transcribed RNA
agcggtgctcctgcgggccga

SEQ ID NO: 27 - RNA, 22 nt, Homo sapiens
  source 1..22: mol_type = transcribed RNA
ggaggggtcccgcactgggagg

SEQ ID NO: 28 - RNA, 21 nt, Homo sapiens
  source 1..21: mol_type = transcribed RNA
aagggacagggagggtcgtgg

SEQ ID NO: 29 - RNA, 21 nt, Homo sapiens
  source 1..21: mol_type = transcribed RNA
cgggagctggggtctgcaggt

SEQ ID NO: 30 - RNA, 23 nt, Homo sapiens
  source 1..23: mol_type = transcribed RNA
gggggccgatacactgtacgaga

SEQ ID NO: 31 - RNA, 20 nt, Homo sapiens
  source 1..20: mol_type = transcribed RNA
cggggtgggtgaggtcgggc

SEQ ID NO: 32 - RNA, 21 nt, Homo sapiens
  source 1..21: mol_type = transcribed RNA
tcacctggctggcccgcccag

SEQ ID NO: 33 - RNA, 23 nt, Homo sapiens
  source 1..23: mol_type = transcribed RNA
agaagaaggcggtcggtctgcgg

SEQ ID NO: 34 - RNA, 18 nt, Homo sapiens
  source 1..18: mol_type = transcribed RNA
cggtgagcgctcgctggc

SEQ ID NO: 35 - RNA, 23 nt, Homo sapiens
  source 1..23: mol_type = transcribed RNA
tggggggacagatggagaggaca

SEQ ID NO: 36 - RNA, 21 nt, Homo sapiens
  source 1..21: mol_type = transcribed RNA
gttggggtgcaggggtctgct

SEQ ID NO: 37 - RNA, 22 nt, Homo sapiens
  source 1..22: mol_type = transcribed RNA
gggaccatcctgcctgctgtgg

SEQ ID NO: 38 - RNA, 23 nt, Homo sapiens
  source 1..23: mol_type = transcribed RNA
tggggaaggcttggcagggaaga

SEQ ID NO: 39 - RNA, 23 nt, Homo sapiens
  source 1..23: mol_type = transcribed RNA
gccccggcgcgggcgggttctgg

SEQ ID NO: 40 - RNA, 22 nt, Homo sapiens
  source 1..22: mol_type = transcribed RNA
tcaaaatcaggagtcggggctt

SEQ ID NO: 41 - RNA, 20 nt, Homo sapiens
  source 1..20: mol_type = transcribed RNA
ggatccgagtcacggcacca

SEQ ID NO: 42 - RNA, 21 nt, Homo sapiens
  source 1..21: mol_type = transcribed RNA
ggggggatgtgcatgctggtt

SEQ ID NO: 43 - RNA, 18 nt, Homo sapiens
  source 1..18: mol_type = transcribed RNA
atcctagtcacggcacca

SEQ ID NO: 44 - RNA, 22 nt, Homo sapiens
  source 1..22: mol_type = transcribed RNA
tgcggggctagggctaacagca

SEQ ID NO: 45 - RNA, 22 nt, Homo sapiens
  source 1..22: mol_type = transcribed RNA
ggctggagcgagtgcagtggtg

SEQ ID NO: 46 - RNA, 20 nt, Homo sapiens
  source 1..20: mol_type = transcribed RNA
tgtgggactgcaaatgggag

SEQ ID NO: 47 - RNA, 21 nt, Homo sapiens
  source 1..21: mol_type = transcribed RNA
tggggcggggcaggtccctgc

SEQ ID NO: 48 - RNA, 23 nt, Homo sapiens
  source 1..23: mol_type = transcribed RNA
cgggctgtccggaggggtcggct

SEQ ID NO: 49 - RNA, 18 nt, Homo sapiens
  source 1..18: mol_type = transcribed RNA
ctggcagggggagaggta

SEQ ID NO: 50 - RNA, 22 nt, Homo sapiens
  source 1..22: mol_type = transcribed RNA
cccctggggctgggcaggcgga

SEQ ID NO: 51 - RNA, 24 nt, Homo sapiens
  source 1..24: mol_type = transcribed RNA
ccccgggaacgtcgagactggagc

SEQ ID NO: 52 - RNA, 18 nt, Homo sapiens
  source 1..18: mol_type = transcribed RNA
ttcccagccaacgcacca

SEQ ID NO: 53 - RNA, 17 nt, Homo sapiens
  source 1..17: mol_type = transcribed RNA
ctccgggacggctgggc

SEQ ID NO: 54 - RNA, 19 nt, Homo sapiens
  source 1..19: mol_type = transcribed RNA
ggggagcgaggggcggggc

SEQ ID NO: 55 - RNA, 21 nt, Homo sapiens
  source 1..21: mol_type = transcribed RNA
cgggccggaggtcaagggcgt

SEQ ID NO: 56 - RNA, 19 nt, Homo sapiens
  source 1..19: mol_type = transcribed RNA
tgggggagatgggggttga

SEQ ID NO: 57 - RNA, 22 nt, Homo sapiens
  source 1..22: mol_type = transcribed RNA
tgggcgagggcggctgagcggc

SEQ ID NO: 58 - RNA, 18 nt, Homo sapiens
  source 1..18: mol_type = transcribed RNA
cccagcaggacgggagcg

SEQ ID NO: 59 - RNA, 21 nt, Homo sapiens
  source 1..21: mol_type = transcribed RNA
cgtggaggacgaggaggaggc

SEQ ID NO: 60 - RNA, 22 nt, Homo sapiens
  source 1..22: mol_type = transcribed RNA
tggggaggtgtggagtcagcat

SEQ ID NO: 61 - RNA, 21 nt, Homo sapiens
  source 1..21: mol_type = transcribed RNA
ctgggctcgggacgcgcggct

SEQ ID NO: 62 - RNA, 20 nt, Homo sapiens
  source 1..20: mol_type = transcribed RNA
gctcggactgagcaggtggg

SEQ ID NO: 63 - RNA, 22 nt, Homo sapiens
  source 1..22: mol_type = transcribed RNA
agcccgccccagccgaggttct

SEQ ID NO: 64 - RNA, 25 nt, Homo sapiens
  source 1..25: mol_type = transcribed RNA
aggggggcactgcgcaagcaaagcc

SEQ ID NO: 65 - RNA, 22 nt, Homo sapiens
  source 1..22: mol_type = transcribed RNA
tgcaggggtcgggtgggccagg

SEQ ID NO: 66 - RNA, 17 nt, Homo sapiens
  source 1..17: mol_type = transcribed RNA
gggagaagggtcggggc

SEQ ID NO: 67 - RNA, 22 nt, Homo sapiens
  source 1..22: mol_type = transcribed RNA
tagggatgggaggccaggatga

SEQ ID NO: 68 - RNA, 22 nt, Homo sapiens
  source 1..22: mol_type = transcribed RNA
gcccaggactttgtgcggggtg

SEQ ID NO: 69 - RNA, 21 nt, Homo sapiens
  source 1..21: mol_type = transcribed RNA
atccagttctctgagggggct

SEQ ID NO: 70 - RNA, 23 nt, Homo sapiens
  source 1..23: mol_type = transcribed RNA
agggtggggctggaggtggggct

SEQ ID NO: 71 - RNA, 21 nt, Homo sapiens
  source 1..21: mol_type = transcribed RNA
gtaggtgacagtcaggggcgg

SEQ ID NO: 72 - RNA, 23 nt, Homo sapiens
  source 1..23: mol_type = transcribed RNA
ctcggggcaggcggctgggagcg

SEQ ID NO: 73 - RNA, 18 nt, Homo sapiens
  source 1..18: mol_type = transcribed RNA
gtgaaggcccggcggaga

SEQ ID NO: 74 - RNA, 21 nt, Homo sapiens
  source 1..21: mol_type = transcribed RNA
cccatgcctcctgccgcggtc

SEQ ID NO: 75 - RNA, 22 nt, Homo sapiens
  source 1..22: mol_type = transcribed RNA
cagggctggcagtgacatgggt

SEQ ID NO: 76 - RNA, 18 nt, Homo sapiens
  source 1..18: mol_type = transcribed RNA
cgggcgtggtggtggggg

SEQ ID NO: 77 - RNA, 21 nt, Homo sapiens
  source 1..21: mol_type = transcribed RNA
ccggccgccggctccgccccg

SEQ ID NO: 78 - RNA, 23 nt, Homo sapiens
  source 1..23: mol_type = transcribed RNA
tgaggatatggcagggaagggga

SEQ ID NO: 79 - RNA, 17 nt, Homo sapiens
  source 1..17: mol_type = transcribed RNA
ggatggaggaggggtct

SEQ ID NO: 80 - RNA, 22 nt, Homo sapiens
  source 1..22: mol_type = transcribed RNA
ggggctgtgattgaccagcagg

SEQ ID NO: 81 - RNA, 21 nt, Homo sapiens
  source 1..21: mol_type = transcribed RNA
gtgtggccggcaggcgggtgg

SEQ ID NO: 82 - RNA, 20 nt, Homo sapiens
  source 1..20: mol_type = transcribed RNA
ggggaggtgtgcagggctgg

SEQ ID NO: 83 - RNA, 22 nt, Homo sapiens
  source 1..22: mol_type = transcribed RNA
caggaggcagtgggcgagcagg

SEQ ID NO: 84 - RNA, 17 nt, Homo sapiens
  source 1..17: mol_type = transcribed RNA
ggggcgcggccggatcg

SEQ ID NO: 85 - RNA, 23 nt, Homo sapiens
  source 1..23: mol_type = transcribed RNA
ccggggcagattggtgtagggtg

SEQ ID NO: 86 - RNA, 19 nt, Homo sapiens
  source 1..19: mol_type = transcribed RNA
tggggcggagcttccggag

SEQ ID NO: 87 - RNA, 21 nt, Homo sapiens
  source 1..21: mol_type = transcribed RNA
cgggggcggggccgaagcgcg

SEQ ID NO: 88 - RNA, 23 nt, Homo sapiens
  source 1..23: mol_type = transcribed RNA
gtgagtgggagccggtggggctg

SEQ ID NO: 89 - RNA, 24 nt, Homo sapiens
  source 1..24: mol_type = transcribed RNA
tgaggggcctcagaccgagctttt

SEQ ID NO: 90 - RNA, 17 nt, Homo sapiens
  source 1..17: mol_type = transcribed RNA
accccactcctggtacc

SEQ ID NO: 91 - RNA, 20 nt, Homo sapiens
  source 1..20: mol_type = transcribed RNA
gccggggctttgggtgaggg

SEQ ID NO: 92 - RNA, 23 nt, Homo sapiens
  source 1..23: mol_type = transcribed RNA
cacacaggaaaagcggggccctg

SEQ ID NO: 93 - RNA, 22 nt, Homo sapiens
  source 1..22: mol_type = transcribed RNA
tgggagggcgtggatgatggtg

SEQ ID NO: 94 - RNA, 25 nt, Homo sapiens
  source 1..25: mol_type = transcribed RNA
agcggggaggaagtgggcgctgctt

SEQ ID NO: 95 - RNA, 22 nt, Homo sapiens
  source 1..22: mol_type = transcribed RNA
gggacccagggagagacgtaag

SEQ ID NO: 96 - RNA, 18 nt, Homo sapiens
  source 1..18: mol_type = transcribed RNA
atcccacctctgccacca

SEQ ID NO: 97 - RNA, 22 nt, Homo sapiens
  source 1..22: mol_type = transcribed RNA
ccgggagaaggaggtggcctgg

SEQ ID NO: 98 - RNA, 24 nt, Homo sapiens
  source 1..24: mol_type = transcribed RNA
ggaggccggggtggggcggggcgg

SEQ ID NO: 99 - RNA, 23 nt, Homo sapiens
  source 1..23: mol_type = transcribed RNA
gtgcgtggtggctcgaggcgggg

SEQ ID NO: 100 - RNA, 22 nt, Homo sapiens
  source 1..22: mol_type = transcribed RNA
actggggagcagaaggagaacc

SEQ ID NO: 101 - RNA, 23 nt, Homo sapiens
  source 1..23: mol_type = transcribed RNA
ggatggttgggggcggtcggcgt

SEQ ID NO: 102 - RNA, 23 nt, Homo sapiens
  source 1..23: mol_type = transcribed RNA
agggccagaggagcctggagtgg

SEQ ID NO: 103 - RNA, 20 nt, Homo sapiens
  source 1..20: mol_type = transcribed RNA
gggaaaaggaagggggagga

SEQ ID NO: 104 - RNA, 17 nt, Homo sapiens
  source 1..17: mol_type = transcribed RNA
ccccggggagcccggcg

SEQ ID NO: 105 - RNA, 17 nt, Homo sapiens
  source 1..17: mol_type = transcribed RNA
gctgggcgaggctggca

SEQ ID NO: 106 - RNA, 23 nt, Homo sapiens
  source 1..23: mol_type = transcribed RNA
tgggaggggagaggcagcaagca

SEQ ID NO: 107 - RNA, 17 nt, Homo sapiens
  source 1..17: mol_type = transcribed RNA
gcggggctgggcgcgcg

SEQ ID NO: 108 - RNA, 23 nt, Homo sapiens
  source 1..23: mol_type = transcribed RNA
cggggccgtagcactgtctgaga

SEQ ID NO: 109 - RNA, 22 nt, Homo sapiens
  source 1..22: mol_type = transcribed RNA
agactgacggctggaggcccat

SEQ ID NO: 110 - RNA, 24 nt, Homo sapiens
  source 1..24: mol_type = transcribed RNA
tggggggacaggatgagaggctgt

SEQ ID NO: 111 - RNA, 22 nt, Homo sapiens
  source 1..22: mol_type = transcribed RNA
ttgaggagacatggtgggggcc

SEQ ID NO: 112 - RNA, 19 nt, Homo sapiens
  source 1..19: mol_type = transcribed RNA
ctggggagtggctggggag

SEQ ID NO: 113 - RNA, 20 nt, Homo sapiens
  source 1..20: mol_type = transcribed RNA
ggcggcggggaggtaggcag

SEQ ID NO: 114 - RNA, 25 nt, Homo sapiens
  source 1..25: mol_type = transcribed RNA
gtgaggcggggccaggagggtgtgt

SEQ ID NO: 115 - RNA, 18 nt, Homo sapiens
  source 1..18: mol_type = transcribed RNA
gaggctgaaggaagatgg

SEQ ID NO: 116 - RNA, 19 nt, Homo sapiens
  source 1..19: mol_type = transcribed RNA
ttgatctcggaagctaagc

SEQ ID NO: 117 - RNA, 22 nt, Homo sapiens
  source 1..22: mol_type = transcribed RNA
ggaggaaccttggagcttcggc

SEQ ID NO: 118 - RNA, 17 nt, Homo sapiens
  source 1..17: mol_type = transcribed RNA
gtggggccaggcggtgg

SEQ ID NO: 119 - RNA, 17 nt, Homo sapiens
  source 1..17: mol_type = transcribed RNA
ggggctgggcgcgcgcc

SEQ ID NO: 120 - RNA, 22 nt, Homo sapiens
  source 1..22: mol_type = transcribed RNA
gagggttgggtggaggctctcc

SEQ ID NO: 121 - RNA, 21 nt, Homo sapiens
  source 1..21: mol_type = transcribed RNA
aggtgggtatggaggagccct

SEQ ID NO: 122 - RNA, 23 nt, Homo sapiens
  source 1..23: mol_type = transcribed RNA
tcggggagtctggggtccggaat

SEQ ID NO: 123 - RNA, 23 nt, Homo sapiens
  source 1..23: mol_type = transcribed RNA
gagggcagcgtgggtgtggcgga

SEQ ID NO: 124 - RNA, 18 nt, Homo sapiens
  source 1..18: mol_type = transcribed RNA
aggctgggctgggacgga

SEQ ID NO: 125 - RNA, 22 nt, Homo sapiens
  source 1..22: mol_type = transcribed RNA
cagaaggggagttgggagcaga

SEQ ID NO: 126 - RNA, 21 nt, Homo sapiens
  source 1..21: mol_type = transcribed RNA
agccgcggggatcgccgaggg

SEQ ID NO: 127 - RNA, 17 nt, Homo sapiens
  source 1..17: mol_type = transcribed RNA
gccggacaagagggagg

SEQ ID NO: 128 - RNA, 19 nt, Homo sapiens
  source 1..19: mol_type = transcribed RNA
gagggcgggtggaggagga

SEQ ID NO: 129 - RNA, 23 nt, Homo sapiens
  source 1..23: mol_type = transcribed RNA
cctccctgcccgcctctctgcag

SEQ ID NO: 130 - RNA, 22 nt, Homo sapiens
  source 1..22: mol_type = transcribed RNA
ttggggtggagggccaaggagc

SEQ ID NO: 131 - RNA, 17 nt, Homo sapiens
  source 1..17: mol_type = transcribed RNA
cgcgccgggcccgggtt

SEQ ID NO: 132 - RNA, 20 nt, Homo sapiens
  source 1..20: mol_type = transcribed RNA
cctgagcccgggccgcgcag

SEQ ID NO: 133 - RNA, 23 nt, Homo sapiens
  source 1..23: mol_type = transcribed RNA
cgaggggtagaagagcacagggg

SEQ ID NO: 134 - RNA, 23 nt, Homo sapiens
  source 1..23: mol_type = transcribed RNA
tgggctgagggcaggaggcctgt

SEQ ID NO: 135 - RNA, 19 nt, Homo sapiens
  source 1..19: mol_type = transcribed RNA
tgcggcagagctggggtca

SEQ ID NO: 136 - RNA, 22 nt, Homo sapiens
  source 1..22: mol_type = transcribed RNA
gggggtccccggtgctcggatc

SEQ ID NO: 137 - RNA, 21 nt, Homo sapiens
  source 1..21: mol_type = transcribed RNA
cggggcagctcagtacaggat

SEQ ID NO: 138 - RNA, 22 nt, Homo sapiens
  source 1..22: mol_type = transcribed RNA
tgagcccctgtgccgcccccag

SEQ ID NO: 139 - RNA, 20 nt, Homo sapiens
  source 1..20: mol_type = transcribed RNA
cggctctgggtctgtgggga

SEQ ID NO: 140 - RNA, 22 nt, Homo sapiens
  source 1..22: mol_type = transcribed RNA
ggctacaacacaggacccgggc

SEQ ID NO: 141 - RNA, 21 nt, Homo sapiens
  source 1..21: mol_type = transcribed RNA
cccggagccaggatgcagctc

SEQ ID NO: 142 - RNA, 21 nt, Homo sapiens
  source 1..21: mol_type = transcribed RNA
tgggggtgtggggagagagag

SEQ ID NO: 143 - RNA, 20 nt, Homo sapiens
  source 1..20: mol_type = transcribed RNA
actcaaactgtgggggcact

SEQ ID NO: 144 - RNA, 24 nt, Homo sapiens
  source 1..24: mol_type = transcribed RNA
tggggagctgaggctctgggggtg

SEQ ID NO: 145 - RNA, 19 nt, Homo sapiens
  source 1..19: mol_type = transcribed RNA
gagccagttggacaggagc

SEQ ID NO: 146 - RNA, 22 nt, Homo sapiens
  source 1..22: mol_type = transcribed RNA
agggacgggacgcggtgcagtg

SEQ ID NO: 147 - RNA, 22 nt, Homo sapiens
  source 1..22: mol_type = transcribed RNA
gtgaacgggcgccatcccgagg

SEQ ID NO: 148 - RNA, 20 nt, Homo sapiens
  source 1..20: mol_type = transcribed RNA
ggggagctgtggaagcagta

SEQ ID NO: 149 - RNA, 22 nt, Homo sapiens
  source 1..22: mol_type = transcribed RNA
accttgccttgctgcccgggcc

SEQ ID NO: 150 - RNA, 20 nt, Homo sapiens
  source 1..20: mol_type = transcribed RNA
gtgtctgggcggacagctgc

SEQ ID NO: 151 - RNA, 22 nt, Homo sapiens
  source 1..22: mol_type = transcribed RNA
ggtggcccggccgtgcctgagg

SEQ ID NO: 152 - RNA, 22 nt, Homo sapiens
  source 1..22: mol_type = transcribed RNA
gtgggtacggcccagtgggggg

SEQ ID NO: 153 - RNA, 24 nt, Homo sapiens
  source 1..24: mol_type = transcribed RNA
aggcaggggctggtgctgggcggg

SEQ ID NO: 154 - RNA, 17 nt, Homo sapiens
  source 1..17: mol_type = transcribed RNA
ggcgggtgcgggggtgg

SEQ ID NO: 155 - RNA, 18 nt, Homo sapiens
  source 1..18: mol_type = transcribed RNA
agggggcgggctccggcg

SEQ ID NO: 156 - RNA, 22 nt, Homo sapiens
  source 1..22: mol_type = transcribed RNA
acaggtgaggttcttgggagcc

SEQ ID NO: 157 - RNA, 22 nt, Homo sapiens
  source 1..22: mol_type = transcribed RNA
ctcggcgcggggcgcgggctcc

SEQ ID NO: 158 - RNA, 21 nt, Homo sapiens
  source 1..21: mol_type = transcribed RNA
gtgggcgggggcaggtgtgtg

SEQ ID NO: 159 - RNA, 23 nt, Homo sapiens
  source 1..23: mol_type = transcribed RNA
ccagggggatgggcgagcttggg

SEQ ID NO: 160 - RNA, 20 nt, Homo sapiens
  source 1..20: mol_type = transcribed RNA
cgggcgtggtggtgggggtg

SEQ ID NO: 161 - RNA, 20 nt, Homo sapiens
  source 1..20: mol_type = transcribed RNA
tagggggtggcaggctggcc

SEQ ID NO: 162 - RNA, 20 nt, Homo sapiens
  source 1..20: mol_type = transcribed RNA
ccccagggcgacgcggcggg

SEQ ID NO: 163 - RNA, 21 nt, Homo sapiens
  source 1..21: mol_type = transcribed RNA
caggagtggggggtgggacgt

SEQ ID NO: 164 - RNA, 21 nt, Homo sapiens
  source 1..21: mol_type = transcribed RNA
tggcagggaggctgggagggg

SEQ ID NO: 165 - RNA, 21 nt, Homo sapiens
  source 1..21: mol_type = transcribed RNA
acaggagtgggggtgggacat

SEQ ID NO: 166 - RNA, 22 nt, Homo sapiens
  source 1..22: mol_type = transcribed RNA
cagggcagggaaggtgggagag

SEQ ID NO: 167 - RNA, 18 nt, Homo sapiens
  source 1..18: mol_type = transcribed RNA
ggtgggcttcccggaggg

SEQ ID NO: 168 - RNA, 22 nt, Homo sapiens
  source 1..22: mol_type = transcribed RNA
ctgggagagggttgtttactcc

SEQ ID NO: 169 - RNA, 21 nt, Homo sapiens
  source 1..21: mol_type = transcribed RNA
actcggctgcggtggacaagt

SEQ ID NO: 170 - RNA, 20 nt, Homo sapiens
  source 1..20: mol_type = transcribed RNA
agagatgaagcgggggggcg

SEQ ID NO: 171 - RNA, 20 nt, Homo sapiens
  source 1..20: mol_type = transcribed RNA
tcagggagtcaggggagggc

SEQ ID NO: 172 - RNA, 25 nt, Homo sapiens
  source 1..25: mol_type = transcribed RNA
taggggtgggggaattcaggggtgt

SEQ ID NO: 173 - RNA, 20 nt, Homo sapiens
  source 1..20: mol_type = transcribed RNA
accaggaggctgaggcccct

SEQ ID NO: 174 - RNA, 22 nt, Homo sapiens
  source 1..22: mol_type = transcribed RNA
tcctgtactgagctgccccgag

SEQ ID NO: 175 - RNA, 22 nt, Homo sapiens
  source 1..22: mol_type = transcribed RNA
caccggggatggcagagggtcg

SEQ ID NO: 176 - RNA, 17 nt, Homo sapiens
  source 1..17: mol_type = transcribed RNA
gtgggggagaggctgtc

SEQ ID NO: 177 - RNA, 24 nt, Homo sapiens
  source 1..24: mol_type = transcribed RNA
tgtaggcatgaggcagggcccagg

SEQ ID NO: 178 - RNA, 23 nt, Homo sapiens
  source 1..23: mol_type = transcribed RNA
gaacgcctgttcttgccaggtgg

SEQ ID NO: 179 - RNA, 23 nt, Homo sapiens
  source 1..23: mol_type = transcribed RNA
acaggcggctgtagcaatggggg

SEQ ID NO: 180 - RNA, 22 nt, Homo sapiens
  source 1..22: mol_type = transcribed RNA
ggggggtgtggagccagggggc

SEQ ID NO: 181 - RNA, 22 nt, Homo sapiens
  source 1..22: mol_type = transcribed RNA
ctcgggagggcatgggccaggc

SEQ ID NO: 182 - RNA, 22 nt, Homo sapiens
  source 1..22: mol_type = transcribed RNA
tggggatttggagaagtggtga

SEQ ID NO: 183 - RNA, 21 nt, Homo sapiens
  source 1..21: mol_type = transcribed RNA
gcagggacagcaaaggggtgc

SEQ ID NO: 184 - RNA, 22 nt, Homo sapiens
  source 1..22: mol_type = transcribed RNA
aggcggggcgccgcgggaccgc

SEQ ID NO: 185 - RNA, 22 nt, Homo sapiens
  source 1..22: mol_type = transcribed RNA
tgggggtggtctctagccaagg

SEQ ID NO: 186 - RNA, 21 nt, Homo sapiens
  source 1..21: mol_type = transcribed RNA
tctgtggagtggggtgcctgt

SEQ ID NO: 187 - RNA, 21 nt, Homo sapiens
  source 1..21: mol_type = transcribed RNA
ctgggaggggctgggtttggc

SEQ ID NO: 188 - RNA, 53 nt, Homo sapiens
  source 1..53: mol_type = transcribed RNA
ggtgggggttggaggcgtgggttttagaacctatccctttctagccctgagca

SEQ ID NO: 189 - RNA, 80 nt, Homo sapiens
  source 1..80: mol_type = transcribed RNA
cgggaatgccgcggcggggacggcgattggtccgtatgtgtggtgccaccggccgccggctccgccccggcccccgcccc

SEQ ID NO: 190 - RNA, 86 nt, Homo sapiens
  source 1..86: mol_type = transcribed RNA
ggcttagaaacagtccctaggtaggatttggggaggagctaagaagcccctacagggcccagaggtggggactgagccttagttgg

SEQ ID NO: 191 - RNA, 73 nt, Homo sapiens
  source 1..73: mol_type = transcribed RNA
ggcgaggggaggcgcaggctcggaaaggcgcgcgaggctccaggctccttcccgatccaccgctctcctcgct

SEQ ID NO: 192 - RNA, 85 nt, Homo sapiens
  source 1..85: mol_type = transcribed RNA
ggcgcctcctgctctgctgtgccgccagggcctcccctagcgcgccttctggagaggctttgtgcggatacggggctggaggcct

SEQ ID NO: 193 - RNA, 64 nt, Homo sapiens
  source 1..64: mol_type = transcribed RNA
tctgggcgaggggtgggctctcagaggggctggcagtactgctctgaggcctgcctctccccag

SEQ ID NO: 194 - RNA, 84 nt, Homo sapiens
  source 1..84: mol_type = transcribed RNA
gctggcgtcggtgctggggagcggcccccgggtgggcctctgctctggcccctcctggggcccgcactctcgctctgggcccgc

SEQ ID NO: 195 - RNA, 64 nt, Homo sapiens
  source 1..64: mol_type = transcribed RNA
gaggcactgggtaggtggggctccagggctcctgacacctggacctctcctccccaggcccaca

SEQ ID NO: 196 - RNA, 77 nt, Homo sapiens
  source 1..77: mol_type = transcribed RNA
gagttgggaggttccctctccaaatgtgtcttgatcccccaccccaagacacatttggagagggaccctcccaactc

SEQ ID NO: 197 - RNA, 63 nt, Homo sapiens
  source 1..63: mol_type = transcribed RNA
aatgggtgggtgctggtgggagccgtgccctggccactcattcggctctctccctcaccctag

SEQ ID NO: 198 - RNA, 62 nt, Homo sapiens
  source 1..62: mol_type = transcribed RNA
gtgagtggggctcccgggacggcgcccgccctggccctggcccggcgacgtctcacggtccc

SEQ ID NO: 199 - RNA, 98 nt, Homo sapiens
  source 1..98: mol_type = transcribed RNA
cttggtcaataggaaagaggtgggacctcctggcttttcctctgcagcatggctcggacctagtgcaatgtttaagctcccctctctttcctgttcag

SEQ ID NO: 200 - RNA, 97 nt, Homo sapiens
  source 1..97: mol_type = transcribed RNA
cccgggaccttggtccaggcgctggtctgcgtggtgctcgggtggataagtctgatctgagcaccacacaggccgggcgccgggaccaagggggctc

SEQ ID NO: 201 - RNA, 63 nt, Homo sapiens
  source 1..63: mol_type = transcribed RNA
gagtcgaggactggtggaagggcctttcccctcagaccaaggccctggccccagcttcttctc

SEQ ID NO: 202 - RNA, 75 nt, Homo sapiens
  source 1..75: mol_type = transcribed RNA
tcatccctgggtggggatttgttgcattacttgtgttctatataaagtattgcacttgtcccggcctgtggaaga

SEQ ID NO: 203 - RNA, 91 nt, Homo sapiens
  source 1..91: mol_type = transcribed RNA
acgccccccgccccgccaccgccttggaggctgacctcttactttcggtcggtcttcttccctgggcttggtttgggggcgggggagtgtc

SEQ ID NO: 204 - RNA, 86 nt, Homo sapiens
  source 1..86: mol_type = transcribed RNA
aggagtgaccaaaagacaagagtgcgagccttctattatgcccagacagggccaccagagggctccttggtctaggggtaatgcca

SEQ ID NO: 205 - RNA, 96 nt, Homo sapiens
  source 1..96: mol_type = transcribed RNA
gctctggggcgtgccgccgccgtcgctgccacctcccctaccgctagtggaagaagatggcggaaggcggagcggcggatctggacacccagcggt

SEQ ID NO: 206 - RNA, 62 nt, Homo sapiens
  source 1..62: mol_type = transcribed RNA
gagggtggtggaggaagagggcagctcccatgactgcctgaccgccttctctcctcccccag

SEQ ID NO: 207 - RNA, 109 nt, Homo sapiens
  source 1..109: mol_type = transcribed RNA
tgctattgtcttactgctacagcagggctggggattgcagtatccgctgttgctgctgctcccagtcctgcccctgctgctacctagtccagcctcaccgcatcccaga

SEQ ID NO: 208 - RNA, 63 nt, Homo sapiens
  source 1..63: mol_type = transcribed RNA
tggtggcggcggtagttatgggcttctctttctcaccagcagcccctgggccgccgcctccct

SEQ ID NO: 209 - RNA, 69 nt, Homo sapiens
  source 1..69: mol_type = transcribed RNA
ggccctcgggcctggggttgggggagctctgtcctgtctcactcattgctcctcccctgcctggcccag

SEQ ID NO: 210 - RNA, 84 nt, Homo sapiens
  source 1..84: mol_type = transcribed RNA
atggtccctcccaatccagccattcctcagaccaggtggctcccgagccaccccaggctgtaggatgggggtgagaggtgctag

SEQ ID NO: 211 - RNA, 100 nt, Homo sapiens
  source 1..100: mol_type = transcribed RNA
gaggtgggaggattgcttgagtcagggtggttgaggctgcagtaagttgtgatcataccactgcactccagcctgagtgacagagcaagaccttgtctca

SEQ ID NO: 212 - RNA, 71 nt, Homo sapiens
  source 1..71: mol_type = transcribed RNA
gtgtctgtgccggtcccaggagaacctgcagaggcatcgggtcagcggtgctcctgcgggccgacactcac

SEQ ID NO: 213 - RNA, 80 nt, Homo sapiens
  source 1..80: mol_type = transcribed RNA
cgtgtgagcccgccctgtgcccggcccacttctgcttcctcttagcgcaggaggggtcccgcactgggaggggccctcac

SEQ ID NO: 214 - RNA, 99 nt, Homo sapiens
  source 1..99: mol_type = transcribed RNA
gcaagggacagggagggtcgtggcgacactcgcgccagctcccgggacggctgggctcgggctggtcgccgacctccgaccctccactagatgcctggc

SEQ ID NO: 215 - RNA, 61 nt, Homo sapiens
  source 1..61: mol_type = transcribed RNA
gggggcgggagctggggtctgcaggttcgcactgatgcctgctcgccctgtctcccgctag

SEQ ID NO: 216 - RNA, 84 nt, Homo sapiens
  source 1..84: mol_type = transcribed RNA
tgtgcagtgggaaggggggccgatacactgtacgagagtgagtagcaggtctcacagtgaaccggtctctttccctactgtgtc

SEQ ID NO: 217 - RNA, 73 nt, Homo sapiens
  source 1..73: mol_type = transcribed RNA
cggcgacggcggggtgggtgaggtcgggccccaagactcggggtttgccgggcgcctcagttcaccgcggccg

SEQ ID NO: 218 - RNA, 87 nt, Homo sapiens
  source 1..87: mol_type = transcribed RNA
gtgaggcggggccaggagggtgtgtggcgtgggtgctgcggggccgtcagggtgcctgcgggacgctcacctggctggcccgcccag

SEQ ID NO: 219 - RNA, 68 nt, Homo sapiens
  source 1..68: mol_type = transcribed RNA
gaatggaagaagaaggcggtcggtctgcgggagccaggccgcagagccatccgccttctgtccatgtc

SEQ ID NO: 220 - RNA, 74 nt, Homo sapiens
  source 1..74: mol_type = transcribed RNA
gcagcccggtgagcgctcgctggcctggcagtgcgtcggaagaacagggcgggtggggccgcgcacatctctgc

SEQ ID NO: 221 - RNA, 65 nt, Homo sapiens
  source 1..65: mol_type = transcribed RNA
gagaatggggggacagatggagaggacacaggctggcactgaggtcccctccactttcctcctag

SEQ ID NO: 222 - RNA, 137 nt, Homo sapiens
  source 1..137: mol_type = transcribed RNA
agccagacaagagggtcatggggagtcactgtcaacccagagcaggcactgcccctgcgaccagcctggggcatcggttggggtgcaggggtctgctggtgatgctttccatctctttgctttgtcctgattgtagc

SEQ ID NO: 223 - RNA, 83 nt, Homo sapiens
  source 1..83: mol_type = transcribed RNA
acggcatctttgcactcagcaggcaggctggtgcagcccgtggtgggggaccatcctgcctgctgtggggtaaggacggctgt

SEQ ID NO: 224 - RNA, 79 nt, Homo sapiens
  source 1..79: mol_type = transcribed RNA
cagcctggggaaggcttggcagggaagacacatgagcagtgcctccacttcacgcctctcccttgtctcctttccctag

SEQ ID NO: 225 - RNA, 80 nt, Homo sapiens
  source 1..80: mol_type = transcribed RNA
ggttccggagccccggcgcgggcgggttctggggtgtagacgctgctggccagcccgccccagccgaggttctcggcacc

SEQ ID NO: 226 - RNA, 81 nt, Homo sapiens
  source 1..81: mol_type = transcribed RNA
tagaggcagtttcaacagatgtgtagacttttgatatgagaaattggtttcaaaatcaggagtcggggctttactgctttt

SEQ ID NO: 227 - RNA, 55 nt, Homo sapiens
  source 1..55: mol_type = transcribed RNA
ccggatccgagtcacggcaccaaatttcatgcgtgtccgtgtgaagagaccacca

SEQ ID NO: 228 - RNA, 75 nt, Homo sapiens
  source 1..75: mol_type = transcribed RNA
gtcagaggggggatgtgcatgctggttggggtgggctgcctgtggaccaatcagcgtgcacttccccaccctgaa

SEQ ID NO: 229 - RNA, 68 nt, Homo sapiens
  source 1..68: mol_type = transcribed RNA
gtgcaaagagcaggaggacaggggatttatctcccaagggaggtcccctgatcctagtcacggcacca

SEQ ID NO: 230 - RNA, 98 nt, Homo sapiens
  source 1..98: mol_type = transcribed RNA
ttgggcaaggtgcggggctagggctaacagcagtcttactgaaggtttcctggaaaccacgcacatgctgttgccactaacctcaaccttactcggtc

SEQ ID NO: 231 - RNA, 68 nt, Homo sapiens
  source 1..68: mol_type = transcribed RNA
tgcccaggctggagcgagtgcagtggtgcagtcagtcctagctcactgcagcctcgaactcctgggct

SEQ ID NO: 232 - RNA, 72 nt, Homo sapiens
  source 1..72: mol_type = transcribed RNA
tgtgggactgcaaatgggagctcagcacctgcctgccacccacgcagaccagcccctgctctgttcccacag

SEQ ID NO: 233 - RNA, 66 nt, Homo sapiens
  source 1..66: mol_type = transcribed RNA
ccgagtggggcggggcaggtccctgcagggactgtgacactgaaggacctgcaccttcgcccacag

SEQ ID NO: 234 - RNA, 90 nt, Homo sapiens
  source 1..90: mol_type = transcribed RNA
cgggcggggcgggtccggccgcctccgagcccggccggcagcccccggccttaaagcgcgggctgtccggaggggtcggctttcccaccg

SEQ ID NO: 235 - RNA, 94 nt, Homo sapiens
  source 1..94: mol_type = transcribed RNA
cacggtgtcccctggtggaacctggcagggggagaggtaaggtctttcagcctctccaaagcccatggtcaggtactcaggtgggggagccctg

SEQ ID NO: 236 - RNA, 67 nt, Homo sapiens
  source 1..67: mol_type = transcribed RNA
ccagacccctggggctgggcaggcggaaagaggtctgaactgcctctgcctccttggtctccggcag

SEQ ID NO: 237 - RNA, 136 nt, Homo sapiens
  source 1..136: mol_type = transcribed RNA
ccgcttgcctcgcccagcgcagccccggccgctgggcgcacccgtcccgttcgtccccggacgttgctctctaccccgggaacgtcgagactggagcgcccgaactgagccaccttcgcggaccccgagagcggcg

SEQ ID NO: 238 - RNA, 49 nt, Homo sapiens
  source 1..49: mol_type = transcribed RNA
ttcccagccaacgcaccaaaaatgatatgggtctgttgtctggagaaac

SEQ ID NO: 239 - RNA, 89 nt, Homo sapiens
  source 1..89: mol_type = transcribed RNA
acctccgggacggctgggcgccggcggccgggagatccgcgcttcctgaatcccggccggcccgcccggcgcccgtccgcccgcgggtc

SEQ ID NO: 240 - RNA, 60 nt, Homo sapiens
  source 1..60: mol_type = transcribed RNA
cgctgggtccgcgcgccctgggccgggcgatgtccgcttgggggagcgaggggcggggcg

SEQ ID NO: 241 - RNA, 64 nt, Homo sapiens
  source 1..64: mol_type = transcribed RNA
aaccccgggccggaggtcaagggcgtcgcttctccctaatgttgcctcttttccacggcctcag

SEQ ID NO: 242 - RNA, 60 nt, Homo sapiens
  source 1..60: mol_type = transcribed RNA
caaggtgggggagatgggggttgaacttcatttctcatgctcatccccatctcctttcag

SEQ ID NO: 243 - RNA, 65 nt, Homo sapiens
  source 1..65: mol_type = transcribed RNA
gagggtgggcgagggcggctgagcggctccatcccccggcctgctcatccccctcgccctctcag

SEQ ID NO: 244 - RNA, 56 nt, Homo sapiens
  source 1..56: mol_type = transcribed RNA
cgaccgcacccgcccgaagctgggtcaaggagcccagcaggacgggagcgcggcgc

SEQ ID NO: 245 - RNA, 103 nt, Homo sapiens
  source 1..103: mol_type = transcribed RNA
gtgtcggctgtggcgtgactgtccctctgtgtcccccactaggcccactgctcagtggagcgtggaggacgaggaggaggccgtccacgagcaatgccagcat

SEQ ID NO: 246 - RNA, 66 nt, Homo sapiens
  source 1..66: mol_type = transcribed RNA
gggcatggggaggtgtggagtcagcatggggctaggaggccccgcgctgacccgccttctccgcag

SEQ ID NO: 247 - RNA, 87 nt, Homo sapiens
  source 1..87: mol_type = transcribed RNA
cccaggcgcccgctcccgacccacgccgcgccgccgggtccctcctccccggagaggctgggctcgggacgcgcggctcagctcggg

SEQ ID NO: 248 - RNA, 93 nt, Homo sapiens
  source 1..93: mol_type = transcribed RNA
ggcgcttttgtgcgcgcccgggtctgttggtgctcagagtgtggtcaggcggctcggactgagcaggtgggtgcggggctcggaggaggcggc

SEQ ID NO: 249 - RNA, 66 nt, Homo sapiens
  source 1..66: mol_type = transcribed RNA
cctggaggggggcactgcgcaagcaaagccagggaccctgagaggctttgcttcctgctcccctag

SEQ ID NO: 250 - RNA, 78 nt, Homo sapiens
  source 1..78: mol_type = transcribed RNA
ggcctcaggcaggcgcacccgaccacatgcatggctggtggcggcgtgcaggggtcgggtgggccaggctgtggggcg

SEQ ID NO: 251 - RNA, 86 nt, Homo sapiens
  source 1..86: mol_type = transcribed RNA
agggagaagggtcggggcagggagggcagggcaggctctggggtggggggtctgtgagtcagccacggctctgcccacgtctcccc

SEQ ID NO: 252 - RNA, 69 nt, Homo sapiens
  source 1..69: mol_type = transcribed RNA
gggcttagggatgggaggccaggatgaagattaatccctaatccccaacactggccttgctatccccag

SEQ ID NO: 253 - RNA, 71 nt, Homo sapiens
  source 1..71: mol_type = transcribed RNA
tgaccacccccgggcaaagacctgcagatcccctgttagagacgggcccaggactttgtgcggggtgccca

SEQ ID NO: 254 - RNA, 115 nt, Homo sapiens
  source 1..115: mol_type = transcribed RNA
gagcaaaaaccagagaacaacatgggagcgttcctaacccctaaggcaactggatgggagacctgacccatccagttctctgagggggctcttgtgtgttctacaaggttgttca

SEQ ID NO: 255 - RNA, 63 nt, Homo sapiens
  source 1..63: mol_type = transcribed RNA
accctagggtggggctggaggtggggctgaggctgagtcttcctccccttcctccctgcccag

SEQ ID NO: 256 - RNA, 82 nt, Homo sapiens
  source 1..82: mol_type = transcribed RNA
acctgtaggtgacagtcaggggcggggtgtggtggggctggggctggccccctcctcacacctctcctggcatcgcccccag

SEQ ID NO: 257 - RNA, 65 nt, Homo sapiens
  source 1..65: mol_type = transcribed RNA
gggtgctcggggcaggcggctgggagcggccctcacattgatggctcctgccacctcctccgcag

SEQ ID NO: 258 - RNA, 89 nt, Homo sapiens
  source 1..89: mol_type = transcribed RNA
agcctgtgggaaagagaagagcagggcagggtgaaggcccggcggagacactctgcccaccccacaccctgcctatgggccacacagct

SEQ ID NO: 259 - RNA, 62 nt, Homo sapiens
  source 1..62: mol_type = transcribed RNA
gtgggtctcgcatcaggaggcaaggccaggacccgctgacccatgcctcctgccgcggtcag

SEQ ID NO: 260 - RNA, 67 nt, Homo sapiens
  source 1..67: mol_type = transcribed RNA
ctggtccatttccctgccattcccttggcttcaatttactcccagggctggcagtgacatgggtcaa

SEQ ID NO: 261 - RNA, 52 nt, Homo sapiens
  source 1..52: mol_type = transcribed RNA
tagccgggcgtggtggtgggggcctgtggtcccagctactttggaggctgag

SEQ ID NO: 262 - RNA, 68 nt, Homo sapiens
  source 1..68: mol_type = transcribed RNA
cgtggtgaggatatggcagggaaggggagtttccctctattcccttccccccagtaatcttcatcatg

SEQ ID NO: 263 - RNA, 60 nt, Homo sapiens
  source 1..60: mol_type = transcribed RNA
tgtgaatgacccccttccagagccaaaatcaccagggatggaggaggggtcttgggtact

SEQ ID NO: 264 - RNA, 77 nt, Homo sapiens
  source 1..77: mol_type = transcribed RNA
catgagaaatcctgctggtcaaccatagccctggtcagactctccggggctgtgattgaccagcaggacttctcatg

SEQ ID NO: 265 - RNA, 87 nt, Homo sapiens
  source 1..87: mol_type = transcribed RNA
gtgtggccggcaggcgggtgggcgggggcggccggtgggaaccccgccccgccccgcgcccgcactcacccgcccgtctccccacag

SEQ ID NO: 266 - RNA, 69 nt, Homo sapiens
  source 1..69: mol_type = transcribed RNA
gaggaggggaggtgtgcagggctggggtcactgactctgcttcccctgccctgcatggtgtccccacag

SEQ ID NO: 267 - RNA, 74 nt, Homo sapiens
  source 1..74: mol_type = transcribed RNA
cctgcaggaggcagtgggcgagcaggcggggcagcccaatgccatgggcctgatctcaccgctgcctccttccc

SEQ ID NO: 268 - RNA, 84 nt, Homo sapiens
  source 1..84: mol_type = transcribed RNA
gaggctgggcggggcgcggccggatcggtcgagagcgtcctggctgatgacggtctcccgtgcccacgccccaaacgcagtctc

SEQ ID NO: 269 - RNA, 85 nt, Homo sapiens
  source 1..85: mol_type = transcribed RNA
tctgaggtacccggggcagattggtgtagggtgcaaagcctgcccgccccctaagccttctgcccccaactccagcctgtcagga

SEQ ID NO: 270 - RNA, 153 nt, Homo sapiens
  source 1..153: mol_type = transcribed RNA
gctccgccccacgtcgcatgcgccccgggaacgcgtggggcggagcttccggaggccccgctctgctgccgaccctgtggagcggagggtgaagcctccggatgccagtccctcatcgctggcctggtcgcgctgtggcgaagggggcggagc

SEQ ID NO: 271 - RNA, 153 nt, Homo sapiens
  source 1..153: mol_type = transcribed RNA
gctccgccccacgtcgcatgcgccccgggaacgcgtggggcggagcttccggaggccccgccctgctgccgaccctgtggagcggagggtgaagcctccggatgccagtccctcatcgctggcccggtcgcgctgtggcgaagggggcggagc

SEQ ID NO: 272 - RNA, 102 nt, Homo sapiens
  source 1..102: mol_type = transcribed RNA
gtgggagggcccaggcgcgggcaggggtgggggtggcagagcgctgtcccgggggcggggccgaagcgcggcgaccgtaactccttctgctccgtcccccag

SEQ ID NO: 273 - RNA, 71 nt, Homo sapiens
  source 1..71: mol_type = transcribed RNA
ggtgagtgggagccggtggggctggagtaagggcacgcccggggctgccccacctgctgaccaccctcccc

SEQ ID NO: 274 - RNA, 75 nt, Homo sapiens
  source 1..75: mol_type = transcribed RNA
aagcaagactgaggggcctcagaccgagcttttggaaaatagaaaagtctcgctctctgcccctcagcctaactt

SEQ ID NO: 275 - RNA, 93 nt, Homo sapiens
  source 1..93: mol_type = transcribed RNA
tacttatggcaccccactcctggtaccatagtcataagttaggagatgttagagctgtgagtaccatgacttaagtgtggtggcttaaacatg

SEQ ID NO: 276 - RNA, 67 nt, Homo sapiens
  source 1..67: mol_type = transcribed RNA
tacaggccggggctttgggtgagggacccccggagtctgtcacggtctcaccccaactctgccccag

SEQ ID NO: 277 - RNA, 72 nt, Homo sapiens
  source 1..72: mol_type = transcribed RNA
ccaggcacacaggaaaagcggggccctgggttcggctgctaccccaaaggccacattctcctgtgcacacag

SEQ ID NO: 278 - RNA, 81 nt, Homo sapiens
  source 1..81: mol_type = transcribed RNA
ctccctgggagggcgtggatgatggtgggagaggagccccactgtggaagtctgacccccacatcgccccaccttccccag

SEQ ID NO: 279 - RNA, 82 nt, Homo sapiens
  source 1..82: mol_type = transcribed RNA
gctacggggagcggggaggaagtgggcgctgcttctgcgttatctggaaggagcagcccactcctgtcctgggctctgtggt

SEQ ID NO: 280 - RNA, 76 nt, Homo sapiens
  source 1..76: mol_type = transcribed RNA
actgactttgagtctctcctcagggtgctgcaggcaaagctggggacccagggagagacgtaagtgaggggagatg

SEQ ID NO: 281 - RNA, 73 nt, Homo sapiens
  source 1..73: mol_type = transcribed RNA
acctttccagctcatcccacctctgccaccaaaacactcatcgcggggtcagagggagtgccaaaaaaggtaa

SEQ ID NO: 282 - RNA, 63 nt, Homo sapiens
  source 1..63: mol_type = transcribed RNA
cttgcccgggagaaggaggtggcctggagagctgctgtctccagccgccgcctgtctccacag

SEQ ID NO: 283 - RNA, 64 nt, Homo sapiens
  source 1..64: mol_type = transcribed RNA
ccccgggcccggcgttccctccccttccgtgcgccagtggaggccggggtggggcggggcgggg

SEQ ID NO: 284 - RNA, 64 nt, Homo sapiens; identical to SEQ ID NO: 283
  source 1..64: mol_type = transcribed RNA

SEQ ID NO: 285 - RNA, 74 nt, Homo sapiens
  source 1..74: mol_type = transcribed RNA
gtgcgtggtggctcgaggcgggggtgggggcctcgccctgcttgggccctccctgacctctccgctccgcacag

SEQ ID NO: 286 - RNA, 66 nt, Homo sapiens
  source 1..66: mol_type = transcribed RNA
tgactggggagcagaaggagaacccaagaaaagctgacttggaggtccctccttctgtccccacag

SEQ ID NO: 287 - RNA, 86 nt, Homo sapiens
  source 1..86: mol_type = transcribed RNA
cgcctgagcgtgcagcaggacatcttcctgacctggtaataattaggtgagaaggatggttgggggcggtcggcgtaactcaggga

SEQ ID NO: 288 - RNA, 58 nt, Homo sapiens
  source 1..58: mol_type = transcribed RNA
agggccagaggagcctggagtggtcgggtcgactgaacccaggttccctctggccgca

SEQ ID NO: 289 - RNA, 85 nt, Homo sapiens
  source 1..85: mol_type = transcribed RNA
ggggaggtagggaaaaggaagggggaggagaaggtgagaccaatgtcctgggtgccactcctgcccagtgcctcccttcctcgtt

SEQ ID NO: 290 - RNA, 51 nt, Homo sapiens
  source 1..51: mol_type = transcribed RNA
acagaccccggggagcccggcggtgaagctcctggtatcctgggtgtctga

SEQ ID NO: 291 - RNA, 63 nt, Homo sapiens
  source 1..63: mol_type = transcribed RNA
gcatgctgggcgaggctggcatctagcacaggcggtagatgcttgctcttgccattgcaatga

SEQ ID NO: 292 - RNA, 67 nt, Homo sapiens
  source 1..67: mol_type = transcribed RNA
gtgggaggggagaggcagcaagcacacagggcctgggactagcatgctgacctccctcctgccccag

SEQ ID NO: 293 - RNA, 70 nt, Homo sapiens
  source 1..70: mol_type = transcribed RNA
aggacccagcggggctgggcgcgcggagcagcgctgggtgcagcgcctgcgccggcagctgcaagggccg

SEQ ID NO: 294 - RNA, 82 nt, Homo sapiens
  source 1..82: mol_type = transcribed RNA
tgagctgttggattcggggccgtagcactgtctgagaggtttacatttctcacagtgaaccggtctctttttcagctgcttc

SEQ ID NO: 295 - RNA, 86 nt, Homo sapiens
  source 1..86: mol_type = transcribed RNA
attctaggtggggagactgacggctggaggcccataagctgtctaaaacttcggcccccagatttctggtctccccacttcagaac

SEQ ID NO: 296 - RNA, 68 nt, Homo sapiens
  source 1..68: mol_type = transcribed RNA
agggttggggggacaggatgagaggctgtcttcattccctcttgaccacccctcgtttcttcccccag

SEQ ID NO: 297 - RNA, 70 nt, Homo sapiens
  source 1..70: mol_type = transcribed RNA
ggtttctccttgaggagacatggtgggggccggtcaggcagcccatgccatgtgtcctcatggagaggcc

SEQ ID NO: 298 - RNA, 65 nt, Homo sapiens
  source 1..65: mol_type = transcribed RNA
ttctcctggggagtggctggggagcagacagacccaacctcatgctccccggcctctgcccccag

SEQ ID NO: 299 - RNA, 80 nt, Homo sapiens
  source 1..80: mol_type = transcribed RNA
gcgtcaagatggcggcggggaggtaggcagagcaggacgccgctgctgccgccgccaccgccgcctccgctccagtcgcc

SEQ ID NO: 300 - RNA, 68 nt, Homo sapiens
  source 1..68: mol_type = transcribed RNA
ctcaggctcagtggtgcatgcttatagtcccagccactctggaggctgaaggaagatggcttgagcct

SEQ ID NO: 301 - RNA, 61 nt, Homo sapiens
  source 1..61: mol_type = transcribed RNA
tctcgtttgatctcggaagctaagcagggttgggcctggttagtacttggatgggaaactt

SEQ ID NO: 302 - RNA, 53 nt, Homo sapiens
  source 1..53: mol_type = transcribed RNA
gtttgatctcggaagctaagcagggtcgggcctggttagtacttggatgggag

SEQ ID NO: 303 - RNA, 58 nt, Homo sapiens
  source 1..58: mol_type = transcribed RNA
gctgaagctctaaggttccgcctgcgggcaggaagcggaggaaccttggagcttcggc

SEQ ID NO: 304 - RNA, 88 nt, Homo sapiens
  source 1..88: mol_type = transcribed RNA
gtggggccaggcggtggtgggcactgctggggtgggcacagcagccatgcagagcgggcatttgaccccgtgccacccttttccccag

SEQ ID NO: 305 - RNA, 80 nt, Homo sapiens
  source 1..80: mol_type = transcribed RNA
ctgcagcgtgcttctccaggccccgcgcgcggacagacacacggacaagtcccgccaggggctgggcgcgcgccagccgg

SEQ ID NO: 306 - RNA, 80 nt, Homo sapiens
  source 1..80: mol_type = transcribed RNA
aggacccttccagagggccccccctcaatcctgttgtgcctaattcagagggttgggtggaggctctcctgaagggctct

SEQ ID NO: 307 - RNA, 73 nt, Homo sapiens
  source 1..73: mol_type = transcribed RNA
aggccaggtgggtatggaggagccctcatatggcagttggcgagggcccagtgagcccctctctgctctccag

SEQ ID NO: 308 - RNA, 59 nt, Homo sapiens
  source 1..59: mol_type = transcribed RNA
ctgtgtcggggagtctggggtccggaattctccagagcctctgtgcccctacttcccag

SEQ ID NO: 309 - RNA, 61 nt, Homo sapiens
  source 1..61: mol_type = transcribed RNA
gagggcagcgtgggtgtggcggaggcaggcgtgaccgtttgccgccctctcgctgctctag

SEQ ID NO: 310 - RNA, 73 nt, Homo sapiens
  source 1..73: mol_type = transcribed RNA
ggaggctgggctgggacggacacccggcctccactttctgtggcaggtacctcctccatgtcggcccgccttg

SEQ ID NO: 311 - RNA, 84 nt, Homo sapiens
  source 1..84: mol_type = transcribed RNA
ggcccctccttctcagccccagctcccgctcacccctgccacgtcaaaggaggcagaaggggagttgggagcagagaggggacc

SEQ ID NO: 312 - RNA, 180 nt, Homo sapiens
  source 1..180: mol_type = transcribed RNA
cgcgactgcggcggcggtggtggggggagccgcggggatcgccgagggccggtcggccgccccgggtgccgcgcggtgccgccggcggcggtgaggccccgcgcgtgtgtcccggctgcggtcggccgcgctcgaggggtccccgtggcgtccccttccccgccggccgcctttctcgcg

SEQ ID NO: 313 - RNA, 67 nt, Homo sapiens
  source 1..67: mol_type = transcribed RNA
gcgccctccctctctccccggtgtgcaaatgtgtgtgtgcggtgttatgccggacaagagggaggtg

SEQ ID NO: 314 - RNA, 61 nt, Homo sapiens
  source 1..61: mol_type = transcribed RNA
tcacccggtgagggcgggtggaggaggagggtccccaccatcagccttcactgggacggga

SEQ ID NO: 315 - RNA, 59 nt, Homo sapiens
  source 1..59: mol_type = transcribed RNA
ctccagggagacagtgtgtgaggcctcttgccatggcctccctgcccgcctctctgcag

SEQ ID NO: 316 - RNA, 61 nt, Homo sapiens
  source 1..61: mol_type = transcribed RNA
gagggttggggtggagggccaaggagctgggtggggtgccaagcctctgtccccaccccag

SEQ ID NO: 317 - RNA, 84 nt, Homo sapiens
  source 1..84: mol_type = transcribed RNA
ccgcagccgccgcgccgggcccgggttggccgctgacccccgcggggcccccggcggccggggcgggggcgggggctgccccgg

SEQ ID NO: 318 - RNA, 119 nt, Homo sapiens
  source 1..119: mol_type = transcribed RNA
agcctgcgccggagccggggcctgagcccgggccgcgcaggccgtgaactcgtcgagctgcgcgtgcggccggtgctcaacctgccgggtcctggccccgcgctcccgcgcgccctgga

SEQ ID NO: 319 - RNA, 64 nt, Homo sapiens
  source 1..64: mol_type = transcribed RNA
gaaggcgaggggtagaagagcacaggggttctgataaacccttctgcctgcattctactcccag

SEQ ID NO: 320 - RNA, 75 nt, Homo sapiens
  source 1..75: mol_type = transcribed RNA
aggctggcgtgggctgagggcaggaggcctgtggccggtcccaggcctcctgcttcctgggctcaggctcggttt

SEQ ID NO: 321 - RNA, 62 nt, Homo sapiens
  source 1..62: mol_type = transcribed RNA
ccttctgcggcagagctggggtcaccagccctcatgtacttgtgacttctcccctgccacag

SEQ ID NO: 322 - RNA, 96 nt, Homo sapiens
  source 1..96: mol_type = transcribed RNA
ctcgggaggggcgggaggggggtccccggtgctcggatctcgagggtgcttattgttcggtccgagcctgggtctccctcttccccccaacccccc

SEQ ID NO: 323 - RNA, 68 nt, Homo sapiens
  source 1..68: mol_type = transcribed RNA
gcatcctgtactgagctgccccgaggcccttcatgctgcccagctcggggcagctcagtacaggatac

SEQ ID NO: 324 - RNA, 64 nt, Homo sapiens
  source 1..64: mol_type = transcribed RNA
tcctgtactgagctgccccgagctgggcagcatgaagggcctcggggcagctcagtacaggatg

SEQ ID NO: 325 - RNA, 90 nt, Homo sapiens
  source 1..90: mol_type = transcribed RNA
gtgggtacggcccagtgggggggagagggacacgccctgggctctgcccagggtgcagccggactgactgagcccctgtgccgcccccag

SEQ ID NO: 326 - RNA, 80 nt, Homo sapiens
  source 1..80: mol_type = transcribed RNA
ggcgcgtcgcccccctcagtccaccagagcccggatacctcagaaattcggctctgggtctgtggggagcgaaatgcaac

SEQ ID NO: 327 - RNA, 109 nt, Homo sapiens
  source 1..109: mol_type = transcribed RNA
ggtcgggctcaccatgacacagtgtgagacctcgggctacaacacaggacccgggcgctgctctgacccctcgtgtcttgtgttgcagccggagggacgcaggtccgca

SEQ ID NO: 328 - RNA, 85 nt, Homo sapiens
  source 1..85: mol_type = transcribed RNA
tcctccccggagccaggatgcagctcaagccacagcagggtgtttagcgctcttcagtggctccagattgtggcgctggtgcagg

SEQ ID NO: 329 - RNA, 86 nt, Homo sapiens
  source 1..86: mol_type = transcribed RNA
ggggctgggggtgtggggagagagagtgcacagccagctcagggattaaagctctttctctctctctctctcccacttccctgcag

SEQ ID NO: 330 - RNA, 67 nt, Homo sapiens
  source 1..67: mol_type = transcribed RNA
gtggcactcaaactgtgggggcactttctgctctctggtgaaagtgccgccatcttttgagtgttac

SEQ ID NO: 331 - RNA, 82 nt, Homo sapiens
  source 1..82: mol_type = transcribed RNA
tgtgggcagggccctggggagctgaggctctgggggtggccggggctgaccctgggcctctgctccccagtgtctgaccgcg

SEQ ID NO: 332 - RNA, 94 nt, Homo sapiens
  source 1..94: mol_type = transcribed RNA
aattcagccctgccactggcttatgtcatgaccttgggctactcaggctgtctgcacaatgagccagttggacaggagcagtgccactcaactc

SEQ ID NO: 333 - RNA, 96 nt, Homo sapiens
  source 1..96: mol_type = transcribed RNA
cgggccccgggcgggcgggagggacgggacgcggtgcagtgttgttttttcccccgccaatattgcactcgtcccggcctccggcccccccggccc

SEQ ID NO: 334 - RNA, 79 nt, Homo sapiens
  source 1..79: mol_type = transcribed RNA
gtgcagatccttgggagccctgttagactctggattttacacttggagtgaacgggcgccatcccgaggctttgcacag

SEQ ID NO: 335 - RNA, 75 nt, Homo sapiens
  source 1..75: mol_type = transcribed RNA
gtagttgttctacagaagacctggatgtgtaggagctaagacacactccaggggagctgtggaagcagtaacacg

SEQ ID NO: 336 - RNA, 80 nt, Homo sapiens
  source 1..80: mol_type = transcribed RNA
tgagaggccgcaccttgccttgctgcccgggccgtgcacccgtgggccccagggcgacgcggcgggggcggccctagcga

SEQ ID NO: 337 - RNA, 92 nt, Homo sapiens
  source 1..92: mol_type = transcribed RNA
gtcagtgtctgggcggacagctgcaggaaagggaagaccaaggcttgctgtctgtccagtctgccaccctaccctgtctgttcttgccacag

SEQ ID NO: 338 - RNA, 115 nt, Homo sapiens
  source 1..115: mol_type = transcribed RNA
ggtgccgagggccgtccggcatcctaggcgggtcgctgcggtacctccctcctgtctgtggcggtgggatcccgtggccgtgttttcctggtggcccggccgtgcctgaggtttc

SEQ ID NO: 339 - RNA, 92 nt, Homo sapiens
  source 1..92: mol_type = transcribed RNA
cctgtccctcctgccctgcgcctgcccagccctcctgctctggtgactgaggaccgccaggcaggggctggtgctgggcggggggcggcggg

SEQ ID NO: 340 - RNA, 69 nt, Homo sapiens
  source 1..69: mol_type = transcribed RNA
ctttcggccagcgggacggcatccgaggtgggctaggctcgggcccgtggcgggtgcgggggtgggagg

SEQ ID NO: 341 - RNA, 62 nt, Homo sapiens
  source 1..62: mol_type = transcribed RNA
ggtagggggcgggctccggcgctgggaccccactagggtggcgccttggccccgccccgccc

SEQ ID NO: 342 - RNA, 86 nt, Homo sapiens
  source 1..86: mol_type = transcribed RNA
tgccagtctctaggtccctgagaccctttaacctgtgaggacatccagggtcacaggtgaggttcttgggagcctggcgtctggcc

SEQ ID NO: 343 - RNA, 47 nt, Homo sapiens
  source 1..47: mol_type = transcribed RNA
ctcggcgcggggcgcgggctccgggttggggcgagccaacgccgggg

SEQ ID NO: 344 - RNA, 73 nt, Homo sapiens
  source 1..73: mol_type = transcribed RNA
gtgggcgggggcaggtgtgtggtgggtggtggcctgcggtgagcagggccctcacacctgcctcgccccccag

SEQ ID NO: 345 - RNA, 67 nt, Homo sapiens
  source 1..67: mol_type = transcribed RNA
ggcagccagggggatgggcgagcttgggcccattcctttccttaccctaccccccatccccctgtag

SEQ ID NO: 346 - RNA, 50 nt, Homo sapiens
  source 1..50: mol_type = transcribed RNA
acccgggcgtggtggtgggggtgggtgcctgtaattccagctagttggga

SEQ ID NO: 347 - RNA, 60 nt, Homo sapiens
  source 1..60: mol_type = transcribed RNA
aggcctagggggtggcaggctggccatcagtgtgggctaaccctgtcctctccctcccag

SEQ ID NO: 348 - RNA, 102 nt, Homo sapiens
  source 1..102: mol_type = transcribed RNA
tgtgttccctatcctccttatgtcccacccccactcctgtttgaatatttcaccagaaacaggagtggggggtgggacgtaaggaggatgggggaaagaaca

SEQ ID NO: 349 - RNA, 87 nt, Homo sapiens
  source 1..87: mol_type = transcribed RNA
gcagggctggcagggaggctgggaggggctggctgggtctggtagtgggcatcagctggccctcatttcttaagacagcacttctgt

SEQ ID NO: 350 - RNA, 81 nt, Homo sapiens
  source 1..81: mol_type = transcribed RNA
catcctccttacgtcccaccccccactcctgtttctggtgaaatattcaaacaggagtgggggtgggacataaggaggata

SEQ ID NO: 351 - RNA, 66 nt, Homo sapiens
  source 1..66: mol_type = transcribed RNA
cagagcagggcagggaaggtgggagaggggcccagctgaccctcctgtcacccgctccttgcccag

SEQ ID NO: 352 - RNA, 73 nt, Homo sapiens
  source 1..73: mol_type = transcribed RNA
gaaaacaaccaggtgggcttcccggagggcggaacacccagccccagcatccagggctcacctaccacgtttg

SEQ ID NO: 353 - RNA, 89 nt, Homo sapiens
  source 1..89: mol_type = transcribed RNA
accatgctgtagtgtgtgtaaacatcctacactctcagctgtgagctcaaggtggctgggagagggttgtttactccttctgccatgga

SEQ ID NO: 354 - RNA, 68 nt, Homo sapiens
  source 1..68: mol_type = transcribed RNA
gactcggctgcggtggacaagtccggctccagaacctggacaccgctcagccggccgcggcaggggtc

SEQ ID NO: 355 - RNA, 51 nt, Homo sapiens
  source 1..51: mol_type = transcribed RNA
agagatgaagcgggggggcggggtcttgctctattgcctacgctgatctca

SEQ ID NO: 356 - RNA, 70 nt, Homo sapiens
  source 1..70: mol_type = transcribed RNA
acaaatagcttcagggagtcaggggagggcagaaatagatggccttcccctgctgggaagaaagtgggtc

SEQ ID NO: 357 - RNA, 69 nt, Homo sapiens
  source 1..69: mol_type = transcribed RNA
tggggtaggggtgggggaattcaggggtgtcgaactcatggctgccacctttgtgtccccatcctgcag

SEQ ID NO: 358 - RNA, 72 nt, Homo sapiens
  source 1..72: mol_type = transcribed RNA
tctcctcgaggggtctctgcctctacccaggactctttcatgaccaggaggctgaggcccctcacaggcggc

SEQ ID NO: 359 - RNA, 74 nt, Homo sapiens
  source 1..74: mol_type = transcribed RNA
ccaagggcacaccggggatggcagagggtcgtgggaaagtgttgaccctcgtcaggtccccggggagcccctgg

SEQ ID NO: 360 - RNA, 80 nt, Homo sapiens
  source 1..80: mol_type = transcribed RNA
cctctgtgagaaagggtgtgggggagaggctgtcttgtgtctgtaagtatgccaaacttattttccccaaggcagaggga

SEQ ID NO: 361 - RNA, 64 nt, Homo sapiens
  source 1..64: mol_type = transcribed RNA
tgctctgtaggcatgaggcagggcccaggttccatgtgatgctgaagctctgacattcctgcag

SEQ ID NO: 362 - RNA, 90 nt, Homo sapiens
  source 1..90: mol_type = transcribed RNA
tctaagaaacgcagtggtctctgaagcctgcaggggcaggccagccctgcactgaacgcctgttcttgccaggtggcagaaggttgctgc

SEQ ID NO: 363 - RNA, 106 nt, Homo sapiens
  source 1..106: mol_type = transcribed RNA
agaagaatgcccaaccagccctcagttgctacagttccctgttgtttcagctcgacaacaacaggcggctgtagcaatggggggctggatgggcatctcaatgtgc

SEQ ID NO: 364 - RNA, 71 nt, Homo sapiens
  source 1..71: mol_type = transcribed RNA
atggaggggggtgtggagccagggggcccaggtctacagcttctccccgctccctgcccccatactcccag

SEQ ID NO: 365 - RNA, 60 nt, Homo sapiens
  source 1..60: mol_type = transcribed RNA
ggtgcctcgggagggcatgggccaggccacataatgagccaaacccctgtctacccgcag

SEQ ID NO: 366 - RNA, 65 nt, Homo sapiens
  source 1..65: mol_type = transcribed RNA
tgtctggggatttggagaagtggtgagcgcaggtctttggcaccatctcccctggtcccttggct

SEQ ID NO: 367 - RNA, 110 nt, Homo sapiens
  source 1..110: mol_type = transcribed RNA
tcacctggccatgtgacttgtgggcttccctttgtcatccttcgcctagggctctgagcagggcagggacagcaaaggggtgctcagttgtcacttcccacagcacggag

SEQ ID NO: 368 - RNA, 93 nt, Homo sapiens
  source 1..93: mol_type = transcribed RNA
ccttccggcgtcccaggcggggcgccgcgggaccgccctcgtgtctgtggcggtgggatcccgcggccgtgttttcctggtggcccggccatg

SEQ ID NO: 369 - RNA, 65 nt, Homo sapiens
  source 1..65: mol_type = transcribed RNA
agccctgggggtggtctctagccaaggctctggggtctcacccttggctggtctctgctccgcag

SEQ ID NO: 370 - RNA, 61 nt, Homo sapiens
  source 1..61: mol_type = transcribed RNA
tccgctctgtggagtggggtgcctgtcccctgccactgggtgacccacccctctccaccag

SEQ ID NO: 371 - RNA, 64 nt, Homo sapiens
  source 1..64: mol_type = transcribed RNA
gagctctgggaggggctgggtttggcaggacagtttccaagccctgtctcctcccatcttccag

SEQ ID NO: 372 - RNA, 21 nt, Homo sapiens
  source 1..21: mol_type = transcribed RNA
gttggaggcgtgggttttaga

SEQ ID NO: 373 - RNA, 15 nt, Homo sapiens
  source 1..15: mol_type = transcribed RNA
gttggaggcgtgggt

SEQ ID NO: 374 - RNA, 22 nt, Homo sapiens
  source 1..22: mol_type = transcribed RNA
cgcggcggggacggcgattggt

SEQ ID NO: 375 - RNA, 17 nt, Homo sapiens
  source 1..17: mol_type = transcribed RNA
cggcggggacggcgatt

SEQ ID NO: 376 - RNA, 23 nt, Homo sapiens
  source 1..23: mol_type = transcribed RNA
ggaggcgcaggctcggaaaggcg

SEQ ID NO: 377 - RNA, 15 nt, Homo sapiens
  source 1..15: mol_type = transcribed RNA
gcaggctcggaaagg

SEQ ID NO: 378 - RNA, 26 nt, Homo sapiens
  source 1..26: mol_type = transcribed RNA
ccttctggagaggctttgtgcggata

SEQ ID NO: 379 - RNA, 15 nt, Homo sapiens
  source 1..15: mol_type = transcribed RNA
ccttctggagaggct

SEQ ID NO: 380 - RNA, 15 nt, Homo sapiens
  source 1..15: mol_type = transcribed RNA
tctgggcgaggggtg

SEQ ID NO: 381 - RNA, 15 nt, Homo sapiens
  source 1..15: mol_type = transcribed RNA
tctgggcgaggggtg

SEQ ID NO: 382 - RNA, 23 nt, Homo sapiens
  source 1..23: mol_type = transcribed RNA
ctcctggggcccgcactctcgct

SEQ ID NO: 383 - RNA, 18 nt, Homo sapiens
  source 1..18: mol_type = transcribed RNA
ctcctggggcccgcactc

SEQ ID NO: 384 - RNA, 20 nt, Homo sapiens
  source 1..20: mol_type = transcribed RNA
aagacacatttggagaggga

SEQ ID NO: 385 - RNA, 16 nt, Homo sapiens
  source 1..16: mol_type = transcribed RNA
agacacatttggagag

SEQ ID NO: 386 - RNA, 20 nt, Homo sapiens
  source 1..20: mol_type = transcribed RNA
tgagtggggctcccgggacg

SEQ ID NO: 387 - RNA, 20 nt, Homo sapiens
  source 1..20: mol_type = transcribed RNA
tgagtggggctcccgggacg

SEQ ID NO: 388 - RNA, 24 nt, Homo sapiens
  source 1..24: mol_type = transcribed RNA
tcgaggactggtggaagggccttt

SEQ ID NO: 389 - RNA, 16 nt, Homo sapiens
  source 1..16: mol_type = transcribed RNA
tcgaggactggtggaa

SEQ ID NO: 390 - RNA, 25 nt, Homo sapiens
  source 1..25: mol_type = transcribed RNA
gggtggggatttgttgcattacttg

SEQ ID NO: 391 - RNA, 20 nt, Homo sapiens
  source 1..20: mol_type = transcribed RNA
gggtggggatttgttgcatt

SEQ ID NO: 392 - RNA, 20 nt, Homo sapiens
  source 1..20: mol_type = transcribed RNA
ggctccttggtctaggggta

SEQ ID NO: 393 - RNA, 15 nt, Homo sapiens
  source 1..15: mol_type = transcribed RNA
cttggtctaggggta

SEQ ID NO: 394 - RNA, 23 nt, Homo sapiens
  source 1..23: mol_type = transcribed RNA
ctagtggaagaagatggcggaag

SEQ ID NO: 395 - RNA, 15 nt, Homo sapiens
  source 1..15: mol_type = transcribed RNA
tagtggaagaagatg

SEQ ID NO: 396 - RNA, 23 nt, Homo sapiens
  source 1..23: mol_type = transcribed RNA
acagcagggctggggattgcagt

SEQ ID NO: 397 - RNA, 19 nt, Homo sapiens
  source 1..19: mol_type = transcribed RNA
tgctgctcccagtcctgcc

SEQ ID NO: 398 - RNA, 25 nt, Homo sapiens
  source 1..25: mol_type = transcribed RNA
tggcggcggtagttatgggcttctc

SEQ ID NO: 399 - RNA, 25 nt, Homo sapiens
  source 1..25: mol_type = transcribed RNA
tggcggcggtagttatgggcttctc

SEQ ID NO: 400 - RNA, 20 nt, Homo sapiens
  source 1..20: mol_type = transcribed RNA
taggatgggggtgagaggtg

SEQ ID NO: 401 - RNA, 18 nt, Homo sapiens
  source 1..18: mol_type = transcribed RNA
taggatgggggtgagagg

SEQ ID NO: 402 - RNA, 21 nt, Homo sapiens
  source 1..21: mol_type = transcribed RNA
cagcctgagtgacagagcaag

SEQ ID NO: 403 - RNA, 15 nt, Homo sapiens
  source 1..15: mol_type = transcribed RNA
actgcactccagcct

SEQ ID NO: 404 - RNA, 23 nt, Homo sapiens
  source 1..23: mol_type = transcribed RNA
aggaggggtcccgcactgggagg

SEQ ID NO: 405 - RNA, 15 nt, Homo sapiens
  source 1..15: mol_type = transcribed RNA
tgggaggggccctca

SEQ ID NO: 406 - RNA, 23 nt, Homo sapiens
  source 1..23: mol_type = transcribed RNA
gggggccgatacactgtacgaga

SEQ ID NO: 407 - RNA, 20 nt, Homo sapiens
  source 1..20: mol_type = transcribed RNA
gggggccgatacactgtacg

SEQ ID NO: 408 - RNA, 23 nt, Homo sapiens
  source 1..23: mol_type = transcribed RNA
ggtgggtgaggtcgggccccaag

SEQ ID NO: 409 - RNA, 20 nt, Homo sapiens
  source 1..20: mol_type = transcribed RNA
cggggtgggtgaggtcgggc

SEQ ID NO: 410 - RNA, 23 nt, Homo sapiens
  source 1..23: mol_type = transcribed RNA
agaagaaggcggtcggtctgcgg

SEQ ID NO: 411 - RNA, 21 nt, Homo sapiens
  source 1..21: mol_type = transcribed RNA
aagaaggcggtcggtctgcgg

SEQ ID NO: 412 - RNA, 17 nt, Homo sapiens
  source 1..17: mol_type = transcribed RNA
ggtgagcgctcgctggc

SEQ ID NO: 413 - RNA, 15 nt, Homo sapiens
  source 1..15: mol_type = transcribed RNA
cggtgagcgctcgct

SEQ ID NO: 414 - RNA, 16 nt, Homo sapiens
  source 1..16: mol_type = transcribed RNA
tgctggtgatgctttc

SEQ ID NO: 415 - RNA, 16 nt, Homo sapiens
  source 1..16: mol_type = transcribed RNA
tgctggtgatgctttc

SEQ ID NO: 416 - RNA, 23 nt, Homo sapiens
  source 1..23: mol_type = transcribed RNA
gccccggcgcgggcgggttctgg

SEQ ID NO: 417 - RNA, 16 nt, Homo sapiens
  source 1..16: mol_type = transcribed RNA
ggagccccggcgcggg

SEQ ID NO: 418 - RNA, 21 nt, Homo sapiens
  source 1..21: mol_type = transcribed RNA
cggatccgagtcacggcacca

SEQ ID NO: 419 - RNA, 15 nt, Homo sapiens
  source 1..15: mol_type = transcribed RNA
ggatccgagtcacgg

SEQ ID NO: 420 - RNA, 23 nt, Homo sapiens
  source 1..23: mol_type = transcribed RNA
ggggggatgtgcatgctggttgg

SEQ ID NO: 421 - RNA, 15 nt, Homo sapiens
  source 1..15: mol_type = transcribed RNA
atcagcgtgcacttc

SEQ ID NO: 422 - RNA, 17 nt, Homo sapiens
  source 1..17: mol_type = transcribed RNA
tcctagtcacggcacca

SEQ ID NO: 423 - RNA, 17 nt, Homo sapiens
  source 1..17: mol_type = transcribed RNA
tcctagtcacggcacca

SEQ ID NO: 424 - RNA, 25 nt, Homo sapiens
  source 1..25: mol_type = transcribed RNA
tgcggggctagggctaacagcagtc

SEQ ID NO: 425 - RNA, 15 nt, Homo sapiens
  source 1..15: mol_type = transcribed RNA
tgcggggctagggct

SEQ ID NO: 426 - RNA, 21 nt, Homo sapiens
  source 1..21: mol_type = transcribed RNA
cccaggctggagcgagtgcag

SEQ ID NO: 427 - RNA, 15 nt, Homo sapiens
  source 1..15: mol_type = transcribed RNA
agctcactgcagcct

SEQ ID NO: 428 - RNA, 22 nt, Homo sapiens
  source 1..22: mol_type = transcribed RNA
tgtgggactgcaaatgggagct

SEQ ID NO: 429 - RNA, 22 nt, Homo sapiens
  source 1..22: mol_type = transcribed RNA
tgtgggactgcaaatgggagct

SEQ ID NO: 430 - RNA, 26 nt, Homo sapiens
  source 1..26: mol_type = transcribed RNA
gcgggctgtccggaggggtcggcttt

SEQ ID NO: 431 - RNA, 16 nt, Homo sapiens
  source 1..16: mol_type = transcribed RNA
gctgtccggaggggtc

SEQ ID NO: 432 - RNA, 22 nt, Homo sapiens
  source 1..22: mol_type = transcribed RNA
ccgggaacgtcgagactggagc

SEQ ID NO: 433 - RNA, 15 nt, Homo sapiens
  source 1..15: mol_type = transcribed RNA
cgggaacgtcgagac

SEQ ID NO: 434 - RNA, 17 nt, Homo sapiens
  source 1..17: mol_type = transcribed RNA
cctccgggacggctggg

SEQ ID NO: 435 - RNA, 15 nt, Homo sapiens
  source 1..15: mol_type = transcribed RNA
ctccgggacggctgg

SEQ ID NO: 436 - RNA, 21 nt, Homo sapiens
  source 1..21: mol_type = transcribed RNA
cccagcaggacgggagcgcgg

SEQ ID NO: 437 - RNA, 16 nt, Homo sapiens
  source 1..16: mol_type = transcribed RNA
aagctgggtcaaggag

SEQ ID NO: 438 - RNA, 22 nt, Homo sapiens
  source 1..22: mol_type = transcribed RNA
ctgggctcgggacgcgcggctc

SEQ ID NO: 439 - RNA, 19 nt, Homo sapiens
  source 1..19: mol_type = transcribed RNA
ctgggctcgggacgcgcgg

SEQ ID NO: 440 - RNA, 29 nt, Homo sapiens
  source 1..29: mol_type = transcribed RNA
ggtcaggcggctcggactgagcaggtggg

SEQ ID NO: 441 - RNA, 15 nt, Homo sapiens
  source 1..15: mol_type = transcribed RNA
agagtgtggtcaggc

SEQ ID NO: 442 - RNA, 23 nt, Homo sapiens
  source 1..23: mol_type = transcribed RNA
cagcccgccccagccgaggttct

SEQ ID NO: 443 - RNA, 17 nt, Homo sapiens
  source 1..17: mol_type = transcribed RNA
agcccgccccagccgag

SEQ ID NO: 444 - RNA, 23 nt, Homo sapiens
  source 1..23: mol_type = transcribed RNA
agggtcggggcagggagggcagg

SEQ ID NO: 445 - RNA, 15 nt, Homo sapiens
  source 1..15: mol_type = transcribed RNA
gggagaagggtcggg

SEQ ID NO: 446 - RNA, 21 nt, Homo sapiens
  source 1..21: mol_type = transcribed RNA
atccagttctctgagggggct

SEQ ID NO: 447 - RNA, 21 nt, Homo sapiens
  source 1..21: mol_type = transcribed RNA
atccagttctctgagggggct

SEQ ID NO: 448 - RNA, 16 nt, Homo sapiens
  source 1..16: mol_type = transcribed RNA
gtgaaggcccggcgga

SEQ ID NO: 449 - RNA, 15 nt, Homo sapiens
  source 1..15: mol_type = transcribed RNA
gtgaaggcccggcgg

SEQ ID NO: 450 - RNA, 23 nt, Homo sapiens
  source 1..23: mol_type = transcribed RNA
ccagggctggcagtgacatgggt

SEQ ID NO: 451 - RNA, 19 nt, Homo sapiens
  source 1..19: mol_type = transcribed RNA
cagggctggcagtgacatg

SEQ ID NO: 452 - RNA, 21 nt, Homo sapiens
  source 1..21: mol_type = transcribed RNA
gccgggcgtggtggtgggggc

SEQ ID NO: 453 - RNA, 15 nt, Homo sapiens
  source 1..15: mol_type = transcribed RNA
tagccgggcgtggtg

SEQ ID NO: 454 - RNA, 21 nt, Homo sapiens
  source 1..21: mol_type = transcribed RNA
ccggccgccggctccgccccg

SEQ ID NO: 455 - RNA, 17 nt, Homo sapiens
  source 1..17: mol_type = transcribed RNA
ccggccgccggctccgc

SEQ ID NO: 456 - RNA, 23 nt, Homo sapiens
  source 1..23: mol_type = transcribed RNA
tgaggatatggcagggaagggga

SEQ ID NO: 457 - RNA, 19 nt, Homo sapiens
  source 1..19: mol_type = transcribed RNA
tgaggatatggcagggaag

SEQ ID NO: 458 - RNA, 21 nt, Homo sapiens
  source 1..21: mol_type = transcribed RNA
aggaggcagtgggcgagcagg

SEQ ID NO: 459 - RNA, 21 nt, Homo sapiens
  source 1..21: mol_type = transcribed RNA
aggaggcagtgggcgagcagg

SEQ ID NO: 460 - RNA, 24 nt, Homo sapiens
  source 1..24: mol_type = transcribed RNA
gatcggtcgagagcgtcctggctg

SEQ ID NO: 461 - RNA, 15 nt, Homo sapiens
  source 1..15: mol_type = transcribed RNA
gctgggcggggcgcg

SEQ ID NO: 462 - RNA, 24 nt, Homo sapiens
  source 1..24: mol_type = transcribed RNA
cccggggcagattggtgtagggtg

SEQ ID NO: 463 - RNA, 17 nt, Homo sapiens
  source 1..17: mol_type = transcribed RNA
cggggcagattggtgta

SEQ ID NO: 464 - RNA, 23 nt, Homo sapiens
  source 1..23: mol_type = transcribed RNA
tggggcggagcttccggaggccc

SEQ ID NO: 465 - RNA, 16 nt, Homo sapiens
  source 1..16: mol_type = transcribed RNA
atcgctggcctggtcg

SEQ ID NO: 466 - RNA, 15 nt, Homo sapiens
  source 1..15: mol_type = transcribed RNA
tggcagagcgctgtc

SEQ ID NO: 467 - RNA, 15 nt, Homo sapiens
  source 1..15: mol_type = transcribed RNA
tggcagagcgctgtc

SEQ ID NO: 468 - RNA, 24 nt, Homo sapiens
  source 1..24: mol_type = transcribed RNA
gtgagtgggagccggtggggctgg

SEQ ID NO: 469 - RNA, 15 nt, Homo sapiens
  source 1..15: mol_type = transcribed RNA
ggggctggagtaagg

SEQ ID NO: 470 - RNA, 22 nt, Homo sapiens
  source 1..22: mol_type = transcribed RNA
accccactcctggtaccatagt

SEQ ID NO: 471 - RNA, 15 nt, Homo sapiens
  source 1..15: mol_type = transcribed RNA
accccactcctggta

SEQ ID NO: 472 - RNA, 25 nt, Homo sapiens
  source 1..25: mol_type = transcribed RNA
agcggggaggaagtgggcgctgctt

SEQ ID NO: 473 - RNA, 21 nt, Homo sapiens
  source 1..21: mol_type = transcribed RNA
agcggggaggaagtgggcgct

SEQ ID NO: 474 - RNA, 16 nt, Homo sapiens
  source 1..16: mol_type = transcribed RNA
ggacccagggagagac

SEQ ID NO: 475 - RNA, 16 nt, Homo sapiens
  source 1..16: mol_type = transcribed RNA
ggacccagggagagac

SEQ ID NO: 476 - RNA, 20 nt, Homo sapiens
  source 1..20: mol_type = transcribed RNA
atcccacctctgccaccaaa

SEQ ID NO: 477 - RNA, 15 nt, Homo sapiens
  source 1..15: mol_type = transcribed RNA
atcccacctctgcca

SEQ ID NO: 478 - RNA, 16 nt, Homo sapiens
  source 1..16: mol_type = transcribed RNA
cgggcccggcgttccc

SEQ ID NO: 479 - RNA, 15 nt, Homo sapiens
  source 1..15: mol_type = transcribed RNA
ccgggcccggcgttc

SEQ ID NO: 480 - RNA, 24 nt, Homo sapiens
  source 1..24: mol_type = transcribed RNA
tgactggggagcagaaggagaacc

SEQ ID NO: 481 - RNA, 15 nt, Homo sapiens
  source 1..15: mol_type = transcribed RNA
gactggggagcagaa

SEQ ID NO: 482 - RNA, 27 nt, Homo sapiens
  source 1..27: mol_type = transcribed RNA
agggccagaggagcctggagtggtcgg

SEQ ID NO: 483 - RNA, 23 nt, Homo sapiens
  source 1..23: mol_type = transcribed RNA
agggccagaggagcctggagtgg

SEQ ID NO: 484 - RNA, 18 nt, Homo sapiens
  source 1..18: mol_type = transcribed RNA
aaaaggaagggggaggag

SEQ ID NO: 485 - RNA, 16 nt, Homo sapiens
  source 1..16: mol_type = transcribed RNA
aaggaagggggaggag

SEQ ID NO: 486 - RNA, 20 nt, Homo sapiens
  source 1..20: mol_type = transcribed RNA
ccccggggagcccggcggtg

SEQ ID NO: 487 - RNA, 15 nt, Homo sapiens
  source 1..15: mol_type = transcribed RNA
accccggggagcccg

SEQ ID NO: 488 - RNA, 19 nt, Homo sapiens
  source 1..19: mol_type = transcribed RNA
gctgggcgaggctggcatc

SEQ ID NO: 489 - RNA, 17 nt, Homo sapiens
  source 1..17: mol_type = transcribed RNA
gctgggcgaggctggca

SEQ ID NO: 490 - RNA, 22 nt, Homo sapiens
  source 1..22: mol_type = transcribed RNA
tgggaggggagaggcagcaagc

SEQ ID NO: 491 - RNA, 22 nt, Homo sapiens
  source 1..22: mol_type = transcribed RNA
tgggaggggagaggcagcaagc

SEQ ID NO: 492 - RNA, 18 nt, Homo sapiens
  source 1..18: mol_type = transcribed RNA
cagcggggctgggcgcgc

SEQ ID NO: 493 - RNA, 15 nt, Homo sapiens
  source 1..15: mol_type = transcribed RNA
cagcggggctgggcg

SEQ ID NO: 494 - RNA, 23 nt, Homo sapiens
  source 1..23: mol_type = transcribed RNA
cggggccgtagcactgtctgaga

SEQ ID NO: 495 - RNA, 20 nt, Homo sapiens
  source 1..20: mol_type = transcribed RNA
cggggccgtagcactgtctg

SEQ ID NO: 496 - RNA, 18 nt, Homo sapiens
  source 1..18: mol_type = transcribed RNA
tctaggtggggagactga

SEQ ID NO: 497 - RNA, 16 nt, Homo sapiens
  source 1..16: mol_type = transcribed RNA
gtggggagactgacgg

SEQ ID NO: 498 - RNA, 21 nt, Homo sapiens
  source 1..21: mol_type = transcribed RNA
ttgaggagacatggtgggggc

SEQ ID NO: 499 - RNA, 15 nt, Homo sapiens
  source 1..15: mol_type = transcribed RNA
ttgaggagacatggt

SEQ ID NO: 500 - RNA, 18 nt, Homo sapiens
  source 1..18: mol_type = transcribed RNA
gaggctgaaggaagatgg

SEQ ID NO: 501 - RNA, 15 nt, Homo sapiens
  source 1..15: mol_type = transcribed RNA
gaggctgaaggaaga

SEQ ID NO: 502 - RNA, 22 nt, Homo sapiens
  source 1..22: mol_type = transcribed RNA
ggcaggaagcggaggaaccttg

SEQ ID NO: 503 - RNA, 17 nt, Homo sapiens
  source 1..17: mol_type = transcribed RNA
ggaggaaccttggagct

SEQ ID NO: 504 - RNA, 17 nt, Homo sapiens
  source 1..17: mol_type = transcribed RNA
aggggctgggcgcgcgc

SEQ ID NO: 505 - RNA, 15 nt, Homo sapiens
  source 1..15: mol_type = transcribed RNA
caggggctgggcgcg

SEQ ID NO: 506 - RNA, 22 nt, Homo sapiens
  source 1..22: mol_type = transcribed RNA
gagggttgggtggaggctctcc

SEQ ID NO: 507 - RNA, 15 nt, Homo sapiens
  source 1..15: mol_type = transcribed RNA
gagggttgggtggag

SEQ ID NO: 508 - RNA, 21 nt, Homo sapiens
  source 1..21: mol_type = transcribed RNA
gagggcagcgtgggtgtggcg

SEQ ID NO: 509 - RNA, 21 nt, Homo sapiens
  source 1..21: mol_type = transcribed RNA
gagggcagcgtgggtgtggcg

SEQ ID NO: 510 - RNA, 30 nt, Homo sapiens
  source 1..30: mol_type = transcribed RNA
gctgggctgggacggacacccggcctccac

SEQ ID NO: 511 - RNA, 19 nt, Homo sapiens
  source 1..19: mol_type = transcribed RNA
gaggctgggctgggacgga

SEQ ID NO: 512 - RNA, 22 nt, Homo sapiens
  source 1..22: mol_type = transcribed RNA
cagaaggggagttgggagcaga

SEQ ID NO: 513 - RNA, 16 nt, Homo sapiens
  source 1..16: mol_type = transcribed RNA
gaaggggagttgggag

SEQ ID NO: 514 - RNA, 29 nt, Homo sapiens
  source 1..29: mol_type = transcribed RNA
gggagccgcggggatcgccgagggccggt

SEQ ID NO: 515 - RNA, 15 nt, Homo sapiens
  source 1..15: mol_type = transcribed RNA
ggcggcggtggtggg

SEQ ID NO: 516 - RNA, 20 nt, Homo sapiens
  source 1..20: mol_type = transcribed RNA
ctccccggtgtgcaaatgtg

SEQ ID NO: 517 - RNA, 15 nt, Homo sapiens
  source 1..15: mol_type = transcribed RNA
gtgtgcggtgttatg

SEQ ID NO: 518 - RNA, 19 nt, Homo sapiens
  source 1..19: mol_type = transcribed RNA
gagggcgggtggaggagga

SEQ ID NO: 519 - RNA, 15 nt, Homo sapiens
  source 1..15: mol_type = transcribed RNA
gcgggtggaggagga

SEQ ID NO: 520 - RNA, 17 nt, Homo sapiens
  source 1..17: mol_type = transcribed RNA
ggggcgggggcgggggc

SEQ ID NO: 521 - RNA, 15 nt, Homo sapiens
  source 1..15: mol_type = transcribed RNA
cgcgccgggcccggg

SEQ ID NO: 522 - RNA, 23 nt, Homo sapiens
  source 1..23: mol_type = transcribed RNA
gggggtccccggtgctcggatct

SEQ ID NO: 523 - RNA, 16 nt, Homo sapiens
  source 1..16: mol_type = transcribed RNA
tcgggaggggcgggag

SEQ ID NO: 524 - RNA, 23 nt, Homo sapiens
  source 1..23: mol_type = transcribed RNA
cggggcagctcagtacaggatac

SEQ ID NO: 525 - RNA, 15 nt, Homo sapiens
  source 1..15: mol_type = transcribed RNA
agctcagtacaggat

SEQ ID NO: 526 - RNA, 23 nt, Homo sapiens
  source 1..23: mol_type = transcribed RNA
tcggctctgggtctgtggggagc

SEQ ID NO: 527 - RNA, 15 nt, Homo sapiens
  source 1..15: mol_type = transcribed RNA
gcccggatacctcag

SEQ ID NO: 528 - RNA, 23 nt, Homo sapiens
  source 1..23: mol_type = transcribed RNA
ggctacaacacaggacccgggcg

SEQ ID NO: 529 - RNA, 21 nt, Homo sapiens
  source 1..21: mol_type = transcribed RNA
ggctacaacacaggacccggg

SEQ ID NO: 530 - RNA, 22 nt, Homo sapiens
  source 1..22: mol_type = transcribed RNA
actcaaactgtgggggcacttt

SEQ ID NO: 531 - RNA, 19 nt, Homo sapiens
  source 1..19: mol_type = transcribed RNA
actcaaactgtgggggcac

SEQ ID NO: 532 - RNA, 24 nt, Homo sapiens
  source 1..24: mol_type = transcribed RNA
tggggagctgaggctctgggggtg

SEQ ID NO: 533 - RNA, 15 nt, Homo sapiens
  source 1..15: mol_type = transcribed RNA
ggccctggggagctg

SEQ ID NO: 534 - RNA, 27 nt, Homo sapiens
  source 1..27: mol_type = transcribed RNA
gtgaacgggcgccatcccgaggctttg

SEQ ID NO: 535 - RNA, 16 nt, Homo sapiens
  source 1..16: mol_type = transcribed RNA
gtgaacgggcgccatc

SEQ ID NO: 536 - RNA, 23 nt, Homo sapiens
  source 1..23: mol_type = transcribed RNA
caccttgccttgctgcccgggcc

SEQ ID NO: 537 - RNA, 22 nt, Homo sapiens
  source 1..22: mol_type = transcribed RNA
caccttgccttgctgcccgggc

SEQ ID NO: 538 - RNA, 23 nt, Homo sapiens
  source 1..23: mol_type = transcribed RNA
ggcccggccgtgcctgaggtttc

SEQ ID NO: 539 - RNA, 15 nt, Homo sapiens
  source 1..15: mol_type = transcribed RNA
ggcggtgggatcccg

SEQ ID NO: 540 - RNA, 24 nt, Homo sapiens
  source 1..24: mol_type = transcribed RNA
aggcaggggctggtgctgggcggg

SEQ ID NO: 541 - RNA, 15 nt, Homo sapiens
  source 1..15: mol_type = transcribed RNA
gggcggggggcggcg

SEQ ID NO: 542 - RNA, 19 nt, Homo sapiens
  source 1..19: mol_type = transcribed RNA
tggcgggtgcgggggtggg

SEQ ID NO: 543 - RNA, 15 nt, Homo sapiens
  source 1..15: mol_type = transcribed RNA
tggcgggtgcggggg

SEQ ID NO: 544 - RNA, 19 nt, Homo sapiens
  source 1..19: mol_type = transcribed RNA
agggggcgggctccggcgc

SEQ ID NO: 545 - RNA, 15 nt, Homo sapiens
  source 1..15: mol_type = transcribed RNA
gtagggggcgggctc

SEQ ID NO: 546 - RNA, 23 nt, Homo sapiens
  source 1..23: mol_type = transcribed RNA
cacaggtgaggttcttgggagcc

SEQ ID NO: 547 - RNA, 15 nt, Homo sapiens
  source 1..15: mol_type = transcribed RNA
acaggtgaggttctt

SEQ ID NO: 548 - RNA, 22 nt, Homo sapiens
  source 1..22: mol_type = transcribed RNA
gtgggcgggggcaggtgtgtgg

SEQ ID NO: 549 - RNA, 15 nt, Homo sapiens
  source 1..15: mol_type = transcribed RNA
cgggggcaggtgtgt

SEQ ID NO: 550 - RNA, 24 nt, Homo sapiens
  source 1..24: mol_type = transcribed RNA
cgggcgtggtggtgggggtgggtg

SEQ ID NO: 551 - RNA, 15 nt, Homo sapiens
  source 1..15: mol_type = transcribed RNA
cgggcgtggtggtgg

SEQ ID NO: 552 - RNA, 20 nt, Homo sapiens
  source 1..20: mol_type = transcribed RNA
ccccagggcgacgcggcggg

SEQ ID NO: 553 - RNA, 15 nt, Homo sapiens
  source 1..15: mol_type = transcribed RNA
cgcggcgggggcggc

SEQ ID NO: 554 - RNA, 23 nt, Homo sapiens
  source 1..23: mol_type = transcribed RNA
acaggagtgggggtgggacataa

SEQ ID NO: 555 - RNA, 20 nt, Homo sapiens
  source 1..20: mol_type = transcribed RNA
acaggagtgggggtgggaca

SEQ ID NO: 556 - RNA, 18 nt, Homo sapiens
  source 1..18: mol_type = transcribed RNA
ggtgggcttcccggaggg

SEQ ID NO: 557 - RNA, 15 nt, Homo sapiens
  source 1..15: mol_type = transcribed RNA
ggtgggcttcccgga

SEQ ID NO: 558 - RNA, 27 nt, Homo sapiens
  source 1..27: mol_type = transcribed RNA
caaggtggctgggagagggttgtttac

SEQ ID NO: 559 - RNA, 15 nt, Homo sapiens
  source 1..15: mol_type = transcribed RNA
gtgagctcaaggtgg

SEQ ID NO: 560 - RNA, 22 nt, Homo sapiens
  source 1..22: mol_type = transcribed RNA
actcggctgcggtggacaagtc

SEQ ID NO: 561 - RNA, 20 nt, Homo sapiens
  source 1..20: mol_type = transcribed RNA
actcggctgcggtggacaag

SEQ ID NO: 562 - RNA, 15 nt, Homo sapiens
  source 1..15: mol_type = transcribed RNA
tgaagcgggggggcg

SEQ ID NO: 563 - RNA, 15 nt, Homo sapiens
  source 1..15: mol_type = transcribed RNA
tgaagcgggggggcg

SEQ ID NO: 564 - RNA, 22 nt, Homo sapiens
  source 1..22: mol_type = transcribed RNA
accaggaggctgaggcccctca

SEQ ID NO: 565 - RNA, 15 nt, Homo sapiens
  source 1..15: mol_type = transcribed RNA
accaggaggctgagg

SEQ ID NO: 566 - RNA, 25 nt, Homo sapiens
  source 1..25: mol_type = transcribed RNA
tcctgtactgagctgccccgaggcc

SEQ ID NO: 567 - RNA, 15 nt, Homo sapiens
  source 1..15: mol_type = transcribed RNA
tcctgtactgagctg

SEQ ID NO: 568 - RNA, 22 nt, Homo sapiens
  source 1..22: mol_type = transcribed RNA
acaccggggatggcagagggtc

SEQ ID NO: 569 - RNA, 20 nt, Homo sapiens
  source 1..20: mol_type = transcribed RNA
caccggggatggcagagggt

SEQ ID NO: 570 - RNA, 23 nt, Homo sapiens
  source 1..23: mol_type = transcribed RNA
gtgggggagaggctgtcttgtgt

SEQ ID NO: 571 - RNA, 15 nt, Homo sapiens
  source 1..15: mol_type = transcribed RNA
gtgtgggggagaggc

SEQ ID NO: 572 - RNA, 17 nt, Homo sapiens
  source 1..17: mol_type = transcribed RNA
tgcaggggcaggccagc

SEQ ID NO: 573 - RNA, 17 nt, Homo sapiens
  source 1..17: mol_type = transcribed RNA
tgcaggggcaggccagc

SEQ ID NO: 574 - RNA, 22 nt, Homo sapiens
  source 1..22: mol_type = transcribed RNA
tggggatttggagaagtggtga

SEQ ID NO: 575 - RNA, 22 nt, Homo sapiens
  source 1..22: mol_type = transcribed RNA
tggggatttggagaagtggtga

SEQ ID NO: 576 - RNA, 22 nt, Homo sapiens
  source 1..22: mol_type = transcribed RNA
ggcagggacagcaaaggggtgc

SEQ ID NO: 577 - RNA, 18 nt, Homo sapiens
  source 1..18: mol_type = transcribed RNA
gcagggacagcaaagggg

SEQ ID NO: 578 - RNA, 26 nt, Homo sapiens
  source 1..26: mol_type = transcribed RNA
cggtgggatcccgcggccgtgttttc

SEQ ID NO: 579 - RNA, 15 nt, Homo sapiens
  source 1..15: mol_type = transcribed RNA
ggggcgccgcgggac

SEQ ID NO: 580 - RNA, 21 nt, Homo sapiens
  source 1..21: mol_type = transcribed RNA
gctgggaaggcaaagggacgt

SEQ ID NO: 581 - RNA, 22 nt, Homo sapiens
  source 1..22: mol_type = transcribed RNA
agacacatttggagagggaacc

SEQ ID NO: 582 - RNA, 22 nt, Homo sapiens
  source 1..22: mol_type = transcribed RNA
ggggctggggccggggccgagc

SEQ ID NO: 583 - RNA, 21 nt, Homo sapiens
  source 1..21: mol_type = transcribed RNA
gtgccagctgcagtgggggag

SEQ ID NO: 584 - RNA, 23 nt, Homo sapiens
  source 1..23: mol_type = transcribed RNA
ttagggagtagaagggtggggag

SEQ ID NO: 585 - RNA, 18 nt, Homo sapiens
  source 1..18: mol_type = transcribed RNA
cggggcggcaggggcctc

SEQ ID NO: 586 - RNA, 22 nt, Homo sapiens
  source 1..22: mol_type = transcribed RNA
caggcacgggagctcaggtgag

SEQ ID NO: 587 - RNA, 18 nt, Homo sapiens
  source 1..18: mol_type = transcribed RNA
agcaggtgcggggcggcg

SEQ ID NO: 588 - RNA, 20 nt, Homo sapiens
  source 1..20: mol_type = transcribed RNA
gtgggttggggcgggctctg

SEQ ID NO: 589 - RNA, 17 nt, Homo sapiens
  source 1..17: mol_type = transcribed RNA
gggagtctacagcaggg

SEQ ID NO: 590 - RNA, 18 nt, Homo sapiens
  source 1..18: mol_type = transcribed RNA
gggtgcgggccggcgggg

SEQ ID NO: 591 - RNA, 22 nt, Homo sapiens
  source 1..22: mol_type = transcribed RNA
caggaaggatttagggacaggc

SEQ ID NO: 592 - RNA, 24 nt, Homo sapiens
  source 1..24: mol_type = transcribed RNA
tgggggagccatgagataagagca

SEQ ID NO: 593 - RNA, 22 nt, Homo sapiens
  source 1..22: mol_type = transcribed RNA
tggggaaggcgtcagtgtcggg

SEQ ID NO: 594 - RNA, 23 nt, Homo sapiens
  source 1..23: mol_type = transcribed RNA
ctggcggagcccattccatgcca

SEQ ID NO: 595 - RNA, 25 nt, Homo sapiens
  source 1..25: mol_type = transcribed RNA
aagggaggaggagcggaggggccct

SEQ ID NO: 596 - RNA, 22 nt, Homo sapiens
  source 1..22: mol_type = transcribed RNA
gcgggggtggcggcggcatccc

SEQ ID NO: 597 - RNA, 20 nt, Homo sapiens
  source 1..20: mol_type = transcribed RNA
gggctggggcgcggggaggt

SEQ ID NO: 598 - RNA, 19 nt, Homo sapiens
  source 1..19: mol_type = transcribed RNA
aaggggctgggggagcaca

SEQ ID NO: 599 - RNA, 22 nt, Homo sapiens
  source 1..22: mol_type = transcribed RNA
aggcgatgtggggatgtagaga

SEQ ID NO: 600 - RNA, 23 nt, Homo sapiens
  source 1..23: mol_type = transcribed RNA
ctgggcccgcggcgggcgtgggg

SEQ ID NO: 601 - RNA, 23 nt, Homo sapiens
  source 1..23: mol_type = transcribed RNA
acggggagtcaggcagtggtgga

SEQ ID NO: 602 - RNA, 22 nt, Homo sapiens
  source 1..22: mol_type = transcribed RNA
agtgggaggacaggaggcaggt

SEQ ID NO: 603 - RNA, 22 nt, Homo sapiens
  source 1..22: mol_type = transcribed RNA
tggcgggggtagagctggctgc

SEQ ID NO: 604 - RNA, 24 nt, Homo sapiens
  source 1..24: mol_type = transcribed RNA
gtaggggcgtcccgggcgcgcggg

SEQ ID NO: 605 - RNA, 19 nt, Homo sapiens
  source 1..19: mol_type = transcribed RNA
cggggccagagcagagagc

SEQ ID NO: 606 - RNA, 21 nt, Homo sapiens
  source 1..21: mol_type = transcribed RNA
caggcaggtgtagggtggagc

SEQ ID NO: 607 - RNA, 22 nt, Homo sapiens
  source 1..22: mol_type = transcribed RNA
tagcagcacgtaaatattggcg

SEQ ID NO: 608 - RNA, 23 nt, Homo sapiens
  source 1..23: mol_type = transcribed RNA
tgaggggcagagagcgagacttt

SEQ ID NO: 609 - RNA, 22 nt, Homo sapiens
  source 1..22: mol_type = transcribed RNA
aaaccgttaccattactgagtt

SEQ ID NO: 610 - RNA, 19 nt, Homo sapiens
  source 1..19: mol_type = transcribed RNA
aggcacggtgtcagcaggc

SEQ ID NO: 611 - RNA, 23 nt, Homo sapiens
  source 1..23: mol_type = transcribed RNA
aggaagccctggaggggctggag

SEQ ID NO: 612 - RNA, 110 nt, Homo sapiens
  source 1..110: mol_type = transcribed RNA
ggctacagtctttcttcatgtgactcgtggacttccctttgtcatcctatgcctgagaatatatgaaggaggctgggaaggcaaagggacgttcaattgtcatcactggc

SEQ ID NO: 613 - RNA, 97 nt, Homo sapiens
  source 1..97: mol_type = transcribed RNA
atctgagttgggagggtccctctccaaatgtgtcttggggtgggggatcaagacacatttggagagggaacctcccaactcggcctctgccatcatt

SEQ ID NO: 614 - RNA, 83 nt, Homo sapiens
  source 1..83: mol_type = transcribed RNA
ggcccggctccgggtctcggcccgtacagtccggccggccatgctggcggggctggggccggggccgagcccgcggcggggcc

SEQ ID NO: 615 - RNA, 83 nt, Homo sapiens
  source 1..83: mol_type = transcribed RNA
cctgctgcagaggtgccagctgcagtgggggaggcactgccagggctgcccactctgcttagccagcaggtgccaagaacagg

SEQ ID NO: 616 - RNA, 82 nt, Homo sapiens
  source 1..82: mol_type = transcribed RNA
ctgacttttttagggagtagaagggtggggagcatgaacaatgtttctcactccctacccctccactccccaaaaaagtcag

SEQ ID NO: 617 - RNA, 64 nt, Homo sapiens
  source 1..64: mol_type = transcribed RNA
gggtgggggcggggcggcaggggcctcccccagtgccaggccccattctgcttctctcccagct

SEQ ID NO: 618 - RNA, 83 nt, Homo sapiens
  source 1..83: mol_type = transcribed RNA
aatagagggtgcacaggcacgggagctcaggtgaggcagggagctgagctcacctgacctcccatgcctgtgcaccctctatt

SEQ ID NO: 619 - RNA, 105 nt, Homo sapiens
  source 1..105: mol_type = transcribed RNA
gcgggcggcggcggcggcagcagcagcaggtgcggggcggcggccgcgctggccgctcgactccgcagctgctcgttctgcttctccagcttgcgcaccagctcc

SEQ ID NO: 620 - RNA, 102 nt, Homo sapiens
  source 1..102: mol_type = transcribed RNA
gcttatcgaggaaaagatcgaggtgggttggggcgggctctggggatttggtctcacagcccggatcccagcccacttaccttggttactctccttccttct

SEQ ID NO: 621 - RNA, 76 nt, Homo sapiens
  source 1..76: mol_type = transcribed RNA
ccgatgcctcgggagtctacagcagggccatgtctgtgagggcccaagggtgcatgtgtctcccaggtttcggtgc

SEQ ID NO: 622 - RNA, 54 nt, Homo sapiens
  source 1..54: mol_type = transcribed RNA
acgcgggtgcgggccggcggggtagaagccacccggcccggcccggcccggcga

SEQ ID NO: 623 - RNA, 70 nt, Homo sapiens
  source 1..70: mol_type = transcribed RNA
aaaagcctgtccctaagtccctcccagccttccagagttggtgccaggaaggatttagggacaggctttg

SEQ ID NO: 624 - RNA, 81 nt, Homo sapiens
  source 1..81: mol_type = transcribed RNA
agttggtgggggagccatgagataagagcacctcctagagaatgttgaactaaaggtgccctctctggctcctccccaaag

SEQ ID NO: 625 - RNA, 90 nt, Homo sapiens
  source 1..90: mol_type = transcribed RNA
gtgtctctctggagaccctgcagccttcccacccaccagggagctttccatgggctgtggggaaggcgtcagtgtcgggtgagggaacac

SEQ ID NO: 626 - RNA, 76 nt, Homo sapiens
  source 1..76: mol_type = transcribed RNA
cgcaggcctctggcggagcccattccatgccagatgctgagcgatggctggtgtgtgctgctccacaggcctggtg

SEQ ID NO: 627 - RNA, 74 nt, Homo sapiens
  source 1..74: mol_type = transcribed RNA
gggaggaagaagggaggaggagcggaggggcccttgtcttcccagagcctctcccttcctcccctccccctccc

SEQ ID NO: 628 - RNA, 84 nt, Homo sapiens
  source 1..84: mol_type = transcribed RNA
cggtccagacgtggcgggggtggcggcggcatcccggacggcctgtgagggatgcgccgcccactgccccgcgccgcctgaccg

SEQ ID NO: 629 - RNA, 55 nt, Homo sapiens
  source 1..55: mol_type = transcribed RNA
gggggctggggcgcggggaggtgctaggtcggcctcggctcccgcgccgcacccc

SEQ ID NO: 630 - RNA, 110 nt, Homo sapiens
  source 1..110: mol_type = transcribed RNA
gtctaccaggtgtgggcccagctttacatagttcatgctgaggccgggatttcatgcagaaaactggttgcaaaaggtgctgaaggggctgggggagcacaagggagaag

SEQ ID NO: 631 - RNA, 73 nt, Homo sapiens
  source 1..73: mol_type = transcribed RNA
ctggtgtttgaggcgatgtggggatgtagagacaacttcccagtctcatttcctcatcctgccaggccaccat

SEQ ID NO: 632 - RNA, 92 nt, Homo sapiens
  source 1..92: mol_type = transcribed RNA
cgctgcgcttctgggcccgcggcgggcgtggggctgcccgggccggtcgaccagcgcgccgtagctcccgaggcccgagccgcgacccgcgg

SEQ ID NO: 633 - RNA, 66 nt, Homo sapiens
  source 1..66: mol_type = transcribed RNA
tcaagacggggagtcaggcagtggtggagatggagagccctgagcctccactctcctggcccccag

SEQ ID NO: 634 - RNA, 73 nt, Homo sapiens
  source 1..73: mol_type = transcribed RNA
gttcaagtgggaggacaggaggcaggtgtggttggaggaagcagcctgaacctgcctccctgacattccacag

SEQ ID NO: 635 - RNA, 61 nt, Homo sapiens
  source 1..61: mol_type = transcribed RNA
tcggctggcgggggtagagctggctgcaggcccggcccctctcagctgctgccctctccag

SEQ ID NO: 636 - RNA, 98 nt, Homo sapiens
  source 1..98: mol_type = transcribed RNA
cgaggtaggggcgtcccgggcgcgcgggcgggtcccaggctgggcccctcggaggccgggtgctcactgccccgtcccggcgcccgtgtctcctccag

SEQ ID NO: 637 - RNA, 61 nt, Homo sapiens
  source 1..61: mol_type = transcribed RNA
aactgcggggccagagcagagagcccttgcacaccaccagcctctcctccctgtgccccag

SEQ ID NO: 638 - RNA, 69 nt, Homo sapiens
  source 1..69: mol_type = transcribed RNA
ccgggcaggcaggtgtagggtggagcccactgtggctcctgactcagccctgctgccttcacctgccag

SEQ ID NO: 639 - RNA, 89 nt, Homo sapiens
  source 1..89: mol_type = transcribed RNA
gtcagcagtgccttagcagcacgtaaatattggcgttaagattctaaaattatctccagtattaactgtgctgctgaagtaaggttgac

SEQ ID NO: 640 - RNA, 81 nt, Homo sapiens
  source 1..81: mol_type = transcribed RNA
gttccactctagcagcacgtaaatattggcgtagtgaaatatatattaaacaccaatattactgtgctgctttagtgtgac

SEQ ID NO: 641 - RNA, 94 nt, Homo sapiens
  source 1..94: mol_type = transcribed RNA
ataaaggaagttaggctgaggggcagagagcgagacttttctattttccaaaagctcggtctgaggcccctcagtcttgcttcctaacccgcgc

SEQ ID NO: 642 - RNA, 72 nt, Homo sapiens
  source 1..72: mol_type = transcribed RNA
cttgggaatggcaaggaaaccgttaccattactgagtttagtaatggtaatggttctcttgctatacccaga

SEQ ID NO: 643 - RNA, 94 nt, Homo sapiens
  source 1..94: mol_type = transcribed RNA
cgggcagcgggtgccaggcacggtgtcagcaggcaacatggccgagaggccggggcctccgggcggcgccgtgtccgcgaccgcgtaccctgac

SEQ ID NO: 644 - RNA, 118 nt, Homo sapiens
  source 1..118: mol_type = transcribed RNA
gcaggtgaactggcaggccaggaagaggaggaagccctggaggggctggaggtgatggatgttttcctccggttctcagggctccacctctttcgggccgtagagccagggctggtgc

SEQ ID NO: 645 - RNA, 24 nt, Homo sapiens
  source 1..24: mol_type = transcribed RNA
gaggctgggaaggcaaagggacgt

SEQ ID NO: 646 - RNA, 15 nt, Homo sapiens
  source 1..15: mol_type = transcribed RNA
ctgggaaggcaaagg

SEQ ID NO: 647 - RNA, 24 nt, Homo sapiens
  source 1..24: mol_type = transcribed RNA
agacacatttggagagggaacctc

SEQ ID NO: 648 - RNA, 16 nt, Homo sapiens
  source 1..16: mol_type = transcribed RNA
agacacatttggagag

SEQ ID NO: 649 - RNA, 15 nt, Homo sapiens
  source 1..15: mol_type = transcribed RNA
gctgcagtgggggag

SEQ ID NO: 650 - RNA, 23 nt, Homo sapiens
  source 1..23: mol_type = transcribed RNA
agggagtagaagggtggggagca

SEQ ID NO: 651 - RNA, 16 nt, Homo sapiens
  source 1..16: mol_type = transcribed RNA
tagggagtagaagggt

SEQ ID NO: 652 - RNA, 17 nt, Homo sapiens
  source 1..17: mol_type = transcribed RNA
gcggggcggcaggggcc

SEQ ID NO: 653 - RNA, 15 nt, Homo sapiens
  source 1..15: mol_type = transcribed RNA
gggggcggggcggca

SEQ ID NO: 654 - RNA, 18 nt, Homo sapiens
  source 1..18: mol_type = transcribed RNA
gcacgggagctcaggtga

SEQ ID NO: 655 - RNA, 18 nt, Homo sapiens
  source 1..18: mol_type = transcribed RNA
gcggcggcggcggcagca

SEQ ID NO: 656 - RNA, 15 nt, Homo sapiens
  source 1..15: mol_type = transcribed RNA
gcgggcggcggcggc

SEQ ID NO: 657 - RNA, 19 nt, Homo sapiens
  source 1..19: mol_type = transcribed RNA
gtgggttggggcgggctct

SEQ ID NO: 658 - RNA, 19 nt, Homo sapiens
  source 1..19: mol_type = transcribed RNA
gtgggttggggcgggctct

SEQ ID NO: 659 - RNA, 19 nt, Homo sapiens
  source 1..19: mol_type = transcribed RNA
gggtgcgggccggcggggt

SEQ ID NO: 660 - RNA, 15 nt, Homo sapiens
  source 1..15: mol_type = transcribed RNA
tgcgggccggcgggg

SEQ ID NO: 661 - RNA, 22 nt, Homo sapiens
  source 1..22: mol_type = transcribed RNA
aaggatttagggacaggctttg

SEQ ID NO: 662 - RNA, 19 nt, Homo sapiens
  source 1..19: mol_type = transcribed RNA
caggaaggatttagggaca

SEQ ID NO: 663 - RNA, 22 nt, Homo sapiens
  source 1..22: mol_type = transcribed RNA
gttggtgggggagccatgagat

SEQ ID NO: 664 - RNA, 22 nt, Homo sapiens
  source 1..22: mol_type = transcribed RNA
ggggagccatgagataagagca

SEQ ID NO: 665 - RNA, 23 nt, Homo sapiens
  source 1..23: mol_type = transcribed RNA
tggggaaggcgtcagtgtcgggt

SEQ ID NO: 666 - RNA, 16 nt, Homo sapiens
  source 1..16: mol_type = transcribed RNA
tggggaaggcgtcagt

SEQ ID NO: 667 - RNA, 22 nt, Homo sapiens
  source 1..22: mol_type = transcribed RNA
tggcggagcccattccatgcca

SEQ ID NO: 668 - RNA, 21 nt, Homo sapiens
  source 1..21: mol_type = transcribed RNA
ctggcggagcccattccatgc

SEQ ID NO: 669 - RNA, 23 nt, Homo sapiens
  source 1..23: mol_type = transcribed RNA
aagggaggaggagcggaggggcc

SEQ ID NO: 670 - RNA, 15 nt, Homo sapiens
  source 1..15: mol_type = transcribed RNA
gggaggaggagcgga

SEQ ID NO: 671 - RNA, 15 nt, Homo sapiens
  source 1..15: mol_type = transcribed RNA
ggcgcggggaggtgc

SEQ ID NO: 672 - RNA, 15 nt, Homo sapiens
  source 1..15: mol_type = transcribed RNA
ggcgcggggaggtgc

SEQ ID NO: 673 - RNA, 21 nt, Homo sapiens
  source 1..21: mol_type = transcribed RNA
gaggcgatgtggggatgtaga

SEQ ID NO: 674 - RNA, 20 nt, Homo sapiens
  source 1..20: mol_type = transcribed RNA
cccagtctcatttcctcatc

SEQ ID NO: 675 - RNA, 25 nt, Homo sapiens
  source 1..25: mol_type = transcribed RNA
ttctgggcccgcggcgggcgtgggg

SEQ ID NO: 676 - RNA, 15 nt, Homo sapiens
  source 1..15: mol_type = transcribed RNA
cgcggcgggcgtggg

SEQ ID NO: 677 - RNA, 27 nt, Homo sapiens
  source 1..27: mol_type = transcribed RNA
tagcagcacgtaaatattggcgttaag

SEQ ID NO: 678 - RNA, 15 nt, Homo sapiens
  source 1..15: mol_type = transcribed RNA
cacgtaaatattggc

SEQ ID NO: 679 - RNA, 30 nt, Homo sapiens
  source 1..30: mol_type = transcribed RNA
tgaggggcagagagcgagacttttctattt

SEQ ID NO: 680 - RNA, 15 nt, Homo sapiens
  source 1..15: mol_type = transcribed RNA
cagagagcgagactt

SEQ ID NO: 681 - RNA, 27 nt, Homo sapiens
  source 1..27: mol_type = transcribed RNA
aaaccgttaccattactgagtttagta

SEQ ID NO: 682 - RNA, 15 nt, Homo sapiens
  source 1..15: mol_type = transcribed RNA
taccattactgagtt

SEQ ID NO: 683 - RNA, 25 nt, Homo sapiens
  source 1..25: mol_type = transcribed RNA
aggaagccctggaggggctggaggt

SEQ ID NO: 684 - RNA, 15 nt, Homo sapiens
  source 1..15: mol_type = transcribed RNA
aggaagaggaggaag

(19) United States
(12) Patent Application Publication
(10) Pub. No.: US 2026/0035752 A1
(43) Pub. Date: Feb. 5, 2026

(54) PROSTATE CANCER DETECTION KIT OR DEVICE, AND DETECTION METHOD

(71) Applicants: TORAY INDUSTRIES, INC., Tokyo (JP); NATIONAL CANCER CENTER, Tokyo (JP)

(72) Inventors: Satoshi KONDOU, Kamakura-shi (JP); Hitoshi NOBUMASA, Kamakura-shi (JP); Satoko KOZONO, Kamakura-shi (JP); Hiroko SUDO, Kamakura-shi (JP); Junpei KAWAUCHI, Kamakura-shi (JP); Takahiro OCHIYA, Tokyo (JP); Nobuyoshi KOSAKA, Tokyo (JP)

(73) Assignees: TORAY INDUSTRIES, INC., Tokyo (JP); NATIONAL CANCER CENTER, Tokyo (JP)

(21) Appl. No.: 19/364,495

(22) Filed: Oct. 21, 2025

Related U.S. Application Data
(62) Division of application No. 18/604,659, filed on Mar. 14, 2024, now Pat. No. 12,480,165.
Application No. 18/604,659 is a division of application No. 17/977,451, filed on Oct. 31, 2022, now Pat. No. 11,959,144.
Application No. 17/977,451 is a division of application No. 16/808,095, filed on Mar. 3, 2020, now Pat. No. 11,519,038.
Application No. 16/808,095 is a division of application No. 15/317,882, now Pat. No. 10,619,213.

(30) Foreign Application Priority Data
Jun. 12, 2014 (JP) 2014-121377
Mar. 31, 2015 (JP) 2015-071756

(51) Int. Cl. C12Q 1/6886; C12M 1/00; C12M 1/34; C12N 15/09; C12Q 1/68; G01N 33/53; G01N 33/574; G01N 37/00

(52) U.S. Cl. CPC C12Q 1/6886; C12M 1/00; C12M 1/34; C12N 15/09; C12Q 1/68; G01N 33/53; G01N 33/574; G01N 37/00; C12Q 2600/156; C12Q 2600/158; C12Q 2600/16; C12Q 2600/178

(57) ABSTRACT

An object of the present invention is to provide a kit or a device for the detection of prostate cancer and a method for detecting prostate cancer. The present invention provides a kit or a device for the detection of prostate cancer, comprising a nucleic acid capable of specifically binding to a miRNA in a sample of a subject, and a method for detecting prostate cancer, comprising measuring the miRNA in vitro.

### CROSS-REFERENCE TO RELATED APPLICATIONS

This application is a Divisional of application Ser. No. 18/604,659 filed Mar. 14, 2024, which is a Divisional of application Ser. No. 17/977,451 filed Oct. 31, 2022 (now U.S. Pat. No. 11,959,144), which is a Divisional of application Ser. No. 16/808,095 filed Mar. 3, 2020 (now U.S. Pat. No. 11,519,038), which is a Divisional of application Ser. No. 15/317,882, filed on Dec. 9, 2016 (now U.S. Pat. No. 10,619,213), which was filed as a national phase application of PCT International Application No. PCT/JP2015/066964 on Jun. 12, 2015, which claims the benefit under 35 U.S.C. § 119(a) to Patent Application No. 2014-121377, filed in Japan on Jun. 12, 2014 and Patent Application No. 2015-071756, filed in Japan on Mar. 31, 2015, all of which are hereby expressly incorporated by reference into the present application.

### REFERENCE TO ELECTRONIC SEQUENCE LISTING

The application contains a Sequence Listing which has been submitted electronically in .XML format and is hereby incorporated by reference in its entirety. Said .XML copy, created on Oct. 14, 2025, is named “PH-6233-PCT-US04.xml” and is 616,120 bytes in size. The sequence listing contained in this .XML file is part of the specification and is hereby incorporated by reference herein in its entirety.

### TECHNICAL FIELD

The present invention relates to a kit or a device for the detection of prostate cancer, comprising a nucleic acid capable of specifically binding to a particular miRNA, which is used for examining the presence or absence of prostate cancer in a subject, and a method for detecting prostate cancer, comprising measuring an expression level of the miRNA using the nucleic acid.

### BACKGROUND ART

The prostate is an organ that produces a component of the semen in males, and is positioned underneath the urinary bladder and in front of the rectum. Prostate cancer is a disease caused by the disorganized and repeated proliferation of cells of this prostate. According to the 2011 statistics of cancer type specific mortality in Japan disclosed by the Center for Cancer Control and Information Services, National Cancer Center, the number of individuals affected by prostate cancer was 51,534 people. Namely, it is estimated that one out of 14 Japanese males will experience prostate cancer. The number of incidences of this cancer in males takes the 4th place by cancer type. Also, the number of prostate cancer deaths climbed to 10,823 people and takes the 6th place by cancer type in males. It is estimated that one out of 7 American males will experience prostate cancer. Prostate cancer is particularly common in elderly people, and 6 out of 10 men aged 65 or older are diagnosed with prostate cancer (Non-Patent Literature 1). The estimated number of American individuals affected by prostate cancer climbed to 233,000 people in 2014, among which approximately 29,480 people reportedly died (Non-Patent Literature 1).

The progression stages of prostate cancer are specified in Non-Patent Literature 2 and classified into stage I (T1 to T2a/N0/M0), stage II (T2b to T2c/N0/M0), stage III (T3/N0/M0), and stage IV (T4/N0/M0 and N1 and cM1) according to tumor spread (T1a to T1c, T2a to T2c, T3a to T3b, and T4), lymph node metastasis (N0 and N1), distant metastasis (M0 and M1a to M1c), etc.

Since prostate cancer progresses relatively slowly in most cases, its 5-year relative survival rate is almost 100%, indicating one of cancers having the best prognosis (Non-Patent Literature 1). Some of prostate cancer cases, however, progress relatively fast and cause various disorders or symptoms. Prostate cancer found to have distant metastasis at stage 4 exhibits a 5-year relative survival rate as significantly low as 28% (Non-Patent Literature 1).

The treatment of prostate cancer in regular protocols includes surgical treatment, radiotherapy, endocrine therapy (hormone therapy), and palliative treatment which continues follow-up while monitoring a tumor marker PSA without special treatment. Particularly, the treatment of early prostate cancer has some options such as external beam radiotherapy, internal radiotherapy (brachytherapy), radical prostatectomy, and cryosurgery, in addition to palliative treatment (Non-Patent Literature 1).

As described in Non-Patent Literature 1, a test of PSA, a tumor marker in blood, is widely used as a primary test for prostate cancer. Rectal examination or transrectal ultrasonography of the prostate is carried out when the PSA measurement value is high. Biopsy is further carried out as definite diagnosis when a subject is suspected of having prostate cancer. An imaging test such as CT scan, MRI scan, or bone scintigraphy is also conducted when a subject is suspected of having distant metastasis.

The prostate-specific antigen (PSA) is produced by the prostate and contained in the semen, but is also present in blood, albeit slightly. The PSA concentration in blood of ordinary males is usually 4 ng/mL or lower, and a subject is suspected of having prostate cancer when the measurement value exceeds this reference value (Non-Patent Literature 1). The PSA concentration in blood is reportedly useful and widely implemented, for example, because this concentration elevates even in asymptomatic early prostate cancer and correlates with the stages of cancer progression. The American Cancer Society promotes the early detection of prostate cancer and recommends that subjects who desire screening of prostate cancer should undergo the PSA test (Non-Patent Literature 1).

As shown in Patent Literatures 1 to 3, there are reports, albeit at a research stage, on the detection of prostate cancer using the expression levels of microRNAs (miRNAs) or combinations of the expression levels of miRNAs and the expression levels of additional markers in biological samples including blood.

Patent Literature 1 discloses a method for detecting prostate cancer as well as Wilms tumor and COPD using hsa-miR-760, hsa-miR-920, has-miR-887-3p, hsa-miR-486-3p, hsa-miR-663b, hsa-miR-187-5p, hsa-miR-1231, hsa-miR-371a-5p, has-miR-575, hsa-miR-615-5p, hsa-miR-711, hsa-miR-939-5p, hsa-miR-1203, hsa-miR-1225-3p, hsa-miR-1225-5p, hsa-miR-1915-5p and the like in blood.

Patent Literature 2 discloses a method for detecting prostate cancer, etc., comprising isolating a vesicle from blood using EpCam and using a miRNA such as hsa-miR-92b-5p contained in the vesicle, for the detection.

Patent Literature 3 has reported that prostate cancer is determined by combining the expression level of PCA3 gene with the expression level of miR-141.

### CITATION LIST

### Patent Literature

Patent Literature 1: European Patent Application Publication No. 2341145
Patent Literature 2: International Publication No. WO 2013/022995
Patent Literature 3: International Publication No. WO 2010/062706

### Non-Patent Literature

Non-Patent Literature 1: American Cancer Society “Prostate Cancer”, 2013, p. 5, 14 to 26, 32 to 54, and 68 to 70
Non-Patent Literature 2: Sobin, L. et al., “TNM Classification of Malignant Tumours, the 7th edition”, 2010, p. 230 to 234
Non-Patent Literature 3: Wolf, A M. et al., 2010, A Cancer Journal for Clinicians, Vol. 60 (2), p. 70-98
Non-Patent Literature 4: Mitchell P S. et al., 2008, Proceedings of the National Academy of Sciences of the United States of America, Vol. 105 (30), p. 10513-10518

### SUMMARY OF INVENTION

### Technical Problem

An object of the present invention is to find a novel tumor marker for prostate cancer and to provide a method that can effectively detect prostate cancer using a nucleic acid capable of specifically binding to the marker. The PSA test is widely used as a tumor marker test for prostate cancer. The PSA test is, however, known that 15% of males having a PSA concentration in blood corresponding to the reference value 4 ng/mL or lower are confirmed to be prostate cancer-positive as a result of biopsy. On the other hand, it is also known that the PSA concentration in blood elevates in males having benign prostatic hyperplasia or prostatitis and in ordinary elderly men, leading to a high probability of false positives even in the absence of cancer (Non-Patent Literature 1). Furthermore, the false detection of a cancer other than prostate cancer also leads to false positives. Such a high probability of false positives in the PSA test leads to overdiagnosis and overtreatment, and various aftereffects ascribable to the unnecessary treatment of prostate cancer has been viewed as problems in recent years (Non-Patent Literature 3). According to the large-scale research using 5000 or more recruited subjects (Non-Patent Literature 3), the specific performance of the PSA test showed the sensitivity as low as 20.5% for the overall prostate cancer cases and the sensitivity of merely 51% even limited for highly malignant prostate cancer cases, suggesting that the tumor marker measurement is less significant as a preoperative test.

As described below, there are reports, albeit at a research stage, on the determination of prostate cancer using the expression levels of microRNAs (miRNAs) in biological samples including blood, none of which, however, have yet been brought into practical use.

Patent Literature 1 discloses a method for detecting prostate cancer as well as Wilms tumor and COPD using hsa-miR-760, hsa-miR-920, has-miR-887-3p, hsa-miR-486-3p, hsa-miR-663b, hsa-miR-187-5p, hsa-miR-1231, hsa-miR-371a-5p, has-miR-575, hsa-miR-615-5p, hsa-miR-711, hsa-miR-939-5p, hsa-miR-1203, hsa-miR-1225-3p, hsa-miR-1225-5p, hsa-miR-1915-5p and the like in blood. Patent Literature 1 describes many miRNAs, whereas this literature lacks a direct statement showing that these miRNA markers are markers for prostate cancer, and includes insufficient evidence for the usefulness of the miRNA markers as prostate cancer markers.

Patent Literature 2 discloses a method for detecting prostate cancer, etc., comprising isolating a vesicle from blood using EpCam and using a miRNA such as hsa-miR-92b-5p contained in the vesicle, for the detection. This literature, however, is less reliable because the miRNA marker was not reproducibly validated in an independent sample group and the literature has no mention about a threshold for detecting prostate cancer.

Patent Literature 3 specifically states that prostate cancer can be determined with 100% sensitivity and specificity by combining the expression levels of miR-141 and PCA3. This literature, however, does not state that prostate cancer can be determined conveniently and highly accurately using a single marker. In fact, Non-Patent Literature 4 is cited in Patent Literature 3. Non-Patent Literature 4 has reported the determination of prostate cancer using miR-141 in serum and states that the accuracy of the determination is 60% sensitivity when the specificity is 100%. In addition, a sample that is subjected to the PCA3 test currently used generally is urine, particularly, urine after digital rectal examination. On the other hand, the sample that is subjected to the determination of prostate cancer using miR-141 is blood (serum) as mentioned above. Thus, for obtaining highly sensitive and specific results by combining them, it is necessary to collect two samples.

### Solution to Problem

The present inventors have conducted diligent studies to attain the object and consequently completed the present invention by finding several genes usable as markers for the detection of prostate cancer from blood, which can be collected with limited invasiveness, and finding that prostate cancer can be significantly detected by using nucleic acids capable of specifically binding to any of these markers.

### SUMMARY OF INVENTION

Specifically, the present invention has the following features:
(1) A kit for the detection of prostate cancer, comprising a nucleic acid capable of specifically binding to at least one or more polynucleotide(s) selected from the group consisting of prostate cancer markers miR-4443, miR-1908-5p, miR-4257, miR-3197, miR-3188, miR-4649-5p, miR-1343-3p, miR-6861-5p, miR-1343-5p, miR-642b-3p, miR-6741-5p, miR-4745-5p, miR-6826-5p, miR-3663-3p, miR-3131, miR-92a-2-5p, miR-4258, miR-4448, miR-6125, miR-6880-5p, miR-6132, miR-4467, miR-6749-5p, miR-2392, miR-1273g-3p, miR-4746-3p, miR-1914-3p, miR-7845-5p, miR-6726-5p, miR-128-2-5p, miR-4651, miR-6765-3p, miR-3185, miR-4792, miR-6887-5p, miR-5572, miR-3619-3p, miR-6780b-5p, miR-4707-5p, miR-8063, miR-4454, miR-4525, miR-7975, miR-744-5p, miR-3135b, miR-4648, miR-6816-5p, miR-4741, miR-7150, miR-6791-5p, miR-1247-3p, miR-7977, miR-4497, miR-6090, miR-6781-5p, miR-6870-5p, miR-6729-5p, miR-4530, miR-7847-3p, miR-6825-5p, miR-4674, miR-3917, miR-4707-3p, miR-6885-5p, miR-6722-3p, miR-4516, miR-6757-5p, miR-6840-3p, miR-5195-3p, miR-6756-5p, miR-6800-5p, miR-6727-5p, miR-6126, miR-6872-3p, miR-4446-3p, miR-1268a, miR-1908-3p, miR-3679-5p, miR-4534, miR-4675, miR-7108-5p, miR-6799-5p, miR-4695-5p, miR-3178, miR-5090, miR-3180, miR-1237-5p, miR-4758-5p, miR-3184-5p, miR-4286, miR-6784-5p, miR-6768-5p, miR-6785-5p, miR-4706, miR-711, miR-1260a, miR-6746-5p, miR-6089, miR-6821-5p, miR-4667-5p, miR-8069, miR-4726-5p, miR-6124, miR-4532, miR-4486, miR-4728-5p, miR-4508, miR-128-1-5p, miR-4513, miR-6795-5p, miR-4689, miR-6763-5p, miR-8072, miR-6765-5p, miR-4419b, miR-7641, miR-3928-3p, miR-1227-5p, miR-4492, miR-296-3p, miR-6769a-5p, miR-6889-5p, miR-4632-5p, miR-4505, miR-3154, miR-3648, miR-4442, miR-3141, miR-7113-3p, miR-6819-5p, miR-3195, miR-1199-5p, miR-6738-5p, miR-4656, miR-6820-5p, miR-204-3p, miR-642a-3p, miR-762, miR-1202, miR-3162-5p, miR-3196, miR-3622a-5p, miR-3665, miR-3940-5p, miR-4294, miR-4466, miR-4476, miR-4723-5p, miR-4725-3p, miR-4730, miR-4739, miR-4787-5p, miR-5787, miR-6085, miR-6717-5p, miR-6724-5p, miR-6777-5p, miR-6778-5p, miR-6787-5p, miR-6789-5p, miR-6845-5p and miR-6893-5p.
(2) The kit according to (1), wherein miR-4443 is hsa-miR-4443, miR-1908-5p is hsa-miR-1908-5p, miR-4257 is hsa-miR-4257, miR-3197 is hsa-miR-3197, miR-3188 is hsa-miR-3188, miR-4649-5p is hsa-miR-4649-5p, miR-1343-3p is hsa-miR-1343-3p, miR-6861-5p is hsa-miR-6861-5p, miR-1343-5p is hsa-miR-1343-5p, miR-642b-3p is hsa-miR-642b-3p, miR-6741-5p is hsa-miR-6741-5p, miR-4745-5p is hsa-miR-4745-5p, miR-6826-5p is hsa-miR-6826-5p, miR-3663-3p is hsa-miR-3663-3p, miR-3131 is hsa-miR-3131, miR-92a-2-5p is hsa-miR-92a-2-5p, miR-4258 is hsa-miR-4258, miR-4448 is hsa-miR-4448, miR-6125 is hsa-miR-6125, miR-6880-5p is hsa-miR-6880-5p, miR-6132 is hsa-miR-6132, miR-4467 is hsa-miR-4467, miR-6749-5p is hsa-miR-6749-5p, miR-2392 is hsa-miR-2392, miR-1273g-3p is hsa-miR-1273g-3p, miR-4746-3p is hsa-miR-4746-3p, miR-1914-3p is hsa-miR-1914-3p, miR-7845-5p is hsa-miR-7845-5p, miR-6726-5p is hsa-miR-6726-5p, miR-128-2-5p is hsa-miR-128-2-5p, miR-4651 is hsa-miR-4651, miR-6765-3p is hsa-miR-6765-3p, miR-3185 is hsa-miR-3185, miR-4792 is hsa-miR-4792, miR-6887-5p is hsa-miR-6887-5p, miR-5572 is hsa-miR-5572, miR-3619-3p is hsa-miR-3619-3p, miR-6780b-5p is hsa-miR-6780b-5p, miR-4707-5p is hsa-miR-4707-5p, miR-8063 is hsa-miR-8063, miR-4454 is hsa-miR-4454, miR-4525 is hsa-miR-4525, miR-7975 is hsa-miR-7975, miR-744-5p is hsa-miR-744-5p, miR-3135b is hsa-miR-3135b, miR-4648 is hsa-miR-4648, miR-6816-5p is hsa-miR-6816-5p, miR-4741 is hsa-miR-4741, miR-7150 is hsa-miR-7150, miR-6791-5p is hsa-miR-6791-5p, miR-1247-3p is hsa-miR-1247-3p, miR-7977 is hsa-miR-7977, miR-4497 is hsa-miR-4497, miR-6090 is hsa-miR-6090, miR-6781-5p is hsa-miR-6781-5p, miR-6870-5p is hsa-miR-6870-5p, miR-6729-5p is hsa-miR-6729-5p, miR-4530 is hsa-miR-4530, miR-7847-3p is hsa-miR-7847-3p, miR-6825-5p is hsa-miR-6825-5p, miR-4674 is hsa-miR-4674, miR-3917 is hsa-miR-3917, miR-4707-3p is hsa-miR-4707-3p, miR-6885-5p is hsa-miR-6885-5p, miR-6722-3p is hsa-miR-6722-3p, miR-4516 is hsa-miR-4516, miR-6757-5p is hsa-miR-6757-5p, miR-6840-3p is hsa-miR-6840-3p, miR-5195-3p is hsa-miR-5195-3p, miR-6756-5p is hsa-miR-6756-5p, miR-6800-5p is hsa-miR-6800-5p, miR-6727-5p is hsa-miR-6727-5p, miR-6126 is hsa-miR-6126, miR-6872-3p is hsa-miR-6872-3p, miR-4446-3p is hsa-miR-4446-3p, miR-1268a is hsa-miR-1268a, miR-1908-3p is hsa-miR-1908-3p, miR-3679-5p is hsa-miR-3679-5p, miR-4534 is hsa-miR-4534, miR-4675 is hsa-miR-4675, miR-7108-5p is hsa-miR-7108-5p, miR-6799-5p is hsa-miR-6799-5p, miR-4695-5p is hsa-miR-4695-5p, miR-3178 is hsa-miR-3178, miR-5090 is hsa-miR-5090, miR-3180 is hsa-miR-3180, miR-1237-5p is hsa-miR-1237-5p, miR-4758-5p is hsa-miR-4758-5p, miR-3184-5p is hsa-miR-3184-5p, miR-4286 is hsa-miR-4286, miR-6784-5p is hsa-miR-6784-5p, miR-6768-5p is hsa-miR-6768-5p, miR-6785-5p is hsa-miR-6785-5p, miR-4706 is hsa-miR-4706, miR-711 is hsa-miR-711, miR-1260a is hsa-miR-1260a, miR-6746-5p is hsa-miR-6746-5p, miR-6089 is hsa-miR-6089, miR-6821-5p is hsa-miR-6821-5p, miR-4667-5p is hsa-miR-4667-5p, miR-8069 is hsa-miR-8069, miR-4726-5p is hsa-miR-4726-5p, miR-6124 is hsa-miR-6124, miR-4532 is hsa-miR-4532, miR-4486 is hsa-miR-4486, miR-4728-5p is hsa-miR-4728-5p, miR-4508 is hsa-miR-4508, miR-128-1-5p is hsa-miR-128-1-5p, miR-4513 is hsa-miR-4513, miR-6795-5p is hsa-miR-6795-5p, miR-4689 is hsa-miR-4689, miR-6763-5p is hsa-miR-6763-5p, miR-8072 is hsa-miR-8072, miR-6765-5p is hsa-miR-6765-5p, miR-4419b is hsa-miR-4419b, miR-7641 is hsa-miR-7641, miR-3928-3p is hsa-miR-3928-3p, miR-1227-5p is hsa-miR-1227-5p, miR-4492 is hsa-miR-4492, miR-296-3p is hsa-miR-296-3p, miR-6769a-5p is hsa-miR-6769a-5p, miR-6889-5p is hsa-miR-6889-5p, miR-4632-5p is hsa-miR-4632-5p, miR-4505 is hsa-miR-4505, miR-3154 is hsa-miR-3154, miR-3648 is hsa-miR-3648, miR-4442 is hsa-miR-4442, miR-3141 is hsa-miR-3141, miR-7113-3p is hsa-miR-7113-3p, miR-6819-5p is hsa-miR-6819-5p, miR-3195 is hsa-miR-3195, miR-1199-5p is hsa-miR-1199-5p, miR-6738-5p is hsa-miR-6738-5p, miR-4656 is hsa-miR-4656, miR-6820-5p is hsa-miR-6820-5p, miR-204-3p is hsa-miR-204-3p, miR-642a-3p is hsa-miR-642a-3p, miR-762 is hsa-miR-762, miR-1202 is hsa-miR-1202, miR-3162-5p is hsa-miR-3162-5p, miR-3196 is hsa-miR-3196, miR-3622a-5p is hsa-miR-3622a-5p, miR-3665 is hsa-miR-3665, miR-3940-5p is hsa-miR-3940-5p, miR-4294 is hsa-miR-4294, miR-4466 is hsa-miR-4466, miR-4476 is hsa-miR-4476, miR-4723-5p is hsa-miR-4723-5p, miR-4725-3p is hsa-miR-4725-3p, miR-4730 is hsa-miR-4730, miR-4739 is hsa-miR-4739, miR-4787-5p is hsa-miR-4787-5p, miR-5787 is hsa-miR-5787, miR-6085 is hsa-miR-6085, miR-6717-5p is hsa-miR-6717-5p, miR-6724-5p is hsa-miR-6724-5p, miR-6777-5p is hsa-miR-6777-5p, miR-6778-5p is hsa-miR-6778-5p, miR-6787-5p is hsa-miR-6787-5p, miR-6789-5p is hsa-miR-6789-5p, miR-6845-5p is hsa-miR-6845-5p, and miR-6893-5p is hsa-miR-6893-5p.
(3) The kit according to (1) or (2), wherein the nucleic acid is a polynucleotide selected from the group consisting of the following polynucleotides (a) to (e):
   (a) a polynucleotide consisting of a nucleotide sequence represented by any of SEQ ID NOs: 1 to 135 and 580 to 606 or a nucleotide sequence derived from the nucleotide sequence by the replacement of u with t, a variant thereof, a derivative thereof, or a fragment thereof comprising 15 or more consecutive nucleotides,
   (b) a polynucleotide comprising a nucleotide sequence represented by any of SEQ ID NOs: 1 to 135 and 580 to 606,
   (c) a polynucleotide consisting of a nucleotide sequence complementary to a nucleotide sequence represented by any of SEQ ID NOs: 1 to 135 and 580 to 606 or a nucleotide sequence derived from the nucleotide sequence by the replacement of u with t, a variant thereof, a derivative thereof, or a fragment thereof comprising 15 or more consecutive nucleotides,
   (d) a polynucleotide comprising a nucleotide sequence complementary to a nucleotide sequence represented by any of SEQ ID NOs: 1 to 135 and 580 to 606 or a nucleotide sequence derived from the nucleotide sequence by the replacement of u with t, and
   (e) a polynucleotide hybridizing under stringent conditions to any of the polynucleotides (a) to (d).
(4) The kit according to any of (1) to (3), wherein the kit further comprises a nucleic acid capable of specifically binding to at least one or more polynucleotide(s) selected from the group consisting of other prostate cancer markers miR-615-5p, miR-486-3p, miR-1225-3p, miR-760, miR-187-5p, miR-1203, miR-7110-5p, miR-371a-5p, miR-939-5p, miR-575, miR-92b-5p, miR-887-3p, miR-920, miR-1915-5p, miR-1231, miR-663b, miR-1225-5p, miR-16-5p, miR-423-5p, miR-451a, miR-564 and miR-671-5p.
(5) The kit according to (4), wherein miR-615-5p is hsa-miR-615-5p, miR-486-3p is hsa-miR-486-3p, miR-1225-3p is hsa-miR-1225-3p, miR-760 is hsa-miR-760, miR-187-5p is hsa-miR-187-5p, miR-1203 is hsa-miR-1203, miR-7110-5p is hsa-miR-7110-5p, miR-371a-5p is hsa-miR-371a-5p, miR-939-5p is hsa-miR-939-5p, miR-575 is hsa-miR-575, miR-92b-5p is hsa-miR-92b-5p, miR-887-3p is hsa-miR-887-3p, miR-920 is hsa-miR-920, miR-1915-5p is hsa-miR-1915-5p, miR-1231 is hsa-miR-1231, miR-663b is hsa-miR-663b, miR-1225-5p is hsa-miR-1225-5p, miR-16-5p is hsa-miR-16-5p, miR-423-5p is hsa-miR-423-5p, miR-451a is hsa-miR-451a, miR-564 is hsa-miR-564, and miR-671-5p is hsa-miR-671-5p.
(6) The kit according to (4) or (5), wherein the nucleic acid is a polynucleotide selected from the group consisting of the following polynucleotides (f) to (j):
   (f) a polynucleotide consisting of a nucleotide sequence represented by any of SEQ ID NOs: 136 to 152 and 607 to 611 or a nucleotide sequence derived from the nucleotide sequence by the replacement of u with t, any variant thereof, any derivative thereof, or any fragment thereof comprising 15 or more consecutive nucleotides,
   (g) a polynucleotide comprising a nucleotide sequence represented by any of SEQ ID NOs: 136 to 152 and 607 to 611,
   (h) a polynucleotide consisting of a nucleotide sequence complementary to a nucleotide sequence represented by any of SEQ ID NOs: 136 to 152 and 607 to 611 or a nucleotide sequence derived from the nucleotide sequence by the replacement of u with t, a variant thereof, a derivative thereof, or a fragment thereof comprising 15 or more consecutive nucleotides,
   (i) a polynucleotide comprising a nucleotide sequence complementary to a nucleotide sequence represented by any of SEQ ID NOs: 136 to 152 and 607 to 611 or a nucleotide sequence derived from the nucleotide sequence by the replacement of u with t, and
   (j) a polynucleotide hybridizing under stringent conditions to any of the polynucleotides (f) to (i).
(7) The kit according to any of (1) to (6), wherein the kit further comprises a nucleic acid capable of specifically binding to at least one or more polynucleotide(s) selected from the group consisting of other prostate cancer markers miR-4763-3p, miR-3656, miR-4488, miR-125a-3p, miR-1469, miR-1228-5p, miR-6798-5p, miR-1268b, miR-6732-5p, miR-1915-3p, miR-4433b-3p, miR-1207-5p, miR-4433-3p, miR-6879-5p, miR-4417, miR-30c-1-3p, miR-4638-5p, miR-6088, miR-4270, miR-6782-5p, miR-665, miR-486-5p, miR-4655-5p, miR-1275, miR-6806-5p, miR-614, miR-3937, miR-6752-5p, miR-6771-5p, miR-4450, miR-211-3p, miR-663a, miR-6842-5p, miR-7114-5p and miR-6779-5p.
(8) The kit according to (7), wherein miR-4763-3p is hsa-miR-4763-3p, miR-3656 is hsa-miR-3656, miR-4488 is hsa-miR-4488, miR-125a-3p is hsa-miR-125a-3p, miR-1469 is hsa-miR-1469, miR-1228-5p is hsa-miR-1228-5p, miR-6798-5p is hsa-miR-6798-5p, miR-1268b is hsa-miR-1268b, miR-6732-5p is hsa-miR-6732-5p, miR-1915-3p is hsa-miR-1915-3p, miR-4433b-3p is hsa-miR-4433b-3p, miR-1207-5p is hsa-miR-1207-5p, miR-4433-3p is hsa-miR-4433-3p, miR-6879-5p is hsa-miR-6879-5p, miR-4417 is hsa-miR-4417, miR-30c-1-3p is hsa-miR-30c-1-3p, miR-4638-5p is hsa-miR-4638-5p, miR-6088 is hsa-miR-6088, miR-4270 is hsa-miR-4270, miR-6782-5p is hsa-miR-6782-5p, miR-665 is hsa-miR-665, miR-486-5p is hsa-miR-486-5p, miR-4655-5p is hsa-miR-4655-5p, miR-1275 is hsa-miR-1275, miR-6806-5p is hsa-miR-6806-5p, miR-614 is hsa-miR-614, miR-3937 is hsa-miR-3937, miR-6752-5p is hsa-miR-6752-5p, miR-6771-5p is hsa-miR-6771-5p, miR-4450 is hsa-miR-4450, miR-211-3p is hsa-miR-211-3p, miR-663a is hsa-miR-663a, miR-6842-5p is hsa-miR-6842-5p, miR-7114-5p is hsa-miR-7114-5p, and miR-6779-5p is hsa-miR-6779-5p.
(9) The kit according to (7) or (8), wherein the nucleic acid is a polynucleotide selected from the group consisting of the following polynucleotides (k) to (o):
   (k) a polynucleotide consisting of a nucleotide sequence represented by any of SEQ ID NOs: 153 to 187 or a nucleotide sequence derived from the nucleotide sequence by the replacement of u with t, a variant thereof, a derivative thereof, or a fragment thereof comprising 15 or more consecutive nucleotides,
   (l) a polynucleotide comprising a nucleotide sequence represented by any of SEQ ID NOs: 153 to 187,
   (m) a polynucleotide consisting of a nucleotide sequence complementary to a nucleotide sequence represented by any of SEQ ID NOs: 153 to 187 or a nucleotide sequence derived from the nucleotide sequence by the replacement of u with t, a variant thereof, a derivative thereof, or a fragment thereof comprising 15 or more consecutive nucleotides,
   (n) a polynucleotide comprising a nucleotide sequence complementary to a nucleotide sequence represented by any of SEQ ID NOs: 153 to 187 or a nucleotide sequence derived from the nucleotide sequence by the replacement of u with t, and
   (o) a polynucleotide hybridizing under stringent conditions to any of the polynucleotides (k) to (n).
(10) The kit according to any of (1) to (9), wherein the kit comprises at least two or more nucleic acids capable of specifically binding to at least two or more polynucleotides, respectively, selected from all of the prostate cancer markers according to (1) or (2).
(11) A device for the detection of prostate cancer, comprising a nucleic acid capable of specifically binding to at least one or more polynucleotide(s) selected from the group consisting of prostate cancer markers miR-4443, miR-1908-5p, miR-4257, miR-3197, miR-3188, miR-4649-5p, miR-1343-3p, miR-6861-5p, miR-1343-5p, miR-642b-3p, miR-6741-5p, miR-4745-5p, miR-6826-5p, miR-3663-3p, miR-3131, miR-92a-2-5p, miR-4258, miR-4448, miR-6125, miR-6880-5p, miR-6132, miR-4467, miR-6749-5p, miR-2392, miR-1273g-3p, miR-4746-3p, miR-1914-3p, miR-7845-5p, miR-6726-5p, miR-128-2-5p, miR-4651, miR-6765-3p, miR-3185, miR-4792, miR-6887-5p, miR-5572, miR-3619-3p, miR-6780b-5p, miR-4707-5p, miR-8063, miR-4454, miR-4525, miR-7975, miR-744-5p, miR-3135b, miR-4648, miR-6816-5p, miR-4741, miR-7150, miR-6791-5p, miR-1247-3p, miR-7977, miR-4497, miR-6090, miR-6781-5p, miR-6870-5p, miR-6729-5p, miR-4530, miR-7847-3p, miR-6825-5p, miR-4674, miR-3917, miR-4707-3p, miR-6885-5p, miR-6722-3p, miR-4516, miR-6757-5p, miR-6840-3p, miR-5195-3p, miR-6756-5p, miR-6800-5p, miR-6727-5p, miR-6126, miR-6872-3p, miR-4446-3p, miR-1268a, miR-1908-3p, miR-3679-5p, miR-4534, miR-4675, miR-7108-5p, miR-6799-5p, miR-4695-5p, miR-3178, miR-5090, miR-3180, miR-1237-5p, miR-4758-5p, miR-3184-5p, miR-4286, miR-6784-5p, miR-6768-5p, miR-6785-5p, miR-4706, miR-711, miR-1260a, miR-6746-5p, miR-6089, miR-6821-5p, miR-4667-5p, miR-8069, miR-4726-5p, miR-6124, miR-4532, miR-4486, miR-4728-5p, miR-4508, miR-128-1-5p, miR-4513, miR-6795-5p, miR-4689, miR-6763-5p, miR-8072, miR-6765-5p, miR-4419b, miR-7641, miR-3928-3p, miR-1227-5p, miR-4492, miR-296-3p, miR-6769a-5p, miR-6889-5p, miR-4632-5p, miR-4505, miR-3154, miR-3648, miR-4442, miR-3141, miR-7113-3p, miR-6819-5p, miR-3195, miR-1199-5p, miR-6738-5p, miR-4656, miR-6820-5p, miR-204-3p, miR-642a-3p, miR-762, miR-1202, miR-3162-5p, miR-3196, miR-3622a-5p, miR-3665, miR-3940-5p, miR-4294, miR-4466, miR-4476, miR-4723-5p, miR-4725-3p, miR-4730, miR-4739, miR-4787-5p, miR-5787, miR-6085, miR-6717-5p, miR-6724-5p, miR-6777-5p, miR-6778-5p, miR-6787-5p, miR-6789-5p, miR-6845-5p and miR-6893-5p.
(12) The device according to (11), wherein miR-4443 is hsa-miR-4443, miR-1908-5p is hsa-miR-1908-5p, miR-4257 is hsa-miR-4257, miR-3197 is hsa-miR-3197, miR-3188 is hsa-miR-3188, miR-4649-5p is hsa-miR-4649-5p, miR-1343-3p is hsa-miR-1343-3p, miR-6861-5p is hsa-miR-6861-5p, miR-1343-5p is hsa-miR-1343-5p, miR-642b-3p is hsa-miR-642b-3p, miR-6741-5p is hsa-miR-6741-5p, miR-4745-5p is hsa-miR-4745-5p, miR-6826-5p is hsa-miR-6826-5p, miR-3663-3p is hsa-miR-3663-3p, miR-3131 is hsa-miR-3131, miR-92a-2-5p is hsa-miR-92a-2-5p, miR-4258 is hsa-miR-4258, miR-4448 is hsa-miR-4448, miR-6125 is hsa-miR-6125, miR-6880-5p is hsa-miR-6880-5p, miR-6132 is hsa-miR-6132, miR-4467 is hsa-miR-4467, miR-6749-5p is hsa-miR-6749-5p, miR-2392 is hsa-miR-2392, miR-1273g-3p is hsa-miR-1273g-3p, miR-4746-3p is hsa-miR-4746-3p, miR-1914-3p is hsa-miR-1914-3p, miR-7845-5p is hsa-miR-7845-5p, miR-6726-5p is hsa-miR-6726-5p, miR-128-2-5p is hsa-miR-128-2-5p, miR-4651 is hsa-miR-4651, miR-6765-3p is hsa-miR-6765-3p, miR-3185 is hsa-miR-3185, miR-4792 is hsa-miR-4792, miR-6887-5p is hsa-miR-6887-5p, miR-5572 is hsa-miR-5572, miR-3619-3p is hsa-miR-3619-3p, miR-6780b-5p is hsa-miR-6780b-5p, miR-4707-5p is hsa-miR-4707-5p, miR-8063 is hsa-miR-8063, miR-4454 is hsa-miR-4454, miR-4525 is hsa-miR-4525, miR-7975 is hsa-miR-7975, miR-744-5p is hsa-miR-744-5p, miR-3135b is hsa-miR-3135b, miR-4648 is hsa-miR-4648, miR-6816-5p is hsa-miR-6816-5p, miR-4741 is hsa-miR-4741, miR-7150 is hsa-miR-7150, miR-6791-5p is hsa-miR-6791-5p, miR-1247-3p is hsa-miR-1247-3p, miR-7977 is hsa-miR-7977, miR-4497 is hsa-miR-4497, miR-6090 is hsa-miR-6090, miR-6781-5p is hsa-miR-6781-5p, miR-6870-5p is hsa-miR-6870-5p, miR-6729-5p is hsa-miR-6729-5p, miR-4530 is hsa-miR-4530, miR-7847-3p is hsa-miR-7847-3p, miR-6825-5p is hsa-miR-6825-5p, miR-4674 is hsa-miR-4674, miR-3917 is hsa-miR-3917, miR-4707-3p is hsa-miR-4707-3p, miR-6885-5p is hsa-miR-6885-5p, miR-6722-3p is hsa-miR-6722-3p, miR-4516 is hsa-miR-4516, miR-6757-5p is hsa-miR-6757-5p, miR-6840-3p is hsa-miR-6840-3p, miR-5195-3p is hsa-miR-5195-3p, miR-6756-5p is hsa-miR-6756-5p, miR-6800-5p is hsa-miR-6800-5p, miR-6727-5p is hsa-miR-6727-5p, miR-6126 is hsa-miR-6126, miR-6872-3p is hsa-miR-6872-3p, miR-4446-3p is hsa-miR-4446-3p, miR-1268a is hsa-miR-1268a, miR-1908-3p is hsa-miR-1908-3p, miR-3679-5p is hsa-miR-3679-5p, miR-4534 is hsa-miR-4534, miR-4675 is hsa-miR-4675, miR-7108-5p is hsa-miR-7108-5p, miR-6799-5p is hsa-miR-6799-5p, miR-4695-5p is hsa-miR-4695-5p, miR-3178 is hsa-miR-3178, miR-5090 is hsa-miR-5090, miR-3180 is hsa-miR-3180, miR-1237-5p is hsa-miR-1237-5p, miR-4758-5p is hsa-miR-4758-5p, miR-3184-5p is hsa-miR-3184-5p, miR-4286 is hsa-miR-4286, miR-6784-5p is hsa-miR-6784-5p, miR-6768-5p is hsa-miR-6768-5p, miR-6785-5p is hsa-miR-6785-5p, miR-4706 is hsa-miR-4706, miR-711 is hsa-miR-711, miR-1260a is hsa-miR-1260a, miR-6746-5p is hsa-miR-6746-5p, miR-6089 is hsa-miR-6089, miR-6821-5p is hsa-miR-6821-5p, miR-4667-5p is hsa-miR-4667-5p, miR-8069 is hsa-miR-8069, miR-4726-5p is hsa-miR-4726-5p, miR-6124 is hsa-miR-6124, miR-4532 is hsa-miR-4532, miR-4486 is hsa-miR-4486, miR-4728-5p is hsa-miR-4728-5p, miR-4508 is hsa-miR-4508, miR-128-1-5p is hsa-miR-128-1-5p, miR-4513 is hsa-miR-4513, miR-6795-5p is hsa-miR-6795-5p, miR-4689 is hsa-miR-4689, miR-6763-5p is hsa-miR-6763-5p, miR-8072 is hsa-miR-8072, miR-6765-5p is hsa-miR-6765-5p, miR-4419b is hsa-miR-4419b, miR-7641 is hsa-miR-7641, miR-3928-3p is hsa-miR-3928-3p, miR-1227-5p is hsa-miR-1227-5p, miR-4492 is hsa-miR-4492, miR-296-3p is hsa-miR-296-3p, miR-6769a-5p is hsa-miR-6769a-5p, miR-6889-5p is hsa-miR-6889-5p, miR-4632-5p is hsa-miR-4632-5p, miR-4505 is hsa-miR-4505, miR-3154 is hsa-miR-3154, miR-3648 is hsa-miR-3648, miR-4442 is hsa-miR-4442, miR-3141 is hsa-miR-3141, miR-7113-3p is hsa-miR-7113-3p, miR-6819-5p is hsa-miR-6819-5p, miR-3195 is hsa-miR-3195, miR-1199-5p is hsa-miR-1199-5p, miR-6738-5p is hsa-miR-6738-5p, miR-4656 is hsa-miR-4656, miR-6820-5p is hsa-miR-6820-5p, miR-204-3p is hsa-miR-204-3p, miR-642a-3p is hsa-miR-642a-3p, miR-762 is hsa-miR-762, miR-1202 is hsa-miR-1202, miR-3162-5p is hsa-miR-3162-5p, miR-3196 is hsa-miR-3196, miR-3622a-5p is hsa-miR-3622a-5p, miR-3665 is hsa-miR-3665, miR-3940-5p is hsa-miR-3940-5p, miR-4294 is hsa-miR-4294, miR-4466 is hsa-miR-4466, miR-4476 is hsa-miR-4476, miR-4723-5p is hsa-miR-4723-5p, miR-4725-3p is hsa-miR-4725-3p, miR-4730 is hsa-miR-4730, miR-4739 is hsa-miR-4739, miR-4787-5p is hsa-miR-4787-5p, miR-5787 is hsa-miR-5787, miR-6085 is hsa-miR-6085, miR-6717-5p is hsa-miR-6717-5p, miR-6724-5p is hsa-miR-6724-5p, miR-6777-5p is hsa-miR-6777-5p, miR-6778-5p is hsa-miR-6778-5p, miR-6787-5p is hsa-miR-6787-5p, miR-6789-5p is hsa-miR-6789-5p, miR-6845-5p is hsa-miR-6845-5p, and miR-6893-5p is hsa-miR-6893-5p.
(13) The device according to (11) or (12), wherein the nucleic acid is a polynucleotide selected from the group consisting of the following polynucleotides (a) to (e):
   (a) a polynucleotide consisting of a nucleotide sequence represented by any of SEQ ID NOs: 1 to 135 and 580 to 606 or a nucleotide sequence derived from the nucleotide sequence by the replacement of u with t, a variant thereof, a derivative thereof, or a fragment thereof comprising 15 or more consecutive nucleotides,
   (b) a polynucleotide comprising a nucleotide sequence represented by any of SEQ ID NOs: 1 to 135 and 580 to 606,
   (c) a polynucleotide consisting of a nucleotide sequence complementary to a nucleotide sequence represented by any of SEQ ID NOs: 1 to 135 and 580 to 606 or a nucleotide sequence derived from the nucleotide sequence by the replacement of u with t, a variant thereof, a derivative thereof, or a fragment thereof comprising 15 or more consecutive nucleotides,
   (d) a polynucleotide comprising a nucleotide sequence complementary to a nucleotide sequence represented by any of SEQ ID NOs: 1 to 135 and 580 to 606 or a nucleotide sequence derived from the nucleotide sequence by the replacement of u with t, and
   (e) a polynucleotide hybridizing under stringent conditions to any of the polynucleotides (a) to (d).
(14) The device according to any of (11) to (13), wherein the device further comprises a nucleic acid capable of specifically binding to at least one or more polynucleotide(s) selected from the group consisting of other prostate cancer markers miR-615-5p, miR-486-3p, miR-1225-3p, miR-760, miR-187-5p, miR-1203, miR-7110-5p, miR-371a-5p, miR-939-5p, miR-575, miR-92b-5p, miR-887-3p, miR-920, miR-1915-5p, miR-1231, miR-663b, miR-1225-5p, miR-16-5p, miR-423-5p, miR-451a, miR-564, and miR-671-5p.
(15) The device according to (14), wherein miR-615-5p is hsa-miR-615-5p, miR-486-3p is hsa-miR-486-3p, miR-1225-3p is hsa-miR-1225-3p, miR-760 is hsa-miR-760, miR-187-5p is hsa-miR-187-5p, miR-1203 is hsa-miR-1203, miR-7110-5p is hsa-miR-7110-5p, miR-371a-5p is hsa-miR-371a-5p, miR-939-5p is hsa-miR-939-5p, miR-575 is hsa-miR-575, miR-92b-5p is hsa-miR-92b-5p, miR-887-3p is hsa-miR-887-3p, miR-920 is hsa-miR-920, miR-1915-5p is hsa-miR-1915-5p, miR-1231 is hsa-miR-1231, miR-663b is hsa-miR-663b, miR-1225-5p is hsa-miR-1225-5p, miR-16-5p is hsa-miR-16-5p, miR-423-5p is hsa-miR-423-5p, miR-451a is hsa-miR-451a, miR-564 is hsa-miR-564, and miR-671-5p is hsa-miR-671-5p.
(16) The device according to (14) or (15), wherein the nucleic acid is a polynucleotide selected from the group consisting of the following polynucleotides (f) to (j):
   (f) a polynucleotide consisting of a nucleotide sequence represented by any of SEQ ID NOs: 136 to 152 and 607 to 611 or a nucleotide sequence derived from the nucleotide sequence by the replacement of u with t, a variant thereof, a derivative thereof, or a fragment thereof comprising 15 or more consecutive nucleotides,
   (g) a polynucleotide comprising a nucleotide sequence represented by any of SEQ ID NOs: 136 to 152 and 607 to 611,
   (h) a polynucleotide consisting of a nucleotide sequence complementary to a nucleotide sequence represented by any of SEQ ID NOs: 136 to 152 and 607 to 611 or a nucleotide sequence derived from the nucleotide sequence by the replacement of u with t, a variant thereof, a derivative thereof, or a fragment thereof comprising 15 or more consecutive nucleotides,
   (i) a polynucleotide comprising a nucleotide sequence complementary to a nucleotide sequence represented by any of SEQ ID NOs: 136 to 152 and 607 to 611 or a nucleotide sequence derived from the nucleotide sequence by the replacement of u with t, and
   (j) a polynucleotide hybridizing under stringent conditions to any of the polynucleotides (f) to (i).
(17) The device according to any of (11) to (16), wherein the device further comprises a nucleic acid capable of specifically binding to at least one or more polynucleotide(s) selected from the group consisting of other prostate cancer markers miR-4763-3p, miR-3656, miR-4488, miR-125a-3p, miR-1469, miR-1228-5p, miR-6798-5p, miR-1268b, miR-6732-5p, miR-1915-3p, miR-4433b-3p, miR-1207-5p, miR-4433-3p, miR-6879-5p, miR-4417, miR-30c-1-3p, miR-4638-5p, miR-6088, miR-4270, miR-6782-5p, miR-665, miR-486-5p, miR-4655-5p, miR-1275, miR-6806-5p, miR-614, miR-3937, miR-6752-5p, miR-6771-5p, miR-4450, miR-211-3p, miR-663a, miR-6842-5p, miR-7114-5p and miR-6779-5p.
(18) The device according to (17), wherein miR-4763-3p is hsa-miR-4763-3p, miR-3656 is hsa-miR-3656, miR-4488 is hsa-miR-4488, miR-125a-3p is hsa-miR-125a-3p, miR-1469 is hsa-miR-1469, miR-1228-5p is hsa-miR-1228-5p, miR-6798-5p is hsa-miR-6798-5p, miR-1268b is hsa-miR-1268b, miR-6732-5p is hsa-miR-6732-5p, miR-1915-3p is hsa-miR-1915-3p, miR-4433b-3p is hsa-miR-4433b-3p, miR-1207-5p is hsa-miR-1207-5p, miR-4433-3p is hsa-miR-4433-3p, miR-6879-5p is hsa-miR-6879-5p, miR-4417 is hsa-miR-4417, miR-30c-1-3p is hsa-miR-30c-1-3p, miR-4638-5p is hsa-miR-4638-5p, miR-6088 is hsa-miR-6088, miR-4270 is hsa-miR-4270, miR-6782-5p is hsa-miR-6782-5p, miR-665 is hsa-miR-665, miR-486-5p is hsa-miR-486-5p, miR-4655-5p is hsa-miR-4655-5p, miR-1275 is hsa-miR-1275, miR-6806-5p is hsa-miR-6806-5p, miR-614 is hsa-miR-614, miR-3937 is hsa-miR-3937, miR-6752-5p is hsa-miR-6752-5p, miR-6771-5p is hsa-miR-6771-5p, miR-4450 is hsa-miR-4450, miR-211-3p is hsa-miR-211-3p, miR-663a is hsa-miR-663a, miR-6842-5p is hsa-miR-6842-5p, miR-7114-5p is hsa-miR-7114-5p, and miR-6779-5p is hsa-miR-6779-5p.
(19) The device according to (17) or (18), wherein the nucleic acid is a polynucleotide selected from the group consisting of the following polynucleotides (k) to (o):
   (k) a polynucleotide consisting of a nucleotide sequence represented by any of SEQ ID NOs: 153 to 187 or a nucleotide sequence derived from the nucleotide sequence by the replacement of u with t, a variant thereof, a derivative thereof, or a fragment thereof comprising 15 or more consecutive nucleotides,
   (l) a polynucleotide comprising a nucleotide sequence represented by any of SEQ ID NOs: 153 to 187,
   (m) a polynucleotide consisting of a nucleotide sequence complementary to a nucleotide sequence represented by any of SEQ ID NOs: 153 to 187 or a nucleotide sequence derived from the nucleotide sequence by the replacement of u with t, a variant thereof, a derivative thereof, or a fragment thereof comprising 15 or more consecutive nucleotides,
   (n) a polynucleotide comprising a nucleotide sequence complementary to a nucleotide sequence represented by any of SEQ ID NOs: 153 to 187 or a nucleotide sequence derived from the nucleotide sequence by the replacement of u with t, and
   (o) a polynucleotide hybridizing under stringent conditions to any of the polynucleotides (k) to (n).
(20) The device according to any one of (11) to (19), wherein the device is a device for measurement by a hybridization technique.
(21) The device according to (20), wherein the hybridization technique is a nucleic acid array technique.
(22) The device according to any one of (11) to (21), wherein the device comprises at least two or more nucleic acids capable of specifically binding to at least two or more polynucleotides, respectively, selected from all of the prostate cancer markers according to (11) or (12).
(23) A method for detecting prostate cancer, comprising measuring an expression level of a target nucleic acid in a sample from a subject using a kit according to any one of (1) to (10) or a device according to any one of (11) to (22), and evaluating in vitro whether or not the subject has prostate cancer using the measured expression level and a control expression level in a sample from a healthy subject measured in the same way as above.
(24) The method according to (23), wherein the subject is a human.
(25) The method according to (23) or (24), wherein the sample is blood, serum, or plasma.

### Definition of Term

The terms used in the present specification are defined as follows.

Abbreviations or terms such as nucleotide, polynucleotide, DNA, and RNA abide by “Guidelines for the preparation of specification which contain nucleotide and/or amino acid sequences” (edited by Japan Patent Office) and common use in the art.

In the present specification, the term “polynucleotide” is used for a nucleic acid including all of RNA, DNA, and RNA/DNA (chimera). The DNA includes all of cDNA, genomic DNA, and synthetic DNA. The RNA includes all of total RNA, mRNA, rRNA, miRNA, siRNA, snoRNA, snRNA, non-coding RNA and synthetic RNA. In the present specification, the “synthetic DNA” and the “synthetic RNA” refer to a DNA and an RNA artificially prepared using, for example, an automatic nucleic acid synthesizer, on the basis of predetermined nucleotide sequences (which may be any of natural and non-natural sequences). In the present specification, the “non-natural sequence” is intended to be used in a broad sense and includes, for example, a sequence containing substitution, deletion, insertion, and/or addition of one or more nucleotide(s) (i.e., a variant sequence) and a sequence containing one or more modified nucleotide(s) (i.e., a modified sequence), which are different from the natural sequence. In the present specification, the polynucleotide is used interchangeably with a nucleic acid.

In the present specification, the term “fragment” is a polynucleotide having a nucleotide sequence having a consecutive portion of a polynucleotide and desirably has a length of 15 or more nucleotides, preferably 17 or more nucleotides, more preferably 19 or more nucleotides.

In the present specification, the term “gene” is intended to include not only RNA and double-stranded DNA but also each single-stranded DNA such as a plus strand (or a sense strand) or a complementary strand (or an antisense strand) constituting the duplex. The gene is not particularly limited by its length.

Thus, in the present specification, the “gene” includes all of double-stranded DNA including human genomic DNA, single-stranded DNA (plus strand) including cDNA, single-stranded DNA having a sequence complementary to the plus strand (complementary strand), microRNA (miRNA), and their fragments, and their transcripts, unless otherwise specified. The “gene” includes not only a “gene” represented by a particular nucleotide sequence (or SEQ ID NO) but “nucleic acids” encoding RNAs having biological functions equivalent to an RNA encoded by the gene, for example, a congener (i.e., a homolog or an ortholog), a variant (e.g., a genetic polymorph), and a derivative. Specific examples of such a “nucleic acid” encoding a congener, a variant, or a derivative can include a “nucleic acid” having a nucleotide sequence hybridizing under stringent conditions described later to a complementary sequence of a nucleotide sequence represented by any of SEQ ID NOs: 1 to 684 or a nucleotide sequence derived from the nucleotide sequence by the replacement of u with t. The “gene” is not particularly limited by its functional region and can contain, for example, an expression control region, a coding region, an exon, or an intron. The “gene” may be contained in a cell or may exist alone after being released into the outside of a cell. Alternatively, the “gene” may be in a state enclosed in a vesicle called exosome.

In the present specification, the term “exosome” is a vesicle that is capsulated with a lipid bilayer and secreted from a cell. The exosome is derived from a multivesicular endosome and may incorporate a biomaterial such as a “gene” (e.g., RNA or DNA) or a protein when released into an extracellular environment. The exosome is known to be contained in a body fluid such as blood, serum, plasma, serum, or lymph.

In the present specification, the term “transcript” refers to an RNA synthesized with the DNA sequence of a gene as a template. RNA polymerase binds to a site called promoter located upstream of the gene and adds ribonucleotides complementary to the nucleotide sequence of the DNA to the 3′ end to synthesize an RNA. This RNA contains not only the gene itself but also the whole sequence from a transcription initiation site to the end of a poly A sequence, including an expression regulatory region, a coding region, an exon, or an intron.

In the present specification, the term “microRNA (miRNA)” is intended to mean a 15- to 25-nucleotide non-coding RNA that is transcribed as an RNA precursor having a hairpin-like structure, cleaved by a dsRNA-cleaving enzyme which has RNase III cleavage activity, integrated into a protein complex called RISC, and involved in the suppression of translation of mRNA, unless otherwise specified. The term “miRNA” used in the present specification includes not only a “miRNA” represented by a particular nucleotide sequence (or SEQ ID NO) but a precursor of the “miRNA” (pre-miRNA or pri-miRNA), and miRNAs having biological functions equivalent thereto, for example, a congener (i.e., a homolog or an ortholog), a variant (e.g., a genetic polymorph), and a derivative. Such a precursor, a congener, a variant, or a derivative can be specifically identified using miRBase Release 20 (http://www.mirbase.org/), and examples thereof can include a “miRNA” having a nucleotide sequence hybridizing under stringent conditions described later to a complementary sequence of any particular nucleotide sequence represented by any of SEQ ID NOs: 1 to 684. The term “miRNA” used in the present specification may be a gene product of a miR gene. Such a gene product includes a mature miRNA (e.g., a 15- to 25-nucleotide or 19- to 25-nucleotide non-coding RNA involved in the suppression of translation of mRNA as described above) or a miRNA precursor (e.g., pre-miRNA or pri-miRNA as described above).

In the present specification, the term “probe” includes a polynucleotide that is used for specifically detecting an RNA resulting from the expression of a gene or a polynucleotide derived from the RNA, and/or a polynucleotide complementary thereto.

In the present specification, the term “primer” includes a polynucleotide that specifically recognizes and amplifies an RNA resulting from the expression of a gene or a polynucleotide derived from the RNA, and/or a polynucleotide complementary thereto.

In this context, the complementary polynucleotide (complementary strand or reverse strand) means a polynucleotide in a complementary base relationship based on A:T (U) and G:C base pairs with the full-length sequence of a polynucleotide consisting of a nucleotide sequence defined by any of SEQ ID NOs: 1 to 684 or a nucleotide sequence derived from the nucleotide sequence by the replacement of u with t, or a partial sequence thereof (here, this full-length or partial sequence is referred to as a plus strand for the sake of convenience). However, such a complementary strand is not limited to a sequence completely complementary to the nucleotide sequence of the target plus strand and may have a complementary relationship to an extent that permits hybridization under stringent conditions to the target plus strand.

In the present specification, the term “stringent conditions” refers to conditions under which a nucleic acid probe hybridizes to its target sequence to a larger extent (e.g., a measurement value equal to or larger than a mean of background measurement values+a standard deviation of the background measurement values×2) than that for other sequences. The stringent conditions are dependent on a sequence and differ depending on an environment where hybridization is performed. A target sequence complementary 100% to the nucleic acid probe can be identified by controlling the stringency of hybridization and/or washing conditions. Specific examples of the “stringent conditions” will be mentioned later.

In the present specification, the term “Tm value” means a temperature at which the double-stranded moiety of a polynucleotide is denatured into single strands so that the double strands and the single strands exist at a ratio of 1:1.

In the present specification, the term “variant” means, in the case of a nucleic acid, a natural variant attributed to polymorphism, mutation, or the like; a variant containing the deletion, substitution, addition, or insertion of 1, 2, or 3 or more nucleotides in a nucleotide sequence represented by any of SEQ ID NOs: 1 to 684 or a nucleotide sequence derived from the nucleotide sequence by the replacement of u with t, or a partial sequence thereof, a variant containing the deletion, substitution, addition, or insertion of 1 or 2 or more nucleotides in a nucleotide sequence of a premature miRNA of a sequence represented by any of SEQ ID NOs: 1 to 684 or a nucleotide sequence derived from the nucleotide sequence by the replacement of u with t, or a partial sequence thereof, a variant that exhibits % identity of approximately 90% or higher, approximately 95% or higher, approximately 97% or higher, approximately 98% or higher, approximately 99% or higher to each of these nucleotide sequences or the partial sequences thereof, or a nucleic acid hybridizing under the stringent conditions defined above to a polynucleotide or an oligonucleotide comprising each of these nucleotide sequences or the partial sequences thereof.

In the present specification, the term “several” means an integer of approximately 10, 9, 8, 7, 6, 5, 4, 3, or 2.

In the present specification, the variant can be prepared by use of a well-known technique such as site-directed mutagenesis or PCR-based mutagenesis.

In the present specification, the term “percent (%) identity” can be determined with or without an introduced gap, using a protein or gene search system based on BLAST or FASTA described above (Zheng Zhang et al., 2000, J. Comput. Biol., Vol. 7, p. 203-214; Altschul, S. F. et al., 1990, Journal of Molecular Biology, Vol. 215, p. 403-410; and Pearson, W. R. et al., 1988, Proc. Natl. Acad. Sci. U.S.A, Vol. 85, p. 2444-2448).

In the present specification, the term “derivative” is meant to include a modified nucleic acid, for example, a derivative labeled with a fluorophore or the like, a derivative containing a modified nucleotide (e.g., a nucleotide containing a group such as halogen, alkyl such as methyl, alkoxy such as methoxy, thio, or carboxymethyl, and a nucleotide that has undergone base rearrangement, double bond saturation, deamination, replacement of an oxygen molecule with a sulfur atom, etc.), PNA (peptide nucleic acid; Nielsen, P. E. et al., 1991, Science, Vol. 254, p. 1497-500), and LNA (locked nucleic acid; Obika, S. et al., 1998, Tetrahedron Lett., Vol. 39, p. 5401-5404) without any limitation.

In the present specification, the “nucleic acid” capable of specifically binding to a polynucleotide selected from the prostate cancer marker miRNAs described above is a synthesized or prepared nucleic acid and specifically includes a “nucleic acid probe” or a “primer”. The “nucleic acid” is utilized directly or indirectly for detecting the presence or absence of prostate cancer in a subject, for diagnosing the severity, the degree of amelioration, or the therapeutic sensitivity of prostate cancer, or for screening for a candidate substance useful in the prevention, amelioration, or treatment of prostate cancer. The “nucleic acid” includes a nucleotide, an oligonucleotide, and a polynucleotide capable of specifically recognizing and binding to a transcript represented by any of SEQ ID NOs: 1 to 684, or a synthetic cDNA nucleic acid thereof in vivo, particularly, in a sample such as a body fluid (e.g., blood or urine), in relation to the development of prostate cancer. The nucleotide, the oligonucleotide, and the polynucleotide can be effectively used as probes for detecting the aforementioned gene expressed in vivo, in tissues, in cells, or the like on the basis of the properties described above, or as primers for amplifying the aforementioned gene expressed in vivo.

The term “detection” used in the present specification is interchangeable with the term “examination”, “measurement”, or “detection or decision support”. In the present specification, the term “evaluation” is meant to include diagnosis or evaluation support on the basis of examination results or measurement results.

The term “subject” used in the present specification means a mammal such as a primate including a human and a chimpanzee, a pet animal including a dog and a cat, a livestock animal including cattle, a horse, sheep, and a goat, and a rodent including a mouse and a rat. The term “healthy subject” also means such a mammal without the cancer to be detected.

The term “P” or “P value” used in the present specification refers to a probability at which a more extreme statistic than that actually calculated from data under a null hypothesis is observed in a statistical test. Thus, smaller “P” or “P value” means more significant difference between subjects to be compared.

In the present specification, the term “sensitivity” means a value of (the number of true positives)/(the number of true positives+the number of false negatives). High sensitivity allows prostate cancer to be detected early, leading to the complete resection of cancer sites and reduction in the rate of recurrence.

In the present specification, the term “specificity” means a value of (the number of true negatives)/(the number of true negatives+the number of false positives). High specificity prevents needless extra examination for healthy subjects misjudged as being prostate cancer patients, leading to reduction in burden on patients and reduction in medical expense.

In the present specification, the term “accuracy” means a value of (the number of true positives+the number of true negatives)/(the total number of cases). The accuracy indicates the ratio of samples that were correctly identified to all samples and serves as a primary index to evaluate detection performance.

In the present specification, the “sample” that is subjected to determination, detection, or diagnosis refers to a tissue and a biological material in which the expression of the gene of the present invention varies as prostate cancer develops, prostate cancer progresses, and therapeutic effects on prostate cancer are exerted. Specifically, the “sample” refers to a prostatic tissue, a periprostatic vascular channel, lymph node, and organ, an organ suspected of having metastasis, the skin, a body fluid such as blood, urine, saliva, sweat, or tissue exudates, serum or plasma prepared from blood, feces, hair, and the like. The “sample” further refers to a biological sample extracted therefrom, specifically, a gene such as RNA or miRNA.

The term “hsa-miR-4443 gene” or “hsa-miR-4443” used in the present specification includes the hsa-miR-4443 gene (miRBase Accession No. MIMAT0018961) described in SEQ ID NO: 1, a homolog or an ortholog of a different organism species, and the like. The hsa-miR-4443 gene can be obtained by a method described in Jima D D et al., 2010, Blood, Vol. 116, e118-e127. Also, “hsa-mir-4443” (miRBase Accession No. MI0016786, SEQ ID NO: 188) having a hairpin-like structure is known as a precursor of “hsa-miR-4443”.

The term “hsa-miR-1908-5p gene” or “hsa-miR-1908-5p” used in the present specification includes the hsa-miR-1908-5p gene (miRBase Accession No. MIMAT0007881) described in SEQ ID NO: 2, a homolog or an ortholog of a different organism species, and the like. The hsa-miR-1908-5p gene can be obtained by a method described in Bar M et al., 2008, Stem Cells, Vol. 26, p. 2496-2505. Also, “hsa-mir-1908” (miRBase Accession No. MI0008329, SEQ ID NO: 189) having a hairpin-like structure is known as a precursor of “hsa-miR-1908-5p”.

The term “hsa-miR-4257 gene” or “hsa-miR-4257” used in the present specification includes the hsa-miR-4257 gene (miRBase Accession No. MIMAT0016878) described in SEQ ID NO: 3, a homolog or an ortholog of a different organism species, and the like. The hsa-miR-4257 gene can be obtained by a method described in Goff L A et al., 2009, PLoS One, Vol. 4, e7192. Also, “hsa-mir-4257” (miRBase Accession No. MI0015856, SEQ ID NO: 190) having a hairpin-like structure is known as a precursor of “hsa-miR-4257”.

The term “hsa-miR-3197 gene” or “hsa-miR-3197” used in the present specification includes the hsa-miR-3197 gene (miRBase Accession No. MIMAT0015082) described in SEQ ID NO: 4, a homolog or an ortholog of a different organism species, and the like. The hsa-miR-3197 gene can be obtained by a method described in Stark M S et al., 2010, PLoS One, Vol. 5, e9685. Also, “hsa-mir-3197” (miRBase Accession No. MI0014245, SEQ ID NO: 191) having a hairpin-like structure is known as a precursor of “hsa-miR-3197”.

The term “hsa-miR-3188 gene” or “hsa-miR-3188” used in the present specification includes the hsa-miR-3188 gene (miRBase Accession No. MIMAT0015070) described in SEQ ID NO: 5, a homolog or an ortholog of a different organism species, and the like. The hsa-miR-3188 gene can be obtained by a method described in Stark M S et al., 2010, PLoS One, Vol. 5, e9685. Also, “hsa-mir-3188” (miRBase Accession No. MI0014232, SEQ ID NO: 192) having a hairpin-like structure is known as a precursor of “hsa-miR-3188”.

The term “hsa-miR-4649-5p gene” or “hsa-miR-4649-5p” used in the present specification includes the hsa-miR-4649-5p gene (miRBase Accession No. MIMAT0019711) described in SEQ ID NO: 6, a homolog or an ortholog of a different organism species, and the like. The hsa-miR-4649-5p gene can be obtained by a method described in Persson H et al., 2011, CancerRes, Vol. 71, p. 78-86. Also, “hsa-mir-4649” (miRBase Accession No. MI0017276, SEQ ID NO: 193) having a hairpin-like structure is known as a precursor of “hsa-miR-4649-5p”.

The term “hsa-miR-1343-3p gene” or “hsa-miR-1343-3p” used in the present specification includes the hsa-miR-1343-3p gene (miRBase Accession No. MIMAT0019776) described in SEQ ID NO: 7, a homolog or an ortholog of a different organism species, and the like. The hsa-miR-1343-3p gene can be obtained by a method described in Persson H et al., 2011, CancerRes, Vol. 71, p. 78-86. Also, “hsa-mir-1343” (miRBase Accession No. MI0017320, SEQ ID NO: 194) having a hairpin-like structure is known as a precursor of “hsa-miR-1343-3p”.

The term “hsa-miR-6861-5p gene” or “hsa-miR-6861-5p” used in the present specification includes the hsa-miR-6861-5p gene (miRBase Accession No. MIMAT0027623) described in SEQ ID NO: 8, a homolog or an ortholog of a different organism species, and the like. The hsa-miR-6861-5p gene can be obtained by a method described in Ladewig E et al., 2012, Genome Res, Vol. 22, p. 1634-1645. Also, “hsa-mir-6861” (miRBase Accession No. MI0022708, SEQ ID NO: 195) having a hairpin-like structure is known as a precursor of “hsa-miR-6861-5p”.

The term “hsa-miR-1343-5p gene” or “hsa-miR-1343-5p” used in the present specification includes the hsa-miR-1343-5p gene (miRBase Accession No. MIMAT0027038) described in SEQ ID NO: 9, a homolog or an ortholog of a different organism species, and the like. The hsa-miR-1343-5p gene can be obtained by a method described in Persson H et al., 2011, CancerRes, Vol. 71, p. 78-86. Also, “hsa-mir-1343” (miRBase Accession No. MI0017320, SEQ ID NO: 194) having a hairpin-like structure is known as a precursor of “hsa-miR-1343-5p”.

The term “hsa-miR-642b-3p gene” or “hsa-miR-642b-3p” used in the present specification includes the hsa-miR-642b-3p gene (miRBase Accession No. MIMAT0018444) described in SEQ ID NO: 10, a homolog or an ortholog of a different organism species, and the like. The hsa-miR-642b-3p gene can be obtained by a method described in Witten D et al., 2010, BMC Biol, Vol. 8, p. 58. Also, “hsa-mir-642b” (miRBase Accession No. MI0016685, SEQ ID NO: 196) having a hairpin-like structure is known as a precursor of “hsa-miR-642b-3p”.

The term “hsa-miR-6741-5p gene” or “hsa-miR-6741-5p” used in the present specification includes the hsa-miR-6741-5p gene (miRBase Accession No. MIMAT0027383) described in SEQ ID NO: 11, a homolog or an ortholog of a different organism species, and the like. The hsa-miR-6741-5p gene can be obtained by a method described in Ladewig E et al., 2012, Genome Res, Vol. 22, p. 1634-1645. Also, “hsa-mir-6741” (miRBase Accession No. MI0022586, SEQ ID NO: 197) having a hairpin-like structure is known as a precursor of “hsa-miR-6741-5p”.

The term “hsa-miR-4745-5p gene” or “hsa-miR-4745-5p” used in the present specification includes the hsa-miR-4745-5p gene (miRBase Accession No. MIMAT0019878) described in SEQ ID NO: 12, a homolog or an ortholog of a different organism species, and the like. The hsa-miR-4745-5p gene can be obtained by a method described in Persson H et al., 2011, Cancer Res, Vol. 71, p. 78-86. Also, “hsa-mir-4745” (miRBase Accession No. MI0017384, SEQ ID NO: 198) having a hairpin-like structure is known as a precursor of “hsa-miR-4745-5p”.

The term “hsa-miR-6826-5p gene” or “hsa-miR-6826-5p” used in the present specification includes the hsa-miR-6826-5p gene (miRBase Accession No. MIMAT0027552) described in SEQ ID NO: 13, a homolog or an ortholog of a different organism species, and the like. The hsa-miR-6826-5p gene can be obtained by a method described in Ladewig E et al., 2012, Genome Res, Vol. 22, p. 1634-1645. Also, “hsa-mir-6826” (miRBase Accession No. MI0022671, SEQ ID NO: 199) having a hairpin-like structure is known as a precursor of “hsa-miR-6826-5p”.

The term “hsa-miR-3663-3p gene” or “hsa-miR-3663-3p” used in the present specification includes the hsa-miR-3663-3p gene (miRBase Accession No. MIMAT0018085) described in SEQ ID NO: 14, a homolog or an ortholog of a different organism species, and the like. The hsa-miR-3663-3p gene can be obtained by a method described in Liao J Y et al., 2010, PLoS One, Vol. 5, e10563. Also, “hsa-mir-3663” (miRBase Accession No. MI0016064, SEQ ID NO: 200) having a hairpin-like structure is known as a precursor of “hsa-miR-3663-3p”.

The term “hsa-miR-3131 gene” or “hsa-miR-3131” used in the present specification includes the hsa-miR-3131 gene (miRBase Accession No. MIMAT0014996) described in SEQ ID NO: 15, a homolog or an ortholog of a different organism species, and the like. The hsa-miR-3131 gene can be obtained by a method described in Stark M S et al., 2010, PLoS One, Vol. 5, e9685. Also, “hsa-mir-3131” (miRBase Accession No. MI0014151, SEQ ID NO: 201) having a hairpin-like structure is known as a precursor of “hsa-miR-3131”.

The term “hsa-miR-92a-2-5p gene” or “hsa-miR-92a-2-5p” used in the present specification includes the hsa-miR-92a-2-5p gene (miRBase Accession No. MIMAT0004508) described in SEQ ID NO: 16, a homolog or an ortholog of a different organism species, and the like. The hsa-miR-92a-2-5p gene can be obtained by a method described in Mourelatos Z et al., 2002, Genes Dev, Vol. 16, p. 720-728. Also, “hsa-mir-92a-2” (miRBase Accession No. MI0000094, SEQ ID NO: 202) having a hairpin-like structure is known as a precursor of “hsa-miR-92a-2-5p”.

The term “hsa-miR-4258 gene” or “hsa-miR-4258” used in the present specification includes the hsa-miR-4258 gene (miRBase Accession No. MIMAT0016879) described in SEQ ID NO: 17, a homolog or an ortholog of a different organism species, and the like. The hsa-miR-4258 gene can be obtained by a method described in Goff L A et al., 2009, PLoS One, Vol. 4, e7192. Also, “hsa-mir-4258” (miRBase Accession No. MI0015857, SEQ ID NO: 203) having a hairpin-like structure is known as a precursor of “hsa-miR-4258”.

The term “hsa-miR-4448 gene” or “hsa-miR-4448” used in the present specification includes the hsa-miR-4448 gene (miRBase Accession No. MIMAT0018967) described in SEQ ID NO: 18, a homolog or an ortholog of a different organism species, and the like. The hsa-miR-4448 gene can be obtained by a method described in Jima D D et al., 2010, Blood, Vol. 116, e118-e127. Also, “hsa-mir-4448” (miRBase Accession No. MI0016791, SEQ ID NO: 204) having a hairpin-like structure is known as a precursor of “hsa-miR-4448”.

The term “hsa-miR-6125 gene” or “hsa-miR-6125” used in the present specification includes the hsa-miR-6125 gene (miRBase Accession No. MIMAT0024598) described in SEQ ID NO: 19, a homolog or an ortholog of a different organism species, and the like. The hsa-miR-6125 gene can be obtained by a method described in Smith J L et al., 2012, J Virol, Vol. 86, p. 5278-5287. Also, “hsa-mir-6125” (miRBase Accession No. MI0021259, SEQ ID NO: 205) having a hairpin-like structure is known as a precursor of “hsa-miR-6125”.

The term “hsa-miR-6880-5p gene” or “hsa-miR-6880-5p” used in the present specification includes the hsa-miR-6880-5p gene (miRBase Accession No. MIMAT0027660) described in SEQ ID NO: 20, a homolog or an ortholog of a different organism species, and the like. The hsa-miR-6880-5p gene can be obtained by a method described in Ladewig E et al., 2012, Genome Res, Vol. 22, p. 1634-1645. Also, “hsa-mir-6880” (miRBase Accession No. MI0022727, SEQ ID NO: 206) having a hairpin-like structure is known as a precursor of “hsa-miR-6880-5p”.

The term “hsa-miR-6132 gene” or “hsa-miR-6132” used in the present specification includes the hsa-miR-6132 gene (miRBase Accession No. MIMAT0024616) described in SEQ ID NO: 21, a homolog or an ortholog of a different organism species, and the like. The hsa-miR-6132 gene can be obtained by a method described in Dannemann M et al., 2012, Genome Biol Evol, Vol. 4, p. 552-564. Also, “hsa-mir-6132” (miRBase Accession No. MI0021277, SEQ ID NO: 207) having a hairpin-like structure is known as a precursor of “hsa-miR-6132”.

The term “hsa-miR-4467 gene” or “hsa-miR-4467” used in the present specification includes the hsa-miR-4467 gene (miRBase Accession No. MIMAT0018994) described in SEQ ID NO: 22, a homolog or an ortholog of a different organism species, and the like. The hsa-miR-4467 gene can be obtained by a method described in Jima D D et al., 2010, Blood, Vol. 116, e118-e127. Also, “hsa-mir-4467” (miRBase Accession No. MI0016818, SEQ ID NO: 208) having a hairpin-like structure is known as a precursor of “hsa-miR-4467”.

The term “hsa-miR-6749-5p gene” or “hsa-miR-6749-5p” used in the present specification includes the hsa-miR-6749-5p gene (miRBase Accession No. MIMAT0027398) described in SEQ ID NO: 23, a homolog or an ortholog of a different organism species, and the like. The hsa-miR-6749-5p gene can be obtained by a method described in Ladewig E et al., 2012, Genome Res, Vol. 22, p. 1634-1645. Also, “hsa-mir-6749” (miRBase Accession No. MI0022594, SEQ ID NO: 209) having a hairpin-like structure is known as a precursor of “hsa-miR-6749-5p”.

The term “hsa-miR-2392 gene” or “hsa-miR-2392” used in the present specification includes the hsa-miR-2392 gene (miRBase Accession No. MIMAT0019043) described in SEQ ID NO: 24, a homolog or an ortholog of a different organism species, and the like. The hsa-miR-2392 gene can be obtained by a method described in Jima D D et al., 2010, Blood, Vol. 116, e118-e127. Also, “hsa-mir-2392” (miRBase Accession No. MI0016870, SEQ ID NO: 210) having a hairpin-like structure is known as a precursor of “hsa-miR-2392”.

The term “hsa-miR-1273g-3p gene” or “hsa-miR-1273g-3p” used in the present specification includes the hsa-miR-1273g-3p gene (miRBase Accession No. MIMAT0022742) described in SEQ ID NO: 25, a homolog or an ortholog of a different organism species, and the like. The hsa-miR-1273g-3p gene can be obtained by a method described in Reshmi G et al., 2011, Genomics, Vol. 97, p. 333-340. Also, “hsa-mir-1273g” (miRBase Accession No. MI0018003, SEQ ID NO: 211) having a hairpin-like structure is known as a precursor of “hsa-miR-1273g-3p”.

The term “hsa-miR-4746-3p gene” or “hsa-miR-4746-3p” used in the present specification includes the hsa-miR-4746-3p gene (miRBase Accession No. MIMAT0019881) described in SEQ ID NO: 26, a homolog or an ortholog of a different organism species, and the like. The hsa-miR-4746-3p gene can be obtained by a method described in Persson H et al., 2011, CancerRes, Vol. 71, p. 78-86. Also, “hsa-mir-4746” (miRBase Accession No. MI0017385, SEQ ID NO: 212) having a hairpin-like structure is known as a precursor of “hsa-miR-4746-3p”.

The term “hsa-miR-1914-3p gene” or “hsa-miR-1914-3p” used in the present specification includes the hsa-miR-1914-3p gene (miRBase Accession No. MIMAT0007890) described in SEQ ID NO: 27, a homolog or an ortholog of a different organism species, and the like. The hsa-miR-1914-3p gene can be obtained by a method described in Bar M et al., 2008, Stem Cells, Vol. 26, p. 2496-2505. Also, “hsa-mir-1914” (miRBase Accession No. MI0008335, SEQ ID NO: 213) having a hairpin-like structure is known as a precursor of “hsa-miR-1914-3p”.

The term “hsa-miR-7845-5p gene” or “hsa-miR-7845-5p” used in the present specification includes the hsa-miR-7845-5p gene (miRBase Accession No. MIMAT0030420) described in SEQ ID NO: 28, a homolog or an ortholog of a different organism species, and the like. The hsa-miR-7845-5p gene can be obtained by a method described in Ple H et al., 2012, PLoS One, Vol. 7, e50746. Also, “hsa-mir-7845” (miRBase Accession No. MI0025515, SEQ ID NO: 214) having a hairpin-like structure is known as a precursor of “hsa-miR-7845-5p”.

The term “hsa-miR-6726-5p gene” or “hsa-miR-6726-5p” used in the present specification includes the hsa-miR-6726-5p gene (miRBase Accession No. MIMAT0027353) described in SEQ ID NO: 29, a homolog or an ortholog of a different organism species, and the like. The hsa-miR-6726-5p gene can be obtained by a method described in Ladewig E et al., 2012, Genome Res, Vol. 22, p. 1634-1645. Also, “hsa-mir-6726” (miRBase Accession No. MI0022571, SEQ ID NO: 215) having a hairpin-like structure is known as a precursor of “hsa-miR-6726-5p”.

The term “hsa-miR-128-2-5p gene” or “hsa-miR-128-2-5p” used in the present specification includes the hsa-miR-128-2-5p gene (miRBase Accession No. MIMAT0031095) described in SEQ ID NO: 30, a homolog or an ortholog of a different organism species, and the like. The hsa-miR-128-2-5p gene can be obtained by a method described in Lagos-Quintana M et al., 2002, Curr Biol, Vol. 12, p. 735-739. Also, “hsa-mir-128-2” (miRBase Accession No. MI0000727, SEQ ID NO: 216) having a hairpin-like structure is known as a precursor of “hsa-miR-128-2-5p”.

The term “hsa-miR-4651 gene” or “hsa-miR-4651” used in the present specification includes the hsa-miR-4651 gene (miRBase Accession No. MIMAT0019715) described in SEQ ID NO: 31, a homolog or an ortholog of a different organism species, and the like. The hsa-miR-4651 gene can be obtained by a method described in Persson H et al., 2011, Cancer Res, Vol. 71, p. 78-86. Also, “hsa-mir-4651” (miRBase Accession No. MI0017279, SEQ ID NO: 217) having a hairpin-like structure is known as a precursor of “hsa-miR-4651”.

The term “hsa-miR-6765-3p gene” or “hsa-miR-6765-3p” used in the present specification includes the hsa-miR-6765-3p gene (miRBase Accession No. MIMAT0027431) described in SEQ ID NO: 32, a homolog or an ortholog of a different organism species, and the like. The hsa-miR-6765-3p gene can be obtained by a method described in Ladewig E et al., 2012, Genome Res, Vol. 22, p. 1634-1645. Also, “hsa-mir-6765” (miRBase Accession No. MI0022610, SEQ ID NO: 218) having a hairpin-like structure is known as a precursor of “hsa-miR-6765-3p”.

The term “hsa-miR-3185 gene” or “hsa-miR-3185” used in the present specification includes the hsa-miR-3185 gene (miRBase Accession No. MIMAT0015065) described in SEQ ID NO: 33, a homolog or an ortholog of a different organism species, and the like. The hsa-miR-3185 gene can be obtained by a method described in Stark M S et al., 2010, PLoS One, Vol. 5, e9685. Also, “hsa-mir-3185” (miRBase Accession No. MI0014227, SEQ ID NO: 219) having a hairpin-like structure is known as a precursor of “hsa-miR-3185”.

The term “hsa-miR-4792 gene” or “hsa-miR-4792” used in the present specification includes the hsa-miR-4792 gene (miRBase Accession No. MIMAT0019964) described in SEQ ID NO: 34, a homolog or an ortholog of a different organism species, and the like. The hsa-miR-4792 gene can be obtained by a method described in Persson H et al., 2011, Cancer Res, Vol. 71, p. 78-86. Also, “hsa-mir-4792” (miRBase Accession No. MI0017439, SEQ ID NO: 220) having a hairpin-like structure is known as a precursor of “hsa-miR-4792”.

The term “hsa-miR-6887-5p gene” or “hsa-miR-6887-5p” used in the present specification includes the hsa-miR-6887-5p gene (miRBase Accession No. MIMAT0027674) described in SEQ ID NO: 35, a homolog or an ortholog of a different organism species, and the like. The hsa-miR-6887-5p gene can be obtained by a method described in Ladewig E et al., 2012, Genome Res, Vol. 22, p. 1634-1645. Also, “hsa-mir-6887” (miRBase Accession No. MI0022734, SEQ ID NO: 221) having a hairpin-like structure is known as a precursor of “hsa-miR-6887-5p”.

The term “hsa-miR-5572 gene” or “hsa-miR-5572” used in the present specification includes the hsa-miR-5572 gene (miRBase Accession No. MIMAT0022260) described in SEQ ID NO: 36, a homolog or an ortholog of a different organism species, and the like. The hsa-miR-5572 gene can be obtained by a method described in Tandon M et al., 2012, Oral Dis, Vol. 18, p. 127-131. Also, “hsa-mir-5572” (miRBase Accession No. MI0019117, SEQ ID NO: 222) having a hairpin-like structure is known as a precursor of “hsa-miR-5572”.

The term “hsa-miR-3619-3p gene” or “hsa-miR-3619-3p” used in the present specification includes the hsa-miR-3619-3p gene (miRBase Accession No. MIMAT0019219) described in SEQ ID NO: 37, a homolog or an ortholog of a different organism species, and the like. The hsa-miR-3619-3p gene can be obtained by a method described in Witten D et al., 2010, BMC Biol, Vol. 8, p. 58. Also, “hsa-mir-3619” (miRBase Accession No. MI0016009, SEQ ID NO: 223) having a hairpin-like structure is known as a precursor of “hsa-miR-3619-3p”.

The term “hsa-miR-6780b-5p gene” or “hsa-miR-6780b-5p” used in the present specification includes the hsa-miR-6780b-5p gene (miRBase Accession No. MIMAT0027572) described in SEQ ID NO: 38, a homolog or an ortholog of a different organism species, and the like. The hsa-miR-6780b-5p gene can be obtained by a method described in Ladewig E et al., 2012, Genome Res, Vol. 22, p. 1634-1645. Also, “hsa-mir-6780b” (miRBase Accession No. MI0022681, SEQ ID NO: 224) having a hairpin-like structure is known as a precursor of “hsa-miR-6780b-5p”.

The term “hsa-miR-4707-5p gene” or “hsa-miR-4707-5p” used in the present specification includes the hsa-miR-4707-5p gene (miRBase Accession No. MIMAT0019807) described in SEQ ID NO: 39, a homolog or an ortholog of a different organism species, and the like. The hsa-miR-4707-5p gene can be obtained by a method described in Persson H et al., 2011, Cancer Res, Vol. 71, p. 78-86. Also, “hsa-mir-4707” (miRBase Accession No. MI0017340, SEQ ID NO: 225) having a hairpin-like structure is known as a precursor of “hsa-miR-4707-5p”.

The term “hsa-miR-8063 gene” or “hsa-miR-8063” used in the present specification includes the hsa-miR-8063 gene (miRBase Accession No. MIMAT0030990) described in SEQ ID NO: 40, a homolog or an ortholog of a different organism species, and the like. The hsa-miR-8063 gene can be obtained by a method described in Wang H J et al., 2013, Shock, Vol. 39, p. 480-487. Also, “hsa-mir-8063” (miRBase Accession No. MI0025899, SEQ ID NO: 226) having a hairpin-like structure is known as a precursor of “hsa-miR-8063”.

The term “hsa-miR-4454 gene” or “hsa-miR-4454” used in the present specification includes the hsa-miR-4454 gene (miRBase Accession No. MIMAT0018976) described in SEQ ID NO: 41, a homolog or an ortholog of a different organism species, and the like. The hsa-miR-4454 gene can be obtained by a method described in Jima D D et al., 2010, Blood, Vol. 116, e118-e127. Also, “hsa-mir-4454” (miRBase Accession No. MI0016800, SEQ ID NO: 227) having a hairpin-like structure is known as a precursor of “hsa-miR-4454”.

The term “hsa-miR-4525 gene” or “hsa-miR-4525” used in the present specification includes the hsa-miR-4525 gene (miRBase Accession No. MIMAT0019064) described in SEQ ID NO: 42, a homolog or an ortholog of a different organism species, and the like. The hsa-miR-4525 gene can be obtained by a method described in Jima D D et al., 2010, Blood, Vol. 116, e118-e127. Also, “hsa-mir-4525” (miRBase Accession No. MI0016892, SEQ ID NO: 228) having a hairpin-like structure is known as a precursor of “hsa-miR-4525”.

The term “hsa-miR-7975 gene” or “hsa-miR-7975” used in the present specification includes the hsa-miR-7975 gene (miRBase Accession No. MIMAT0031178) described in SEQ ID NO: 43, a homolog or an ortholog of a different organism species, and the like. The hsa-miR-7975 gene can be obtained by a method described in Velthut-Meikas A et al., 2013, Mol Endocrinol, online. Also, “hsa-mir-7975” (miRBase Accession No. MI0025751, SEQ ID NO: 229) having a hairpin-like structure is known as a precursor of “hsa-miR-7975”.

The term “hsa-miR-744-5p gene” or “hsa-miR-744-5p” used in the present specification includes the hsa-miR-744-5p gene (miRBase Accession No. MIMAT0004945) described in SEQ ID NO: 44, a homolog or an ortholog of a different organism species, and the like. The hsa-miR-744-5p gene can be obtained by a method described in Berezikov E et al., 2006, Genome Res, Vol. 16, p. 1289-1298. Also, “hsa-mir-744” (miRBase Accession No. MI0005559, SEQ ID NO: 230) having a hairpin-like structure is known as a precursor of “hsa-miR-744-5p”.

The term “hsa-miR-3135b gene” or “hsa-miR-3135b” used in the present specification includes the hsa-miR-3135b gene (miRBase Accession No. MIMAT0018985) described in SEQ ID NO: 45, a homolog or an ortholog of a different organism species, and the like. The hsa-miR-3135b gene can be obtained by a method described in Jima D D et al., 2010, Blood, Vol. 116, e118-e127. Also, “hsa-mir-3135b” (miRBase Accession No. MI0016809, SEQ ID NO: 231) having a hairpin-like structure is known as a precursor of “hsa-miR-3135b”.

The term “hsa-miR-4648 gene” or “hsa-miR-4648” used in the present specification includes the hsa-miR-4648 gene (miRBase Accession No. MIMAT0019710) described in SEQ ID NO: 46, a homolog or an ortholog of a different organism species, and the like. The hsa-miR-4648 gene can be obtained by a method described in Persson H et al., 2011, Cancer Res, Vol. 71, p. 78-86. Also, “hsa-mir-4648” (miRBase Accession No. MI0017275, SEQ ID NO: 232) having a hairpin-like structure is known as a precursor of “hsa-miR-4648”.

The term “hsa-miR-6816-5p gene” or “hsa-miR-6816-5p” used in the present specification includes the hsa-miR-6816-5p gene (miRBase Accession No. MIMAT0027532) described in SEQ ID NO: 47, a homolog or an ortholog of a different organism species, and the like. The hsa-miR-6816-5p gene can be obtained by a method described in Ladewig E et al., 2012, Genome Res, Vol. 22, p. 1634-1645. Also, “hsa-mir-6816” (miRBase Accession No. MI0022661, SEQ ID NO: 233) having a hairpin-like structure is known as a precursor of “hsa-miR-6816-5p”.

The term “hsa-miR-4741 gene” or “hsa-miR-4741” used in the present specification includes the hsa-miR-4741 gene (miRBase Accession No. MIMAT0019871) described in SEQ ID NO: 48, a homolog or an ortholog of a different organism species, and the like. The hsa-miR-4741 gene can be obtained by a method described in Persson H et al., 2011, Cancer Res, Vol. 71, p. 78-86. Also, “hsa-mir-4741” (miRBase Accession No. MI0017379, SEQ ID NO: 234) having a hairpin-like structure is known as a precursor of “hsa-miR-4741”.

The term “hsa-miR-7150 gene” or “hsa-miR-7150” used in the present specification includes the hsa-miR-7150 gene (miRBase Accession No. MIMAT0028211) described in SEQ ID NO: 49, a homolog or an ortholog of a different organism species, and the like. The hsa-miR-7150 gene can be obtained by a method described in Oulas A et al., 2009, Nucleic Acids Res, Vol. 37, p. 3276-3287. Also, “hsa-mir-7150” (miRBase Accession No. MI0023610, SEQ ID NO: 235) having a hairpin-like structure is known as a precursor of “hsa-miR-7150”.

The term “hsa-miR-6791-5p gene” or “hsa-miR-6791-5p” used in the present specification includes the hsa-miR-6791-5p gene (miRBase Accession No. MIMAT0027482) described in SEQ ID NO: 50, a homolog or an ortholog of a different organism species, and the like. The hsa-miR-6791-5p gene can be obtained by a method described in Ladewig E et al., 2012, Genome Res, Vol. 22, p. 1634-1645. Also, “hsa-mir-6791” (miRBase Accession No. MI0022636, SEQ ID NO: 236) having a hairpin-like structure is known as a precursor of “hsa-miR-6791-5p”.

The term “hsa-miR-1247-3p gene” or “hsa-miR-1247-3p” used in the present specification includes the hsa-miR-1247-3p gene (miRBase Accession No. MIMAT0022721) described in SEQ ID NO: 51, a homolog or an ortholog of a different organism species, and the like. The hsa-miR-1247-3p gene can be obtained by a method described in Morin R D et al., 2008, Genome Res, Vol. 18, p. 610-621. Also, “hsa-mir-1247” (miRBase Accession No. MI0006382, SEQ ID NO: 237) having a hairpin-like structure is known as a precursor of “hsa-miR-1247-3p”.

The term “hsa-miR-7977 gene” or “hsa-miR-7977” used in the present specification includes the hsa-miR-7977 gene (miRBase Accession No. MIMAT0031180) described in SEQ ID NO: 52, a homolog or an ortholog of a different organism species, and the like. The hsa-miR-7977 gene can be obtained by a method described in Velthut-Meikas A et al., 2013, Mol Endocrinol, online. Also, “hsa-mir-7977” (miRBase Accession No. MI0025753, SEQ ID NO: 238) having a hairpin-like structure is known as a precursor of “hsa-miR-7977”.

The term “hsa-miR-4497 gene” or “hsa-miR-4497” used in the present specification includes the hsa-miR-4497 gene (miRBase Accession No. MIMAT0019032) described in SEQ ID NO: 53, a homolog or an ortholog of a different organism species, and the like. The hsa-miR-4497 gene can be obtained by a method described in Jima D D et al., 2010, Blood, Vol. 116, e118-e127. Also, “hsa-mir-4497” (miRBase Accession No. MI0016859, SEQ ID NO: 239) having a hairpin-like structure is known as a precursor of “hsa-miR-4497”.

The term “hsa-miR-6090 gene” or “hsa-miR-6090” used in the present specification includes the hsa-miR-6090 gene (miRBase Accession No. MIMAT0023715) described in SEQ ID NO: 54, a homolog or an ortholog of a different organism species, and the like. The hsa-miR-6090 gene can be obtained by a method described in Yoo J K et al., 2012, Stem Cells Dev, Vol. 21, p. 2049-2057. Also, “hsa-mir-6090” (miRBase Accession No. MI0020367, SEQ ID NO: 240) having a hairpin-like structure is known as a precursor of “hsa-miR-6090”.

The term “hsa-miR-6781-5p gene” or “hsa-miR-6781-5p” used in the present specification includes the hsa-miR-6781-5p gene (miRBase Accession No. MIMAT0027462) described in SEQ ID NO: 55, a homolog or an ortholog of a different organism species, and the like. The hsa-miR-6781-5p gene can be obtained by a method described in Ladewig E et al., 2012, Genome Res, Vol. 22, p. 1634-1645. Also, “hsa-mir-6781” (miRBase Accession No. MI0022626, SEQ ID NO: 241) having a hairpin-like structure is known as a precursor of “hsa-miR-6781-5p”.

The term “hsa-miR-6870-5p gene” or “hsa-miR-6870-5p” used in the present specification includes the hsa-miR-6870-5p gene (miRBase Accession No. MIMAT0027640) described in SEQ ID NO: 56, a homolog or an ortholog of a different organism species, and the like. The hsa-miR-6870-5p gene can be obtained by a method described in Ladewig E et al., 2012, Genome Res, Vol. 22, p. 1634-1645. Also, “hsa-mir-6870” (miRBase Accession No. MI0022717, SEQ ID NO: 242) having a hairpin-like structure is known as a precursor of “hsa-miR-6870-5p”.

The term “hsa-miR-6729-5p gene” or “hsa-miR-6729-5p” used in the present specification includes the hsa-miR-6729-5p gene (miRBase Accession No. MIMAT0027359) described in SEQ ID NO: 57, a homolog or an ortholog of a different organism species, and the like. The hsa-miR-6729-5p gene can be obtained by a method described in Ladewig E et al., 2012, Genome Res, Vol. 22, p. 1634-1645. Also, “hsa-mir-6729” (miRBase Accession No. MI0022574, SEQ ID NO: 243) having a hairpin-like structure is known as a precursor of “hsa-miR-6729-5p”.

The term “hsa-miR-4530 gene” or “hsa-miR-4530” used in the present specification includes the hsa-miR-4530 gene (miRBase Accession No. MIMAT0019069) described in SEQ ID NO: 58, a homolog or an ortholog of a different organism species, and the like. The hsa-miR-4530 gene can be obtained by a method described in Jima D D et al., 2010, Blood, Vol. 116, e118-e127. Also, “hsa-mir-4530” (miRBase Accession No. MI0016897, SEQ ID NO: 244) having a hairpin-like structure is known as a precursor of “hsa-miR-4530”.

The term “hsa-miR-7847-3p gene” or “hsa-miR-7847-3p” used in the present specification includes the hsa-miR-7847-3p gene (miRBase Accession No. MIMAT0030422) described in SEQ ID NO: 59, a homolog or an ortholog of a different organism species, and the like. The hsa-miR-7847-3p gene can be obtained by a method described in Ple H et al., 2012, PLoS One, Vol. 7, e50746. Also, “hsa-mir-7847” (miRBase Accession No. MI0025517, SEQ ID NO: 245) having a hairpin-like structure is known as a precursor of “hsa-miR-7847-3p”.

The term “hsa-miR-6825-5p gene” or “hsa-miR-6825-5p” used in the present specification includes the hsa-miR-6825-5p gene (miRBase Accession No. MIMAT0027550) described in SEQ ID NO: 60, a homolog or an ortholog of a different organism species, and the like. The hsa-miR-6825-5p gene can be obtained by a method described in Ladewig E et al., 2012, Genome Res, Vol. 22, p. 1634-1645. Also, “hsa-mir-6825” (miRBase Accession No. MI0022670, SEQ ID NO: 246) having a hairpin-like structure is known as a precursor of “hsa-miR-6825-5p”.

The term “hsa-miR-4674 gene” or “hsa-miR-4674” used in the present specification includes the hsa-miR-4674 gene (miRBase Accession No. MIMAT0019756) described in SEQ ID NO: 61, a homolog or an ortholog of a different organism species, and the like. The hsa-miR-4674 gene can be obtained by a method described in Persson H et al., 2011, Cancer Res, Vol. 71, p. 78-86. Also, “hsa-mir-4674” (miRBase Accession No. MI0017305, SEQ ID NO: 247) having a hairpin-like structure is known as a precursor of “hsa-miR-4674”.

The term “hsa-miR-3917 gene” or “hsa-miR-3917” used in the present specification includes the hsa-miR-3917 gene (miRBase Accession No. MIMAT0018191) described in SEQ ID NO: 62, a homolog or an ortholog of a different organism species, and the like. The hsa-miR-3917 gene can be obtained by a method described in Creighton C J et al., 2010, PLoS One, Vol. 5, e9637. Also, “hsa-mir-3917” (miRBase Accession No. MI0016423, SEQ ID NO: 248) having a hairpin-like structure is known as a precursor of “hsa-miR-3917”.

The term “hsa-miR-4707-3p gene” or “hsa-miR-4707-3p” used in the present specification includes the hsa-miR-4707-3p gene (miRBase Accession No. MIMAT0019808) described in SEQ ID NO: 63, a homolog or an ortholog of a different organism species, and the like. The hsa-miR-4707-3p gene can be obtained by a method described in Persson H et al., 2011, Cancer Res, Vol. 71, p. 78-86. Also, “hsa-mir-4707” (miRBase Accession No. MI0017340, SEQ ID NO: 225) having a hairpin-like structure is known as a precursor of “hsa-miR-4707-3p”.

The term “hsa-miR-6885-5p gene” or “hsa-miR-6885-5p” used in the present specification includes the hsa-miR-6885-5p gene (miRBase Accession No. MIMAT0027670) described in SEQ ID NO: 64, a homolog or an ortholog of a different organism species, and the like. The hsa-miR-6885-5p gene can be obtained by a method described in Ladewig E et al., 2012, Genome Res, Vol. 22, p. 1634-1645. Also, “hsa-mir-6885” (miRBase Accession No. MI0022732, SEQ ID NO: 249) having a hairpin-like structure is known as a precursor of “hsa-miR-6885-5p”.

The term “hsa-miR-6722-3p gene” or “hsa-miR-6722-3p” used in the present specification includes the hsa-miR-6722-3p gene (miRBase Accession No. MIMAT0025854) described in SEQ ID NO: 65, a homolog or an ortholog of a different organism species, and the like. The hsa-miR-6722-3p gene can be obtained by a method described in Li Y et al., 2012, Gene, Vol. 497, p. 330-335. Also, “hsa-mir-6722” (miRBase Accession No. MI0022557, SEQ ID NO: 250) having a hairpin-like structure is known as a precursor of “hsa-miR-6722-3p”.

The term “hsa-miR-4516 gene” or “hsa-miR-4516” used in the present specification includes the hsa-miR-4516 gene (miRBase Accession No. MIMAT0019053) described in SEQ ID NO: 66, a homolog or an ortholog of a different organism species, and the like. The hsa-miR-4516 gene can be obtained by a method described in Jima D D et al., 2010, Blood, Vol. 116, e118-e127. Also, “hsa-mir-4516” (miRBase Accession No. MI0016882, SEQ ID NO: 251) having a hairpin-like structure is known as a precursor of “hsa-miR-4516”.

The term “hsa-miR-6757-5p gene” or “hsa-miR-6757-5p” used in the present specification includes the hsa-miR-6757-5p gene (miRBase Accession No. MIMAT0027414) described in SEQ ID NO: 67, a homolog or an ortholog of a different organism species, and the like. The hsa-miR-6757-5p gene can be obtained by a method described in Ladewig E et al., 2012, Genome Res, Vol. 22, p. 1634-1645. Also, “hsa-mir-6757” (miRBase Accession No. MI0022602, SEQ ID NO: 252) having a hairpin-like structure is known as a precursor of “hsa-miR-6757-5p”.

The term “hsa-miR-6840-3p gene” or “hsa-miR-6840-3p” used in the present specification includes the hsa-miR-6840-3p gene (miRBase Accession No. MIMAT0027583) described in SEQ ID NO: 68, a homolog or an ortholog of a different organism species, and the like. The hsa-miR-6840-3p gene can be obtained by a method described in Ladewig E et al., 2012, Genome Res, Vol. 22, p. 1634-1645. Also, “hsa-mir-6840” (miRBase Accession No. MI0022686, SEQ ID NO: 253) having a hairpin-like structure is known as a precursor of “hsa-miR-6840-3p”.

The term “hsa-miR-5195-3p gene” or “hsa-miR-5195-3p” used in the present specification includes the hsa-miR-5195-3p gene (miRBase Accession No. MIMAT0021127) described in SEQ ID NO: 69, a homolog or an ortholog of a different organism species, and the like. The hsa-miR-5195-3p gene can be obtained by a method described in Schotte D et al., 2011, Leukemia, Vol. 25, p. 1389-1399. Also, “hsa-mir-5195” (miRBase Accession No. MI0018174, SEQ ID NO: 254) having a hairpin-like structure is known as a precursor of “hsa-miR-5195-3p”.

The term “hsa-miR-6756-5p gene” or “hsa-miR-6756-5p” used in the present specification includes the hsa-miR-6756-5p gene (miRBase Accession No. MIMAT0027412) described in SEQ ID NO: 70, a homolog or an ortholog of a different organism species, and the like. The hsa-miR-6756-5p gene can be obtained by a method described in Ladewig E et al., 2012, Genome Res, Vol. 22, p. 1634-1645. Also, “hsa-mir-6756” (miRBase Accession No. MI0022601, SEQ ID NO: 255) having a hairpin-like structure is known as a precursor of “hsa-miR-6756-5p”.

The term “hsa-miR-6800-5p gene” or “hsa-miR-6800-5p” used in the present specification includes the hsa-miR-6800-5p gene (miRBase Accession No. MIMAT0027500) described in SEQ ID NO: 71, a homolog or an ortholog of a different organism species, and the like. The hsa-miR-6800-5p gene can be obtained by a method described in Ladewig E et al., 2012, Genome Res, Vol. 22, p. 1634-1645. Also, “hsa-mir-6800” (miRBase Accession No. MI0022645, SEQ ID NO: 256) having a hairpin-like structure is known as a precursor of “hsa-miR-6800-5p”.

The term “hsa-miR-6727-5p gene” or “hsa-miR-6727-5p” used in the present specification includes the hsa-miR-6727-5p gene (miRBase Accession No. MIMAT0027355) described in SEQ ID NO: 72, a homolog or an ortholog of a different organism species, and the like. The hsa-miR-6727-5p gene can be obtained by a method described in Ladewig E et al., 2012, Genome Res, Vol. 22, p. 1634-1645. Also, “hsa-mir-6727” (miRBase Accession No. MI0022572, SEQ ID NO: 257) having a hairpin-like structure is known as a precursor of “hsa-miR-6727-5p”.

The term “hsa-miR-6126 gene” or “hsa-miR-6126” used in the present specification includes the hsa-miR-6126 gene (miRBase Accession No. MIMAT0024599) described in SEQ ID NO: 73, a homolog or an ortholog of a different organism species, and the like. The hsa-miR-6126 gene can be obtained by a method described in Smith J L et al., 2012, J Virol, Vol. 86, p. 5278-5287. Also, “hsa-mir-6126” (miRBase Accession No. MI0021260, SEQ ID NO: 258) having a hairpin-like structure is known as a precursor of “hsa-miR-6126”.

The term “hsa-miR-6872-3p gene” or “hsa-miR-6872-3p” used in the present specification includes the hsa-miR-6872-3p gene (miRBase Accession No. MIMAT0027645) described in SEQ ID NO: 74, a homolog or an ortholog of a different organism species, and the like. The hsa-miR-6872-3p gene can be obtained by a method described in Ladewig E et al., 2012, Genome Res, Vol. 22, p. 1634-1645. Also, “hsa-mir-6872” (miRBase Accession No. MI0022719, SEQ ID NO: 259) having a hairpin-like structure is known as a precursor of “hsa-miR-6872-3p”.

The term “hsa-miR-4446-3p gene” or “hsa-miR-4446-3p” used in the present specification includes the hsa-miR-4446-3p gene (miRBase Accession No. MIMAT0018965) described in SEQ ID NO: 75, a homolog or an ortholog of a different organism species, and the like. The hsa-miR-4446-3p gene can be obtained by a method described in Jima D D et al., 2010, Blood, Vol. 116, e118-e127. Also, “hsa-mir-4446” (miRBase Accession No. MI0016789, SEQ ID NO: 260) having a hairpin-like structure is known as a precursor of “hsa-miR-4446-3p”.

The term “hsa-miR-1268a gene” or “hsa-miR-1268a” used in the present specification includes the hsa-miR-1268a gene (miRBase Accession No. MIMAT0005922) described in SEQ ID NO: 76, a homolog or an ortholog of a different organism species, and the like. The hsa-miR-1268a gene can be obtained by a method described in Morin R D et al., 2008, Genome Res, Vol. 18, p. 610-621. Also, “hsa-mir-1268a” (miRBase Accession No. MI0006405, SEQ ID NO: 261) having a hairpin-like structure is known as a precursor of “hsa-miR-1268a”.

The term “hsa-miR-1908-3p gene” or “hsa-miR-1908-3p” used in the present specification includes the hsa-miR-1908-3p gene (miRBase Accession No. MIMAT0026916) described in SEQ ID NO: 77, a homolog or an ortholog of a different organism species, and the like. The hsa-miR-1908-3p gene can be obtained by a method described in Bar M et al., 2008, Stem Cells, Vol. 26, p. 2496-2505. Also, “hsa-mir-1908” (miRBase Accession No. MI0008329, SEQ ID NO: 189) having a hairpin-like structure is known as a precursor of “hsa-miR-1908-3p”.

The term “hsa-miR-3679-5p gene” or “hsa-miR-3679-5p” used in the present specification includes the hsa-miR-3679-5p gene (miRBase Accession No. MIMAT0018104) described in SEQ ID NO: 78, a homolog or an ortholog of a different organism species, and the like. The hsa-miR-3679-5p gene can be obtained by a method described in Creighton C J et al., 2010, PLoS One, Vol. 5, e9637. Also, “hsa-mir-3679” (miRBase Accession No. MI0016080, SEQ ID NO: 262) having a hairpin-like structure is known as a precursor of “hsa-miR-3679-5p”.

The term “hsa-miR-4534 gene” or “hsa-miR-4534” used in the present specification includes the hsa-miR-4534 gene (miRBase Accession No. MIMAT0019073) described in SEQ ID NO: 79, a homolog or an ortholog of a different organism species, and the like. The hsa-miR-4534 gene can be obtained by a method described in Jima D D et al., 2010, Blood, Vol. 116, e118-e127. Also, “hsa-mir-4534” (miRBase Accession No. MI0016901, SEQ ID NO: 263) having a hairpin-like structure is known as a precursor of “hsa-miR-4534”.

The term “hsa-miR-4675 gene” or “hsa-miR-4675” used in the present specification includes the hsa-miR-4675 gene (miRBase Accession No. MIMAT0019757) described in SEQ ID NO: 80, a homolog or an ortholog of a different organism species, and the like. The hsa-miR-4675 gene can be obtained by a method described in Persson H et al., 2011, Cancer Res, Vol. 71, p. 78-86. Also, “hsa-mir-4675” (miRBase Accession No. MI0017306, SEQ ID NO: 264) having a hairpin-like structure is known as a precursor of “hsa-miR-4675”.

The term “hsa-miR-7108-5p gene” or “hsa-miR-7108-5p” used in the present specification includes the hsa-miR-7108-5p gene (miRBase Accession No. MIMAT0028113) described in SEQ ID NO: 81, a homolog or an ortholog of a different organism species, and the like. The hsa-miR-7108-5p gene can be obtained by a method described in Ladewig E et al., 2012, Genome Res, Vol. 22, p. 1634-1645. Also, “hsa-mir-7108” (miRBase Accession No. MI0022959, SEQ ID NO: 265) having a hairpin-like structure is known as a precursor of “hsa-miR-7108-5p”.

The term “hsa-miR-6799-5p gene” or “hsa-miR-6799-5p” used in the present specification includes the hsa-miR-6799-5p gene (miRBase Accession No. MIMAT0027498) described in SEQ ID NO: 82, a homolog or an ortholog of a different organism species, and the like. The hsa-miR-6799-5p gene can be obtained by a method described in Ladewig E et al., 2012, Genome Res, Vol. 22, p. 1634-1645. Also, “hsa-mir-6799” (miRBase Accession No. MI0022644, SEQ ID NO: 266) having a hairpin-like structure is known as a precursor of “hsa-miR-6799-5p”.

The term “hsa-miR-4695-5p gene” or “hsa-miR-4695-5p” used in the present specification includes the hsa-miR-4695-5p gene (miRBase Accession No. MIMAT0019788) described in SEQ ID NO: 83, a homolog or an ortholog of a different organism species, and the like. The hsa-miR-4695-5p gene can be obtained by a method described in Persson H et al., 2011, Cancer Res, Vol. 71, p. 78-86. Also, “hsa-mir-4695” (miRBase Accession No. MI0017328, SEQ ID NO: 267) having a hairpin-like structure is known as a precursor of “hsa-miR-4695-5p”.

The term “hsa-miR-3178 gene” or “hsa-miR-3178” used in the present specification includes the hsa-miR-3178 gene (miRBase Accession No. MIMAT0015055) described in SEQ ID NO: 84, a homolog or an ortholog of a different organism species, and the like. The hsa-miR-3178 gene can be obtained by a method described in Stark M S et al., 2010, PLoS One, Vol. 5, e9685. Also, “hsa-mir-3178” (miRBase Accession No. MI0014212, SEQ ID NO: 268) having a hairpin-like structure is known as a precursor of “hsa-miR-3178”.

The term “hsa-miR-5090 gene” or “hsa-miR-5090” used in the present specification includes the hsa-miR-5090 gene (miRBase Accession No. MIMAT0021082) described in SEQ ID NO: 85, a homolog or an ortholog of a different organism species, and the like. The hsa-miR-5090 gene can be obtained by a method described in Ding N et al., 2011, J Radiat Res, Vol. 52, p. 425-432. Also, “hsa-mir-5090” (miRBase Accession No. MI0017979, SEQ ID NO: 269) having a hairpin-like structure is known as a precursor of “hsa-miR-5090”.

The term “hsa-miR-3180 gene” or “hsa-miR-3180” used in the present specification includes the hsa-miR-3180 gene (miRBase Accession No. MIMAT0018178) described in SEQ ID NO: 86, a homolog or an ortholog of a different organism species, and the like. The hsa-miR-3180 gene can be obtained by a method described in Creighton C J et al., 2010, PLoS One, Vol. 5, e9637. Also, “hsa-mir-3180-4 and hsa-mir-3180-5” (miRBase Accession Nos. MI0016408 and MI0016409, SEQ ID NOs: 270 and 271) having a hairpin-like structure is known as precursors of “hsa-miR-3180”.

The term “hsa-miR-1237-5p gene” or “hsa-miR-1237-5p” used in the present specification includes the hsa-miR-1237-5p gene (miRBase Accession No. MIMAT0022946) described in SEQ ID NO: 87, a homolog or an ortholog of a different organism species, and the like. The hsa-miR-1237-5p gene can be obtained by a method described in Berezikov E et al., 2007, Mol Cell, Vol. 28, p. 328-336. Also, “hsa-mir-1237” (miRBase Accession No. MI0006327, SEQ ID NO: 272) having a hairpin-like structure is known as a precursor of “hsa-miR-1237-5p”.

The term “hsa-miR-4758-5p gene” or “hsa-miR-4758-5p” used in the present specification includes the hsa-miR-4758-5p gene (miRBase Accession No. MIMAT0019903) described in SEQ ID NO: 88, a homolog or an ortholog of a different organism species, and the like. The hsa-miR-4758-5p gene can be obtained by a method described in Persson H et al., 2011, CancerRes, Vol. 71, p. 78-86. Also, “hsa-mir-4758” (miRBase Accession No. MI0017399, SEQ ID NO: 273) having a hairpin-like structure is known as a precursor of “hsa-miR-4758-5p”.

The term “hsa-miR-3184-5p gene” or “hsa-miR-3184-5p” used in the present specification includes the hsa-miR-3184-5p gene (miRBase Accession No. MIMAT0015064) described in SEQ ID NO: 89, a homolog or an ortholog of a different organism species, and the like. The hsa-miR-3184-5p gene can be obtained by a method described in Stark M S et al., 2010, PLoS One, Vol. 5, e9685. Also, “hsa-mir-3184” (miRBase Accession No. MI0014226, SEQ ID NO: 274) having a hairpin-like structure is known as a precursor of “hsa-miR-3184-5p”.

The term “hsa-miR-4286 gene” or “hsa-miR-4286” used in the present specification includes the hsa-miR-4286 gene (miRBase Accession No. MIMAT0016916) described in SEQ ID NO: 90, a homolog or an ortholog of a different organism species, and the like. The hsa-miR-4286 gene can be obtained by a method described in Goff L A et al., 2009, PLoS One, Vol. 4, e7192. Also, “hsa-mir-4286” (miRBase Accession No. MI0015894, SEQ ID NO: 275) having a hairpin-like structure is known as a precursor of “hsa-miR-4286”.

The term “hsa-miR-6784-5p gene” or “hsa-miR-6784-5p” used in the present specification includes the hsa-miR-6784-5p gene (miRBase Accession No. MIMAT0027468) described in SEQ ID NO: 91, a homolog or an ortholog of a different organism species, and the like. The hsa-miR-6784-5p gene can be obtained by a method described in Ladewig E et al., 2012, Genome Res, Vol. 22, p. 1634-1645. Also, “hsa-mir-6784” (miRBase Accession No. MI0022629, SEQ ID NO: 276) having a hairpin-like structure is known as a precursor of “hsa-miR-6784-5p”.

The term “hsa-miR-6768-5p gene” or “hsa-miR-6768-5p” used in the present specification includes the hsa-miR-6768-5p gene (miRBase Accession No. MIMAT0027436) described in SEQ ID NO: 92, a homolog or an ortholog of a different organism species, and the like. The hsa-miR-6768-5p gene can be obtained by a method described in Ladewig E et al., 2012, Genome Res, Vol. 22, p. 1634-1645. Also, “hsa-mir-6768” (miRBase Accession No. MI0022613, SEQ ID NO: 277) having a hairpin-like structure is known as a precursor of “hsa-miR-6768-5p”.

The term “hsa-miR-6785-5p gene” or “hsa-miR-6785-5p” used in the present specification includes the hsa-miR-6785-5p gene (miRBase Accession No. MIMAT0027470) described in SEQ ID NO: 93, a homolog or an ortholog of a different organism species, and the like. The hsa-miR-6785-5p gene can be obtained by a method described in Ladewig E et al., 2012, Genome Res, Vol. 22, p. 1634-1645. Also, “hsa-mir-6785” (miRBase Accession No. MI0022630, SEQ ID NO: 278) having a hairpin-like structure is known as a precursor of “hsa-miR-6785-5p”.

The term “hsa-miR-4706 gene” or “hsa-miR-4706” used in the present specification includes the hsa-miR-4706 gene (miRBase Accession No. MIMAT0019806) described in SEQ ID NO: 94, a homolog or an ortholog of a different organism species, and the like. The hsa-miR-4706 gene can be obtained by a method described in Persson H et al., 2011, Cancer Res, Vol. 71, p. 78-86. Also, “hsa-mir-4706” (miRBase Accession No. MI0017339, SEQ ID NO: 279) having a hairpin-like structure is known as a precursor of “hsa-miR-4706”.

The term “hsa-miR-711 gene” or “hsa-miR-711” used in the present specification includes the hsa-miR-711 gene (miRBase Accession No. MIMAT0012734) described in SEQ ID NO: 95, a homolog or an ortholog of a different organism species, and the like. The hsa-miR-711 gene can be obtained by a method described in Artzi S et al., 2008, BMC Bioinformatics, Vol. 9, p. 39. Also, “hsa-mir-711” (miRBase Accession No. MI0012488, SEQ ID NO: 280) having a hairpin-like structure is known as a precursor of “hsa-miR-711”.

The term “hsa-miR-1260a gene” or “hsa-miR-1260a” used in the present specification includes the hsa-miR-1260a gene (miRBase Accession No. MIMAT0005911) described in SEQ ID NO: 96, a homolog or an ortholog of a different organism species, and the like. The hsa-miR-1260a gene can be obtained by a method described in Morin R D et al., 2008, Genome Res, Vol. 18, p. 610-621. Also, “hsa-mir-1260a” (miRBase Accession No. MI0006394, SEQ ID NO: 281) having a hairpin-like structure is known as a precursor of “hsa-miR-1260a”.

The term “hsa-miR-6746-5p gene” or “hsa-miR-6746-5p” used in the present specification includes the hsa-miR-6746-5p gene (miRBase Accession No. MIMAT0027392) described in SEQ ID NO: 97, a homolog or an ortholog of a different organism species, and the like. The hsa-miR-6746-5p gene can be obtained by a method described in Ladewig E et al., 2012, Genome Res, Vol. 22, p. 1634-1645. Also, “hsa-mir-6746” (miRBase Accession No. MI0022591, SEQ ID NO: 282) having a hairpin-like structure is known as a precursor of “hsa-miR-6746-5p”.

The term “hsa-miR-6089 gene” or “hsa-miR-6089” used in the present specification includes the hsa-miR-6089 gene (miRBase Accession No. MIMAT0023714) described in SEQ ID NO: 98, a homolog or an ortholog of a different organism species, and the like. The hsa-miR-6089 gene can be obtained by a method described in Yoo J K et al., 2012, Stem Cells Dev, Vol. 21, p. 2049-2057. Also, “hsa-mir-6089-1 and hsa-mir-6089-2” (miRBase Accession Nos. MI0020366 and MI0023563, SEQ ID NOs: 283 and 284) having a hairpin-like structure are known as precursors of “hsa-miR-6089”.

The term “hsa-miR-6821-5p gene” or “hsa-miR-6821-5p” used in the present specification includes the hsa-miR-6821-5p gene (miRBase Accession No. MIMAT0027542) described in SEQ ID NO: 99, a homolog or an ortholog of a different organism species, and the like. The hsa-miR-6821-5p gene can be obtained by a method described in Ladewig E et al., 2012, Genome Res, Vol. 22, p. 1634-1645. Also, “hsa-mir-6821” (miRBase Accession No. MI0022666, SEQ ID NO: 285) having a hairpin-like structure is known as a precursor of “hsa-miR-6821-5p”.

The term “hsa-miR-4667-5p gene” or “hsa-miR-4667-5p” used in the present specification includes the hsa-miR-4667-5p gene (miRBase Accession No. MIMAT0019743) described in SEQ ID NO: 100, a homolog or an ortholog of a different organism species, and the like. The hsa-miR-4667-5p gene can be obtained by a method described in Persson H et al., 2011, Cancer Res, Vol. 71, p. 78-86. Also, “hsa-mir-4667” (miRBase Accession No. MI0017297, SEQ ID NO: 286) having a hairpin-like structure is known as a precursor of “hsa-miR-4667-5p”.

The term “hsa-miR-8069 gene” or “hsa-miR-8069” used in the present specification includes the hsa-miR-8069 gene (miRBase Accession No. MIMAT0030996) described in SEQ ID NO: 101, a homolog or an ortholog of a different organism species, and the like. The hsa-miR-8069 gene can be obtained by a method described in Wang H J et al., 2013, Shock, Vol. 39, p. 480-487. Also, “hsa-mir-8069” (miRBase Accession No. MI0025905, SEQ ID NO: 287) having a hairpin-like structure is known as a precursor of “hsa-miR-8069”.

The term “hsa-miR-4726-5p gene” or “hsa-miR-4726-5p” used in the present specification includes the hsa-miR-4726-5p gene (miRBase Accession No. MIMAT0019845) described in SEQ ID NO: 102, a homolog or an ortholog of a different organism species, and the like. The hsa-miR-4726-5p gene can be obtained by a method described in Persson H et al., 2011, Cancer Res, Vol. 71, p. 78-86. Also, “hsa-mir-4726” (miRBase Accession No. MI0017363, SEQ ID NO: 288) having a hairpin-like structure is known as a precursor of “hsa-miR-4726-5p”.

The term “hsa-miR-6124 gene” or “hsa-miR-6124” used in the present specification includes the hsa-miR-6124 gene (miRBase Accession No. MIMAT0024597) described in SEQ ID NO: 103, a homolog or an ortholog of a different organism species, and the like. The hsa-miR-6124 gene can be obtained by a method described in Smith J L et al., 2012, J Virol, Vol. 86, p. 5278-5287. Also, “hsa-mir-6124” (miRBase Accession No. MI0021258, SEQ ID NO: 289) having a hairpin-like structure is known as a precursor of “hsa-miR-6124”.

The term “hsa-miR-4532 gene” or “hsa-miR-4532” used in the present specification includes the hsa-miR-4532 gene (miRBase Accession No. MIMAT0019071) described in SEQ ID NO: 104, a homolog or an ortholog of a different organism species, and the like. The hsa-miR-4532 gene can be obtained by a method described in Jima D D et al., 2010, Blood, Vol. 116, e118-e127. Also, “hsa-mir-4532” (miRBase Accession No. MI0016899, SEQ ID NO: 290) having a hairpin-like structure is known as a precursor of “hsa-miR-4532”.

The term “hsa-miR-4486 gene” or “hsa-miR-4486” used in the present specification includes the hsa-miR-4486 gene (miRBase Accession No. MIMAT0019020) described in SEQ ID NO: 105, a homolog or an ortholog of a different organism species, and the like. The hsa-miR-4486 gene can be obtained by a method described in Jima D D et al., 2010, Blood, Vol. 116, e118-e127. Also, “hsa-mir-4486” (miRBase Accession No. MI0016847, SEQ ID NO: 291) having a hairpin-like structure is known as a precursor of “hsa-miR-4486”.

The term “hsa-miR-4728-5p gene” or “hsa-miR-4728-5p” used in the present specification includes the hsa-miR-4728-5p gene (miRBase Accession No. MIMAT0019849) described in SEQ ID NO: 106, a homolog or an ortholog of a different organism species, and the like. The hsa-miR-4728-5p gene can be obtained by a method described in Persson H et al., 2011, Cancer Res, Vol. 71, p. 78-86. Also, “hsa-mir-4728” (miRBase Accession No. MI0017365, SEQ ID NO: 292) having a hairpin-like structure is known as a precursor of “hsa-miR-4728-5p”.

The term “hsa-miR-4508 gene” or “hsa-miR-4508” used in the present specification includes the hsa-miR-4508 gene (miRBase Accession No. MIMAT0019045) described in SEQ ID NO: 107, a homolog or an ortholog of a different organism species, and the like. The hsa-miR-4508 gene can be obtained by a method described in Jima D D et al., 2010, Blood, Vol. 116, e118-e127. Also, “hsa-mir-4508” (miRBase Accession No. MI0016872, SEQ ID NO: 293) having a hairpin-like structure is known as a precursor of “hsa-miR-4508”.

The term “hsa-miR-128-1-5p gene” or “hsa-miR-128-1-5p” used in the present specification includes the hsa-miR-128-1-5p gene (miRBase Accession No. MIMAT0026477) described in SEQ ID NO: 108, a homolog or an ortholog of a different organism species, and the like. The hsa-miR-128-1-5p gene can be obtained by a method described in Lagos-Quintana M et al., 2002, Curr Biol, Vol. 12, p. 735-739. Also, “hsa-mir-128-1” (miRBase Accession No. MI0000447, SEQ ID NO: 294) having a hairpin-like structure is known as a precursor of “hsa-miR-128-1-5p”.

The term “hsa-miR-4513 gene” or “hsa-miR-4513” used in the present specification includes the hsa-miR-4513 gene (miRBase Accession No. MIMAT0019050) described in SEQ ID NO: 109, a homolog or an ortholog of a different organism species, and the like. The hsa-miR-4513 gene can be obtained by a method described in Jima D D et al., 2010, Blood, Vol. 116, e118-e127. Also, “hsa-mir-4513” (miRBase Accession No. MI0016879, SEQ ID NO: 295) having a hairpin-like structure is known as a precursor of “hsa-miR-4513”.

The term “hsa-miR-6795-5p gene” or “hsa-miR-6795-5p” used in the present specification includes the hsa-miR-6795-5p gene (miRBase Accession No. MIMAT0027490) described in SEQ ID NO: 110, a homolog or an ortholog of a different organism species, and the like. The hsa-miR-6795-5p gene can be obtained by a method described in Ladewig E et al., 2012, Genome Res, Vol. 22, p. 1634-1645. Also, “hsa-mir-6795” (miRBase Accession No. MI0022640, SEQ ID NO: 296) having a hairpin-like structure is known as a precursor of “hsa-miR-6795-5p”.

The term “hsa-miR-4689 gene” or “hsa-miR-4689” used in the present specification includes the hsa-miR-4689 gene (miRBase Accession No. MIMAT0019778) described in SEQ ID NO: 111, a homolog or an ortholog of a different organism species, and the like. The hsa-miR-4689 gene can be obtained by a method described in Persson H et al., 2011, Cancer Res, Vol. 71, p. 78-86. Also, “hsa-mir-4689” (miRBase Accession No. MI0017322, SEQ ID NO: 297) having a hairpin-like structure is known as a precursor of “hsa-miR-4689”.

The term “hsa-miR-6763-5p gene” or “hsa-miR-6763-5p” used in the present specification includes the hsa-miR-6763-5p gene (miRBase Accession No. MIMAT0027426) described in SEQ ID NO: 112, a homolog or an ortholog of a different organism species, and the like. The hsa-miR-6763-5p gene can be obtained by a method described in Ladewig E et al., 2012, Genome Res, Vol. 22, p. 1634-1645. Also, “hsa-mir-6763” (miRBase Accession No. MI0022608, SEQ ID NO: 298) having a hairpin-like structure is known as a precursor of “hsa-miR-6763-5p”.

The term “hsa-miR-8072 gene” or “hsa-miR-8072” used in the present specification includes the hsa-miR-8072 gene (miRBase Accession No. MIMAT0030999) described in SEQ ID NO: 113, a homolog or an ortholog of a different organism species, and the like. The hsa-miR-8072 gene can be obtained by a method described in Wang H J et al., 2013, Shock, Vol. 39, p. 480-487. Also, “hsa-mir-8072” (miRBase Accession No. MI0025908, SEQ ID NO: 299) having a hairpin-like structure is known as a precursor of “hsa-miR-8072”.

The term “hsa-miR-6765-5p gene” or “hsa-miR-6765-5p” used in the present specification includes the hsa-miR-6765-5p gene (miRBase Accession No. MIMAT0027430) described in SEQ ID NO: 114, a homolog or an ortholog of a different organism species, and the like. The hsa-miR-6765-5p gene can be obtained by a method described in Ladewig E et al., 2012, Genome Res, Vol. 22, p. 1634-1645. Also, “hsa-mir-6765” (miRBase Accession No. MI0022610, SEQ ID NO: 218) having a hairpin-like structure is known as a precursor of “hsa-miR-6765-5p”.

The term “hsa-miR-4419b gene” or “hsa-miR-4419b” used in the present specification includes the hsa-miR-4419b gene (miRBase Accession No. MIMAT0019034) described in SEQ ID NO: 115, a homolog or an ortholog of a different organism species, and the like. The hsa-miR-4419b gene can be obtained by a method described in Jima D D et al., 2010, Blood, Vol. 116, e118-e127. Also, “hsa-mir-4419b” (miRBase Accession No. MI0016861, SEQ ID NO: 300) having a hairpin-like structure is known as a precursor of “hsa-miR-4419b”.

The term “hsa-miR-7641 gene” or “hsa-miR-7641” used in the present specification includes the hsa-miR-7641 gene (miRBase Accession No. MIMAT0029782) described in SEQ ID NO: 116, a homolog or an ortholog of a different organism species, and the like. The hsa-miR-7641 gene can be obtained by a method described in Yoo J K et al., 2013, Arch Pharm Res, Vol. 36, p. 353-358. Also, “hsa-mir-7641-1 and hsa-mir-7641-2” (miRBase Accession Nos. MI0024975 and MI0024976, SEQ ID NOs: 301 and 302) having a hairpin-like structure are known as precursors of “hsa-miR-7641”.

The term “hsa-miR-3928-3p gene” or “hsa-miR-3928-3p” used in the present specification includes the hsa-miR-3928-3p gene (miRBase Accession No. MIMAT0018205) described in SEQ ID NO: 117, a homolog or an ortholog of a different organism species, and the like. The hsa-miR-3928-3p gene can be obtained by a method described in Creighton C J et al., 2010, PLoS One, Vol. 5, e9637. Also, “hsa-mir-3928” (miRBase Accession No. MI0016438, SEQ ID NO: 303) having a hairpin-like structure is known as a precursor of “hsa-miR-3928-3p”.

The term “hsa-miR-1227-5p gene” or “hsa-miR-1227-5p” used in the present specification includes the hsa-miR-1227-5p gene (miRBase Accession No. MIMAT0022941) described in SEQ ID NO: 118, a homolog or an ortholog of a different organism species, and the like. The hsa-miR-1227-5p gene can be obtained by a method described in Berezikov E et al., 2007, Mol Cell, Vol. 28, p. 328-336. Also, “hsa-mir-1227” (miRBase Accession No. MI0006316, SEQ ID NO: 304) having a hairpin-like structure is known as a precursor of “hsa-miR-1227-5p”.

The term “hsa-miR-4492 gene” or “hsa-miR-4492” used in the present specification includes the hsa-miR-4492 gene (miRBase Accession No. MIMAT0019027) described in SEQ ID NO: 119, a homolog or an ortholog of a different organism species, and the like. The hsa-miR-4492 gene can be obtained by a method described in Jima D D et al., 2010, Blood, Vol. 116, e118-e127. Also, “hsa-mir-4492” (miRBase Accession No. MI0016854, SEQ ID NO: 305) having a hairpin-like structure is known as a precursor of “hsa-miR-4492”.

The term “hsa-miR-296-3p gene” or “hsa-miR-296-3p” used in the present specification includes the hsa-miR-296-3p gene (miRBase Accession No. MIMAT0004679) described in SEQ ID NO: 120, a homolog or an ortholog of a different organism species, and the like. The hsa-miR-296-3p gene can be obtained by a method described in Houbaviy H B et al., 2003, Dev Cell, Vol. 5, p. 351-358. Also, “hsa-mir-296” (miRBase Accession No. MI0000747, SEQ ID NO: 306) having a hairpin-like structure is known as a precursor of “hsa-miR-296-3p”.

The term “hsa-miR-6769a-5p gene” or “hsa-miR-6769a-5p” used in the present specification includes the hsa-miR-6769a-5p gene (miRBase Accession No. MIMAT0027438) described in SEQ ID NO: 121, a homolog or an ortholog of a different organism species, and the like. The hsa-miR-6769a-5p gene can be obtained by a method described in Ladewig E et al., 2012, Genome Res, Vol. 22, p. 1634-1645. Also, “hsa-mir-6769a” (miRBase Accession No. MI0022614, SEQ ID NO: 307) having a hairpin-like structure is known as a precursor of “hsa-miR-6769a-5p”.

The term “hsa-miR-6889-5p gene” or “hsa-miR-6889-5p” used in the present specification includes the hsa-miR-6889-5p gene (miRBase Accession No. MIMAT0027678) described in SEQ ID NO: 122, a homolog or an ortholog of a different organism species, and the like. The hsa-miR-6889-5p gene can be obtained by a method described in Ladewig E et al., 2012, Genome Res, Vol. 22, p. 1634-1645. Also, “hsa-mir-6889” (miRBase Accession No. MI0022736, SEQ ID NO: 308) having a hairpin-like structure is known as a precursor of “hsa-miR-6889-5p”.

The term “hsa-miR-4632-5p gene” or “hsa-miR-4632-5p” used in the present specification includes the hsa-miR-4632-5p gene (miRBase Accession No. MIMAT0022977) described in SEQ ID NO: 123, a homolog or an ortholog of a different organism species, and the like. The hsa-miR-4632-5p gene can be obtained by a method described in Persson H et al., 2011, Cancer Res, Vol. 71, p. 78-86. Also, “hsa-mir-4632” (miRBase Accession No. MI0017259, SEQ ID NO: 309) having a hairpin-like structure is known as a precursor of “hsa-miR-4632-5p”.

The term “hsa-miR-4505 gene” or “hsa-miR-4505” used in the present specification includes the hsa-miR-4505 gene (miRBase Accession No. MIMAT0019041) described in SEQ ID NO: 124, a homolog or an ortholog of a different organism species, and the like. The hsa-miR-4505 gene can be obtained by a method described in Jima D D et al., 2010, Blood, Vol. 116, e118-e127. Also, “hsa-mir-4505” (miRBase Accession No. MI0016868, SEQ ID NO: 310) having a hairpin-like structure is known as a precursor of “hsa-miR-4505”.

The term “hsa-miR-3154 gene” or “hsa-miR-3154” used in the present specification includes the hsa-miR-3154 gene (miRBase Accession No. MIMAT0015028) described in SEQ ID NO: 125, a homolog or an ortholog of a different organism species, and the like. The hsa-miR-3154 gene can be obtained by a method described in Berezikov E et al., 2006, Genome Res, Vol. 16, p. 1289-1298. Also, “hsa-mir-3154” (miRBase Accession No. MI0014182, SEQ ID NO: 311) having a hairpin-like structure is known as a precursor of “hsa-miR-3154”.

The term “hsa-miR-3648 gene” or “hsa-miR-3648” used in the present specification includes the hsa-miR-3648 gene (miRBase Accession No. MIMAT0018068) described in SEQ ID NO: 126, a homolog or an ortholog of a different organism species, and the like. The hsa-miR-3648 gene can be obtained by a method described in Meiri E et al., 2010, Nucleic Acids Res, Vol. 38, p. 6234-6246. Also, “hsa-mir-3648” (miRBase Accession No. MI0016048, SEQ ID NO: 312) having a hairpin-like structure is known as a precursor of “hsa-miR-3648”.

The term “hsa-miR-4442 gene” or “hsa-miR-4442” used in the present specification includes the hsa-miR-4442 gene (miRBase Accession No. MIMAT0018960) described in SEQ ID NO: 127, a homolog or an ortholog of a different organism species, and the like. The hsa-miR-4442 gene can be obtained by a method described in Jima D D et al., 2010, Blood, Vol. 116, e118-e127. Also, “hsa-mir-4442” (miRBase Accession No. MI0016785, SEQ ID NO: 313) having a hairpin-like structure is known as a precursor of “hsa-miR-4442”.

The term “hsa-miR-3141 gene” or “hsa-miR-3141” used in the present specification includes the hsa-miR-3141 gene (miRBase Accession No. MIMAT0015010) described in SEQ ID NO: 128, a homolog or an ortholog of a different organism species, and the like. The hsa-miR-3141 gene can be obtained by a method described in Stark M S et al., 2010, PLoS One, Vol. 5, e9685. Also, “hsa-mir-3141” (miRBase Accession No. MI0014165, SEQ ID NO: 314) having a hairpin-like structure is known as a precursor of “hsa-miR-3141”.

The term “hsa-miR-7113-3p gene” or “hsa-miR-7113-3p” used in the present specification includes the hsa-miR-7113-3p gene (miRBase Accession No. MIMAT0028124) described in SEQ ID NO: 129, a homolog or an ortholog of a different organism species, and the like. The hsa-miR-7113-3p gene can be obtained by a method described in Ladewig E et al., 2012, Genome Res, Vol. 22, p. 1634-1645. Also, “hsa-mir-7113” (miRBase Accession No. MI0022964, SEQ ID NO: 315) having a hairpin-like structure is known as a precursor of “hsa-miR-7113-3p”.

The term “hsa-miR-6819-5p gene” or “hsa-miR-6819-5p” used in the present specification includes the hsa-miR-6819-5p gene (miRBase Accession No. MIMAT0027538) described in SEQ ID NO: 130, a homolog or an ortholog of a different organism species, and the like. The hsa-miR-6819-5p gene can be obtained by a method described in Ladewig E et al., 2012, Genome Res, Vol. 22, p. 1634-1645. Also, “hsa-mir-6819” (miRBase Accession No. MI0022664, SEQ ID NO: 316) having a hairpin-like structure is known as a precursor of “hsa-miR-6819-5p”.

The term “hsa-miR-3195 gene” or “hsa-miR-3195” used in the present specification includes the hsa-miR-3195 gene (miRBase Accession No. MIMAT0015079) described in SEQ ID NO: 131, a homolog or an ortholog of a different organism species, and the like. The hsa-miR-3195 gene can be obtained by a method described in Stark M S et al., 2010, PLoS One, Vol. 5, e9685. Also, “hsa-mir-3195” (miRBase Accession No. MI0014240, SEQ ID NO: 317) having a hairpin-like structure is known as a precursor of “hsa-miR-3195”.

The term “hsa-miR-1199-5p gene” or “hsa-miR-1199-5p” used in the present specification includes the hsa-miR-1199-5p gene (miRBase Accession No. MIMAT0031119) described in SEQ ID NO: 132, a homolog or an ortholog of a different organism species, and the like. The hsa-miR-1199-5p gene can be obtained by a method described in Salvi A et al., 2013, Int J Oncol, Vol. 42, p. 391-402. Also, “hsa-mir-1199” (miRBase Accession No. MI0020340, SEQ ID NO: 318) having a hairpin-like structure is known as a precursor of “hsa-miR-1199-5p”.

The term “hsa-miR-6738-5p gene” or “hsa-miR-6738-5p” used in the present specification includes the hsa-miR-6738-5p gene (miRBase Accession No. MIMAT0027377) described in SEQ ID NO: 133, a homolog or an ortholog of a different organism species, and the like. The hsa-miR-6738-5p gene can be obtained by a method described in Ladewig E et al., 2012, Genome Res, Vol. 22, p. 1634-1645. Also, “hsa-mir-6738” (miRBase Accession No. MI0022583, SEQ ID NO: 319) having a hairpin-like structure is known as a precursor of “hsa-miR-6738-5p”.

The term “hsa-miR-4656 gene” or “hsa-miR-4656” used in the present specification includes the hsa-miR-4656 gene (miRBase Accession No. MIMAT0019723) described in SEQ ID NO: 134, a homolog or an ortholog of a different organism species, and the like. The hsa-miR-4656 gene can be obtained by a method described in Persson H et al., 2011, Cancer Res, Vol. 71, p. 78-86. Also, “hsa-mir-4656” (miRBase Accession No. MI0017284, SEQ ID NO: 320) having a hairpin-like structure is known as a precursor of “hsa-miR-4656”.

The term “hsa-miR-6820-5p gene” or “hsa-miR-6820-5p” used in the present specification includes the hsa-miR-6820-5p gene (miRBase Accession No. MIMAT0027540) described in SEQ ID NO: 135, a homolog or an ortholog of a different organism species, and the like. The hsa-miR-6820-5p gene can be obtained by a method described in Ladewig E et al., 2012, Genome Res, Vol. 22, p. 1634-1645. Also, “hsa-mir-6820” (miRBase Accession No. MI0022665, SEQ ID NO: 321) having a hairpin-like structure is known as a precursor of “hsa-miR-6820-5p”.

The term “hsa-miR-615-5p gene” or “hsa-miR-615-5p” used in the present specification includes the hsa-miR-615-5p gene (miRBase Accession No. MIMAT0004804) described in SEQ ID NO: 136, a homolog or an ortholog of a different organism species, and the like. The hsa-miR-615-5p gene can be obtained by a method described in Cummins J M et al., 2006, Proc Natl Acad Sci USA, Vol. 103, p. 3687-3692. Also, “hsa-mir-615” (miRBase Accession No. MI0003628, SEQ ID NO: 322) having a hairpin-like structure is known as a precursor of “hsa-miR-615-5p”.

The term “hsa-miR-486-3p gene” or “hsa-miR-486-3p” used in the present specification includes the hsa-miR-486-3p gene (miRBase Accession No. MIMAT0004762) described in SEQ ID NO: 137, a homolog or an ortholog of a different organism species, and the like. The hsa-miR-486-3p gene can be obtained by a method described in Fu H et al., 2005, FEBS Lett, Vol. 579, p. 3849-3854. Also, “hsa-mir-486 and hsa-mir-486-2” (miRBase Accession Nos. MI0002470 and MI0023622, SEQ ID NO: 323 and 324) having a hairpin-like structure are known as precursors of “hsa-miR-486-3p”.

The term “hsa-miR-1225-3p gene” or “hsa-miR-1225-3p” used in the present specification includes the hsa-miR-1225-3p gene (miRBase Accession No. MIMAT0005573) described in SEQ ID NO: 138, a homolog or an ortholog of a different organism species, and the like. The hsa-miR-1225-3p gene can be obtained by a method described in Berezikov E et al., 2007, Mol Cell, Vol. 28, p. 328-336. Also, “hsa-mir-1225” (miRBase Accession No. MI0006311, SEQ ID NO: 325) having a hairpin-like structure is known as a precursor of “hsa-miR-1225-3p”.

The term “hsa-miR-760 gene” or “hsa-miR-760” used in the present specification includes the hsa-miR-760 gene (miRBase Accession No. MIMAT0004957) described in SEQ ID NO: 139, a homolog or an ortholog of a different organism species, and the like. The hsa-miR-760 gene can be obtained by a method described in Berezikov E et al., 2006, Genome Res, Vol. 16, p. 1289-1298. Also, “hsa-mir-760” (miRBase Accession No. MI0005567, SEQ ID NO: 326) having a hairpin-like structure is known as a precursor of “hsa-miR-760”.

The term “hsa-miR-187-5p gene” or “hsa-miR-187-5p” used in the present specification includes the hsa-miR-187-5p gene (miRBase Accession No. MIMAT0004561) described in SEQ ID NO: 140, a homolog or an ortholog of a different organism species, and the like. The hsa-miR-187-5p gene can be obtained by a method described in Lim L P et al., 2003, Science, Vol. 299, p. 1540. Also, “hsa-mir-187” (miRBase Accession No. MI0000274, SEQ ID NO: 327) having a hairpin-like structure is known as a precursor of “hsa-miR-187-5p”.

The term “hsa-miR-1203 gene” or “hsa-miR-1203” used in the present specification includes the hsa-miR-1203 gene (miRBase Accession No. MIMAT0005866) described in SEQ ID NO: 141, a homolog or an ortholog of a different organism species, and the like. The hsa-miR-1203 gene can be obtained by a method described in Marton S et al., 2008, Leukemia, Vol. 22, p. 330-338. Also, “hsa-mir-1203” (miRBase Accession No. MI0006335, SEQ ID NO: 328) having a hairpin-like structure is known as a precursor of “hsa-miR-1203”.

The term “hsa-miR-7110-5p gene” or “hsa-miR-7110-5p” used in the present specification includes the hsa-miR-7110-5p gene (miRBase Accession No. MIMAT0028117) described in SEQ ID NO: 142, a homolog or an ortholog of a different organism species, and the like. The hsa-miR-7110-5p gene can be obtained by a method described in Ladewig E et al., 2012, Genome Res, Vol. 22, p. 1634-1645. Also, “hsa-mir-7110” (miRBase Accession No. MI0022961, SEQ ID NO: 329) having a hairpin-like structure is known as a precursor of “hsa-miR-7110-5p”.

The term “hsa-miR-371a-5p gene” or “hsa-miR-371a-5p” used in the present specification includes the hsa-miR-371a-5p gene (miRBase Accession No. MIMAT0004687) described in SEQ ID NO: 143, a homolog or an ortholog of a different organism species, and the like. The hsa-miR-371a-5p gene can be obtained by a method described in Suh M R et al., 2004, Dev Biol, Vol. 270, p. 488-498. Also, “hsa-mir-371a” (miRBase Accession No. MI0000779, SEQ ID NO: 330) having a hairpin-like structure is known as a precursor of “hsa-miR-371a-5p”.

The term “hsa-miR-939-5p gene” or “hsa-miR-939-5p” used in the present specification includes the hsa-miR-939-5p gene (miRBase Accession No. MIMAT0004982) described in SEQ ID NO: 144, a homolog or an ortholog of a different organism species, and the like. The hsa-miR-939-5p gene can be obtained by a method described in Lui W O et al., 2007, Cancer Res, Vol. 67, p. 6031-6043. Also, “hsa-mir-939” (miRBase Accession No. MI0005761, SEQ ID NO: 331) having a hairpin-like structure is known as a precursor of “hsa-miR-939-5p”.

The term “hsa-miR-575 gene” or “hsa-miR-575” used in the present specification includes the hsa-miR-575 gene (miRBase Accession No. MIMAT0003240) described in SEQ ID NO: 145, a homolog or an ortholog of a different organism species, and the like. The hsa-miR-575 gene can be obtained by a method described in Cummins J M et al., 2006, Proc Natl Acad Sci USA, Vol. 103, p. 3687-3692. Also, “hsa-mir-575” (miRBase Accession No. MI0003582, SEQ ID NO: 332) having a hairpin-like structure is known as a precursor of “hsa-miR-575”.

The term “hsa-miR-92b-5p gene” or “hsa-miR-92b-5p” used in the present specification includes the hsa-miR-92b-5p gene (miRBase Accession No. MIMAT0004792) described in SEQ ID NO: 146, a homolog or an ortholog of a different organism species, and the like. The hsa-miR-92b-5p gene can be obtained by a method described in Cummins J M et al., 2006, Proc Natl Acad Sci USA, Vol. 103, p. 3687-3692. Also, “hsa-mir-92b” (miRBase Accession No. MI0003560, SEQ ID NO: 333) having a hairpin-like structure is known as a precursor of “hsa-miR-92b-5p”.

The term “hsa-miR-887-3p gene” or “hsa-miR-887-3p” used in the present specification includes the hsa-miR-887-3p gene (miRBase Accession No. MIMAT0004951) described in SEQ ID NO: 147, a homolog or an ortholog of a different organism species, and the like. The hsa-miR-887-3p gene can be obtained by a method described in Berezikov E et al., 2006, Genome Res, Vol. 16, p. 1289-1298. Also, “hsa-mir-887” (miRBase Accession No. MI0005562, SEQ ID NO: 334) having a hairpin-like structure is known as a precursor of “hsa-miR-887-3p”.

The term “hsa-miR-920 gene” or “hsa-miR-920” used in the present specification includes the hsa-miR-920 gene (miRBase Accession No. MIMAT0004970) described in SEQ ID NO: 148, a homolog or an ortholog of a different organism species, and the like. The hsa-miR-920 gene can be obtained by a method described in Novotny G W et al., 2007, Int J Androl, Vol. 30, p. 316-326. Also, “hsa-mir-920” (miRBase Accession No. MI0005712, SEQ ID NO: 335) having a hairpin-like structure is known as a precursor of “hsa-miR-920”.

The term “hsa-miR-1915-5p gene” or “hsa-miR-1915-5p” used in the present specification includes the hsa-miR-1915-5p gene (miRBase Accession No. MIMAT0007891) described in SEQ ID NO: 149, a homolog or an ortholog of a different organism species, and the like. The hsa-miR-1915-5p gene can be obtained by a method described in Bar M et al., 2008, Stem Cells, Vol. 26, p. 2496-2⁵⁰⁵. Also, “hsa-mir-1915” (miRBase Accession No. MI0008336, SEQ ID NO: 336) having a hairpin-like structure is known as a precursor of “hsa-miR-1915-5p”.

The term “hsa-miR-1231 gene” or “hsa-miR-1231” used in the present specification includes the hsa-miR-1231 gene (miRBase Accession No. MIMAT0005586) described in SEQ ID NO: 150, a homolog or an ortholog of a different organism species, and the like. The hsa-miR-1231 gene can be obtained by a method described in Berezikov E et al., 2007, Mol Cell, Vol. 28, p. 328-336. Also, “hsa-mir-1231” (miRBase Accession No. MI0006321, SEQ ID NO: 337) having a hairpin-like structure is known as a precursor of “hsa-miR-1231”.

The term “hsa-miR-663b gene” or “hsa-miR-663b” used in the present specification includes the hsa-miR-663b gene (miRBase Accession No. MIMAT0005867) described in SEQ ID NO: 151, a homolog or an ortholog of a different organism species, and the like. The hsa-miR-663b gene can be obtained by a method described in Takada S et al., 2008, Leukemia, Vol. 22, p. 1274-1278. Also, “hsa-mir-663b” (miRBase Accession No. MI0006336, SEQ ID NO: 338) having a hairpin-like structure is known as a precursor of “hsa-miR-663b”.

The term “hsa-miR-1225-5p gene” or “hsa-miR-1225-5p” used in the present specification includes the hsa-miR-1225-5p gene (miRBase Accession No. MIMAT0005572) described in SEQ ID NO: 152, a homolog or an ortholog of a different organism species, and the like. The hsa-miR-1225-5p gene can be obtained by a method described in Berezikov E et al., 2007, Mol Cell, Vol. 28, p. 328-336. Also, “hsa-mir-1225” (miRBase Accession No. MI0006311, SEQ ID NO: 325) having a hairpin-like structure is known as a precursor of “hsa-miR-1225-5p”.

The term “hsa-miR-4763-3p gene” or “hsa-miR-4763-3p” used in the present specification includes the hsa-miR-4763-3p gene (miRBase Accession No. MIMAT0019913) described in SEQ ID NO: 153, a homolog or an ortholog of a different organism species, and the like. The hsa-miR-4763-3p gene can be obtained by a method described in Persson H et al., 2011, Cancer Res, Vol. 71, p. 78-86. Also, “hsa-mir-4763” (miRBase Accession No. MI0017404, SEQ ID NO: 339) having a hairpin-like structure is known as a precursor of “hsa-miR-4763-3p”.

The term “hsa-miR-3656 gene” or “hsa-miR-3656” used in the present specification includes the hsa-miR-3656 gene (miRBase Accession No. MIMAT0018076) described in SEQ ID NO: 154, a homolog or an ortholog of a different organism species, and the like. The hsa-miR-3656 gene can be obtained by a method described in Meiri E et al., 2010, Nucleic Acids Res, Vol. 38, p. 6234-6246. Also, “hsa-mir-3656” (miRBase Accession No. MI0016056, SEQ ID NO: 340) having a hairpin-like structure is known as a precursor of “hsa-miR-3656”.

The term “hsa-miR-4488 gene” or “hsa-miR-4488” used in the present specification includes the hsa-miR-4488 gene (miRBase Accession No. MIMAT0019022) described in SEQ ID NO: 155, a homolog or an ortholog of a different organism species, and the like. The hsa-miR-4488 gene can be obtained by a method described in Jima D D et al., 2010, Blood, Vol. 116, e118-e127. Also, “hsa-mir-4488” (miRBase Accession No. MI0016849, SEQ ID NO: 341) having a hairpin-like structure is known as a precursor of “hsa-miR-4488”.

The term “hsa-miR-125a-3p gene” or “hsa-miR-125a-3p” used in the present specification includes the hsa-miR-125a-3p gene (miRBase Accession No. MIMAT0004602) described in SEQ ID NO: 156, a homolog or an ortholog of a different organism species, and the like. The hsa-miR-125a-3p gene can be obtained by a method described in Lagos-Quintana M et al., 2002, Curr Biol, Vol. 12, p. 735-739. Also, “hsa-mir-125a” (miRBase Accession No. MI0000469, SEQ ID NO: 342) having a hairpin-like structure is known as a precursor of “hsa-miR-125a-3p”.

The term “hsa-miR-1469 gene” or “hsa-miR-1469” used in the present specification includes the hsa-miR-1469 gene (miRBase Accession No. MIMAT0007347) described in SEQ ID NO: 157, a homolog or an ortholog of a different organism species, and the like. The hsa-miR-1469 gene can be obtained by a method described in Kawaji H et al., 2008, BMC Genomics, Vol. 9, p. 157. Also, “hsa-mir-1469” (miRBase Accession No. MI0007074, SEQ ID NO: 343) having a hairpin-like structure is known as a precursor of “hsa-miR-1469”.

The term “hsa-miR-1228-5p gene” or “hsa-miR-1228-5p” used in the present specification includes the hsa-miR-1228-5p gene (miRBase Accession No. MIMAT0005582) described in SEQ ID NO: 158, a homolog or an ortholog of a different organism species, and the like. The hsa-miR-1228-5p gene can be obtained by a method described in Berezikov E et al., 2007, Mol Cell, Vol. 28, p. 328-336. Also, “hsa-mir-1228” (miRBase Accession No. MI0006318, SEQ ID NO: 344) having a hairpin-like structure is known as a precursor of “hsa-miR-1228-5p”.

The term “hsa-miR-6798-5p gene” or “hsa-miR-6798-5p” used in the present specification includes the hsa-miR-6798-5p gene (miRBase Accession No. MIMAT0027496) described in SEQ ID NO: 159, a homolog or an ortholog of a different organism species, and the like. The hsa-miR-6798-5p gene can be obtained by a method described in Ladewig E et al., 2012, Genome Res, Vol. 22, p. 1634-1645. Also, “hsa-mir-6798” (miRBase Accession No. MI0022643, SEQ ID NO: 345) having a hairpin-like structure is known as a precursor of “hsa-miR-6798-5p”.

The term “hsa-miR-1268b gene” or “hsa-miR-1268b” used in the present specification includes the hsa-miR-1268b gene (miRBase Accession No. MIMAT0018925) described in SEQ ID NO: 160, a homolog or an ortholog of a different organism species, and the like. The hsa-miR-1268b gene can be obtained by a method described in Jima D D et al., 2010, Blood, Vol. 116, e118-e127. Also, “hsa-mir-1268b” (miRBase Accession No. MI0016748, SEQ ID NO: 346) having a hairpin-like structure is known as a precursor of “hsa-miR-1268b”.

The term “hsa-miR-6732-5p gene” or “hsa-miR-6732-5p” used in the present specification includes the hsa-miR-6732-5p gene (miRBase Accession No. MIMAT0027365) described in SEQ ID NO: 161, a homolog or an ortholog of a different organism species, and the like. The hsa-miR-6732-5p gene can be obtained by a method described in Ladewig E et al., 2012, Genome Res, Vol. 22, p. 1634-1645. Also, “hsa-mir-6732” (miRBase Accession No. MI0022577, SEQ ID NO: 347) having a hairpin-like structure is known as a precursor of “hsa-miR-6732-5p”.

The term “hsa-miR-1915-3p gene” or “hsa-miR-1915-3p” used in the present specification includes the hsa-miR-1915-3p gene (miRBase Accession No. MIMAT0007892) described in SEQ ID NO: 162, a homolog or an ortholog of a different organism species, and the like. The hsa-miR-1915-3p gene can be obtained by a method described in Bar M et al., 2008, Stem Cells, Vol. 26, p. 2496-2⁵⁰⁵. Also, “hsa-mir-1915” (miRBase Accession No. MI0008336, SEQ ID NO: 336) having a hairpin-like structure is known as a precursor of “hsa-miR-1915-3p”.

The term “hsa-miR-4433b-3p gene” or “hsa-miR-4433b-3p” used in the present specification includes the hsa-miR-4433b-3p gene (miRBase Accession No. MIMAT0030414) described in SEQ ID NO: 163, a homolog or an ortholog of a different organism species, and the like. The hsa-miR-4433b-3p gene can be obtained by a method described in Ple H et al., 2012, PLoS One, Vol. 7, e50746. Also, “hsa-mir-4433b” (miRBase Accession No. MI0025511, SEQ ID NO: 348) having a hairpin-like structure is known as a precursor of “hsa-miR-4433b-3p”.

The term “hsa-miR-1207-5p gene” or “hsa-miR-1207-5p” used in the present specification includes the hsa-miR-1207-5p gene (miRBase Accession No. MIMAT0005871) described in SEQ ID NO: 164, a homolog or an ortholog of a different organism species, and the like. The hsa-miR-1207-5p gene can be obtained by a method described in Huppi K et al., 2008, Mol Cancer Res, Vol. 6, p. 212-221. Also, “hsa-mir-1207” (miRBase Accession No. MI0006340, SEQ ID NO: 349) having a hairpin-like structure is known as a precursor of “hsa-miR-1207-5p”.

The term “hsa-miR-4433-3p gene” or “hsa-miR-4433-3p” used in the present specification includes the hsa-miR-4433-3p gene (miRBase Accession No. MIMAT0018949) described in SEQ ID NO: 165, a homolog or an ortholog of a different organism species, and the like. The hsa-miR-4433-3p gene can be obtained by a method described in Jima D D et al., 2010, Blood, Vol. 116, e118-e127. Also, “hsa-mir-4433” (miRBase Accession No. MI0016773, SEQ ID NO: 350) having a hairpin-like structure is known as a precursor of “hsa-miR-4433-3p”.

The term “hsa-miR-6879-5p gene” or “hsa-miR-6879-5p” used in the present specification includes the hsa-miR-6879-5p gene (miRBase Accession No. MIMAT0027658) described in SEQ ID NO: 166, a homolog or an ortholog of a different organism species, and the like. The hsa-miR-6879-5p gene can be obtained by a method described in Ladewig E et al., 2012, Genome Res, Vol. 22, p. 1634-1645. Also, “hsa-mir-6879” (miRBase Accession No. MI0022726, SEQ ID NO: 351) having a hairpin-like structure is known as a precursor of “hsa-miR-6879-5p”.

The term “hsa-miR-4417 gene” or “hsa-miR-4417” used in the present specification includes the hsa-miR-4417 gene (miRBase Accession No. MIMAT0018929) described in SEQ ID NO: 167, a homolog or an ortholog of a different organism species, and the like. The hsa-miR-4417 gene can be obtained by a method described in Jima D D et al., 2010, Blood, Vol. 116, e118-e127. Also, “hsa-mir-4417” (miRBase Accession No. MI0016753, SEQ ID NO: 352) having a hairpin-like structure is known as a precursor of “hsa-miR-4417”.

The term “hsa-miR-30c-1-3p gene” or “hsa-miR-30c-1-3p” used in the present specification includes the hsa-miR-30c-1-3p gene (miRBase Accession No. MIMAT0004674) described in SEQ ID NO: 168, a homolog or an ortholog of a different organism species, and the like. The hsa-miR-30c-1-3p gene can be obtained by a method described in Lagos-Quintana M et al., 2002, Curr Biol, Vol. 12, p. 735-739. Also, “hsa-mir-30c-1” (miRBase Accession No. MI0000736, SEQ ID NO: 353) having a hairpin-like structure is known as a precursor of “hsa-miR-30c-1-3p”.

The term “hsa-miR-4638-5p gene” or “hsa-miR-4638-5p” used in the present specification includes the hsa-miR-4638-5p gene (miRBase Accession No. MIMAT0019695) described in SEQ ID NO: 169, a homolog or an ortholog of a different organism species, and the like. The hsa-miR-4638-5p gene can be obtained by a method described in Persson H et al., 2011, Cancer Res, Vol. 71, p. 78-86. Also, “hsa-mir-4638” (miRBase Accession No. MI0017265, SEQ ID NO: 354) having a hairpin-like structure is known as a precursor of “hsa-miR-4638-5p”.

The term “hsa-miR-6088 gene” or “hsa-miR-6088” used in the present specification includes the hsa-miR-6088 gene (miRBase Accession No. MIMAT0023713) described in SEQ ID NO: 170, a homolog or an ortholog of a different organism species, and the like. The hsa-miR-6088 gene can be obtained by a method described in Yoo J K et al., 2012, Stem Cells Dev, Vol. 21, p. 2049-2057. Also, “hsa-mir-6088” (miRBase Accession No. MI0020365, SEQ ID NO: 355) having a hairpin-like structure is known as a precursor of “hsa-miR-6088”.

The term “hsa-miR-4270 gene” or “hsa-miR-4270” used in the present specification includes the hsa-miR-4270 gene (miRBase Accession No. MIMAT0016900) described in SEQ ID NO: 171, a homolog or an ortholog of a different organism species, and the like. The hsa-miR-4270 gene can be obtained by a method described in Goff L A et al., 2009, PLoS One, Vol. 4, e7192. Also, “hsa-mir-4270” (miRBase Accession No. MI0015878, SEQ ID NO: 356) having a hairpin-like structure is known as a precursor of “hsa-miR-4270”.

The term “hsa-miR-6782-5p gene” or “hsa-miR-6782-5p” used in the present specification includes the hsa-miR-6782-5p gene (miRBase Accession No. MIMAT0027464) described in SEQ ID NO: 172, a homolog or an ortholog of a different organism species, and the like. The hsa-miR-6782-5p gene can be obtained by a method described in Ladewig E et al., 2012, Genome Res, Vol. 22, p. 1634-1645. Also, “hsa-mir-6782” (miRBase Accession No. MI0022627, SEQ ID NO: 357) having a hairpin-like structure is known as a precursor of “hsa-miR-6782-5p”.

The term “hsa-miR-665 gene” or “hsa-miR-665” used in the present specification includes the hsa-miR-665 gene (miRBase Accession No. MIMAT0004952) described in SEQ ID NO: 173, a homolog or an ortholog of a different organism species, and the like. The hsa-miR-665 gene can be obtained by a method described in Berezikov E et al., 2006, Genome Res, Vol. 16, p. 1289-1298. Also, “hsa-mir-665” (miRBase Accession No. MI0005563, SEQ ID NO: 358) having a hairpin-like structure is known as a precursor of “hsa-miR-665”.

The term “hsa-miR-486-5p gene” or “hsa-miR-486-5p” used in the present specification includes the hsa-miR-486-5p gene (miRBase Accession No. MIMAT0002177) described in SEQ ID NO: 174, a homolog or an ortholog of a different organism species, and the like. The hsa-miR-486-5p gene can be obtained by a method described in Fu H et al., 2005, FEBS Lett, Vol. 579, p. 3849-3854. Also, “hsa-mir-486 and hsa-mir-486-2” (miRBase Accession Nos. MI0002470 and MI0023622, SEQ ID NOs: 323 and 324) having a hairpin-like structure are known as precursors of “hsa-miR-486-5p”.

The term “hsa-miR-4655-5p gene” or “hsa-miR-4655-5p” used in the present specification includes the hsa-miR-4655-5p gene (miRBase Accession No. MIMAT0019721) described in SEQ ID NO: 175, a homolog or an ortholog of a different organism species, and the like. The hsa-miR-4655-5p gene can be obtained by a method described in Persson H et al., 2011, Cancer Res, Vol. 71, p. 78-86. Also, “hsa-mir-4655” (miRBase Accession No. MI0017283, SEQ ID NO: 359) having a hairpin-like structure is known as a precursor of “hsa-miR-4655-5p”.

The term “hsa-miR-1275 gene” or “hsa-miR-1275” used in the present specification includes the hsa-miR-1275 gene (miRBase Accession No. MIMAT0005929) described in SEQ ID NO: 176, a homolog or an ortholog of a different organism species, and the like. The hsa-miR-1275 gene can be obtained by a method described in Morin R D et al., 2008, Genome Res, Vol. 18, p. 610-621. Also, “hsa-mir-1275” (miRBase Accession No. MI0006415, SEQ ID NO: 360) having a hairpin-like structure is known as a precursor of “hsa-miR-1275”.

The term “hsa-miR-6806-5p gene” or “hsa-miR-6806-5p” used in the present specification includes the hsa-miR-6806-5p gene (miRBase Accession No. MIMAT0027512) described in SEQ ID NO: 177, a homolog or an ortholog of a different organism species, and the like. The hsa-miR-6806-5p gene can be obtained by a method described in Ladewig E et al., 2012, Genome Res, Vol. 22, p. 1634-1645. Also, “hsa-mir-6806” (miRBase Accession No. MI0022651, SEQ ID NO: 361) having a hairpin-like structure is known as a precursor of “hsa-miR-6806-5p”.

The term “hsa-miR-614 gene” or “hsa-miR-614” used in the present specification includes the hsa-miR-614 gene (miRBase Accession No. MIMAT0003282) described in SEQ ID NO: 178, a homolog or an ortholog of a different organism species, and the like. The hsa-miR-614 gene can be obtained by a method described in Cummins J M et al., 2006, Proc Natl Acad Sci USA, Vol. 103, p. 3687-3692. Also, “hsa-mir-614” (miRBase Accession No. MI0003627, SEQ ID NO: 362) having a hairpin-like structure is known as a precursor of “hsa-miR-614”.

The term “hsa-miR-3937 gene” or “hsa-miR-3937” used in the present specification includes the hsa-miR-3937 gene (miRBase Accession No. MIMAT0018352) described in SEQ ID NO: 179, a homolog or an ortholog of a different organism species, and the like. The hsa-miR-3937 gene can be obtained by a method described in Liao J Y et al., 2010, PLoS One, Vol. 5, e10563. Also, “hsa-mir-3937” (miRBase Accession No. MI0016593, SEQ ID NO: 363) having a hairpin-like structure is known as a precursor of “hsa-miR-3937”.

The term “hsa-miR-6752-5p gene” or “hsa-miR-6752-5p” used in the present specification includes the hsa-miR-6752-5p gene (miRBase Accession No. MIMAT0027404) described in SEQ ID NO: 180, a homolog or an ortholog of a different organism species, and the like. The hsa-miR-6752-5p gene can be obtained by a method described in Ladewig E et al., 2012, Genome Res, Vol. 22, p. 1634-1645. Also, “hsa-mir-6752” (miRBase Accession No. MI0022597, SEQ ID NO: 364) having a hairpin-like structure is known as a precursor of “hsa-miR-6752-5p”.

The term “hsa-miR-6771-5p gene” or “hsa-miR-6771-5p” used in the present specification includes the hsa-miR-6771-5p gene (miRBase Accession No. MIMAT0027442) described in SEQ ID NO: 181, a homolog or an ortholog of a different organism species, and the like. The hsa-miR-6771-5p gene can be obtained by a method described in Ladewig E et al., 2012, Genome Res, Vol. 22, p. 1634-1645. Also, “hsa-mir-6771” (miRBase Accession No. MI0022616, SEQ ID NO: 365) having a hairpin-like structure is known as a precursor of “hsa-miR-6771-5p”.

The term “hsa-miR-4450 gene” or “hsa-miR-4450” used in the present specification includes the hsa-miR-4450 gene (miRBase Accession No. MIMAT0018971) described in SEQ ID NO: 182, a homolog or an ortholog of a different organism species, and the like. The hsa-miR-4450 gene can be obtained by a method described in Jima D D et al., 2010, Blood, Vol. 116, e118-e127. Also, “hsa-mir-4450” (miRBase Accession No. MI0016795, SEQ ID NO: 366) having a hairpin-like structure is known as a precursor of “hsa-miR-4450”.

The term “hsa-miR-211-3p gene” or “hsa-miR-211-3p” used in the present specification includes the hsa-miR-211-3p gene (miRBase Accession No. MIMAT0022694) described in SEQ ID NO: 183, a homolog or an ortholog of a different organism species, and the like. The hsa-miR-211-3p gene can be obtained by a method described in Lim L P et al., 2003, Science, Vol. 299, p. 1540. Also, “hsa-mir-211” (miRBase Accession No. MI0000287, SEQ ID NO: 367) having a hairpin-like structure is known as a precursor of “hsa-miR-211-3p”.

The term “hsa-miR-663a gene” or “hsa-miR-663a” used in the present specification includes the hsa-miR-663a gene (miRBase Accession No. MIMAT0003326) described in SEQ ID NO: 184, a homolog or an ortholog of a different organism species, and the like. The hsa-miR-663a gene can be obtained by a method described in Cummins J M et al., 2006, Proc Natl Acad Sci USA, Vol. 103, p. 3687-3692. Also, “hsa-mir-663a” (miRBase Accession No. MI0003672, SEQ ID NO: 368) having a hairpin-like structure is known as a precursor of “hsa-miR-663a”.

The term “hsa-miR-6842-5p gene” or “hsa-miR-6842-5p” used in the present specification includes the hsa-miR-6842-5p gene (miRBase Accession No. MIMAT0027586) described in SEQ ID NO: 185, a homolog or an ortholog of a different organism species, and the like. The hsa-miR-6842-5p gene can be obtained by a method described in Ladewig E et al., 2012, Genome Res, Vol. 22, p. 1634-1645. Also, “hsa-mir-6842” (miRBase Accession No. MI0022688, SEQ ID NO: 369) having a hairpin-like structure is known as a precursor of “hsa-miR-6842-5p”.

The term “hsa-miR-7114-5p gene” or “hsa-miR-7114-5p” used in the present specification includes the hsa-miR-7114-5p gene (miRBase Accession No. MIMAT0028125) described in SEQ ID NO: 186, a homolog or an ortholog of a different organism species, and the like. The hsa-miR-7114-5p gene can be obtained by a method described in Ladewig E et al., 2012, Genome Res, Vol. 22, p. 1634-1645. Also, “hsa-mir-7114” (miRBase Accession No. MI0022965, SEQ ID NO: 370) having a hairpin-like structure is known as a precursor of “hsa-miR-7114-5p”.

The term “hsa-miR-6779-5p gene” or “hsa-miR-6779-5p” used in the present specification includes the hsa-miR-6779-5p gene (miRBase Accession No. MIMAT0027458) described in SEQ ID NO: 187, a homolog or an ortholog of a different organism species, and the like. The hsa-miR-6779-5p gene can be obtained by a method described in Ladewig E et al., 2012, Genome Res, Vol. 22, p. 1634-1645. Also, “hsa-mir-6779” (miRBase Accession No. MI0022624, SEQ ID NO: 371) having a hairpin-like structure is known as a precursor of “hsa-miR-6779-5p”.

The term “hsa-miR-204-3p gene” or “hsa-miR-204-3p” used in the present specification includes the hsa-miR-204-3p gene (miRBase Accession No. MIMAT0022693) described in SEQ ID NO: 580, a homolog or an ortholog of a different organism species, and the like. The hsa-miR-204-3p gene can be obtained by a method described in Lim L P et al., 2003, Science., Vol. 299, p. 1540. Also, “hsa-mir-204” (miRBase Accession No. MI0000284, SEQ ID NO: 612) having a hairpin-like structure is known as a precursor of “hsa-miR-204-3p”.

The term “hsa-miR-642a-3p gene” or “hsa-miR-642a-3p” used in the present specification includes the hsa-miR-642a-3p gene (miRBase Accession No. MIMAT0020924) described in SEQ ID NO: 581, a homolog or an ortholog of a different organism species, and the like. The hsa-miR-642a-3p gene can be obtained by a method described in Cummins J M et al., 2006, Proc Natl Acad Sci USA, Vol. 103, p. 3687-3692. Also, “hsa-mir-642a” (miRBase Accession No. MI0003657, SEQ ID NO: 613) having a hairpin-like structure is known as a precursor of “hsa-miR-642a-3p”.

The term “hsa-miR-762 gene” or “hsa-miR-762” used in the present specification includes the hsa-miR-762 gene (miRBase Accession No. MIMAT0010313) described in SEQ ID NO: 582, a homolog or an ortholog of a different organism species, and the like. The hsa-miR-762 gene can be obtained by a method described in Berezikov E et al., 2006, Genome Res, Vol. 16, p. 1289-1298. Also, “hsa-mir-762” (miRBase Accession No. MI0003892, SEQ ID NO: 614) having a hairpin-like structure is known as a precursor of “hsa-miR-762”.

The term “hsa-miR-1202 gene” or “hsa-miR-1202” used in the present specification includes the hsa-miR-1202 gene (miRBase Accession No. MIMAT0005865) described in SEQ ID NO: 583, a homolog or an ortholog of a different organism species, and the like. The hsa-miR-1202 gene can be obtained by a method described in Marton S et al., 2008, Leukemia., Vol. 22, p. 330-338. Also, “hsa-mir-1202” (miRBase Accession No. MI0006334, SEQ ID NO: 615) having a hairpin-like structure is known as a precursor of “hsa-miR-1202”.

The term “hsa-miR-3162-5p gene” or “hsa-miR-3162-5p” used in the present specification includes the hsa-miR-3162-5p gene (miRBase Accession No. MIMAT0015036) described in SEQ ID NO: 584, a homolog or an ortholog of a different organism species, and the like. The hsa-miR-3162-5p gene can be obtained by a method described in Stark M S et al., 2010, PLoS One., Vol. 5, e9685. Also, “hsa-mir-3162” (miRBase Accession No. MI0014192, SEQ ID NO: 616) having a hairpin-like structure is known as a precursor of “hsa-miR-3162-5p”.

The term “hsa-miR-3196 gene” or “hsa-miR-3196” used in the present specification includes the hsa-miR-3196 gene (miRBase Accession No. MIMAT0015080) described in SEQ ID NO: 585, a homolog or an ortholog of a different organism species, and the like. The hsa-miR-3196 gene can be obtained by a method described in Stark M S et al., 2010, PLoS One, Vol. 5, e9685. Also, “hsa-mir-3196” (miRBase Accession No. MI0014241, SEQ ID NO: 617) having a hairpin-like structure is known as a precursor of “hsa-miR-3196”.

The term “hsa-miR-3622a-5p gene” or “hsa-miR-3622a-5p” used in the present specification includes the hsa-miR-3622a-5p gene (miRBase Accession No. MIMAT0018003) described in SEQ ID NO: 586, a homolog or an ortholog of a different organism species, and the like. The hsa-miR-3622a-5p gene can be obtained by a method described in Witten D et al., 2010, BMC Biol., Vol. 8, p. 58. Also, “hsa-mir-3622a” (miRBase Accession No. MI0016013, SEQ ID NO: 618) having a hairpin-like structure is known as a precursor of “hsa-miR-3622a-5p”.

The term “hsa-miR-3665 gene” or “hsa-miR-3665” used in the present specification includes the hsa-miR-3665 gene (miRBase Accession No. MIMAT0018087) described in SEQ ID NO: 587, a homolog or an ortholog of a different organism species, and the like. The hsa-miR-3665 gene can be obtained by a method described in Xie X et al., 2005, Nature, Vol. 434, p. 338-345. Also, “hsa-mir-3665” (miRBase Accession No. MI0016066, SEQ ID NO: 619) having a hairpin-like structure is known as a precursor of “hsa-miR-3665”.

The term “hsa-miR-3940-5p gene” or “hsa-miR-3940-5p” used in the present specification includes the hsa-miR-3940-5p gene (miRBase Accession No. MIMAT0019229) described in SEQ ID NO: 588, a homolog or an ortholog of a different organism species, and the like. The hsa-miR-3940-5p gene can be obtained by a method described in Liao J Y et al., 2010, PLoS One, Vol. 5, e10563. Also, “hsa-mir-3940” (miRBase Accession No. MI0016597, SEQ ID NO: 620) having a hairpin-like structure is known as a precursor of “hsa-miR-3940-5p”.

The term “hsa-miR-4294 gene” or “hsa-miR-4294” used in the present specification includes the hsa-miR-4294 gene (miRBase Accession No. MIMAT0016849) described in SEQ ID NO: 589, a homolog or an ortholog of a different organism species, and the like. The hsa-miR-4294 gene can be obtained by a method described in Goff L A et al., 2009, PLoS One., Vol. 4, e7192. Also, “hsa-mir-4294” (miRBase Accession No. MI0015827, SEQ ID NO: 621) having a hairpin-like structure is known as a precursor of “hsa-miR-4294”.

The term “hsa-miR-4466 gene” or “hsa-miR-4466” used in the present specification includes the hsa-miR-4466 gene (miRBase Accession No. MIMAT0018993) described in SEQ ID NO: 590, a homolog or an ortholog of a different organism species, and the like. The hsa-miR-4466 gene can be obtained by a method described in Jima D D et al., 2010, Blood, Vol. 116, e118-e127. Also, “hsa-mir-4466” (miRBase Accession No. MI0016817, SEQ ID NO: 622) having a hairpin-like structure is known as a precursor of “hsa-miR-4466”.

The term “hsa-miR-4476 gene” or “hsa-miR-4476” used in the present specification includes the hsa-miR-4476 gene (miRBase Accession No. MIMAT0019003) described in SEQ ID NO: 591, a homolog or an ortholog of a different organism species, and the like. The hsa-miR-4476 gene can be obtained by a method described in Jima D D et al., 2010, Blood., Vol. 116, e118-e127. Also, “hsa-mir-4476” (miRBase Accession No. MI0016828, SEQ ID NO: 623) having a hairpin-like structure is known as a precursor of “hsa-miR-4476”.

The term “hsa-miR-4723-5p gene” or “hsa-miR-4723-5p” used in the present specification includes the hsa-miR-4723-5p gene (miRBase Accession No. MIMAT0019838) described in SEQ ID NO: 592, a homolog or an ortholog of a different organism species, and the like. The hsa-miR-4723-5p gene can be obtained by a method described in Persson H et al., 2011, Cancer Res., Vol. 71, p. 78-86. Also, “hsa-mir-4723” (miRBase Accession No. MI0017359, SEQ ID NO: 624) having a hairpin-like structure is known as a precursor of “hsa-miR-4723-5p”.

The term “hsa-miR-4725-3p gene” or “hsa-miR-4725-3p” used in the present specification includes the hsa-miR-4725-3p gene (miRBase Accession No. MIMAT0019844) described in SEQ ID NO: 593, a homolog or an ortholog of a different organism species, and the like. The hsa-miR-4725-3p gene can be obtained by a method described in Persson H et al., 2011, Cancer Res, Vol. 71, p. 78-86. Also, “hsa-mir-4725” (miRBase Accession No. MI0017362, SEQ ID NO: 625) having a hairpin-like structure is known as a precursor of “hsa-miR-4725-3p”.

The term “hsa-miR-4730 gene” or “hsa-miR-4730” used in the present specification includes the hsa-miR-4730 gene (miRBase Accession No. MIMAT0019852) described in SEQ ID NO: 594, a homolog or an ortholog of a different organism species, and the like. The hsa-miR-4730 gene can be obtained by a method described in Persson H et al., 2011, Cancer Res, Vol. 71, p. 78-86. Also, “hsa-mir-4730” (miRBase Accession No. MI0017367, SEQ ID NO: 626) having a hairpin-like structure is known as a precursor of “hsa-miR-4730”.

The term “hsa-miR-4739 gene” or “hsa-miR-4739” used in the present specification includes the hsa-miR-4739 gene (miRBase Accession No. MIMAT0019868) described in SEQ ID NO: 595, a homolog or an ortholog of a different organism species, and the like. The hsa-miR-4739 gene can be obtained by a method described in Persson H et al., 2011, Cancer Res, Vol. 71, p. 78-86. Also, “hsa-mir-4739” (miRBase Accession No. MI0017377, SEQ ID NO: 627) having a hairpin-like structure is known as a precursor of “hsa-miR-4739”.

The term “hsa-miR-4787-5p gene” or “hsa-miR-4787-5p” used in the present specification includes the hsa-miR-4787-5p gene (miRBase Accession No. MIMAT0019956) described in SEQ ID NO: 596, a homolog or an ortholog of a different organism species, and the like. The hsa-miR-4787-5p gene can be obtained by a method described in Persson H et al., 2011, Cancer Res, Vol. 71, p. 78-86. Also, “hsa-mir-4787” (miRBase Accession No. MI0017434, SEQ ID NO: 628) having a hairpin-like structure is known as a precursor of “hsa-miR-4787-5p”.

The term “hsa-miR-5787 gene” or “hsa-miR-5787” used in the present specification includes the hsa-miR-5787 gene (miRBase Accession No. MIMAT0023252) described in SEQ ID NO: 597, a homolog or an ortholog of a different organism species, and the like. The hsa-miR-5787 gene can be obtained by a method described in Yoo H et al., 2011, Biochem Biophys Res Commun, Vol. 415, p. 567-572. Also, “hsa-mir-5787” (miRBase Accession No. MI0019797, SEQ ID NO: 629) having a hairpin-like structure is known as a precursor of “hsa-miR-5787”.

The term “hsa-miR-6085 gene” or “hsa-miR-6085” used in the present specification includes the hsa-miR-6085 gene (miRBase Accession No. MIMAT0023710) described in SEQ ID NO: 598, a homolog or an ortholog of a different organism species, and the like. The hsa-miR-6085 gene can be obtained by a method described in Voellenkle C et al., 2012, RNA., Vol. 18, p. 472-484. Also, “hsa-mir-6085” (miRBase Accession No. MI0020362, SEQ ID NO: 630) having a hairpin-like structure is known as a precursor of “hsa-miR-6085”.

The term “hsa-miR-6717-5p gene” or “hsa-miR-6717-5p” used in the present specification includes the hsa-miR-6717-5p gene (miRBase Accession No. MIMAT0025846) described in SEQ ID NO: 599, a homolog or an ortholog of a different organism species, and the like. The hsa-miR-6717-5p gene can be obtained by a method described in Li Y et al., 2012, Gene, Vol. 497, p. 330-335. Also, “hsa-mir-6717” (miRBase Accession No. MI0022551, SEQ ID NO: 631) having a hairpin-like structure is known as a precursor of “hsa-miR-6717-5p”.

The term “hsa-miR-6724-5p gene” or “hsa-miR-6724-5p” used in the present specification includes the hsa-miR-6724-5p gene (miRBase Accession No. MIMAT0025856) described in SEQ ID NO: 600, a homolog or an ortholog of a different organism species, and the like. The hsa-miR-6724-5p gene can be obtained by a method described in Li Y et al., 2012, Gene., Vol. 497, p. 330-335. Also, “hsa-mir-6724” (miRBase Accession No. MI0022559, SEQ ID NO: 632) having a hairpin-like structure is known as a precursor of “hsa-miR-6724-5p”.

The term “hsa-miR-6777-5p gene” or “hsa-miR-6777-5p” used in the present specification includes the hsa-miR-6777-5p gene (miRBase Accession No. MIMAT0027454) described in SEQ ID NO: 601, a homolog or an ortholog of a different organism species, and the like. The hsa-miR-6777-5p gene can be obtained by a method described in Ladewig E et al., 2012, Genome Res, Vol. 22, p. 1634-1645. Also, “hsa-mir-6777” (miRBase Accession No. MI0022622, SEQ ID NO: 633) having a hairpin-like structure is known as a precursor of “hsa-miR-6777-5p”.

The term “hsa-miR-6778-5p gene” or “hsa-miR-6778-5p” used in the present specification includes the hsa-miR-6778-5p gene (miRBase Accession No. MIMAT0027456) described in SEQ ID NO: 602, a homolog or an ortholog of a different organism species, and the like. The hsa-miR-6778-5p gene can be obtained by a method described in Ladewig E et al., 2012, Genome Res., Vol. 22, p. 1634-1645. Also, “hsa-mir-6778” (miRBase Accession No. MI0022623, SEQ ID NO: 634) having a hairpin-like structure is known as a precursor of “hsa-miR-6778-5p”.

The term “hsa-miR-6787-5p gene” or “hsa-miR-6787-5p” used in the present specification includes the hsa-miR-6787-5p gene (miRBase Accession No. MIMAT0027474) described in SEQ ID NO: 603, a homolog or an ortholog of a different organism species, and the like. The hsa-miR-6787-5p gene can be obtained by a method described in Ladewig E et al., 2012, Genome Res, Vol. 22, p. 1634-1645. Also, “hsa-mir-6787” (miRBase Accession No. MI0022632, SEQ ID NO: 635) having a hairpin-like structure is known as a precursor of “hsa-miR-6787-5p”.

The term “hsa-miR-6789-5p gene” or “hsa-miR-6789-5p” used in the present specification includes the hsa-miR-6789-5p gene (miRBase Accession No. MIMAT0027478) described in SEQ ID NO: 604, a homolog or an ortholog of a different organism species, and the like. The hsa-miR-6789-5p gene can be obtained by a method described in Ladewig E et al., 2012, Genome Res., Vol. 22, p. 1634-1645. Also, “hsa-mir-6789” (miRBase Accession No. MI0022634, SEQ ID NO: 636) having a hairpin-like structure is known as a precursor of “hsa-miR-6789-5p”.

The term “hsa-miR-6845-5p gene” or “hsa-miR-6845-5p” used in the present specification includes the hsa-miR-6845-5p gene (miRBase Accession No. MIMAT0027590) described in SEQ ID NO: 605, a homolog or an ortholog of a different organism species, and the like. The hsa-miR-6845-5p gene can be obtained by a method described in Ladewig E et al., 2012, Genome Res., Vol. 22, p. 1634-1645. Also, “hsa-mir-6845” (miRBase Accession No. MI0022691, SEQ ID NO: 637) having a hairpin-like structure is known as a precursor of “hsa-miR-6845-5p”.

The term “hsa-miR-6893-5p gene” or “hsa-miR-6893-5p” used in the present specification includes the hsa-miR-6893-5p gene (miRBase Accession No. MIMAT0027686) described in SEQ ID NO: 606, a homolog or an ortholog of a different organism species, and the like. The hsa-miR-6893-5p gene can be obtained by a method described in Ladewig E et al., 2012, Genome Res., Vol. 22, p. 1634-1645. Also, “hsa-mir-6893” (miRBase Accession No. MI0022740, SEQ ID NO: 638) having a hairpin-like structure is known as a precursor of “hsa-miR-6893-5p”.

The term “hsa-miR-16-5p gene” or “hsa-miR-16-5p” used in the present specification includes the hsa-miR-16-5p gene (miRBase Accession No. MIMAT0000069) described in SEQ ID NO: 607, a homolog or an ortholog of a different organism species, and the like. The hsa-miR-16-5p gene can be obtained by a method described in Lagos-Quintana M et al., 2002, Curr Biol., Vol. 12, p. 735-739. Also, “hsa-mir-16-1 and hsa-mir-16-2” (miRBase Accession Nos. MI0000070 and MI0000115, SEQ ID NOs: 639 and 640) having a hairpin-like structure are known as precursors of “hsa-miR-16-5p”.

The term “hsa-miR-423-5p gene” or “hsa-miR-423-5p” used in the present specification includes the hsa-miR-423-5p gene (miRBase Accession No. MIMAT0004748) described in SEQ ID NO: 608, a homolog or an ortholog of a different organism species, and the like. The hsa-miR-423-5p gene can be obtained by a method described in Kasashima K et al., 2004, Biochem Biophys Res Commun., Vol. 322, p. 403-410. Also, “hsa-mir-423” (miRBase Accession No. MI0001445, SEQ ID NO: 641) having a hairpin-like structure is known as a precursor of “hsa-miR-423-5p”.

The term “hsa-miR-451a gene” or “hsa-miR-451a” used in the present specification includes the hsa-miR-451a gene (miRBase Accession No. MIMAT0001631) described in SEQ ID NO: 609, a homolog or an ortholog of a different organism species, and the like. The hsa-miR-451a gene can be obtained by a method described in Altuvia Y et al., 2005, Nucleic Acids Res., Vol. 33, p. 2697-2706. Also, “hsa-mir-451a” (miRBase Accession No. MI0001729, SEQ ID NO: 642) having a hairpin-like structure is known as a precursor of “hsa-miR-451a”.

The term “hsa-miR-564 gene” or “hsa-miR-564” used in the present specification includes the hsa-miR-564 gene (miRBase Accession No. MIMAT0003228) described in SEQ ID NO: 610, a homolog or an ortholog of a different organism species, and the like. The hsa-miR-564 gene can be obtained by a method described in Cummins J M, 2006, Proc Natl Acad Sci, Vol. 103, p. 3687-3692. Also, “hsa-mir-564” (miRBase Accession No. MI0003570, SEQ ID NO: 643) having a hairpin-like structure is known as a precursor of “hsa-miR-564”.

The term “hsa-miR-671-5p gene” or “hsa-miR-671-5p” used in the present specification includes the hsa-miR-671-5p gene (miRBase Accession No. MIMAT0003880) described in SEQ ID NO: 611, a homolog or an ortholog of a different organism species, and the like. The hsa-miR-671-5p gene can be obtained by a method described in Berezikov E et al., 2006, Genome Res, Vol. 16, p. 1289-1298. Also, “hsa-mir-671” (miRBase Accession No. MI0003760, SEQ ID NO: 644) having a hairpin-like structure is known as a precursor of “hsa-miR-671-5p”.

A mature miRNA may become a variant due to the sequence cleaved shorter or longer by one to several upstream or downstream bases or base substitution when cleaved as the mature miRNA from its RNA precursor that has a hairpin-like structure. This variant is called isomiR (Morin R D. et al., 2008, Genome Res., Vol. 18, p. 610-621). miRBase Release 20 shows the nucleotide sequences represented by SEQ ID NOs: 1 to 187 and 580 to 611 as well as a large number of the nucleotide sequence variants and fragments represented by SEQ ID NOs: 137 to 579 and 645 to 684, called isomiRs. These variants can also be obtained as miRNAs having a nucleotide sequence represented by any of SEQ ID NOs: 1 to 187 and 580 to 611. Specifically, among the variants of polynucleotides consisting of a nucleotide sequence represented by any of SEQ ID NOs: 1, 2, 4, 5, 6, 7, 10, 12, 15, 16, 18, 19, 21, 22, 24, 25, 27, 30, 31, 33, 34, 36, 39, 41, 42, 43, 44, 45, 46, 48, 51, 53, 58, 61, 62, 63, 66, 69, 73, 75, 76, 77, 78, 83, 84, 85, 86, 87, 88, 90, 94, 95, 96, 98, 100, 102, 103, 104, 105, 106, 107, 108, 109, 111, 115, 117, 119, 120, 123, 124, 125, 126, 127, 128, 131, 136, 137, 139, 140, 143, 144, 147, 149, 151, 153, 154, 155, 156, 158, 160, 162, 165, 167, 168, 169, 170, 173, 174, 175, 176, 178, 182, 183, 184, 580, 581, 584, 585, 587, 588, 590, 591, 592, 593, 594, 595, 597, 599, 600, 607, 608, 609 and 611 or a nucleotide sequence derived from the nucleotide sequence by the replacement of u with t according to the present invention, examples of the longest variants registered in miRBase Release 20 include polynucleotides represented by SEQ ID NOs: 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 645, 647, 650, 652, 655, 657, 659, 661, 663, 665, 667, 669, 671, 673, 675, 677, 679, 681 and 683, respectively. Also, among the variants of polynucleotides consisting of a nucleotide sequence represented by any of SEQ ID NOs: 1, 2, 4, 5, 6, 7, 10, 12, 15, 16, 18, 19, 21, 22, 24, 25, 27, 30, 31, 33, 34, 36, 39, 41, 42, 43, 44, 45, 46, 48, 51, 53, 58, 61, 62, 63, 66, 69, 73, 75, 76, 77, 78, 83, 84, 85, 86, 87, 88, 90, 94, 95, 96, 98, 100, 102, 103, 104, 105, 106, 107, 108, 109, 111, 115, 117, 119, 120, 123, 124, 125, 126, 127, 128, 131, 136, 137, 139, 140, 143, 144, 147, 149, 151, 153, 154, 155, 156, 158, 160, 162, 165, 167, 168, 169, 170, 173, 174, 175, 176, 178, 182, 183, 184, 580, 581, 583, 584, 585, 586, 587, 588, 590, 591, 592, 593, 594, 595, 597, 599, 600, 607, 608, 609 and 611 or a nucleotide sequence derived from the nucleotide sequence by the replacement of u with t according to the present invention, examples of the shortest variants registered in miRBase Release 20 include polynucleotides having sequences represented by SEQ ID NOs: 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 646, 648, 649, 651, 653, 654, 656, 658, 660, 662, 664, 666, 668, 670, 672, 674, 676, 678, 680, 682 and 684, respectively. In addition to these variants and fragments, examples thereof include a large number of isomiR polynucleotides of SEQ ID NOs: 1 to 187 and 580 to 611 registered in miRBase. Examples of the polynucleotide comprising a nucleotide sequence represented by any of SEQ ID NOs: 1 to 187 and 580 to 611 include a polynucleotide represented by any of SEQ ID NOs: 188 to 371, and 612 to 644, which are their respective precursors.

The names and miRBase Accession Nos. (registration numbers) of the genes represented by SEQ ID NOs: 1 to 684 are shown in Table 1.

In the present specification, the term “capable of specifically binding” means that the nucleic acid probe or the primer used in the present invention binds to a particular target nucleic acid and cannot substantially bind to other nucleic acids.

TABLE 1

| SEQ | | miR Base |
|---|---|---|
| ID NO: | Gene name | registration No. |
| 1 | hsa-miR-4443 | MIMAT0018961 |
| 2 | hsa-miR-1908-5p | MIMAT0007881 |
| 3 | hsa-miR-4257 | MIMAT0016878 |
| 4 | hsa-miR-3197 | MIMAT0015082 |
| 5 | hsa-miR-3188 | MIMAT0015070 |
| 6 | hsa-miR-4649-5p | MIMAT0019711 |
| 7 | hsa-miR-1343-3p | MIMAT0019776 |
| 8 | hsa-miR-6861-5p | MIMAT0027623 |
| 9 | hsa-miR-1343-5p | MIMAT0027038 |
| 10 | hsa-miR-642b-3p | MIMAT0018444 |
| 11 | hsa-miR-6741-5p | MIMAT0027383 |
| 12 | hsa-miR-4745-5p | MIMAT0019878 |
| 13 | hsa-miR-6826-5p | MIMAT0027552 |
| 14 | hsa-miR-3663-3p | MIMAT0018085 |
| 15 | hsa-miR-3131 | MIMAT0014996 |
| 16 | hsa-miR-92a-2-5p | MIMAT0004508 |
| 17 | hsa-miR-4258 | MIMAT0016879 |
| 18 | hsa-miR-4448 | MIMAT0018967 |
| 19 | hsa-miR-6125 | MIMAT0024598 |
| 20 | hsa-miR-6880-5p | MIMAT0027660 |
| 21 | hsa-miR-6132 | MIMAT0024616 |
| 22 | hsa-miR-4467 | MIMAT0018994 |
| 23 | hsa-miR-6749-5p | MIMAT0027398 |
| 24 | hsa-miR-2392 | MIMAT0019043 |
| 25 | hsa-miR-1273g-3p | MIMAT0022742 |
| 26 | hsa-miR-4746-3p | MIMAT0019881 |
| 27 | hsa-miR-1914-3p | MIMAT0007890 |
| 28 | hsa-miR-7845-5p | MIMAT0030420 |
| 29 | hsa-miR-6726-5p | MIMAT0027353 |
| 30 | hsa-miR-128-2-5p | MIMAT0031095 |
| 31 | hsa-miR-4651 | MIMAT0019715 |
| 32 | hsa-miR-6765-3p | MIMAT0027431 |
| 33 | hsa-miR-3185 | MIMAT0015065 |
| 34 | hsa-miR-4792 | MIMAT0019964 |
| 35 | hsa-miR-6887-5p | MIMAT0027674 |
| 36 | hsa-miR-5572 | MIMAT0022260 |
| 37 | hsa-miR-3619-3p | MIMAT0019219 |
| 38 | hsa-miR-6780b-5p | MIMAT0027572 |
| 39 | hsa-miR-4707-5p | MIMAT0019807 |
| 40 | hsa-miR-8063 | MIMAT0030990 |
| 41 | hsa-miR-4454 | MIMAT0018976 |
| 42 | hsa-miR-4525 | MIMAT0019064 |
| 43 | hsa-miR-7975 | MIMAT0031178 |
| 44 | hsa-miR-744-5p | MIMAT0004945 |
| 45 | hsa-miR-3135b | MIMAT0018985 |
| 46 | hsa-miR-4648 | MIMAT0019710 |
| 47 | hsa-miR-6816-5p | MIMAT0027532 |
| 48 | hsa-miR-4741 | MIMAT0019871 |
| 49 | hsa-miR-7150 | MIMAT0028211 |
| 50 | hsa-miR-6791-5p | MIMAT0027482 |
| 51 | hsa-miR-1247-3p | MIMAT0022721 |
| 52 | hsa-miR-7977 | MIMAT0031180 |
| 53 | hsa-miR-4497 | MIMAT0019032 |
| 54 | hsa-miR-6090 | MIMAT0023715 |
| 55 | hsa-miR-6781-5p | MIMAT0027462 |
| 56 | hsa-miR-6870-5p | MIMAT0027640 |
| 57 | hsa-miR-6729-5p | MIMAT0027359 |
| 58 | hsa-miR-4530 | MIMAT0019069 |
| 59 | hsa-miR-7847-3p | MIMAT0030422 |
| 60 | hsa-miR-6825-5p | MIMAT0027550 |
| 61 | hsa-miR-4674 | MIMAT0019756 |
| 62 | hsa-miR-3917 | MIMAT0018191 |
| 63 | hsa-miR-4707-3p | MIMAT0019808 |
| 64 | hsa-miR-6885-5p | MIMAT0027670 |
| 65 | hsa-miR-6722-3p | MIMAT0025854 |
| 66 | hsa-miR-4516 | MIMAT0019053 |
| 67 | hsa-miR-6757-5p | MIMAT0027414 |
| 68 | hsa-miR-6840-3p | MIMAT0027583 |
| 69 | hsa-miR-5195-3p | MIMAT0021127 |
| 70 | hsa-miR-6756-5p | MIMAT0027412 |
| 71 | hsa-miR-6800-5p | MIMAT0027500 |
| 72 | hsa-miR-6727-5p | MIMAT0027355 |
| 73 | hsa-miR-6126 | MIMAT0024599 |
| 74 | hsa-miR-6872-3p | MIMAT0027645 |
| 75 | hsa-miR-4446-3p | MIMAT0018965 |
| 76 | hsa-miR-1268a | MIMAT0005922 |
| 77 | hsa-miR-1908-3p | MIMAT0026916 |
| 78 | hsa-miR-3679-5p | MIMAT0018104 |
| 79 | hsa-miR-4534 | MIMAT0019073 |
| 80 | hsa-miR-4675 | MIMAT0019757 |
| 81 | hsa-miR-7108-5p | MIMAT0028113 |
| 82 | hsa-miR-6799-5p | MIMAT0027498 |
| 83 | hsa-miR-4695-5p | MIMAT0019788 |
| 84 | hsa-miR-3178 | MIMAT0015055 |
| 85 | hsa-miR-5090 | MIMAT0021082 |
| 86 | hsa-miR-3180 | MIMAT0018178 |
| 87 | hsa-miR-1237-5p | MIMAT0022946 |
| 88 | hsa-miR-4758-5p | MIMAT0019903 |
| 89 | hsa-miR-3184-5p | MIMAT0015064 |
| 90 | hsa-miR-4286 | MIMAT0016916 |
| 91 | hsa-miR-6784-5p | MIMAT0027468 |
| 92 | hsa-miR-6768-5p | MIMAT0027436 |
| 93 | hsa-miR-6785-5p | MIMAT0027470 |
| 94 | hsa-miR-4706 | MIMAT0019806 |
| 95 | hsa-miR-711 | MIMAT0012734 |
| 96 | hsa-miR-1260a | MIMAT0005911 |
| 97 | hsa-miR-6746-5p | MIMAT0027392 |
| 98 | hsa-miR-6089 | MIMAT0023714 |
| 99 | hsa-miR-6821-5p | MIMAT0027542 |
| 100 | hsa-miR-4667-5p | MIMAT0019743 |
| 101 | hsa-miR-8069 | MIMAT0030996 |
| 102 | hsa-miR-4726-5p | MIMAT0019845 |
| 103 | hsa-miR-6124 | MIMAT0024597 |
| 104 | hsa-miR-4532 | MIMAT0019071 |
| 105 | hsa-miR-4486 | MIMAT0019020 |
| 106 | hsa-miR-4728-5p | MIMAT0019849 |
| 107 | hsa-miR-4508 | MIMAT0019045 |
| 108 | hsa-miR-128-1-5p | MIMAT0026477 |
| 109 | hsa-miR-4513 | MIMAT0019050 |
| 110 | hsa-miR-6795-5p | MIMAT0027490 |
| 111 | hsa-miR-4689 | MIMAT0019778 |
| 112 | hsa-miR-6763-5p | MIMAT0027426 |
| 113 | hsa-miR-8072 | MIMAT0030999 |
| 114 | hsa-miR-6765-5p | MIMAT0027430 |
| 115 | hsa-miR-4419b | MIMAT0019034 |
| 116 | hsa-miR-7641 | MIMAT0029782 |
| 117 | hsa-miR-3928-3p | MIMAT0018205 |
| 118 | hsa-miR-1227-5p | MIMAT0022941 |
| 119 | hsa-miR-4492 | MIMAT0019027 |
| 120 | hsa-miR-296-3p | MIMAT0004679 |
| 121 | hsa-miR-6769a-5p | MIMAT0027438 |
| 122 | hsa-miR-6889-5p | MIMAT0027678 |
| 123 | hsa-miR-4632-5p | MIMAT0022977 |
| 124 | hsa-miR-4505 | MIMAT0019041 |
| 125 | hsa-miR-3154 | MIMAT0015028 |
| 126 | hsa-miR-3648 | MIMAT0018068 |
| 127 | hsa-miR-4442 | MIMAT0018960 |
| 128 | hsa-miR-3141 | MIMAT0015010 |
| 129 | hsa-miR-7113-3p | MIMAT0028124 |
| 130 | hsa-miR-6819-5p | MIMAT0027538 |
| 131 | hsa-miR-3195 | MIMAT0015079 |
| 132 | hsa-miR-1199-5p | MIMAT0031119 |
| 133 | hsa-miR-6738-5p | MIMAT0027377 |
| 134 | hsa-miR-4656 | MIMAT0019723 |
| 135 | hsa-miR-6820-5p | MIMAT0027540 |
| 136 | hsa-miR-615-5p | MIMAT0004804 |
| 137 | hsa-miR-486-3p | MIMAT0004762 |
| 138 | hsa-miR-1225-3p | MIMAT0005573 |
| 139 | hsa-miR-760 | MIMAT0004957 |
| 140 | hsa-miR-187-5p | MIMAT0004561 |
| 141 | hsa-miR-1203 | MIMAT0005866 |
| 142 | hsa-miR-7110-5p | MIMAT0028117 |
| 143 | hsa-miR-371a-5p | MIMAT0004687 |
| 144 | hsa-miR-939-5p | MIMAT0004982 |
| 145 | hsa-miR-575 | MIMAT0003240 |
| 146 | hsa-miR-92b-5p | MIMAT0004792 |
| 147 | hsa-miR-887-3p | MIMAT0004951 |
| 148 | hsa-miR-920 | MIMAT0004970 |
| 149 | hsa-miR-1915-5p | MIMAT0007891 |
| 150 | hsa-miR-1231 | MIMAT0005586 |
| 151 | hsa-miR-663b | MIMAT0005867 |
| 152 | hsa-miR-1225-5p | MIMAT0005572 |
| 153 | hsa-miR-4763-3p | MIMAT0019913 |
| 154 | hsa-miR-3656 | MIMAT0018076 |
| 155 | hsa-miR-4488 | MIMAT0019022 |
| 156 | hsa-miR-125a-3p | MIMAT0004602 |
| 157 | hsa-miR-1469 | MIMAT0007347 |
| 158 | hsa-miR-1228-5p | MIMAT0005582 |
| 159 | hsa-miR-6798-5p | MIMAT0027496 |
| 160 | hsa-miR-1268b | MIMAT0018925 |
| 161 | hsa-miR-6732-5p | MIMAT0027365 |
| 162 | hsa-miR-1915-3p | MIMAT0007892 |
| 163 | hsa-miR-4433b-3p | MIMAT0030414 |
| 164 | hsa-miR-1207-5p | MIMAT0005871 |
| 165 | hsa-miR-4433-3p | MIMAT0018949 |
| 166 | hsa-miR-6879-5p | MIMAT0027658 |
| 167 | hsa-miR-4417 | MIMAT0018929 |
| 168 | hsa-miR-30c-1-3p | MIMAT0004674 |
| 169 | hsa-miR-4638-5p | MIMAT0019695 |
| 170 | hsa-miR-6088 | MIMAT0023713 |
| 171 | hsa-miR-4270 | MIMAT0016900 |
| 172 | hsa-miR-6782-5p | MIMAT0027464 |
| 173 | hsa-miR-665 | MIMAT0004952 |
| 174 | hsa-miR-486-5p | MIMAT0002177 |
| 175 | hsa-miR-4655-5p | MIMAT0019721 |
| 176 | hsa-miR-1275 | MIMAT0005929 |
| 177 | hsa-miR-6806-5p | MIMAT0027512 |
| 178 | hsa-miR-614 | MIMAT0003282 |
| 179 | hsa-miR-3937 | MIMAT0018352 |
| 180 | hsa-miR-6752-5p | MIMAT0027404 |
| 181 | hsa-miR-6771-5p | MIMAT0027442 |
| 182 | hsa-miR-4450 | MIMAT0018971 |
| 183 | hsa-miR-211-3p | MIMAT0022694 |
| 184 | hsa-miR-663a | MIMAT0003326 |
| 185 | hsa-miR-6842-5p | MIMAT0027586 |
| 186 | hsa-miR-7114-5p | MIMAT0028125 |
| 187 | hsa-miR-6779-5p | MIMAT0027458 |
| 188 | hsa-mir-4443 | MI0016786 |
| 189 | hsa-mir-1908 | MI0008329 |
| 190 | hsa-mir-4257 | MI0015856 |
| 191 | hsa-mir-3197 | MI0014245 |
| 192 | hsa-mir-3188 | MI0014232 |
| 193 | hsa-mir-4649 | MI0017276 |
| 194 | hsa-mir-1343 | MI0017320 |
| 195 | hsa-mir-6861 | MI0022708 |
| 196 | hsa-mir-642b | MI0016685 |
| 197 | hsa-mir-6741 | MI0022586 |
| 198 | hsa-mir-4745 | MI0017384 |
| 199 | hsa-mir-6826 | MI0022671 |
| 200 | hsa-mir-3663 | MI0016064 |
| 201 | hsa-mir-3131 | MI0014151 |
| 202 | hsa-mir-92a-2 | MI0000094 |
| 203 | hsa-mir-4258 | MI0015857 |
| 204 | hsa-mir-4448 | MI0016791 |
| 205 | hsa-mir-6125 | MI0021259 |
| 206 | hsa-mir-6880 | MI0022727 |
| 207 | hsa-mir-6132 | MI0021277 |
| 208 | hsa-mir-4467 | MI0016818 |
| 209 | hsa-mir-6749 | MI0022594 |
| 210 | hsa-mir-2392 | MI0016870 |
| 211 | hsa-mir-1273g | MI0018003 |
| 212 | hsa-mir-4746 | MI0017385 |
| 213 | hsa-mir-1914 | MI0008335 |
| 214 | hsa-mir-7845 | MI0025515 |
| 215 | hsa-mir-6726 | MI0022571 |
| 216 | hsa-mir-128-2 | MI0000727 |
| 217 | hsa-mir-4651 | MI0017279 |
| 218 | hsa-mir-6765 | MI0022610 |
| 219 | hsa-mir-3185 | MI0014227 |
| 220 | hsa-mir-4792 | MI0017439 |
| 221 | hsa-mir-6887 | MI0022734 |
| 222 | hsa-mir-5572 | MI0019117 |
| 223 | hsa-mir-3619 | MI0016009 |
| 224 | hsa-mir-6780b | MI0022681 |
| 225 | hsa-mir-4707 | MI0017340 |
| 226 | hsa-mir-8063 | MI0025899 |
| 227 | hsa-mir-4454 | MI0016800 |
| 228 | hsa-mir-4525 | MI0016892 |
| 229 | hsa-mir-7975 | MI0025751 |
| 230 | hsa-mir-744 | MI0005559 |
| 231 | hsa-mir-3135b | MI0016809 |
| 232 | hsa-mir-4648 | MI0017275 |
| 233 | hsa-mir-6816 | MI0022661 |
| 234 | hsa-mir-4741 | MI0017379 |
| 235 | hsa-mir-7150 | MI0023610 |
| 236 | hsa-mir-6791 | MI0022636 |
| 237 | hsa-mir-1247 | MI0006382 |
| 238 | hsa-mir-7977 | MI0025753 |
| 239 | hsa-mir-4497 | MI0016859 |
| 240 | hsa-mir-6090 | MI0020367 |
| 241 | hsa-mir-6781 | MI0022626 |
| 242 | hsa-mir-6870 | MI0022717 |
| 243 | hsa-mir-6729 | MI0022574 |
| 244 | hsa-mir-4530 | MI0016897 |
| 245 | hsa-mir-7847 | MI0025517 |
| 246 | hsa-mir-6825 | MI0022670 |
| 247 | hsa-mir-4674 | MI0017305 |
| 248 | hsa-mir-3917 | MI0016423 |
| 249 | hsa-mir-6885 | MI0022732 |
| 250 | hsa-mir-6722 | MI0022557 |
| 251 | hsa-mir-4516 | MI0016882 |
| 252 | hsa-mir-6757 | MI0022602 |
| 253 | hsa-mir-6840 | MI0022686 |
| 254 | hsa-mir-5195 | MI0018174 |
| 255 | hsa-mir-6756 | MI0022601 |
| 256 | hsa-mir-6800 | MI0022645 |
| 257 | hsa-mir-6727 | MI0022572 |
| 258 | hsa-mir-6126 | MI0021260 |
| 259 | hsa-mir-6872 | MI0022719 |
| 260 | hsa-mir-4446 | MI0016789 |
| 261 | hsa-mir-1268a | MI0006405 |
| 262 | hsa-mir-3679 | MI0016080 |
| 263 | hsa-mir-4534 | MI0016901 |
| 264 | hsa-mir-4675 | MI0017306 |
| 265 | hsa-mir-7108 | MI0022959 |
| 266 | hsa-mir-6799 | MI0022644 |
| 267 | hsa-mir-4695 | MI0017328 |
| 268 | hsa-mir-3178 | MI0014212 |
| 269 | hsa-mir-5090 | MI0017979 |
| 270 | hsa-mir-3180-4 | MI0016408 |
| 271 | hsa-mir-3180-5 | MI0016409 |
| 272 | hsa-mir-1237 | MI0006327 |
| 273 | hsa-mir-4758 | MI0017399 |
| 274 | hsa-mir-3184 | MI0014226 |
| 275 | hsa-mir-4286 | MI0015894 |
| 276 | hsa-mir-6784 | MI0022629 |
| 277 | hsa-mir-6768 | MI0022613 |
| 278 | hsa-mir-6785 | MI0022630 |
| 279 | hsa-mir-4706 | MI0017339 |
| 280 | hsa-mir-711 | MI0012488 |
| 281 | hsa-mir-1260a | MI0006394 |
| 282 | hsa-mir-6746 | MI0022591 |
| 283 | hsa-mir-6089-1 | MI0020366 |
| 284 | hsa-mir-6089-2 | MI0023563 |
| 285 | hsa-mir-6821 | MI0022666 |
| 286 | hsa-mir-4667 | MI0017297 |
| 287 | hsa-mir-8069 | MI0025905 |
| 288 | hsa-mir-4726 | MI0017363 |
| 289 | hsa-mir-6124 | MI0021258 |
| 290 | hsa-mir-4532 | MI0016899 |
| 291 | hsa-mir-4486 | MI0016847 |
| 292 | hsa-mir-4728 | MI0017365 |
| 293 | hsa-mir-4508 | MI0016872 |
| 294 | hsa-mir-128-1 | MI0000447 |
| 295 | hsa-mir-4513 | MI0016879 |
| 296 | hsa-mir-6795 | MI0022640 |
| 297 | hsa-mir-4689 | MI0017322 |
| 298 | hsa-mir-6763 | MI0022608 |
| 299 | hsa-mir-8072 | MI0025908 |
| 300 | hsa-mir-4419b | MI0016861 |
| 301 | hsa-mir-7641-1 | MI0024975 |
| 302 | hsa-mir-7641-2 | MI0024976 |
| 303 | hsa-mir-3928 | MI0016438 |
| 304 | hsa-mir-1227 | MI0006316 |
| 305 | hsa-mir-4492 | MI0016854 |
| 306 | hsa-mir-296 | MI0000747 |
| 307 | hsa-mir-6769a | MI0022614 |
| 308 | hsa-mir-6889 | MI0022736 |
| 309 | hsa-mir-4632 | MI0017259 |
| 310 | hsa-mir-4505 | MI0016868 |
| 311 | hsa-mir-3154 | MI0014182 |
| 312 | hsa-mir-3648 | MI0016048 |
| 313 | hsa-mir-4442 | MI0016785 |
| 314 | hsa-mir-3141 | MI0014165 |
| 315 | hsa-mir-7113 | MI0022964 |
| 316 | hsa-mir-6819 | MI0022664 |
| 317 | hsa-mir-3195 | MI0014240 |
| 318 | hsa-mir-1199 | MI0020340 |
| 319 | hsa-mir-6738 | MI0022583 |
| 320 | hsa-mir-4656 | MI0017284 |
| 321 | hsa-mir-6820 | MI0022665 |
| 322 | hsa-mir-615 | MI0003628 |
| 323 | hsa-mir-486 | MI0002470 |
| 324 | hsa-mir-486-2 | MI0023622 |
| 325 | hsa-mir-1225 | MI0006311 |
| 326 | hsa-mir-760 | MI0005567 |
| 327 | hsa-mir-187 | MI0000274 |
| 328 | hsa-mir-1203 | MI0006335 |
| 329 | hsa-mir-7110 | MI0022961 |
| 330 | hsa-mir-371a | MI0000779 |
| 331 | hsa-mir-939 | MI0005761 |
| 332 | hsa-mir-575 | MI0003582 |
| 333 | hsa-mir-92b | MI0003560 |
| 334 | hsa-mir-887 | MI0005562 |
| 335 | hsa-mir-920 | MI0005712 |
| 336 | hsa-mir-1915 | MI0008336 |
| 337 | hsa-mir-1231 | MI0006321 |
| 338 | hsa-mir-663b | MI0006336 |
| 339 | hsa-mir-4763 | MI0017404 |
| 340 | hsa-mir-3656 | MI0016056 |
| 341 | hsa-mir-4488 | MI0016849 |
| 342 | hsa-mir-125a | MI0000469 |
| 343 | hsa-mir-1469 | MI0007074 |
| 344 | hsa-mir-1228 | MI0006318 |
| 345 | hsa-mir-6798 | MI0022643 |
| 346 | hsa-mir-1268b | MI0016748 |
| 347 | hsa-mir-6732 | MI0022577 |
| 348 | hsa-mir-4433b | MI0025511 |
| 349 | hsa-mir-1207 | MI0006340 |
| 350 | hsa-mir-4433 | MI0016773 |
| 351 | hsa-mir-6879 | MI0022726 |
| 352 | hsa-mir-4417 | MI0016753 |
| 353 | hsa-mir-30c-1 | MI0000736 |
| 354 | hsa-mir-4638 | MI0017265 |
| 355 | hsa-mir-6088 | MI0020365 |
| 356 | hsa-mir-4270 | MI0015878 |
| 357 | hsa-mir-6782 | MI0022627 |
| 358 | hsa-mir-665 | MI0005563 |
| 359 | hsa-mir-4655 | MI0017283 |
| 360 | hsa-mir-1275 | MI0006415 |
| 361 | hsa-mir-6806 | MI0022651 |
| 362 | hsa-mir-614 | MI0003627 |
| 363 | hsa-mir-3937 | MI0016593 |
| 364 | hsa-mir-6752 | MI0022597 |
| 365 | hsa-mir-6771 | MI0022616 |
| 366 | hsa-mir-4450 | MI0016795 |
| 367 | hsa-mir-211 | MI0000287 |
| 368 | hsa-mir-663a | MI0003672 |
| 369 | hsa-mir-6842 | MI0022688 |
| 370 | hsa-mir-7114 | MI0022965 |
| 371 | hsa-mir-6779 | MI0022624 |
| 372 | isomiR example 1 of SEQ ID NO: 1 | — |
| 373 | isomiR example 2 of SEQ ID NO: 1 | — |
| 374 | isomiR example 1 of SEQ ID NO: 2 | — |
| 375 | isomiR example 2 of SEQ ID NO: 2 | — |
| 376 | isomiR example 1 of SEQ ID NO: 4 | — |
| 377 | isomiR example 2 of SEQ ID NO: 4 | — |
| 378 | isomiR example 1 of SEQ ID NO: 5 | — |
| 379 | isomiR example 2 of SEQ ID NO: 5 | — |
| 380 | isomiR example 1 of SEQ ID NO: 6 | — |
| 381 | isomiR example 2 of SEQ ID NO: 6 | — |
| 382 | isomiR example 1 of SEQ ID NO: 7 | — |
| 383 | isomiR example 2 of SEQ ID NO: 7 | — |
| 384 | isomiR example 1 of SEQ ID NO: 10 | — |
| 385 | isomiR example 2 of SEQ lD NO: 10 | — |
| 386 | isomiR example 1 of SEQ ID NO: 12 | — |
| 387 | isomiR example 2 of SEQ lD NO: 12 | — |
| 388 | isomiR example 1 of SEQ ID NO: 15 | — |
| 389 | isomiR example 2 of SEQ lD NO: 15 | — |
| 390 | isomiR example 1 of SEQ lD NO: 16 | — |
| 391 | isomiR example 2 of SEQ lD NO: 16 | — |
| 392 | isomiR example 1 of SEQ lD NO: 18 | — |
| 393 | isomiR example 2 of SEQ lD NO: 18 | — |
| 394 | isomiR example 1 of SEQ ID NO: 19 | — |
| 395 | isomiR example 2 of SEQ lD NO: 19 | — |
| 396 | isomiR example 1 of SEQ ID NO: 21 | — |
| 397 | isomiR example 2 of SEQ ID NO: 21 | — |
| 398 | isomiR example 1 of SEQ ID NO: 22 | — |
| 399 | isomiR example 2 of SEQ ID NO: 22 | — |
| 400 | isomiR example 1 of SEQ ID NO: 24 | — |
| 401 | isomiR example 2 of SEQ ID NO: 24 | — |
| 402 | isomiR example 1 of SEQ ID NO: 25 | — |
| 403 | isomiR example 2 of SEQ ID NO: 25 | — |
| 404 | isomiR example 1 of SEQ ID NO: 27 | — |
| 405 | isomiR example 2 of SEQ ID NO: 27 | — |
| 406 | isomiR example 1 of SEQ ID NO: 30 | — |
| 407 | isomiR example 2 of SEQ ID NO: 30 | — |
| 408 | isomiR example 1 of SEQ ID NO: 31 | — |
| 409 | isomiR example 2 of SEQ ID NO: 31 | — |
| 410 | isomiR example 1 of SEQ ID NO: 33 | — |
| 411 | isomiR example 2 of SEQ ID NO: 33 | — |
| 412 | isomiR example 1 of SEQ ID NO: 34 | — |
| 413 | isomiR example 2 of SEQ ID NO: 34 | — |
| 414 | isomiR example 1 of SEQ ID NO: 36 | — |
| 415 | isomiR example 2 of SEQ ID NO: 36 | — |
| 416 | isomiR example 1 of SEQ ID NO: 39 | — |
| 417 | isomiR example 2 of SEQ ID NO: 39 | — |
| 418 | isomiR example 1 of SEQ ID NO: 41 | — |
| 419 | isomiR example 2 of SEQ ID NO: 41 | — |
| 420 | isomiR example 1 of SEQ ID NO: 42 | — |
| 421 | isomiR example 2 of SEQ ID NO: 42 | — |
| 422 | isomiR example 1 of SEQ ID NO: 43 | — |
| 423 | isomiR example 2 of SEQ ID NO: 43 | — |
| 424 | isomiR example 1 of SEQ ID NO: 44 | — |
| 425 | isomiR example 2 of SEQ ID NO: 44 | — |
| 426 | isomiR example 1 of SEQ ID NO: 45 | — |
| 427 | isomiR example 2 of SEQ ID NO: 45 | — |
| 428 | isomiR example 1 of SEQ ID NO: 46 | — |
| 429 | isomiR example 2 of SEQ ID NO: 46 | — |
| 430 | isomiR example 1 of SEQ ID NO: 48 | — |
| 431 | isomiR example 2 of SEQ ID NO: 48 | — |
| 432 | isomiR example 1 of SEQ ID NO: 51 | — |
| 433 | isomiR example 2 of SEQ ID NO: 51 | — |
| 434 | isomiR example 1 of SEQ ID NO: 53 | — |
| 435 | isomiR example 2 of SEQ ID NO: 53 | — |
| 436 | isomiR example 1 of SEQ ID NO: 58 | — |
| 437 | isomiR example 2 of SEQ ID NO: 58 | — |
| 438 | isomiR example 1 of SEQ ID NO: 61 | — |
| 439 | isomiR example 2 of SEQ ID NO: 61 | — |
| 440 | isomiR example 1 of SEQ ID NO: 62 | — |
| 441 | isomiR example 2 of SEQ ID NO: 62 | — |
| 442 | isomiR example 1 of SEQ ID NO: 63 | — |
| 443 | isomiR example 2 of SEQ ID NO: 63 | — |
| 444 | isomiR example 1 of SEQ ID NO: 66 | — |
| 445 | isomiR example 2 of SEQ ID NO: 66 | — |
| 446 | isomiR example 1 of SEQ ID NO: 69 | — |
| 447 | isomiR example 2 of SEQ ID NO: 69 | — |
| 448 | isomiR example 1 of SEQ ID NO: 73 | — |
| 449 | isomiR example 2 of SEQ ID NO: 73 | — |
| 450 | isomiR example 1 of SEQ ID NO: 75 | — |
| 451 | isomiR example 2 of SEQ ID NO: 75 | — |
| 452 | isomiR example 1 of SEQ ID NO: 76 | — |
| 453 | isomiR example 2 of SEQ ID NO: 76 | — |
| 454 | isomiR example 1 of SEQ ID NO: 77 | — |
| 455 | isomiR example 2 of SEQ ID NO: 77 | — |
| 456 | isomiR example 1 of SEQ ID NO: 78 | — |
| 457 | isomiR example 2 of SEQ ID NO: 78 | — |
| 458 | isomiR example 1 of SEQ ID NO: 83 | — |
| 459 | isomiR example 2 of SEQ ID NO: 83 | — |
| 460 | isomiR example 1 of SEQ ID NO: 84 | — |
| 461 | isomiR example 2 of SEQ ID NO: 84 | — |
| 462 | isomiR example 1 of SEQ ID NO: 85 | — |
| 463 | isomiR example 2 of SEQ ID NO: 85 | — |
| 464 | isomiR example 1 of SEQ ID NO: 86 | — |
| 465 | isomiR example 2 of SEQ ID NO: 86 | — |
| 466 | isomiR example 1 of SEQ ID NO: 87 | — |
| 467 | isomiR example 2 of SEQ ID NO: 87 | — |
| 468 | isomiR example 1 of SEQ ID NO: 88 | — |
| 469 | isomiR example 2 of SEQ ID NO: 88 | — |
| 470 | isomiR example 1 of SEQ ID NO: 90 | — |
| 471 | isomiR example 2 of SEQ ID NO: 90 | — |
| 472 | isomiR example 1 of SEQ ID NO: 94 | — |
| 473 | isomiR example 2 of SEQ ID NO: 94 | — |
| 474 | isomiR example 1 of SEQ ID NO: 95 | — |
| 475 | isomiR example 2 of SEQ ID NO: 95 | — |
| 476 | isomiR example 1 of SEQ ID NO: 96 | — |
| 477 | isomiR example 2 of SEQ ID NO: 96 | — |
| 478 | isomiR example 1 of SEQ ID NO: 98 | — |
| 479 | isomiR example 2 of SEQ ID NO: 98 | — |
| 480 | isomiR example 1 of SEQ ID NO: 100 | — |
| 481 | isomiR example 2 of SEQ ID NO: 100 | — |
| 482 | isomiR example 1 of SEQ ID NO: 102 | — |
| 483 | isomiR example 2 of SEQ ID NO: 102 | — |
| 484 | isomiR example 1 of SEQ ID NO: 103 | — |
| 485 | isomiR example 2 of SEQ ID NO: 103 | — |
| 486 | isomiR example 1 of SEQ ID NO: 104 | — |
| 487 | isomiR example 2 of SEQ ID NO: 104 | — |
| 488 | isomiR example 1 of SEQ ID NO: 105 | — |
| 489 | isomiR example 2 of SEQ ID NO: 105 | — |
| 490 | isomiR example 1 of SEQ ID NO: 106 | — |
| 491 | isomiR example 2 of SEQ ID NO: 106 | — |
| 492 | isomiR example 1 of SEQ ID NO: 107 | — |
| 493 | isomiR example 2 of SEQ ID NO: 107 | — |
| 494 | isomiR example 1 of SEQ ID NO: 108 | — |
| 495 | isomiR example 2 of SEQ ID NO: 108 | — |
| 496 | isomiR example 1 of SEQ ID NO: 109 | — |
| 497 | isomiR example 2 of SEQ ID NO: 109 | — |
| 498 | isomiR example 1 of SEQ ID NO: 111 | — |
| 499 | isomiR example 2 of SEQ ID NO: 111 | — |
| 500 | isomiR example 1 of SEQ ID NO: 115 | — |
| 501 | isomiR example 2 of SEQ ID NO: 115 | — |
| 502 | isomiR example 1 of SEQ ID NO: 117 | — |
| 503 | isomiR example 2 of SEQ ID NO: 117 | — |
| 504 | isomiR example 1 of SEQ ID NO: 119 | — |
| 505 | isomiR example 2 of SEQ ID NO: 119 | — |
| 506 | isomiR example 1 of SEQ ID NO: 120 | — |
| 507 | isomiR example 2 of SEQ ID NO: 120 | — |
| 508 | isomiR example 1 of SEQ ID NO: 123 | — |
| 509 | isomiR example 2 of SEQ ID NO: 123 | — |
| 510 | isomiR example 1 of SEQ ID NO: 124 | — |
| 511 | isomiR example 2 of SEQ ID NO: 124 | — |
| 512 | isomiR example 1 of SEQ ID NO: 125 | — |
| 513 | isomiR example 2 of SEQ ID NO: 125 | — |
| 514 | isomiR example 1 of SEQ ID NO: 126 | — |
| 515 | isomiR example 2 of SEQ ID NO: 126 | — |
| 516 | isomiR example 1 of SEQ ID NO: 127 | — |
| 517 | isomiR example 2 of SEQ ID NO: 127 | — |
| 518 | isomiR example 1 of SEQ ID NO: 128 | — |
| 519 | isomiR example 2 of SEQ ID NO: 128 | — |
| 520 | isomiR example 1 of SEQ ID NO: 131 | — |
| 521 | isomiR example 2 of SEQ ID NO: 131 | — |
| 522 | isomiR example 1 of SEQ ID NO: 136 | — |
| 523 | isomiR example 2 of SEQ ID NO: 136 | — |
| 524 | isomiR example 1 of SEQ ID NO: 137 | — |
| 525 | isomiR example 2 of SEQ ID NO: 137 | — |
| 526 | isomiR example 1 of SEQ ID NO: 139 | — |
| 527 | isomiR example 2 of SEQ ID NO: 139 | — |
| 528 | isomiR example 1 of SEQ ID NO: 140 | — |
| 529 | isomiR example 2 of SEQ ID NO: 140 | — |
| 530 | isomiR example 1 of SEQ ID NO: 143 | — |
| 531 | isomiR example 2 of SEQ ID NO: 143 | — |
| 532 | isomiR example 1 of SEQ ID NO: 144 | — |
| 533 | isomiR example 2 of SEQ ID NO: 144 | — |
| 534 | isomiR example 1 of SEQ ID NO: 147 | — |
| 535 | isomiR example 2 of SEQ ID NO: 147 | — |
| 536 | isomiR example 1 of SEQ ID NO: 149 | — |
| 537 | isomiR example 2 of SEQ ID NO: 149 | — |
| 538 | isomiR example 1 of SEQ ID NO: 151 | — |
| 539 | isomiR example 2 of SEQ ID NO: 151 | — |
| 540 | isomiR example 1 of SEQ ID NO: 153 | — |
| 541 | isomiR example 2 of SEQ ID NO: 153 | — |
| 542 | isomiR example 1 of SEQ ID NO: 154 | — |
| 543 | isomiR example 2 of SEQ ID NO: 154 | — |
| 544 | isomiR example 1 of SEQ ID NO: 155 | — |
| 545 | isomiR example 2 of SEQ ID NO: 155 | — |
| 546 | isomiR example 1 of SEQ ID NO: 156 | — |
| 547 | isomiR example 2 of SEQ ID NO: 156 | — |
| 548 | isomiR example 1 of SEQ ID NO: 158 | — |
| 549 | isomiR example 2 of SEQ ID NO: 158 | — |
| 550 | isomiR example 1 of SEQ ID NO: 160 | — |
| 551 | isomiR example 2 of SEQ ID NO: 160 | — |
| 552 | isomiR example 1 of SEQ ID NO: 162 | — |
| 553 | isomiR example 2 of SEQ ID NO: 162 | — |
| 554 | isomiR example 1 of SEQ ID NO: 165 | — |
| 555 | isomiR example 2 of SEQ ID NO: 165 | — |
| 556 | isomiR example 1 of SEQ ID NO: 167 | — |
| 557 | isomiR example 2 of SEQ ID NO: 167 | — |
| 558 | isomiR example 1 of SEQ ID NO: 168 | — |
| 559 | isomiR example 2 of SEQ ID NO: 168 | — |
| 560 | isomiR example 1 of SEQ ID NO: 169 | — |
| 561 | isomiR example 2 of SEQ ID NO: 169 | — |
| 562 | isomiR example 1 of SEQ ID NO: 170 | — |
| 563 | isomiR example 2 of SEQ ID NO: 170 | — |
| 564 | isomiR example 1 of SEQ ID NO: 173 | — |
| 565 | isomiR example 2 of SEQ ID NO: 173 | — |
| 566 | isomiR example 1 of SEQ ID NO: 174 | — |
| 567 | isomiR example 2 of SEQ ID NO: 174 | — |
| 568 | isomiR example 1 of SEQ ID NO: 175 | — |
| 569 | isomiR example 2 of SEQ ID NO: 175 | — |
| 570 | isomiR example 1 of SEQ ID NO: 176 | — |
| 571 | isomiR example 2 of SEQ ID NO: 176 | — |
| 572 | isomiR example 1 of SEQ ID NO: 178 | — |
| 573 | isomiR example 2 of SEQ ID NO: 178 | — |
| 574 | isomiR example 1 of SEQ ID NO: 182 | — |
| 575 | isomiR example 2 of SEQ ID NO: 182 | — |
| 576 | isomiR example 1 of SEQ ID NO: 183 | — |
| 577 | isomiR example 2 of SEQ ID NO: 183 | — |
| 578 | isomiR example 1 of SEQ ID NO: 184 | — |
| 579 | isomiR example 1 of SEQ ID NO: 184 | — |
| 580 | hsa-miR-204-3p | MIMAT0022693 |
| 581 | hsa-miR-642a-3p | MIMAT0020924 |
| 582 | hsa-miR-762 | MIMAT0010313 |
| 583 | hsa-miR-1202 | MIMAT0005865 |
| 584 | hsa-miR-3162-5p | MIMAT0015036 |
| 585 | hsa-miR-3196 | MIMAT0015080 |
| 586 | hsa-miR-3622a-5p | MIMAT0018003 |
| 587 | hsa-miR-3665 | MIMAT0018087 |
| 588 | hsa-miR-3940-5p | MIMAT0019229 |
| 589 | hsa-miR-4294 | MIMAT0016849 |
| 590 | hsa-miR-4466 | MIMAT0018993 |
| 591 | hsa-miR-4476 | MIMAT0019003 |
| 592 | hsa-miR-4723-5p | MIMAT0019838 |
| 593 | hsa-miR-4725-3p | MIMAT0019844 |
| 594 | hsa-miR-4730 | MIMAT0019852 |
| 595 | hsa-miR-4739 | MIMAT0019868 |
| 596 | hsa-miR-4787-5p | MIMAT0019956 |
| 597 | hsa-miR-5787 | MIMAT0023252 |
| 598 | hsa-miR-6085 | MIMAT0023710 |
| 599 | hsa-miR-6717-5p | MIMAT0025846 |
| 600 | hsa-miR-6724-5p | MIMAT0025856 |
| 601 | hsa-miR-6777-5p | MIMAT0027454 |
| 602 | hsa-miR-6778-5p | MIMAT0027456 |
| 603 | hsa-miR-6787-5p | MIMAT0027474 |
| 604 | hsa-miR-6789-5p | MIMAT0027478 |
| 605 | hsa-miR-6845-5p | MIMAT0027590 |
| 606 | hsa-miR-6893-5p | MIMAT0027686 |
| 607 | hsa-miR-16-5p | MIMAT0000069 |
| 608 | hsa-miR-423-5p | MIMAT0004748 |
| 609 | hsa-miR-451a | MIMAT0001631 |
| 610 | hsa-miR-564 | MIMAT0003228 |
| 611 | hsa-miR-671-5p | MIMAT0003880 |
| 612 | hsa-mir-204 | MI0000284 |
| 613 | hsa-mir-642a | MI0003657 |
| 614 | hsa-mir-762 | MI0003892 |
| 615 | hsa-mir-1202 | MI0006334 |
| 616 | hsa-mir-3162 | MI0014192 |
| 617 | hsa-mir-3196 | MI0014241 |
| 618 | hsa-mir-3622a | MI0016013 |
| 619 | hsa-mir-3665 | MI0016066 |
| 620 | hsa-mir-3940 | MI0016597 |
| 621 | hsa-mir-4294 | MI0015827 |
| 622 | hsa-mir-4466 | MI0016817 |
| 623 | hsa-mir-4476 | MI0016828 |
| 624 | hsa-mir-4723 | MI0017359 |
| 625 | hsa-mir-4725 | MI0017362 |
| 626 | hsa-mir-4730 | MI0017367 |
| 627 | hsa-mir-4739 | MI0017377 |
| 628 | hsa-mir-4787 | MI0017434 |
| 629 | hsa-mir-5787 | MI0019797 |
| 630 | hsa-mir-6085 | MI0020362 |
| 631 | hsa-mir-6717 | MI0022551 |
| 632 | hsa-mir-6724 | MI0022559 |
| 633 | hsa-mir-6777 | MI0022622 |
| 634 | hsa-mir-6778 | MI0022623 |
| 635 | hsa-mir-6787 | MI0022632 |
| 636 | hsa-mir-6789 | MI0022634 |
| 637 | hsa-mir-6845 | MI0022691 |
| 638 | hsa-mir-6893 | MI0022740 |
| 639 | hsa-mir-16-1 | MI0000070 |
| 640 | hsa-mir-16-2 | MI0000115 |
| 641 | hsa-mir-423 | MI0001445 |
| 642 | hsa-mir-451a | MI0001729 |
| 643 | hsa-mir-564 | MI0003570 |
| 644 | hsa-mir-671 | MI0003760 |
| 645 | isomiR example 1 of SEQ ID NO: 580 | — |
| 646 | isomiR example 2 of SEQ ID NO: 580 | — |
| 647 | isomiR example 1 of SEQ ID NO: 581 | — |
| 648 | isomiR example 2 of SEQ ID NO: 581 | — |
| 649 | isomiR example 1 of SEQ ID NO: 583 | — |
| 650 | isomiR example 1 of SEQ ID NO: 584 | — |
| 651 | isomiR example 2 of SEQ ID NO: 584 | — |
| 652 | isomiR example 1 of SEQ ID NO: 585 | — |
| 653 | isomiR example 2 of SEQ ID NO: 585 | — |
| 654 | isomiR example 1 of SEQ ID NO: 586 | — |
| 655 | isomiR example 1 of SEQ ID NO: 587 | — |
| 656 | isomiR example 2 of SEQ ID NO: 587 | — |
| 657 | isomiR example 1 of SEQ ID NO: 588 | — |
| 658 | isomiR example 2 of SEQ ID NO: 588 | — |
| 659 | isomiR example 1 of SEQ ID NO: 590 | — |
| 660 | isomiR example 2 of SEQ ID NO: 590 | — |
| 661 | isomiR example 1 of SEQ ID NO: 591 | — |
| 662 | isomiR example 2 of SEQ ID NO: 591 | — |
| 663 | isomiR example 1 of SEQ ID NO: 592 | — |
| 664 | isomiR example 2 of SEQ ID NO: 592 | — |
| 665 | isomiR example 1 of SEQ ID NO: 593 | — |
| 666 | isomiR example 2 of SEQ ID NO: 593 | — |
| 667 | isomiR example 1 of SEQ ID NO: 594 | — |
| 668 | isomiR example 2 of SEQ ID NO: 594 | — |
| 669 | isomiR example 1 of SEQ ID NO: 595 | — |
| 670 | isomiR example 2 of SEQ ID NO: 595 | — |
| 671 | isomiR example 1 of SEQ ID NO: 597 | — |
| 672 | isomiR example 2 of SEQ ID NO: 597 | — |
| 673 | isomiR example 1 of SEQ ID NO: 599 | — |
| 674 | isomiR example 2 of SEQ ID NO: 599 | — |
| 675 | isomiR example 1 of SEQ ID NO: 600 | — |
| 676 | isomiR example 2 of SEQ ID NO: 600 | — |
| 677 | isomiR example 1 of SEQ ID NO: 607 | — |
| 678 | isomiR example 2 of SEQ ID NO: 607 | — |
| 679 | isomiR example 1 of SEQ ID NO: 608 | — |
| 680 | isomiR example 2 of SEQ ID NO: 608 | — |
| 681 | isomiR example 1 of SEQ ID NO: 609 | — |
| 682 | isomiR example 2 of SEQ ID NO: 609 | — |
| 683 | isomiR example 1 of SEQ ID NO: 611 | — |
| 684 | isomiR example 2 of SEQ ID NO: 611 | — |

The present application claims the priority of Japanese Patent Application No. 2014-121377 filed on Jun. 12, 2014 and Japanese Patent Application No. 2015-71756 filed on Mar. 31, 2015, and encompasses the contents described in the specifications of these patent applications.

### Advantageous Effects of Invention

According to the present invention, prostate cancer can be detected easily and highly accurately. For example, the presence or absence of prostate cancer in a patient can be easily detected by using, as an index, the measurement values of several miRNAs in blood, serum, and/or plasma of the patient, which can be collected with limited invasiveness.

### BRIEF DESCRIPTION OF DRAWINGS

FIG. 1 This figure shows the relationship between the nucleotide sequences of hsa-miR-1343-3p represented by SEQ ID NO: 7 and hsa-miR-1343-5p represented by SEQ ID NO: 9, which are formed from a precursor hsa-mir-1343 represented by SEQ ID NO: 194.

FIG. 2 Left diagram: the measurement values of hsa-miR-4443 (SEQ ID NO: 1) in healthy subjects (100 persons) and in prostate cancer patients (35 persons) selected as the training cohort were each plotted on the ordinate. The horizontal line in the diagram depicts a threshold (6.84) that was optimized by Fisher's linear discriminant analysis and discriminated between the two groups. Right diagram: the measurement values of hsa-miR-4443 (SEQ ID NO: 1) in healthy subjects (50 persons) and in prostate cancer patients (17 persons) selected as the validation cohort were each plotted on the ordinate. The horizontal line in the diagram depicts the threshold (6.84) that was set in the training cohort and discriminated between the two groups.

FIG. 3 Left diagram: the measurement values of hsa-miR-4443 (SEQ ID NO: 1) in healthy subjects (100 persons, circles) and in prostate cancer patients (35 persons, triangles) selected as the training cohort were each plotted on the abscissa against their measurement values of hsa-miR-1908-5p (SEQ ID NO: 2) on the ordinate. The line in the diagram depicts a discriminant function (0=1.15x+y+19.53) that was optimized by Fisher's linear discriminant analysis and discriminated between the two groups. Right diagram: the measurement values of hsa-miR-4443 (SEQ ID NO: 1) in healthy subjects (50 persons, circles) and in prostate cancer patients (17 persons, triangles) selected as the validation cohort were each plotted on the abscissa against their measurement values of hsa-miR-1908-5p (SEQ ID NO: 2) on the ordinate. The line in the diagram depicts the threshold (0=1.15x+y+19.53) that was set in the training cohort and discriminated between the two groups.

FIG. 4 Upper diagram: a discriminant (1.34x miR-92a-2-5p+1.56x miR-6820-5p−1.29x miR-4745-5p−0.76x miR-125a-3p−4.31) was prepared by use of Fisher's linear discriminant analysis from the measurement values of hsa-miR-4745-5p (SEQ ID NO: 12), hsa-miR-92a-2-5p (SEQ ID NO: 16), hsa-miR-6820-5p (SEQ ID NO: 135), and hsa-miR-125a-3p (SEQ ID NO: 156) in 35 prostate cancer patients, 99 healthy subjects, and 63 breast cancer patients selected as the training cohort, and discriminant scores obtained from the discriminant were plotted on the ordinate against the sample groups on the abscissa. The dotted line in the diagram depicts a discriminant boundary that offered a discriminant score of 0 and discriminated between the groups. Lower diagram: discriminant scores obtained from the discriminant prepared in the training cohort as to the measurement values of hsa-miR-4745-5p (SEQ ID NO: 12), hsa-miR-92a-2-5p (SEQ ID NO: 16), hsa-miR-6820-5p (SEQ ID NO: 135), and hsa-miR-125a-3p (SEQ ID NO: 156) in 17 prostate cancer patients, 51 healthy subjects, and 30 breast cancer patients selected as the validation cohort were plotted on the ordinate against the sample groups on the abscissa. The dotted line in the diagram depicts the discriminant boundary that offered a discriminant score of 0 and discriminated between the two groups.

### DESCRIPTION OF EMBODIMENTS

Hereinafter, the present invention will be described further specifically.

### 1. Target Nucleic Acid for Prostate Cancer

A primary target nucleic acid as a prostate cancer marker for detecting the presence and/or absence of prostate cancer or prostate cancer cells using the nucleic acid probe or the primer for the detection of prostate cancer defined above according to the present invention comprises at least one or more miRNA(s) selected from the group consisting of hsa-miR-4443, hsa-miR-1908-5p, hsa-miR-4257, hsa-miR-3197, hsa-miR-3188, hsa-miR-4649-5p, hsa-miR-1343-3p, hsa-miR-6861-5p, hsa-miR-1343-5p, hsa-miR-642b-3p, hsa-miR-6741-5p, hsa-miR-4745-5p, hsa-miR-6826-5p, hsa-miR-3663-3p, hsa-miR-3131, hsa-miR-92a-2-5p, hsa-miR-4258, hsa-miR-4448, hsa-miR-6125, hsa-miR-6880-5p, hsa-miR-6132, hsa-miR-4467, hsa-miR-6749-5p, hsa-miR-2392, hsa-miR-1273g-3p, hsa-miR-4746-3p, hsa-miR-1914-3p, hsa-miR-7845-5p, hsa-miR-6726-5p, hsa-miR-128-2-5p, hsa-miR-4651, hsa-miR-6765-3p, hsa-miR-3185, hsa-miR-4792, hsa-miR-6887-5p, hsa-miR-5572, hsa-miR-3619-3p, hsa-miR-6780b-5p, hsa-miR-4707-5p, hsa-miR-8063, hsa-miR-4454, hsa-miR-4525, hsa-miR-7975, hsa-miR-744-5p, hsa-miR-3135b, hsa-miR-4648, hsa-miR-6816-5p, hsa-miR-4741, hsa-miR-7150, hsa-miR-6791-5p, hsa-miR-1247-3p, hsa-miR-7977, hsa-miR-4497, hsa-miR-6090, hsa-miR-6781-5p, hsa-miR-6870-5p, hsa-miR-6729-5p, hsa-miR-4530, hsa-miR-7847-3p, hsa-miR-6825-5p, hsa-miR-4674, hsa-miR-3917, hsa-miR-4707-3p, hsa-miR-6885-5p, hsa-miR-6722-3p, hsa-miR-4516, hsa-miR-6757-5p, hsa-miR-6840-3p, hsa-miR-5195-3p, hsa-miR-6756-5p, hsa-miR-6800-5p, hsa-miR-6727-5p, hsa-miR-6126, hsa-miR-6872-3p, hsa-miR-4446-3p, hsa-miR-1268a, hsa-miR-1908-3p, hsa-miR-3679-5p, hsa-miR-4534, hsa-miR-4675, hsa-miR-7108-5p, hsa-miR-6799-5p, hsa-miR-4695-5p, hsa-miR-3178, hsa-miR-5090, hsa-miR-3180, hsa-miR-1237-5p, hsa-miR-4758-5p, hsa-miR-3184-5p, hsa-miR-4286, hsa-miR-6784-5p, hsa-miR-6768-5p, hsa-miR-6785-5p, hsa-miR-4706, hsa-miR-711, hsa-miR-1260a, hsa-miR-6746-5p, hsa-miR-6089, hsa-miR-6821-5p, hsa-miR-4667-5p, hsa-miR-8069, hsa-miR-4726-5p, hsa-miR-6124, hsa-miR-4532, hsa-miR-4486, hsa-miR-4728-5p, hsa-miR-4508, hsa-miR-128-1-5p, hsa-miR-4513, hsa-miR-6795-5p, hsa-miR-4689, hsa-miR-6763-5p, hsa-miR-8072, hsa-miR-6765-5p, hsa-miR-4419b, hsa-miR-7641, hsa-miR-3928-3p, hsa-miR-1227-5p, hsa-miR-4492, hsa-miR-296-3p, hsa-miR-6769a-5p, hsa-miR-6889-5p, hsa-miR-4632-5p, hsa-miR-4505, hsa-miR-3154, hsa-miR-3648, hsa-miR-4442, hsa-miR-3141, hsa-miR-7113-3p, hsa-miR-6819-5p, hsa-miR-3195, hsa-miR-1199-5p, hsa-miR-6738-5p, hsa-miR-4656, hsa-miR-6820-5p, hsa-miR-204-3p, hsa-miR-642a-3p, hsa-miR-762, hsa-miR-1202, hsa-miR-3162-5p, hsa-miR-3196, hsa-miR-3622a-5p, hsa-miR-3665, hsa-miR-3940-5p, hsa-miR-4294, hsa-miR-4466, hsa-miR-4476, hsa-miR-4723-5p, hsa-miR-4725-3p, hsa-miR-4730, hsa-miR-4739, hsa-miR-4787-5p, hsa-miR-5787, hsa-miR-6085, hsa-miR-6717-5p, hsa-miR-6724-5p, hsa-miR-6777-5p, hsa-miR-6778-5p, hsa-miR-6787-5p, hsa-miR-6789-5p, hsa-miR-6845-5p and hsa-miR-6893-5p. Furthermore, at least one or more miRNA(s) selected from the group consisting of other prostate cancer markers that can be combined with these miRNAs, i.e., hsa-miR-615-5p, hsa-miR-486-3p, hsa-miR-1225-3p, hsa-miR-760, hsa-miR-187-5p, hsa-miR-1203, hsa-miR-7110-5p, hsa-miR-371a-5p, hsa-miR-939-5p, hsa-miR-575, hsa-miR-92b-5p, hsa-miR-887-3p, hsa-miR-920, hsa-miR-1915-5p, hsa-miR-1231, hsa-miR-663b, hsa-miR-1225-5p, hsa-miR-16-5p, hsa-miR-423-5p, hsa-miR-451a, hsa-miR-564 and hsa-miR-671-5p can also be preferably used as a target nucleic acid(s). Moreover, at least one or more miRNA(s) selected from the group consisting of other prostate cancer markers that can be combined with these miRNAs, i.e., hsa-miR-4763-3p, hsa-miR-3656, hsa-miR-4488, hsa-miR-125a-3p, hsa-miR-1469, hsa-miR-1228-5p, hsa-miR-6798-5p, hsa-miR-1268b, hsa-miR-6732-5p, hsa-miR-1915-3p, hsa-miR-4433b-3p, hsa-miR-1207-5p, hsa-miR-4433-3p, hsa-miR-6879-5p, hsa-miR-4417, hsa-miR-30c-1-3p, hsa-miR-4638-5p, hsa-miR-6088, hsa-miR-4270, hsa-miR-6782-5p, hsa-miR-665, hsa-miR-486-5p, hsa-miR-4655-5p, hsa-miR-1275, hsa-miR-6806-5p, hsa-miR-614, hsa-miR-3937, hsa-miR-6752-5p, hsa-miR-6771-5p, hsa-miR-4450, hsa-miR-211-3p, hsa-miR-663a, hsa-miR-6842-5p, hsa-miR-7114-5p and hsa-miR-6779-5p can also be preferably used as a target nucleic acid(s).

These miRNAs include, for example, a human gene comprising a nucleotide sequence represented by any of SEQ ID NOs: 1 to 187 and 580 to 611 (i.e., hsa-miR-4443, hsa-miR-1908-5p, hsa-miR-4257, hsa-miR-3197, hsa-miR-3188, hsa-miR-4649-5p, hsa-miR-1343-3p, hsa-miR-6861-5p, hsa-miR-1343-5p, hsa-miR-642b-3p, hsa-miR-6741-5p, hsa-miR-4745-5p, hsa-miR-6826-5p, hsa-miR-3663-3p, hsa-miR-3131, hsa-miR-92a-2-5p, hsa-miR-4258, hsa-miR-4448, hsa-miR-6125, hsa-miR-6880-5p, hsa-miR-6132, hsa-miR-4467, hsa-miR-6749-5p, hsa-miR-2392, hsa-miR-1273g-3p, hsa-miR-4746-3p, hsa-miR-1914-3p, hsa-miR-7845-5p, hsa-miR-6726-5p, hsa-miR-128-2-5p, hsa-miR-4651, hsa-miR-6765-3p, hsa-miR-3185, hsa-miR-4792, hsa-miR-6887-5p, hsa-miR-5572, hsa-miR-3619-3p, hsa-miR-6780b-5p, hsa-miR-4707-5p, hsa-miR-8063, hsa-miR-4454, hsa-miR-4525, hsa-miR-7975, hsa-miR-744-5p, hsa-miR-3135b, hsa-miR-4648, hsa-miR-6816-5p, hsa-miR-4741, hsa-miR-7150, hsa-miR-6791-5p, hsa-miR-1247-3p, hsa-miR-7977, hsa-miR-4497, hsa-miR-6090, hsa-miR-6781-5p, hsa-miR-6870-5p, hsa-miR-6729-5p, hsa-miR-4530, hsa-miR-7847-3p, hsa-miR-6825-5p, hsa-miR-4674, hsa-miR-3917, hsa-miR-4707-3p, hsa-miR-6885-5p, hsa-miR-6722-3p, hsa-miR-4516, hsa-miR-6757-5p, hsa-miR-6840-3p, hsa-miR-5195-3p, hsa-miR-6756-5p, hsa-miR-6800-5p, hsa-miR-6727-5p, hsa-miR-6126, hsa-miR-6872-3p, hsa-miR-4446-3p, hsa-miR-1268a, hsa-miR-1908-3p, hsa-miR-3679-5p, hsa-miR-4534, hsa-miR-4675, hsa-miR-7108-5p, hsa-miR-6799-5p, hsa-miR-4695-5p, hsa-miR-3178, hsa-miR-5090, hsa-miR-3180, hsa-miR-1237-5p, hsa-miR-4758-5p, hsa-miR-3184-5p, hsa-miR-4286, hsa-miR-6784-5p, hsa-miR-6768-5p, hsa-miR-6785-5p, hsa-miR-4706, hsa-miR-711, hsa-miR-1260a, hsa-miR-6746-5p, hsa-miR-6089, hsa-miR-6821-5p, hsa-miR-4667-5p, hsa-miR-8069, hsa-miR-4726-5p, hsa-miR-6124, hsa-miR-4532, hsa-miR-4486, hsa-miR-4728-5p, hsa-miR-4508, hsa-miR-128-1-5p, hsa-miR-4513, hsa-miR-6795-5p, hsa-miR-4689, hsa-miR-6763-5p, hsa-miR-8072, hsa-miR-6765-5p, hsa-miR-4419b, hsa-miR-7641, hsa-miR-3928-3p, hsa-miR-1227-5p, hsa-miR-4492, hsa-miR-296-3p, hsa-miR-6769a-5p, hsa-miR-6889-5p, hsa-miR-4632-5p, hsa-miR-4505, hsa-miR-3154, hsa-miR-3648, hsa-miR-4442, hsa-miR-3141, hsa-miR-7113-3p, hsa-miR-6819-5p, hsa-miR-3195, hsa-miR-1199-5p, hsa-miR-6738-5p, hsa-miR-4656, hsa-miR-6820-5p, hsa-miR-204-3p, hsa-miR-642a-3p, hsa-miR-762, hsa-miR-1202, hsa-miR-3162-5p, hsa-miR-3196, hsa-miR-3622a-5p, hsa-miR-3665, hsa-miR-3940-5p, hsa-miR-4294, hsa-miR-4466, hsa-miR-4476, hsa-miR-4723-5p, hsa-miR-4725-3p, hsa-miR-4730, hsa-miR-4739, hsa-miR-4787-5p, hsa-miR-5787, hsa-miR-6085, hsa-miR-6717-5p, hsa-miR-6724-5p, hsa-miR-6777-5p, hsa-miR-6778-5p, hsa-miR-6787-5p, hsa-miR-6789-5p, hsa-miR-6845-5p and hsa-miR-6893-5p, hsa-miR-615-5p, hsa-miR-486-3p, hsa-miR-1225-3p, hsa-miR-760, hsa-miR-187-5p, hsa-miR-1203, hsa-miR-7110-5p, hsa-miR-371a-5p, hsa-miR-939-5p, hsa-miR-575, hsa-miR-92b-5p, hsa-miR-887-3p, hsa-miR-920, hsa-miR-1915-5p, hsa-miR-1231, hsa-miR-663b, hsa-miR-1225-5p, hsa-miR-16-5p, hsa-miR-423-5p, hsa-miR-451a, hsa-miR-564 and hsa-miR-671-5p, hsa-miR-4763-3p, hsa-miR-3656, hsa-miR-4488, hsa-miR-125a-3p, hsa-miR-1469, hsa-miR-1228-5p, hsa-miR-6798-5p, hsa-miR-1268b, hsa-miR-6732-5p, hsa-miR-1915-3p, hsa-miR-4433b-3p, hsa-miR-1207-5p, hsa-miR-4433-3p, hsa-miR-6879-5p, hsa-miR-4417, hsa-miR-30c-1-3p, hsa-miR-4638-5p, hsa-miR-6088, hsa-miR-4270, hsa-miR-6782-5p, hsa-miR-665, hsa-miR-486-5p, hsa-miR-4655-5p, hsa-miR-1275, hsa-miR-6806-5p, hsa-miR-614, hsa-miR-3937, hsa-miR-6752-5p, hsa-miR-6771-5p, hsa-miR-4450, hsa-miR-211-3p, hsa-miR-663a, hsa-miR-6842-5p, hsa-miR-7114-5p and hsa-miR-6779-5p, respectively), any congener thereof, any transcript thereof, and any variant or any derivative thereof. In this context, the gene, the congener, the transcript, the variant, and the derivative are as defined above.

The target nucleic acid is preferably a human gene comprising a nucleotide sequence represented by any of SEQ ID NOs: 1 to 684 or a transcript thereof, more preferably the transcript, i.e., a miRNA or its precursor RNA (pri-miRNA or pre-miRNA).

The first target gene is the hsa-miR-4443 gene, a congener thereof, a transcript thereof, or a variant or a derivative thereof. None of the previously known reports show that change in the expression of the gene or the transcript thereof can serve as a marker for prostate cancer.

The second target gene is the hsa-miR-1908-5p gene, a congener thereof, a transcript thereof, or a variant or a derivative thereof. None of the previously known reports show that change in the expression of the gene or the transcript thereof can serve as a marker for prostate cancer.

The third target gene is the hsa-miR-4257 gene, a congener thereof, a transcript thereof, or a variant or a derivative thereof. None of the previously known reports show that change in the expression of the gene or the transcript thereof can serve as a marker for prostate cancer.

The fourth target gene is the hsa-miR-3197 gene, a congener thereof, a transcript thereof, or a variant or a derivative thereof. None of the previously known reports show that change in the expression of the gene or the transcript thereof can serve as a marker for prostate cancer.

The fifth target gene is the hsa-miR-3188 gene, a congener thereof, a transcript thereof, or a variant or a derivative thereof. None of the previously known reports show that change in the expression of the gene or the transcript thereof can serve as a marker for prostate cancer.

The sixth target gene is the hsa-miR-4649-5p gene, a congener thereof, a transcript thereof, or a variant or a derivative thereof. None of the previously known reports show that change in the expression of the gene or the transcript thereof can serve as a marker for prostate cancer.

The seventh target gene is the hsa-miR-1343-3p gene, a congener thereof, a transcript thereof, or a variant or a derivative thereof. None of the previously known reports show that change in the expression of the gene or the transcript thereof can serve as a marker for prostate cancer.

The eighth target gene is the hsa-miR-6861-5p gene, a congener thereof, a transcript thereof, or a variant or a derivative thereof. None of the previously known reports show that change in the expression of the gene or the transcript thereof can serve as a marker for prostate cancer.

The ninth target gene is the hsa-miR-1343-5p gene, a congener thereof, a transcript thereof, or a variant or a derivative thereof. None of the previously known reports show that change in the expression of the gene or the transcript thereof can serve as a marker for prostate cancer.

The 10th target gene is the hsa-miR-642b-3p gene, a congener thereof, a transcript thereof, or a variant or a derivative thereof. None of the previously known reports show that change in the expression of the gene or the transcript thereof can serve as a marker for prostate cancer.

The 11th target gene is the hsa-miR-6741-5p gene, a congener thereof, a transcript thereof, or a variant or a derivative thereof. None of the previously known reports show that change in the expression of the gene or the transcript thereof can serve as a marker for prostate cancer.

The 12th target gene is the hsa-miR-4745-5p gene, a congener thereof, a transcript thereof, or a variant or a derivative thereof. None of the previously known reports show that change in the expression of the gene or the transcript thereof can serve as a marker for prostate cancer.

The 13th target gene is the hsa-miR-6826-5p gene, a congener thereof, a transcript thereof, or a variant or a derivative thereof. None of the previously known reports show that change in the expression of the gene or the transcript thereof can serve as a marker for prostate cancer.

The 14th target gene is the hsa-miR-3663-3p gene, a congener thereof, a transcript thereof, or a variant or a derivative thereof. None of the previously known reports show that change in the expression of the gene or the transcript thereof can serve as a marker for prostate cancer.

The 15th target gene is the hsa-miR-3131 gene, a congener thereof, a transcript thereof, or a variant or a derivative thereof. None of the previously known reports show that change in the expression of the gene or the transcript thereof can serve as a marker for prostate cancer.

The 16th target gene is the hsa-miR-92a-2-5p gene, a congener thereof, a transcript thereof, or a variant or a derivative thereof. None of the previously known reports show that change in the expression of the gene or the transcript thereof can serve as a marker for prostate cancer.

The 17th target gene is the hsa-miR-4258 gene, a congener thereof, a transcript thereof, or a variant or a derivative thereof. None of the previously known reports show that change in the expression of the gene or the transcript thereof can serve as a marker for prostate cancer.

The 18th target gene is the hsa-miR-4448 gene, a congener thereof, a transcript thereof, or a variant or a derivative thereof. None of the previously known reports show that change in the expression of the gene or the transcript thereof can serve as a marker for prostate cancer.

The 19th target gene is the hsa-miR-6125 gene, a congener thereof, a transcript thereof, or a variant or a derivative thereof. None of the previously known reports show that change in the expression of the gene or the transcript thereof can serve as a marker for prostate cancer.

The 20th target gene is the hsa-miR-6880-5p gene, a congener thereof, a transcript thereof, or a variant or a derivative thereof. None of the previously known reports show that change in the expression of the gene or the transcript thereof can serve as a marker for prostate cancer.

The 21st target gene is the hsa-miR-6132 gene, a congener thereof, a transcript thereof, or a variant or a derivative thereof. None of the previously known reports show that change in the expression of the gene or the transcript thereof can serve as a marker for prostate cancer.

The 22nd target gene is the hsa-miR-4467 gene, a congener thereof, a transcript thereof, or a variant or a derivative thereof. None of the previously known reports show that change in the expression of the gene or the transcript thereof can serve as a marker for prostate cancer.

The 23rd target gene is the hsa-miR-6749-5p gene, a congener thereof, a transcript thereof, or a variant or a derivative thereof. None of the previously known reports show that change in the expression of the gene or the transcript thereof can serve as a marker for prostate cancer.

The 24th target gene is the hsa-miR-2392 gene, a congener thereof, a transcript thereof, or a variant or a derivative thereof. None of the previously known reports show that change in the expression of the gene or the transcript thereof can serve as a marker for prostate cancer.

The 25th target gene is the hsa-miR-1273g-3p gene, a congener thereof, a transcript thereof, or a variant or a derivative thereof. None of the previously known reports show that change in the expression of the gene or the transcript thereof can serve as a marker for prostate cancer.

The 26th target gene is the hsa-miR-4746-3p gene, a congener thereof, a transcript thereof, or a variant or a derivative thereof. None of the previously known reports show that change in the expression of the gene or the transcript thereof can serve as a marker for prostate cancer.

The 27th target gene is the hsa-miR-1914-3p gene, a congener thereof, a transcript thereof, or a variant or a derivative thereof. None of the previously known reports show that change in the expression of the gene or the transcript thereof can serve as a marker for prostate cancer.

The 28th target gene is the hsa-miR-7845-5p gene, a congener thereof, a transcript thereof, or a variant or a derivative thereof. None of the previously known reports show that change in the expression of the gene or the transcript thereof can serve as a marker for prostate cancer.

The 29th target gene is the hsa-miR-6726-5p gene, a congener thereof, a transcript thereof, or a variant or a derivative thereof. None of the previously known reports show that change in the expression of the gene or the transcript thereof can serve as a marker for prostate cancer.

The 30th target gene is the hsa-miR-128-2-5p gene, a congener thereof, a transcript thereof, or a variant or a derivative thereof. None of the previously known reports show that change in the expression of the gene or the transcript thereof can serve as a marker for prostate cancer.

The 31st target gene is the hsa-miR-4651 gene, a congener thereof, a transcript thereof, or a variant or a derivative thereof. None of the previously known reports show that change in the expression of the gene or the transcript thereof can serve as a marker for prostate cancer.

The 32nd target gene is the hsa-miR-6765-3p gene, a congener thereof, a transcript thereof, or a variant or a derivative thereof. None of the previously known reports show that change in the expression of the gene or the transcript thereof can serve as a marker for prostate cancer.

The 33rd target gene is the hsa-miR-3185 gene, a congener thereof, a transcript thereof, or a variant or a derivative thereof. None of the previously known reports show that change in the expression of the gene or the transcript thereof can serve as a marker for prostate cancer.

The 34th target gene is the hsa-miR-4792 gene, a congener thereof, a transcript thereof, or a variant or a derivative thereof. None of the previously known reports show that change in the expression of the gene or the transcript thereof can serve as a marker for prostate cancer.

The 35th target gene is the hsa-miR-6887-5p gene, a congener thereof, a transcript thereof, or a variant or a derivative thereof. None of the previously known reports show that change in the expression of the gene or the transcript thereof can serve as a marker for prostate cancer.

The 36th target gene is the hsa-miR-5572 gene, a congener thereof, a transcript thereof, or a variant or a derivative thereof. None of the previously known reports show that change in the expression of the gene or the transcript thereof can serve as a marker for prostate cancer.

The 37th target gene is the hsa-miR-3619-3p gene, a congener thereof, a transcript thereof, or a variant or a derivative thereof. None of the previously known reports show that change in the expression of the gene or the transcript thereof can serve as a marker for prostate cancer.

The 38th target gene is the hsa-miR-6780b-5p gene, a congener thereof, a transcript thereof, or a variant or a derivative thereof. None of the previously known reports show that change in the expression of the gene or the transcript thereof can serve as a marker for prostate cancer.

The 39th target gene is the hsa-miR-4707-5p gene, a congener thereof, a transcript thereof, or a variant or a derivative thereof. None of the previously known reports show that change in the expression of the gene or the transcript thereof can serve as a marker for prostate cancer.

The 40th target gene is the hsa-miR-8063 gene, a congener thereof, a transcript thereof, or a variant or a derivative thereof. None of the previously known reports show that change in the expression of the gene or the transcript thereof can serve as a marker for prostate cancer.

The 41st target gene is the hsa-miR-4454 gene, a congener thereof, a transcript thereof, or a variant or a derivative thereof. None of the previously known reports show that change in the expression of the gene or the transcript thereof can serve as a marker for prostate cancer.

The 42nd target gene is the hsa-miR-4525 gene, a congener thereof, a transcript thereof, or a variant or a derivative thereof. None of the previously known reports show that change in the expression of the gene or the transcript thereof can serve as a marker for prostate cancer.

The 43rd target gene is the hsa-miR-7975 gene, a congener thereof, a transcript thereof, or a variant or a derivative thereof. None of the previously known reports show that change in the expression of the gene or the transcript thereof can serve as a marker for prostate cancer.

The 44th target gene is the hsa-miR-744-5p gene, a congener thereof, a transcript thereof, or a variant or a derivative thereof. None of the previously known reports show that change in the expression of the gene or the transcript thereof can serve as a marker for prostate cancer.

The 45th target gene is the hsa-miR-3135b gene, a congener thereof, a transcript thereof, or a variant or a derivative thereof. None of the previously known reports show that change in the expression of the gene or the transcript thereof can serve as a marker for prostate cancer.

The 46th target gene is the hsa-miR-4648 gene, a congener thereof, a transcript thereof, or a variant or a derivative thereof. None of the previously known reports show that change in the expression of the gene or the transcript thereof can serve as a marker for prostate cancer.

The 47th target gene is the hsa-miR-6816-5p gene, a congener thereof, a transcript thereof, or a variant or a derivative thereof. None of the previously known reports show that change in the expression of the gene or the transcript thereof can serve as a marker for prostate cancer.

The 48th target gene is the hsa-miR-4741 gene, a congener thereof, a transcript thereof, or a variant or a derivative thereof. None of the previously known reports show that change in the expression of the gene or the transcript thereof can serve as a marker for prostate cancer.

The 49th target gene is the hsa-miR-7150 gene, a congener thereof, a transcript thereof, or a variant or a derivative thereof. None of the previously known reports show that change in the expression of the gene or the transcript thereof can serve as a marker for prostate cancer.

The 50th target gene is the hsa-miR-6791-5p gene, a congener thereof, a transcript thereof, or a variant or a derivative thereof. None of the previously known reports show that change in the expression of the gene or the transcript thereof can serve as a marker for prostate cancer.

The 51st target gene is the hsa-miR-1247-3p gene, a congener thereof, a transcript thereof, or a variant or a derivative thereof. None of the previously known reports show that change in the expression of the gene or the transcript thereof can serve as a marker for prostate cancer.

The 52nd target gene is the hsa-miR-7977 gene, a congener thereof, a transcript thereof, or a variant or a derivative thereof. None of the previously known reports show that change in the expression of the gene or the transcript thereof can serve as a marker for prostate cancer.

The 53rd target gene is the hsa-miR-4497 gene, a congener thereof, a transcript thereof, or a variant or a derivative thereof. None of the previously known reports show that change in the expression of the gene or the transcript thereof can serve as a marker for prostate cancer.

The 54th target gene is the hsa-miR-6090 gene, a congener thereof, a transcript thereof, or a variant or a derivative thereof. None of the previously known reports show that change in the expression of the gene or the transcript thereof can serve as a marker for prostate cancer.

The 55th target gene is the hsa-miR-6781-5p gene, a congener thereof, a transcript thereof, or a variant or a derivative thereof. None of the previously known reports show that change in the expression of the gene or the transcript thereof can serve as a marker for prostate cancer.

The 56th target gene is the hsa-miR-6870-5p gene, a congener thereof, a transcript thereof, or a variant or a derivative thereof. None of the previously known reports show that change in the expression of the gene or the transcript thereof can serve as a marker for prostate cancer.

The 57th target gene is the hsa-miR-6729-5p gene, a congener thereof, a transcript thereof, or a variant or a derivative thereof. None of the previously known reports show that change in the expression of the gene or the transcript thereof can serve as a marker for prostate cancer.

The 58th target gene is the hsa-miR-4530 gene, a congener thereof, a transcript thereof, or a variant or a derivative thereof. None of the previously known reports show that change in the expression of the gene or the transcript thereof can serve as a marker for prostate cancer.

The 59th target gene is the hsa-miR-7847-3p gene, a congener thereof, a transcript thereof, or a variant or a derivative thereof. None of the previously known reports show that change in the expression of the gene or the transcript thereof can serve as a marker for prostate cancer.

The 60th target gene is the hsa-miR-6825-5p gene, a congener thereof, a transcript thereof, or a variant or a derivative thereof. None of the previously known reports show that change in the expression of the gene or the transcript thereof can serve as a marker for prostate cancer.

The 61st target gene is the hsa-miR-4674 gene, a congener thereof, a transcript thereof, or a variant or a derivative thereof. None of the previously known reports show that change in the expression of the gene or the transcript thereof can serve as a marker for prostate cancer.

The 62nd target gene is the hsa-miR-3917 gene, a congener thereof, a transcript thereof, or a variant or a derivative thereof. None of the previously known reports show that change in the expression of the gene or the transcript thereof can serve as a marker for prostate cancer.

The 63rd target gene is the hsa-miR-4707-3p gene, a congener thereof, a transcript thereof, or a variant or a derivative thereof. None of the previously known reports show that change in the expression of the gene or the transcript thereof can serve as a marker for prostate cancer.

The 64th target gene is the hsa-miR-6885-5p gene, a congener thereof, a transcript thereof, or a variant or a derivative thereof. None of the previously known reports show that change in the expression of the gene or the transcript thereof can serve as a marker for prostate cancer.

The 65th target gene is the hsa-miR-6722-3p gene, a congener thereof, a transcript thereof, or a variant or a derivative thereof. None of the previously known reports show that change in the expression of the gene or the transcript thereof can serve as a marker for prostate cancer.

The 66th target gene is the hsa-miR-4516 gene, a congener thereof, a transcript thereof, or a variant or a derivative thereof. None of the previously known reports show that change in the expression of the gene or the transcript thereof can serve as a marker for prostate cancer.

The 67th target gene is the hsa-miR-6757-5p gene, a congener thereof, a transcript thereof, or a variant or a derivative thereof. None of the previously known reports show that change in the expression of the gene or the transcript thereof can serve as a marker for prostate cancer.

The 68th target gene is the hsa-miR-6840-3p gene, a congener thereof, a transcript thereof, or a variant or a derivative thereof. None of the previously known reports show that change in the expression of the gene or the transcript thereof can serve as a marker for prostate cancer.

The 69th target gene is the hsa-miR-5195-3p gene, a congener thereof, a transcript thereof, or a variant or a derivative thereof. None of the previously known reports show that change in the expression of the gene or the transcript thereof can serve as a marker for prostate cancer.

The 70th target gene is the hsa-miR-6756-5p gene, a congener thereof, a transcript thereof, or a variant or a derivative thereof. None of the previously known reports show that change in the expression of the gene or the transcript thereof can serve as a marker for prostate cancer.

The 71st target gene is the hsa-miR-6800-5p gene, a congener thereof, a transcript thereof, or a variant or a derivative thereof. None of the previously known reports show that change in the expression of the gene or the transcript thereof can serve as a marker for prostate cancer.

The 72nd target gene is the hsa-miR-6727-5p gene, a congener thereof, a transcript thereof, or a variant or a derivative thereof. None of the previously known reports show that change in the expression of the gene or the transcript thereof can serve as a marker for prostate cancer.

The 73rd target gene is the hsa-miR-6126 gene, a congener thereof, a transcript thereof, or a variant or a derivative thereof. None of the previously known reports show that change in the expression of the gene or the transcript thereof can serve as a marker for prostate cancer.

The 74th target gene is the hsa-miR-6872-3p gene, a congener thereof, a transcript thereof, or a variant or a derivative thereof. None of the previously known reports show that change in the expression of the gene or the transcript thereof can serve as a marker for prostate cancer.

The 75th target gene is the hsa-miR-4446-3p gene, a congener thereof, a transcript thereof, or a variant or a derivative thereof. None of the previously known reports show that change in the expression of the gene or the transcript thereof can serve as a marker for prostate cancer.

The 76th target gene is the hsa-miR-1268a gene, a congener thereof, a transcript thereof, or a variant or a derivative thereof. None of the previously known reports show that change in the expression of the gene or the transcript thereof can serve as a marker for prostate cancer.

The 77th target gene is the hsa-miR-1908-3p gene, a congener thereof, a transcript thereof, or a variant or a derivative thereof. None of the previously known reports show that change in the expression of the gene or the transcript thereof can serve as a marker for prostate cancer.

The 78th target gene is the hsa-miR-3679-5p gene, a congener thereof, a transcript thereof, or a variant or a derivative thereof. None of the previously known reports show that change in the expression of the gene or the transcript thereof can serve as a marker for prostate cancer.

The 79th target gene is the hsa-miR-4534 gene, a congener thereof, a transcript thereof, or a variant or a derivative thereof. None of the previously known reports show that change in the expression of the gene or the transcript thereof can serve as a marker for prostate cancer.

The 80th target gene is the hsa-miR-4675 gene, a congener thereof, a transcript thereof, or a variant or a derivative thereof. None of the previously known reports show that change in the expression of the gene or the transcript thereof can serve as a marker for prostate cancer.

The 81st target gene is the hsa-miR-7108-5p gene, a congener thereof, a transcript thereof, or a variant or a derivative thereof. None of the previously known reports show that change in the expression of the gene or the transcript thereof can serve as a marker for prostate cancer.

The 82nd target gene is the hsa-miR-6799-5p gene, a congener thereof, a transcript thereof, or a variant or a derivative thereof. None of the previously known reports show that change in the expression of the gene or the transcript thereof can serve as a marker for prostate cancer.

The 83rd target gene is the hsa-miR-4695-5p gene, a congener thereof, a transcript thereof, or a variant or a derivative thereof. None of the previously known reports show that change in the expression of the gene or the transcript thereof can serve as a marker for prostate cancer.

The 84th target gene is the hsa-miR-3178 gene, a congener thereof, a transcript thereof, or a variant or a derivative thereof. None of the previously known reports show that change in the expression of the gene or the transcript thereof can serve as a marker for prostate cancer.

The 85th target gene is the hsa-miR-5090 gene, a congener thereof, a transcript thereof, or a variant or a derivative thereof. None of the previously known reports show that change in the expression of the gene or the transcript thereof can serve as a marker for prostate cancer.

The 86th target gene is the hsa-miR-3180 gene, a congener thereof, a transcript thereof, or a variant or a derivative thereof. None of the previously known reports show that change in the expression of the gene or the transcript thereof can serve as a marker for prostate cancer.

The 87th target gene is the hsa-miR-1237-5p gene, a congener thereof, a transcript thereof, or a variant or a derivative thereof. None of the previously known reports show that change in the expression of the gene or the transcript thereof can serve as a marker for prostate cancer.

The 88th target gene is the hsa-miR-4758-5p gene, a congener thereof, a transcript thereof, or a variant or a derivative thereof. None of the previously known reports show that change in the expression of the gene or the transcript thereof can serve as a marker for prostate cancer.

The 89th target gene is the hsa-miR-3184-5p gene, a congener thereof, a transcript thereof, or a variant or a derivative thereof. None of the previously known reports show that change in the expression of the gene or the transcript thereof can serve as a marker for prostate cancer.

The 90th target gene is the hsa-miR-4286 gene, a congener thereof, a transcript thereof, or a variant or a derivative thereof. None of the previously known reports show that change in the expression of the gene or the transcript thereof can serve as a marker for prostate cancer.

The 91st target gene is the hsa-miR-6784-5p gene, a congener thereof, a transcript thereof, or a variant or a derivative thereof. None of the previously known reports show that change in the expression of the gene or the transcript thereof can serve as a marker for prostate cancer.

The 92nd target gene is the hsa-miR-6768-5p gene, a congener thereof, a transcript thereof, or a variant or a derivative thereof. None of the previously known reports show that change in the expression of the gene or the transcript thereof can serve as a marker for prostate cancer.

The 93rd target gene is the hsa-miR-6785-5p gene, a congener thereof, a transcript thereof, or a variant or a derivative thereof. None of the previously known reports show that change in the expression of the gene or the transcript thereof can serve as a marker for prostate cancer.

The 94th target gene is the hsa-miR-4706 gene, a congener thereof, a transcript thereof, or a variant or a derivative thereof. None of the previously known reports show that change in the expression of the gene or the transcript thereof can serve as a marker for prostate cancer.

The 95th target gene is the hsa-miR-711 gene, a congener thereof, a transcript thereof, or a variant or a derivative thereof. None of the previously known reports show that change in the expression of the gene or the transcript thereof can serve as a marker for prostate cancer.

The 96th target gene is the hsa-miR-1260a gene, a congener thereof, a transcript thereof, or a variant or a derivative thereof. None of the previously known reports show that change in the expression of the gene or the transcript thereof can serve as a marker for prostate cancer.

The 97th target gene is the hsa-miR-6746-5p gene, a congener thereof, a transcript thereof, or a variant or a derivative thereof. None of the previously known reports show that change in the expression of the gene or the transcript thereof can serve as a marker for prostate cancer.

The 98th target gene is the hsa-miR-6089 gene, a congener thereof, a transcript thereof, or a variant or a derivative thereof. None of the previously known reports show that change in the expression of the gene or the transcript thereof can serve as a marker for prostate cancer.

The 99th target gene is the hsa-miR-6821-5p gene, a congener thereof, a transcript thereof, or a variant or a derivative thereof. None of the previously known reports show that change in the expression of the gene or the transcript thereof can serve as a marker for prostate cancer.

The 100th target gene is the hsa-miR-4667-5p gene, a congener thereof, a transcript thereof, or a variant or a derivative thereof. None of the previously known reports show that change in the expression of the gene or the transcript thereof can serve as a marker for prostate cancer.

The 101st target gene is the hsa-miR-8069 gene, a congener thereof, a transcript thereof, or a variant or a derivative thereof. None of the previously known reports show that change in the expression of the gene or the transcript thereof can serve as a marker for prostate cancer.

The 102nd target gene is the hsa-miR-4726-5p gene, a congener thereof, a transcript thereof, or a variant or a derivative thereof. None of the previously known reports show that change in the expression of the gene or the transcript thereof can serve as a marker for prostate cancer.

The 103rd target gene is the hsa-miR-6124 gene, a congener thereof, a transcript thereof, or a variant or a derivative thereof. None of the previously known reports show that change in the expression of the gene or the transcript thereof can serve as a marker for prostate cancer.

The 104th target gene is the hsa-miR-4532 gene, a congener thereof, a transcript thereof, or a variant or a derivative thereof. None of the previously known reports show that change in the expression of the gene or the transcript thereof can serve as a marker for prostate cancer.

The 105th target gene is the hsa-miR-4486 gene, a congener thereof, a transcript thereof, or a variant or a derivative thereof. None of the previously known reports show that change in the expression of the gene or the transcript thereof can serve as a marker for prostate cancer.

The 106th target gene is the hsa-miR-4728-5p gene, a congener thereof, a transcript thereof, or a variant or a derivative thereof. None of the previously known reports show that change in the expression of the gene or the transcript thereof can serve as a marker for prostate cancer.

The 107th target gene is the hsa-miR-4508 gene, a congener thereof, a transcript thereof, or a variant or a derivative thereof. None of the previously known reports show that change in the expression of the gene or the transcript thereof can serve as a marker for prostate cancer.

The 108th target gene is the hsa-miR-128-1-5p gene, a congener thereof, a transcript thereof, or a variant or a derivative thereof. None of the previously known reports show that change in the expression of the gene or the transcript thereof can serve as a marker for prostate cancer.

The 109th target gene is the hsa-miR-4513 gene, a congener thereof, a transcript thereof, or a variant or a derivative thereof. None of the previously known reports show that change in the expression of the gene or the transcript thereof can serve as a marker for prostate cancer.

The 110th target gene is the hsa-miR-6795-5p gene, a congener thereof, a transcript thereof, or a variant or a derivative thereof. None of the previously known reports show that change in the expression of the gene or the transcript thereof can serve as a marker for prostate cancer.

The 111th target gene is the hsa-miR-4689 gene, a congener thereof, a transcript thereof, or a variant or a derivative thereof. None of the previously known reports show that change in the expression of the gene or the transcript thereof can serve as a marker for prostate cancer.

The 112th target gene is the hsa-miR-6763-5p gene, a congener thereof, a transcript thereof, or a variant or a derivative thereof. None of the previously known reports show that change in the expression of the gene or the transcript thereof can serve as a marker for prostate cancer.

The 113th target gene is the hsa-miR-8072 gene, a congener thereof, a transcript thereof, or a variant or a derivative thereof. None of the previously known reports show that change in the expression of the gene or the transcript thereof can serve as a marker for prostate cancer.

The 114th target gene is the hsa-miR-6765-5p gene, a congener thereof, a transcript thereof, or a variant or a derivative thereof. None of the previously known reports show that change in the expression of the gene or the transcript thereof can serve as a marker for prostate cancer.

The 115th target gene is the hsa-miR-4419b gene, a congener thereof, a transcript thereof, or a variant or a derivative thereof. None of the previously known reports show that change in the expression of the gene or the transcript thereof can serve as a marker for prostate cancer.

The 116th target gene is the hsa-miR-7641 gene, a congener thereof, a transcript thereof, or a variant or a derivative thereof. None of the previously known reports show that change in the expression of the gene or the transcript thereof can serve as a marker for prostate cancer.

The 117th target gene is the hsa-miR-3928-3p gene, a congener thereof, a transcript thereof, or a variant or a derivative thereof. None of the previously known reports show that change in the expression of the gene or the transcript thereof can serve as a marker for prostate cancer.

The 118th target gene is the hsa-miR-1227-5p gene, a congener thereof, a transcript thereof, or a variant or a derivative thereof. None of the previously known reports show that change in the expression of the gene or the transcript thereof can serve as a marker for prostate cancer.

The 119th target gene is the hsa-miR-4492 gene, a congener thereof, a transcript thereof, or a variant or a derivative thereof. None of the previously known reports show that change in the expression of the gene or the transcript thereof can serve as a marker for prostate cancer.

The 120th target gene is the hsa-miR-296-3p gene, a congener thereof, a transcript thereof, or a variant or a derivative thereof. None of the previously known reports show that change in the expression of the gene or the transcript thereof can serve as a marker for prostate cancer.

The 121st target gene is the hsa-miR-6769a-5p gene, a congener thereof, a transcript thereof, or a variant or a derivative thereof. None of the previously known reports show that change in the expression of the gene or the transcript thereof can serve as a marker for prostate cancer.

The 122nd target gene is the hsa-miR-6889-5p gene, a congener thereof, a transcript thereof, or a variant or a derivative thereof. None of the previously known reports show that change in the expression of the gene or the transcript thereof can serve as a marker for prostate cancer.

The 123rd target gene is the hsa-miR-4632-5p gene, a congener thereof, a transcript thereof, or a variant or a derivative thereof. None of the previously known reports show that change in the expression of the gene or the transcript thereof can serve as a marker for prostate cancer.

The 124th target gene is the hsa-miR-4505 gene, a congener thereof, a transcript thereof, or a variant or a derivative thereof. None of the previously known reports show that change in the expression of the gene or the transcript thereof can serve as a marker for prostate cancer.

The 125th target gene is the hsa-miR-3154 gene, a congener thereof, a transcript thereof, or a variant or a derivative thereof. None of the previously known reports show that change in the expression of the gene or the transcript thereof can serve as a marker for prostate cancer.

The 126th target gene is the hsa-miR-3648 gene, a congener thereof, a transcript thereof, or a variant or a derivative thereof. None of the previously known reports show that change in the expression of the gene or the transcript thereof can serve as a marker for prostate cancer.

The 127th target gene is the hsa-miR-4442 gene, a congener thereof, a transcript thereof, or a variant or a derivative thereof. None of the previously known reports show that change in the expression of the gene or the transcript thereof can serve as a marker for prostate cancer.

The 128th target gene is the hsa-miR-3141 gene, a congener thereof, a transcript thereof, or a variant or a derivative thereof. None of the previously known reports show that change in the expression of the gene or the transcript thereof can serve as a marker for prostate cancer.

The 129th target gene is the hsa-miR-7113-3p gene, a congener thereof, a transcript thereof, or a variant or a derivative thereof. None of the previously known reports show that change in the expression of the gene or the transcript thereof can serve as a marker for prostate cancer.

The 130th target gene is the hsa-miR-6819-5p gene, a congener thereof, a transcript thereof, or a variant or a derivative thereof. None of the previously known reports show that change in the expression of the gene or the transcript thereof can serve as a marker for prostate cancer.

The 131st target gene is the hsa-miR-3195 gene, a congener thereof, a transcript thereof, or a variant or a derivative thereof. None of the previously known reports show that change in the expression of the gene or the transcript thereof can serve as a marker for prostate cancer.

The 132nd target gene is the hsa-miR-1199-5p gene, a congener thereof, a transcript thereof, or a variant or a derivative thereof. None of the previously known reports show that change in the expression of the gene or the transcript thereof can serve as a marker for prostate cancer.

The 133rd target gene is the hsa-miR-6738-5p gene, a congener thereof, a transcript thereof, or a variant or a derivative thereof. None of the previously known reports show that change in the expression of the gene or the transcript thereof can serve as a marker for prostate cancer.

The 134th target gene is the hsa-miR-4656 gene, a congener thereof, a transcript thereof, or a variant or a derivative thereof. None of the previously known reports show that change in the expression of the gene or the transcript thereof can serve as a marker for prostate cancer.

The 135th target gene is the hsa-miR-6820-5p gene, a congener thereof, a transcript thereof, or a variant or a derivative thereof. None of the previously known reports show that change in the expression of the gene or the transcript thereof can serve as a marker for prostate cancer.

The 136th target gene is the hsa-miR-615-5p gene, a congener thereof, a transcript thereof, or a variant or a derivative thereof. The previously known report shows that change in the expression of the gene or the transcript thereof can serve as a marker for prostate cancer (Patent Literature 1).

The 137th target gene is the hsa-miR-486-3p gene, a congener thereof, a transcript thereof, or a variant or a derivative thereof. The previously known report shows that change in the expression of the gene or the transcript thereof can serve as a marker for prostate cancer (Patent Literature 1).

The 138th target gene is the hsa-miR-1225-3p gene, a congener thereof, a transcript thereof, or a variant or a derivative thereof. The previously known report shows that change in the expression of the gene or the transcript thereof can serve as a marker for prostate cancer (Patent Literature 1).

The 139th target gene is the hsa-miR-760 gene, a congener thereof, a transcript thereof, or a variant or a derivative thereof. The previously known report shows that change in the expression of the gene or the transcript thereof can serve as a marker for prostate cancer (Patent Literature 1).

The 140th target gene is the hsa-miR-187-5p gene, a congener thereof, a transcript thereof, or a variant or a derivative thereof. The previously known report shows that change in the expression of the gene or the transcript thereof can serve as a marker for prostate cancer (Patent Literature 1).

The 141st target gene is the hsa-miR-1203 gene, a congener thereof, a transcript thereof, or a variant or a derivative thereof. The previously known report shows that change in the expression of the gene or the transcript thereof can serve as a marker for prostate cancer (Patent Literature 1).

The 142nd target gene is the hsa-miR-7110-5p gene, a congener thereof, a transcript thereof, or a variant or a derivative thereof. The previously known report shows that change in the expression of the gene or the transcript thereof can serve as a marker for prostate cancer (Patent Literature 1).

The 143rd target gene is the hsa-miR-371a-5p gene, a congener thereof, a transcript thereof, or a variant or a derivative thereof. The previously known report shows that change in the expression of the gene or the transcript thereof can serve as a marker for prostate cancer (Patent Literature 1).

The 144th target gene is the hsa-miR-939-5p gene, a congener thereof, a transcript thereof, or a variant or a derivative thereof. The previously known report shows that change in the expression of the gene or the transcript thereof can serve as a marker for prostate cancer (Patent Literature 1).

The 145th target gene is the hsa-miR-575 gene, a congener thereof, a transcript thereof, or a variant or a derivative thereof. The previously known report shows that change in the expression of the gene or the transcript thereof can serve as a marker for prostate cancer (Patent Literature 1).

The 146th target gene is the hsa-miR-92b-5p gene, a congener thereof, a transcript thereof, or a variant or a derivative thereof. The previously known report shows that change in the expression of the gene or the transcript thereof can serve as a marker for prostate cancer (Patent Literature 2).

The 147th target gene is the hsa-miR-887-3p gene, a congener thereof, a transcript thereof, or a variant or a derivative thereof. The previously known report shows that change in the expression of the gene or the transcript thereof can serve as a marker for prostate cancer (Patent Literature 1).

The 148th target gene is the hsa-miR-920 gene, a congener thereof, a transcript thereof, or a variant or a derivative thereof. The previously known report shows that change in the expression of the gene or the transcript thereof can serve as a marker for prostate cancer (Patent Literature 1).

The 149th target gene is the hsa-miR-1915-5p gene, a congener thereof, a transcript thereof, or a variant or a derivative thereof. The previously known report shows that change in the expression of the gene or the transcript thereof can serve as a marker for prostate cancer (Patent Literature 1).

The 150th target gene is the hsa-miR-1231 gene, a congener thereof, a transcript thereof, or a variant or a derivative thereof. The previously known report shows that change in the expression of the gene or the transcript thereof can serve as a marker for prostate cancer (Patent Literature 1).

The 151st target gene is the hsa-miR-663b gene, a congener thereof, a transcript thereof, or a variant or a derivative thereof. The previously known report shows that change in the expression of the gene or the transcript thereof can serve as a marker for prostate cancer (Patent Literature 1).

The 152nd target gene is the hsa-miR-1225-5p gene, a congener thereof, a transcript thereof, or a variant or a derivative thereof. The previously known report shows that change in the expression of the gene or the transcript thereof can serve as a marker for prostate cancer (Patent Literature 1).

The 153rd target gene is the hsa-miR-4763-3p gene, a congener thereof, a transcript thereof, or a variant or a derivative thereof. None of the previously known reports show that change in the expression of the gene or the transcript thereof can serve as a marker for prostate cancer.

The 154th target gene is the hsa-miR-3656 gene, a congener thereof, a transcript thereof, or a variant or a derivative thereof. None of the previously known reports show that change in the expression of the gene or the transcript thereof can serve as a marker for prostate cancer.

The 155th target gene is the hsa-miR-4488 gene, a congener thereof, a transcript thereof, or a variant or a derivative thereof. None of the previously known reports show that change in the expression of the gene or the transcript thereof can serve as a marker for prostate cancer.

The 156th target gene is the hsa-miR-125a-3p gene, a congener thereof, a transcript thereof, or a variant or a derivative thereof. The previously known report shows that change in the expression of the gene or the transcript thereof can serve as a marker for prostate cancer (Patent Literature 1).

The 157th target gene is the hsa-miR-1469 gene, a congener thereof, a transcript thereof, or a variant or a derivative thereof. None of the previously known reports show that change in the expression of the gene or the transcript thereof can serve as a marker for prostate cancer.

The 158th target gene is the hsa-miR-1228-5p gene, a congener thereof, a transcript thereof, or a variant or a derivative thereof. The previously known report shows that change in the expression of the gene or the transcript thereof can serve as a marker for prostate cancer (Patent Literature 1).

The 159th target gene is the hsa-miR-6798-5p gene, a congener thereof, a transcript thereof, or a variant or a derivative thereof. None of the previously known reports show that change in the expression of the gene or the transcript thereof can serve as a marker for prostate cancer.

The 160th target gene is the hsa-miR-1268b gene, a congener thereof, a transcript thereof, or a variant or a derivative thereof. The previously known report shows that change in the expression of the gene or the transcript thereof can serve as a marker for prostate cancer (Patent Literature 1).

The 161st target gene is the hsa-miR-6732-5p gene, a congener thereof, a transcript thereof, or a variant or a derivative thereof. None of the previously known reports show that change in the expression of the gene or the transcript thereof can serve as a marker for prostate cancer.

The 162nd target gene is the hsa-miR-1915-3p gene, a congener thereof, a transcript thereof, or a variant or a derivative thereof. The previously known report shows that change in the expression of the gene or the transcript thereof can serve as a marker for prostate cancer (Patent Literature 1).

The 163rd target gene is the hsa-miR-4433b-3p gene, a congener thereof, a transcript thereof, or a variant or a derivative thereof. None of the previously known reports show that change in the expression of the gene or the transcript thereof can serve as a marker for prostate cancer.

The 164th target gene is the hsa-miR-1207-5p gene, a congener thereof, a transcript thereof, or a variant or a derivative thereof. The previously known report shows that change in the expression of the gene or the transcript thereof can serve as a marker for prostate cancer (Patent Literature 1).

The 165th target gene is the hsa-miR-4433-3p gene, a congener thereof, a transcript thereof, or a variant or a derivative thereof. None of the previously known reports show that change in the expression of the gene or the transcript thereof can serve as a marker for prostate cancer.

The 166th target gene is the hsa-miR-6879-5p gene, a congener thereof, a transcript thereof, or a variant or a derivative thereof. None of the previously known reports show that change in the expression of the gene or the transcript thereof can serve as a marker for prostate cancer.

The 167th target gene is the hsa-miR-4417 gene, a congener thereof, a transcript thereof, or a variant or a derivative thereof. None of the previously known reports show that change in the expression of the gene or the transcript thereof can serve as a marker for prostate cancer.

The 168th target gene is the hsa-miR-30c-1-3p gene, a congener thereof, a transcript thereof, or a variant or a derivative thereof. The previously known report shows that change in the expression of the gene or the transcript thereof can serve as a marker for prostate cancer (Patent Literature 1).

The 169th target gene is the hsa-miR-4638-5p gene, a congener thereof, a transcript thereof, or a variant or a derivative thereof. None of the previously known reports show that change in the expression of the gene or the transcript thereof can serve as a marker for prostate cancer.

The 170th target gene is the hsa-miR-6088 gene, a congener thereof, a transcript thereof, or a variant or a derivative thereof. None of the previously known reports show that change in the expression of the gene or the transcript thereof can serve as a marker for prostate cancer.

The 171st target gene is the hsa-miR-4270 gene, a congener thereof, a transcript thereof, or a variant or a derivative thereof. None of the previously known reports show that change in the expression of the gene or the transcript thereof can serve as a marker for prostate cancer.

The 172nd target gene is the hsa-miR-6782-5p gene, a congener thereof, a transcript thereof, or a variant or a derivative thereof. None of the previously known reports show that change in the expression of the gene or the transcript thereof can serve as a marker for prostate cancer.

The 173rd target gene is the hsa-miR-665 gene, a congener thereof, a transcript thereof, or a variant or a derivative thereof. The previously known report shows that change in the expression of the gene or the transcript thereof can serve as a marker for prostate cancer (Patent Literature 1).

The 174th target gene is the hsa-miR-486-5p gene, a congener thereof, a transcript thereof, or a variant or a derivative thereof. The previously known report shows that change in the expression of the gene or the transcript thereof can serve as a marker for prostate cancer (Patent Literature 1).

The 175th target gene is the hsa-miR-4655-5p gene, a congener thereof, a transcript thereof, or a variant or a derivative thereof. None of the previously known reports show that change in the expression of the gene or the transcript thereof can serve as a marker for prostate cancer.

The 176th target gene is the hsa-miR-1275 gene, a congener thereof, a transcript thereof, or a variant or a derivative thereof. The previously known report shows that change in the expression of the gene or the transcript thereof can serve as a marker for prostate cancer (Patent Literature 1).

The 177th target gene is the hsa-miR-6806-5p gene, a congener thereof, a transcript thereof, or a variant or a derivative thereof. None of the previously known reports show that change in the expression of the gene or the transcript thereof can serve as a marker for prostate cancer.

The 178th target gene is the hsa-miR-614 gene, a congener thereof, a transcript thereof, or a variant or a derivative thereof. The previously known report shows that change in the expression of the gene or the transcript thereof can serve as a marker for prostate cancer (Patent Literature 1).

The 179th target gene is the hsa-miR-3937 gene, a congener thereof, a transcript thereof, or a variant or a derivative thereof. None of the previously known reports show that change in the expression of the gene or the transcript thereof can serve as a marker for prostate cancer.

The 180th target gene is the hsa-miR-6752-5p gene, a congener thereof, a transcript thereof, or a variant or a derivative thereof. None of the previously known reports show that change in the expression of the gene or the transcript thereof can serve as a marker for prostate cancer.

The 181st target gene is the hsa-miR-6771-5p gene, a congener thereof, a transcript thereof, or a variant or a derivative thereof. None of the previously known reports show that change in the expression of the gene or the transcript thereof can serve as a marker for prostate cancer.

The 182nd target gene is the hsa-miR-4450 gene, a congener thereof, a transcript thereof, or a variant or a derivative thereof. None of the previously known reports show that change in the expression of the gene or the transcript thereof can serve as a marker for prostate cancer.

The 183rd target gene is the hsa-miR-211-3p gene, a congener thereof, a transcript thereof, or a variant or a derivative thereof. The previously known report shows that change in the expression of the gene or the transcript thereof can serve as a marker for prostate cancer (Patent Literature 1).

The 184th target gene is the hsa-miR-663a gene, a congener thereof, a transcript thereof, or a variant or a derivative thereof. The previously known report shows that change in the expression of the gene or the transcript thereof can serve as a marker for prostate cancer (Patent Literature 1).

The 185th target gene is the hsa-miR-6842-5p gene, a congener thereof, a transcript thereof, or a variant or a derivative thereof. None of the previously known reports show that change in the expression of the gene or the transcript thereof can serve as a marker for prostate cancer.

The 186th target gene is the hsa-miR-7114-5p gene, a congener thereof, a transcript thereof, or a variant or a derivative thereof. None of the previously known reports show that change in the expression of the gene or the transcript thereof can serve as a marker for prostate cancer.

The 187th target gene is the hsa-miR-6779-5p gene, a congener thereof, a transcript thereof, or a variant or a derivative thereof. None of the previously known reports show that change in the expression of the gene or the transcript thereof can serve as a marker for prostate cancer.

The 580th target gene is the hsa-miR-204-3p gene, a congener thereof, a transcript thereof, or a variant or a derivative thereof. None of the previously known reports show that change in the expression of the gene or the transcript thereof can serve as a marker for prostate cancer.

The 581st target gene is the hsa-miR-642a-3p gene, a congener thereof, a transcript thereof, or a variant or a derivative thereof. None of the previously known reports show that change in the expression of the gene or the transcript thereof can serve as a marker for prostate cancer.

The 582nd target gene is the hsa-miR-762 gene, a congener thereof, a transcript thereof, or a variant or a derivative thereof. None of the previously known reports show that change in the expression of the gene or the transcript thereof can serve as a marker for prostate cancer.

The 583rd target gene is the hsa-miR-1202 gene, a congener thereof, a transcript thereof, or a variant or a derivative thereof. None of the previously known reports show that change in the expression of the gene or the transcript thereof can serve as a marker for prostate cancer.

The 584th target gene is the hsa-miR-3162-5p gene, a congener thereof, a transcript thereof, or a variant or a derivative thereof. None of the previously known reports show that change in the expression of the gene or the transcript thereof can serve as a marker for prostate cancer.

The 585th target gene is the hsa-miR-3196 gene, a congener thereof, a transcript thereof, or a variant or a derivative thereof. None of the previously known reports show that change in the expression of the gene or the transcript thereof can serve as a marker for prostate cancer.

The 586th target gene is the hsa-miR-3622a-5p gene, a congener thereof, a transcript thereof, or a variant or a derivative thereof. None of the previously known reports show that change in the expression of the gene or the transcript thereof can serve as a marker for prostate cancer.

The 587th target gene is the hsa-miR-3665 gene, a congener thereof, a transcript thereof, or a variant or a derivative thereof. None of the previously known reports show that change in the expression of the gene or the transcript thereof can serve as a marker for prostate cancer.

The 588th target gene is the hsa-miR-3940-5p gene, a congener thereof, a transcript thereof, or a variant or a derivative thereof. None of the previously known reports show that change in the expression of the gene or the transcript thereof can serve as a marker for prostate cancer.

The 589th target gene is the hsa-miR-4294 gene, a congener thereof, a transcript thereof, or a variant or a derivative thereof. None of the previously known reports show that change in the expression of the gene or the transcript thereof can serve as a marker for prostate cancer.

The 590th target gene is the hsa-miR-4466 gene, a congener thereof, a transcript thereof, or a variant or a derivative thereof. None of the previously known reports show that change in the expression of the gene or the transcript thereof can serve as a marker for prostate cancer.

The 591st target gene is the hsa-miR-4476 gene, a congener thereof, a transcript thereof, or a variant or a derivative thereof. None of the previously known reports show that change in the expression of the gene or the transcript thereof can serve as a marker for prostate cancer.

The 592nd target gene is the hsa-miR-4723-5p gene, a congener thereof, a transcript thereof, or a variant or a derivative thereof. None of the previously known reports show that change in the expression of the gene or the transcript thereof can serve as a marker for prostate cancer.

The 593rd target gene is the hsa-miR-4725-3p gene, a congener thereof, a transcript thereof, or a variant or a derivative thereof. None of the previously known reports show that change in the expression of the gene or the transcript thereof can serve as a marker for prostate cancer.

The 594th target gene is the hsa-miR-4730 gene, a congener thereof, a transcript thereof, or a variant or a derivative thereof. None of the previously known reports show that change in the expression of the gene or the transcript thereof can serve as a marker for prostate cancer.

The 595th target gene is the hsa-miR-4739 gene, a congener thereof, a transcript thereof, or a variant or a derivative thereof. None of the previously known reports show that change in the expression of the gene or the transcript thereof can serve as a marker for prostate cancer.

The 596th target gene is the hsa-miR-4787-5p gene, a congener thereof, a transcript thereof, or a variant or a derivative thereof. None of the previously known reports show that change in the expression of the gene or the transcript thereof can serve as a marker for prostate cancer.

The 597th target gene is the hsa-miR-5787 gene, a congener thereof, a transcript thereof, or a variant or a derivative thereof. None of the previously known reports show that change in the expression of the gene or the transcript thereof can serve as a marker for prostate cancer.

The 598th target gene is the hsa-miR-6085 gene, a congener thereof, a transcript thereof, or a variant or a derivative thereof. None of the previously known reports show that change in the expression of the gene or the transcript thereof can serve as a marker for prostate cancer.

The 599th target gene is the hsa-miR-6717-5p gene, a congener thereof, a transcript thereof, or a variant or a derivative thereof. None of the previously known reports show that change in the expression of the gene or the transcript thereof can serve as a marker for prostate cancer.

The 600th target gene is the hsa-miR-6724-5p gene, a congener thereof, a transcript thereof, or a variant or a derivative thereof. None of the previously known reports show that change in the expression of the gene or the transcript thereof can serve as a marker for prostate cancer.

The 601st target gene is the hsa-miR-6777-5p gene, a congener thereof, a transcript thereof, or a variant or a derivative thereof. None of the previously known reports show that change in the expression of the gene or the transcript thereof can serve as a marker for prostate cancer.

The 602nd target gene is the hsa-miR-6778-5p gene, a congener thereof, a transcript thereof, or a variant or a derivative thereof. None of the previously known reports show that change in the expression of the gene or the transcript thereof can serve as a marker for prostate cancer.

The 603rd target gene is the hsa-miR-6787-5p gene, a congener thereof, a transcript thereof, or a variant or a derivative thereof. None of the previously known reports show that change in the expression of the gene or the transcript thereof can serve as a marker for prostate cancer.

The 604th target gene is the hsa-miR-6789-5p gene, a congener thereof, a transcript thereof, or a variant or a derivative thereof. None of the previously known reports show that change in the expression of the gene or the transcript thereof can serve as a marker for prostate cancer.

The 605th target gene is the hsa-miR-6845-5p gene, a congener thereof, a transcript thereof, or a variant or a derivative thereof. None of the previously known reports show that change in the expression of the gene or the transcript thereof can serve as a marker for prostate cancer.

The 606th target gene is the hsa-miR-6893-5p gene, a congener thereof, a transcript thereof, or a variant or a derivative thereof. None of the previously known reports show that change in the expression of the gene or the transcript thereof can serve as a marker for prostate cancer.

The 607th target gene is the hsa-miR-16-5p gene, a congener thereof, a transcript thereof, or a variant or a derivative thereof. The previously known report shows that change in the expression of the gene or the transcript thereof can serve as a marker for prostate cancer (Patent Literature 2).

The 608th target gene is the hsa-miR-423-5p gene, a congener thereof, a transcript thereof, or a variant or a derivative thereof. The previously known report shows that change in the expression of the gene or the transcript thereof can serve as a marker for prostate cancer (Patent Literature 2).

The 609th target gene is the hsa-miR-451a gene, a congener thereof, a transcript thereof, or a variant or a derivative thereof. The previously known report shows that change in the expression of the gene or the transcript thereof can serve as a marker for prostate cancer (Patent Literature 2).

The 610th target gene is the hsa-miR-564 gene, a congener thereof, a transcript thereof, or a variant or a derivative thereof. The previously known report shows that change in the expression of the gene or the transcript thereof can serve as a marker for prostate cancer (Patent Literature 2).

The 611th target gene is the hsa-miR-671-5p gene, a congener thereof, a transcript thereof, or a variant or a derivative thereof. The previously known report shows that change in the expression of the gene or the transcript thereof can serve as a marker for prostate cancer (Patent Literature 2).

### 2. Nucleic Acid Probe or Primer for Detection of Prostate Cancer

In the present invention, a nucleic acid capable of specifically binding to any of the target nucleic acids as the prostate cancer markers described above can be used as a nucleic acid, for example, a nucleic acid probe or a primer, for the detection or diagnosis of prostate cancer.

In the present invention, the nucleic acid probe or the primer that can be used for detecting prostate cancer or for diagnosing prostate cancer permits qualitative and/or quantitative measurement of the presence, expression level, or abundance of any of the target nucleic acids as the prostate cancer markers described above, for example, human-derived hsa-miR-4443, hsa-miR-1908-5p, hsa-miR-4257, hsa-miR-3197, hsa-miR-3188, hsa-miR-4649-5p, hsa-miR-1343-3p, hsa-miR-6861-5p, hsa-miR-1343-5p, hsa-miR-642b-3p, hsa-miR-6741-5p, hsa-miR-4745-5p, hsa-miR-6826-5p, hsa-miR-3663-3p, hsa-miR-3131, hsa-miR-92a-2-5p, hsa-miR-4258, hsa-miR-4448, hsa-miR-6125, hsa-miR-6880-5p, hsa-miR-6132, hsa-miR-4467, hsa-miR-6749-5p, hsa-miR-2392, hsa-miR-1273g-3p, hsa-miR-4746-3p, hsa-miR-1914-3p, hsa-miR-7845-5p, hsa-miR-6726-5p, hsa-miR-128-2-5p, hsa-miR-4651, hsa-miR-6765-3p, hsa-miR-3185, hsa-miR-4792, hsa-miR-6887-5p, hsa-miR-5572, hsa-miR-3619-3p, hsa-miR-6780b-5p, hsa-miR-4707-5p, hsa-miR-8063, hsa-miR-4454, hsa-miR-4525, hsa-miR-7975, hsa-miR-744-5p, hsa-miR-3135b, hsa-miR-4648, hsa-miR-6816-5p, hsa-miR-4741, hsa-miR-7150, hsa-miR-6791-5p, hsa-miR-1247-3p, hsa-miR-7977, hsa-miR-4497, hsa-miR-6090, hsa-miR-6781-5p, hsa-miR-6870-5p, hsa-miR-6729-5p, hsa-miR-4530, hsa-miR-7847-3p, hsa-miR-6825-5p, hsa-miR-4674, hsa-miR-3917, hsa-miR-4707-3p, hsa-miR-6885-5p, hsa-miR-6722-3p, hsa-miR-4516, hsa-miR-6757-5p, hsa-miR-6840-3p, hsa-miR-5195-3p, hsa-miR-6756-5p, hsa-miR-6800-5p, hsa-miR-6727-5p, hsa-miR-6126, hsa-miR-6872-3p, hsa-miR-4446-3p, hsa-miR-1268a, hsa-miR-1908-3p, hsa-miR-3679-5p, hsa-miR-4534, hsa-miR-4675, hsa-miR-7108-5p, hsa-miR-6799-5p, hsa-miR-4695-5p, hsa-miR-3178, hsa-miR-5090, hsa-miR-3180, hsa-miR-1237-5p, hsa-miR-4758-5p, hsa-miR-3184-5p, hsa-miR-4286, hsa-miR-6784-5p, hsa-miR-6768-5p, hsa-miR-6785-5p, hsa-miR-4706, hsa-miR-711, hsa-miR-1260a, hsa-miR-6746-5p, hsa-miR-6089, hsa-miR-6821-5p, hsa-miR-4667-5p, hsa-miR-8069, hsa-miR-4726-5p, hsa-miR-6124, hsa-miR-4532, hsa-miR-4486, hsa-miR-4728-5p, hsa-miR-4508, hsa-miR-128-1-5p, hsa-miR-4513, hsa-miR-6795-5p, hsa-miR-4689, hsa-miR-6763-5p, hsa-miR-8072, hsa-miR-6765-5p, hsa-miR-4419b, hsa-miR-7641, hsa-miR-3928-3p, hsa-miR-1227-5p, hsa-miR-4492, hsa-miR-296-3p, hsa-miR-6769a-5p, hsa-miR-6889-5p, hsa-miR-4632-5p, hsa-miR-4505, hsa-miR-3154, hsa-miR-3648, hsa-miR-4442, hsa-miR-3141, hsa-miR-7113-3p, hsa-miR-6819-5p, hsa-miR-3195, hsa-miR-1199-5p, hsa-miR-6738-5p, hsa-miR-4656, hsa-miR-6820-5p, hsa-miR-204-3p, hsa-miR-642a-3p, hsa-miR-762, hsa-miR-1202, hsa-miR-3162-5p, hsa-miR-3196, hsa-miR-3622a-5p, hsa-miR-3665, hsa-miR-3940-5p, hsa-miR-4294, hsa-miR-4466, hsa-miR-4476, hsa-miR-4723-5p, hsa-miR-4725-3p, hsa-miR-4730, hsa-miR-4739, hsa-miR-4787-5p, hsa-miR-5787, hsa-miR-6085, hsa-miR-6717-5p, hsa-miR-6724-5p, hsa-miR-6777-5p, hsa-miR-6778-5p, hsa-miR-6787-5p, hsa-miR-6789-5p, hsa-miR-6845-5p, or hsa-miR-6893-5p, or combinations thereof, congeners thereof, transcripts thereof, or variants or derivatives thereof.

The expression level of each target nucleic acid described above is increased or decreased (hereinafter, referred to as “increased/decreased”) according to the type of the target nucleic acid(s) in a subject having prostate cancer as compared with a healthy subject. Hence, the nucleic acid of the present invention can be effectively used for measuring the expression level of the target nucleic acid(s) in a body fluid derived from a subject (e.g., a human) suspected of having prostate cancer and a body fluid derived from a healthy subject and detecting prostate cancer by the comparison thereof.

The nucleic acid probe or the primer that can be used in the present invention is a nucleic acid probe capable of specifically binding to a polynucleotide consisting of a nucleotide sequence represented by at least one of SEQ ID NOs: 1 to 135 and 580 to 606, or a primer for amplifying a polynucleotide consisting of a nucleotide sequence represented by at least one of SEQ ID NOs: 1 to 135 and 580 to 606.

The nucleic acid probe or the primer that can be further used in the present invention can comprise a nucleic acid probe capable of specifically binding to a polynucleotide consisting of a nucleotide sequence represented by at least one of SEQ ID NOs: 136 to 152 and 607 to 611, or a primer for amplifying a polynucleotide consisting of a nucleotide sequence represented by at least one of SEQ ID NOs: 136 to 152 and 607 to 611.

The nucleic acid probe or the primer that can be further used in the present invention can comprise a nucleic acid probe capable of specifically binding to a polynucleotide consisting of a nucleotide sequence represented by at least one of SEQ ID NOs: 153 to 187, or a primer for amplifying a polynucleotide consisting of a nucleotide sequence represented by at least one of SEQ ID NOs: 153 to 187.

Specifically, these nucleic acid probes or primers comprise a combination of one or more polynucleotides selected from a polynucleotide group comprising nucleotide sequences represented by any of SEQ ID NOs: 1 to 684 or nucleotide sequences derived from the nucleotide sequences by the replacement of u with t, and a complementary polynucleotide group thereof, a polynucleotide group respectively hybridizing under stringent conditions (mentioned later) to DNAs consisting of nucleotide sequences complementary to these nucleotide sequences, and a complementary polynucleotide group thereof, and a polynucleotide group comprising 15 or more, preferably 17 or more consecutive nucleotides in the nucleotide sequences of these polynucleotide groups. These polynucleotides can be used as nucleic acid probes and primers for detecting the prostate cancer markers as target nucleic acids.

More specifically, examples of the nucleic acid probe or the primer that can be used in the present invention include one or more polynucleotide(s) selected from the group consisting of the following polynucleotides (a) to (e):
(a) a polynucleotide consisting of a nucleotide sequence represented by any of SEQ ID NOs: 1 to 135, 580 to 606 or a nucleotide sequence derived from the nucleotide sequence by the replacement of u with t, a variant thereof, a derivative thereof, or a fragment thereof comprising 15 or more consecutive nucleotides,
(b) a polynucleotide comprising a nucleotide sequence represented by any of SEQ ID NOs: 1 to 135, 580 to 606,
(c) a polynucleotide consisting of a nucleotide sequence complementary to a nucleotide sequence represented by any of SEQ ID NOs: 1 to 135, 580 to 606 or a nucleotide sequence derived from the nucleotide sequence by the replacement of u with t, a variant thereof, a derivative thereof, or a fragment thereof comprising 15 or more consecutive nucleotides,
(d) a polynucleotide comprising a nucleotide sequence complementary to a nucleotide sequence represented by any of SEQ ID NOs: 1 to 135, 580 to 606 or a nucleotide sequence derived from the nucleotide sequence by the replacement of u with t, and
(e) a polynucleotide hybridizing under stringent conditions to any of the polynucleotides (a) to (d).

In addition to at least one or more polynucleotide(s) selected from the polynucleotides (a) to (e), the nucleic acid probe or the primer that can be further used in the present invention can comprise any of the following polynucleotides (f) to (j):
(f) a polynucleotide consisting of a nucleotide sequence represented by any of SEQ ID NOs: 136 to 152, 607 to 611 or a nucleotide sequence derived from the nucleotide sequence by the replacement of u with t, a variant thereof, a derivative thereof, or a fragment thereof comprising 15 or more consecutive nucleotides,
(g) a polynucleotide comprising a nucleotide sequence represented by any of SEQ ID NOs: 136 to 152, 607 to 611,
(h) a polynucleotide consisting of a nucleotide sequence complementary to a nucleotide sequence represented by any of SEQ ID NOs: 136 to 152, 607 to 611 or a nucleotide sequence derived from the nucleotide sequence by the replacement of u with t, a variant thereof, a derivative thereof, or a fragment thereof comprising 15 or more consecutive nucleotides,
(i) a polynucleotide comprising a nucleotide sequence complementary to a nucleotide sequence represented by any of SEQ ID NOs: 136 to 152, 607 to 611 or a nucleotide sequence derived from the nucleotide sequence by the replacement of u with t, and
(j) a polynucleotide hybridizing under stringent conditions to any of the polynucleotides (f) to (i).

In addition to at least one or more polynucleotide(s) selected from the polynucleotides (a) to (j), the nucleic acid probe or the primer that can be further used in the present invention can comprise any of the following polynucleotides (k) to (o):
(k) a polynucleotide consisting of a nucleotide sequence represented by any of SEQ ID NOs: 153 to 187 or a nucleotide sequence derived from the nucleotide sequence by the replacement of u with t, a variant thereof, a derivative thereof, or a fragment thereof comprising 15 or more consecutive nucleotides,
(l) a polynucleotide comprising a nucleotide sequence represented by any of SEQ ID NOs: 153 to 187,
(m) a polynucleotide consisting of a nucleotide sequence complementary to a nucleotide sequence represented by any of SEQ ID NOs: 153 to 187 or a nucleotide sequence derived from the nucleotide sequence by the replacement of u with t, a variant thereof, a derivative thereof, or a fragment thereof comprising 15 or more consecutive nucleotides,
(n) a polynucleotide comprising a nucleotide sequence complementary to a nucleotide sequence represented by any of SEQ ID NOs: 153 to 187 or a nucleotide sequence derived from the nucleotide sequence by the replacement of u with t, and
(o) a polynucleotide hybridizing under stringent conditions to any of the polynucleotides (k) to (n).

For these polynucleotides, the “fragment thereof comprising 15 or more consecutive nucleotides” can contain the number of nucleotides in the range of, for example, 15 consecutive nucleotides to less than the total number of nucleotides of the sequence, 17 consecutive nucleotides to less than the total number of nucleotides of the sequence, or 19 consecutive nucleotides to less than the total number of nucleotides of the sequence, in the nucleotide sequence of each polynucleotide, though the fragment is not limited thereto.

These polynucleotides or the fragments thereof used in the present invention may each be DNA or may each be RNA.

The polynucleotides that can be used in the present invention can each be prepared by use of a general technique such as a DNA recombination technique, PCR, or a method using an automatic DNA/RNA synthesizer.

The DNA recombination technique and the PCR can employ a technique described in, for example, Ausubel et al., Current Protocols in Molecular Biology, John Willey & Sons, US (1993); and Sambrook et al., Molecular Cloning—A Laboratory Manual, Cold Spring Harbor Laboratory Press, US (1989).

The human-derived hsa-miR-4443, hsa-miR-1908-5p, hsa-miR-4257, hsa-miR-3197, hsa-miR-3188, hsa-miR-4649-5p, hsa-miR-1343-3p, hsa-miR-6861-5p, hsa-miR-1343-5p, hsa-miR-642b-3p, hsa-miR-6741-5p, hsa-miR-4745-5p, hsa-miR-6826-5p, hsa-miR-3663-3p, hsa-miR-3131, hsa-miR-92a-2-5p, hsa-miR-4258, hsa-miR-4448, hsa-miR-6125, hsa-miR-6880-5p, hsa-miR-6132, hsa-miR-4467, hsa-miR-6749-5p, hsa-miR-2392, hsa-miR-1273g-3p, hsa-miR-4746-3p, hsa-miR-1914-3p, hsa-miR-7845-5p, hsa-miR-6726-5p, hsa-miR-128-2-5p, hsa-miR-4651, hsa-miR-6765-3p, hsa-miR-3185, hsa-miR-4792, hsa-miR-6887-5p, hsa-miR-5572, hsa-miR-3619-3p, hsa-miR-6780b-5p, hsa-miR-4707-5p, hsa-miR-8063, hsa-miR-4454, hsa-miR-4525, hsa-miR-7975, hsa-miR-744-5p, hsa-miR-3135b, hsa-miR-4648, hsa-miR-6816-5p, hsa-miR-4741, hsa-miR-7150, hsa-miR-6791-5p, hsa-miR-1247-3p, hsa-miR-7977, hsa-miR-4497, hsa-miR-6090, hsa-miR-6781-5p, hsa-miR-6870-5p, hsa-miR-6729-5p, hsa-miR-4530, hsa-miR-7847-3p, hsa-miR-6825-5p, hsa-miR-4674, hsa-miR-3917, hsa-miR-4707-3p, hsa-miR-6885-5p, hsa-miR-6722-3p, hsa-miR-4516, hsa-miR-6757-5p, hsa-miR-6840-3p, hsa-miR-5195-3p, hsa-miR-6756-5p, hsa-miR-6800-5p, hsa-miR-6727-5p, hsa-miR-6126, hsa-miR-6872-3p, hsa-miR-4446-3p, hsa-miR-1268a, hsa-miR-1908-3p, hsa-miR-3679-5p, hsa-miR-4534, hsa-miR-4675, hsa-miR-7108-5p, hsa-miR-6799-5p, hsa-miR-4695-5p, hsa-miR-3178, hsa-miR-5090, hsa-miR-3180, hsa-miR-1237-5p, hsa-miR-4758-5p, hsa-miR-3184-5p, hsa-miR-4286, hsa-miR-6784-5p, hsa-miR-6768-5p, hsa-miR-6785-5p, hsa-miR-4706, hsa-miR-711, hsa-miR-1260a, hsa-miR-6746-5p, hsa-miR-6089, hsa-miR-6821-5p, hsa-miR-4667-5p, hsa-miR-8069, hsa-miR-4726-5p, hsa-miR-6124, hsa-miR-4532, hsa-miR-4486, hsa-miR-4728-5p, hsa-miR-4508, hsa-miR-128-1-5p, hsa-miR-4513, hsa-miR-6795-5p, hsa-miR-4689, hsa-miR-6763-5p, hsa-miR-8072, hsa-miR-6765-5p, hsa-miR-4419b, hsa-miR-7641, hsa-miR-3928-3p, hsa-miR-1227-5p, hsa-miR-4492, hsa-miR-296-3p, hsa-miR-6769a-5p, hsa-miR-6889-5p, hsa-miR-4632-5p, hsa-miR-4505, hsa-miR-3154, hsa-miR-3648, hsa-miR-4442, hsa-miR-3141, hsa-miR-7113-3p, hsa-miR-6819-5p, hsa-miR-3195, hsa-miR-1199-5p, hsa-miR-6738-5p, hsa-miR-4656, hsa-miR-6820-5p, hsa-miR-204-3p, hsa-miR-642a-3p, hsa-miR-762, hsa-miR-1202, hsa-miR-3162-5p, hsa-miR-3196, hsa-miR-3622a-5p, hsa-miR-3665, hsa-miR-3940-5p, hsa-miR-4294, hsa-miR-4466, hsa-miR-4476, hsa-miR-4723-5p, hsa-miR-4725-3p, hsa-miR-4730, hsa-miR-4739, hsa-miR-4787-5p, hsa-miR-5787, hsa-miR-6085, hsa-miR-6717-5p, hsa-miR-6724-5p, hsa-miR-6777-5p, hsa-miR-6778-5p, hsa-miR-6787-5p, hsa-miR-6789-5p, hsa-miR-6845-5p, hsa-miR-6893-5p, hsa-miR-615-5p, hsa-miR-486-3p, hsa-miR-1225-3p, hsa-miR-760, hsa-miR-187-5p, hsa-miR-1203, hsa-miR-7110-5p, hsa-miR-371a-5p, hsa-miR-939-5p, hsa-miR-575, hsa-miR-92b-5p, hsa-miR-887-3p, hsa-miR-920, hsa-miR-1915-5p, hsa-miR-1231, hsa-miR-663b, hsa-miR-1225-5p, hsa-miR-16-5p, hsa-miR-423-5p, hsa-miR-451a, hsa-miR-564, hsa-miR-671-5p, hsa-miR-4763-3p, hsa-miR-3656, hsa-miR-4488, hsa-miR-125a-3p, hsa-miR-1469, hsa-miR-1228-5p, hsa-miR-6798-5p, hsa-miR-1268b, hsa-miR-6732-5p, hsa-miR-1915-3p, hsa-miR-4433b-3p, hsa-miR-1207-5p, hsa-miR-4433-3p, hsa-miR-6879-5p, hsa-miR-4417, hsa-miR-30c-1-3p, hsa-miR-4638-5p, hsa-miR-6088, hsa-miR-4270, hsa-miR-6782-5p, hsa-miR-665, hsa-miR-486-5p, hsa-miR-4655-5p, hsa-miR-1275, hsa-miR-6806-5p, hsa-miR-614, hsa-miR-3937, hsa-miR-6752-5p, hsa-miR-6771-5p, hsa-miR-4450, hsa-miR-211-3p, hsa-miR-663a, hsa-miR-6842-5p, hsa-miR-7114-5p and hsa-miR-6779-5p represented by SEQ ID NOs: 1 to 187, and 580 to 611 are known in the art, and their obtainment methods are also known as mentioned above. Therefore, each polynucleotide that can be used as a nucleic acid probe or a primer in the present invention can be prepared by cloning the gene.

Such a nucleic acid probe or a primer can be chemically synthesized using an automatic DNA synthesis apparatus. In general, a phosphoramidite method is used in this synthesis, and single-stranded DNA up to approximately 100 bases can be automatically synthesized by this method. The automatic DNA synthesis apparatus is commercially available from, for example, Polygen GmbH, ABI, or Applied Biosystems, Inc.

Alternatively, the polynucleotide of the present invention can also be prepared by a cDNA cloning method. The cDNA cloning technique can employ, for example, microRNA Cloning Kit Wako.

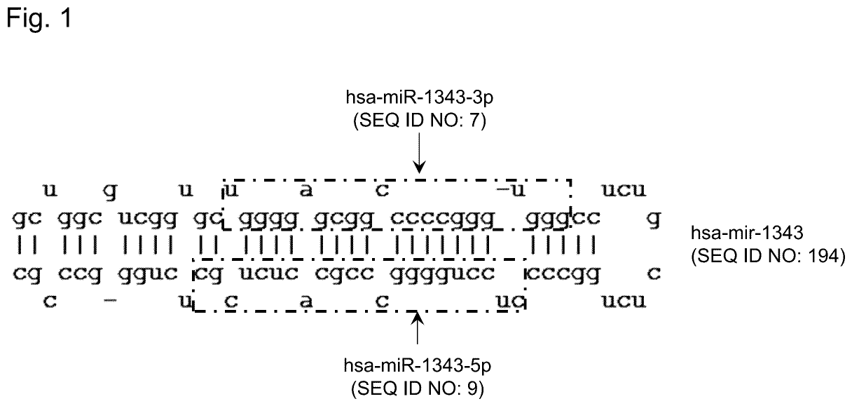

In this context, the sequences of the nucleic acid probes and the primers for detecting the polynucleotides consisting of a nucleotide sequence represented by any of SEQ ID NOs: 1 to 187, and 580 to 611 do not exist as miRNAs or precursors thereof in vivo. For example, the nucleotide sequences represented by SEQ ID NO: 7 and SEQ ID NO: 9 are formed from the precursor represented by SEQ ID NO: 194. This precursor has a hairpin-like structure as shown in FIG. 1, and the nucleotide sequences represented by SEQ ID NO: 7 and SEQ ID NO: 9 have mismatch sequences with each other. Therefore, a nucleotide sequence completely complementary to the nucleotide sequence represented by SEQ ID NO: 7 or SEQ ID NO: 9 does not naturally occur in vivo. Likewise, the nucleic acid probe and the primer for detecting the nucleotide sequence represented by any of SEQ ID NOs: 1 to 187, and 580 to 611 each have an artificial nucleotide sequence that does not exist in vivo.

### 3. Kit or Device for Detection of Prostate Cancer

The present invention also provides a kit or a device for the detection of prostate cancer, comprising one or more polynucleotide(s) (which can include a variant, a fragment, and a derivative; hereinafter, also referred to as a polynucleotide for detection) that can be used as a nucleic acid probe or a primer in the present invention for measuring a target nucleic acid as a prostate cancer marker.

The target nucleic acid as a prostate cancer marker according to the present invention is selected from the following group 1:
miR-4443, miR-1908-5p, miR-4257, miR-3197, miR-3188, miR-4649-5p, miR-1343-3p, miR-6861-5p, miR-1343-5p, miR-642b-3p, miR-6741-5p, miR-4745-5p, miR-6826-5p, miR-3663-3p, miR-3131, miR-92a-2-5p, miR-4258, miR-4448, miR-6125, miR-6880-5p, miR-6132, miR-4467, miR-6749-5p, miR-2392, miR-1273g-3p, miR-4746-3p, miR-1914-3p, miR-7845-5p, miR-6726-5p, miR-128-2-5p, miR-4651, miR-6765-3p, miR-3185, miR-4792, miR-6887-5p, miR-5572, miR-3619-3p, miR-6780b-5p, miR-4707-5p, miR-8063, miR-4454, miR-4525, miR-7975, miR-744-5p, miR-3135b, miR-4648, miR-6816-5p, miR-4741, miR-7150, miR-6791-5p, miR-1247-3p, miR-7977, miR-4497, miR-6090, miR-6781-5p, miR-6870-5p, miR-6729-5p, miR-4530, miR-7847-3p, miR-6825-5p, miR-4674, miR-3917, miR-4707-3p, miR-6885-5p, miR-6722-3p, miR-4516, miR-6757-5p, miR-6840-3p, miR-5195-3p, miR-6756-5p, miR-6800-5p, miR-6727-5p, miR-6126, miR-6872-3p, miR-4446-3p, miR-1268a, miR-1908-3p, miR-3679-5p, miR-4534, miR-4675, miR-7108-5p, miR-6799-5p, miR-4695-5p, miR-3178, miR-5090, miR-3180, miR-1237-5p, miR-4758-5p, miR-3184-5p, miR-4286, miR-6784-5p, miR-6768-5p, miR-6785-5p, miR-4706, miR-711, miR-1260a, miR-6746-5p, miR-6089, miR-6821-5p, miR-4667-5p, miR-8069, miR-4726-5p, miR-6124, miR-4532, miR-4486, miR-4728-5p, miR-4508, miR-128-1-5p, miR-4513, miR-6795-5p, miR-4689, miR-6763-5p, miR-8072, miR-6765-5p, miR-4419b, miR-7641, miR-3928-3p, miR-1227-5p, miR-4492, miR-296-3p, miR-6769a-5p, miR-6889-5p, miR-4632-5p, miR-4505, miR-3154, miR-3648, miR-4442, miR-3141, miR-7113-3p, miR-6819-5p, miR-3195, miR-1199-5p, miR-6738-5p, miR-4656, miR-6820-5p, miR-204-3p, miR-642a-3p, miR-762, miR-1202, miR-3162-5p, miR-3196, miR-3622a-5p, miR-3665, miR-3940-5p, miR-4294, miR-4466, miR-4476, miR-4723-5p, miR-4725-3p, miR-4730, miR-4739, miR-4787-5p, miR-5787, miR-6085, miR-6717-5p, miR-6724-5p, miR-6777-5p, miR-6778-5p, miR-6787-5p, miR-6789-5p, miR-6845-5p and miR-6893-5p.

An additional target nucleic acid that can be optionally used in the measurement is selected from the following group 2: miR-615-5p, miR-486-3p, miR-1225-3p, miR-760, miR-187-5p, miR-1203, miR-7110-5p, miR-371a-5p, miR-939-5p, miR-575, miR-92b-5p, miR-887-3p, miR-920, miR-1915-5p, miR-1231, miR-663b, miR-1225-5p, miR-16-5p, miR-423-5p, miR-451a, miR-564 and miR-671-5p.

An additional target nucleic acid that can be optionally further used in the measurement is selected from the following group 3: miR-4763-3p, miR-3656, miR-4488, miR-125a-3p, miR-1469, miR-1228-5p, miR-6798-5p, miR-1268b, miR-6732-5p, miR-1915-3p, miR-4433b-3p, miR-1207-5p, miR-4433-3p, miR-6879-5p, miR-4417, miR-30c-1-3p, miR-4638-5p, miR-6088, miR-4270, miR-6782-5p, miR-665, miR-486-5p, miR-4655-5p, miR-1275, miR-6806-5p, miR-614, miR-3937, miR-6752-5p, miR-6771-5p, miR-4450, miR-211-3p, miR-663a, miR-6842-5p, miR-7114-5p and miR-6779-5p.

The kit or the device of the present invention comprises nucleic acid(s) capable of specifically binding to any of the target nucleic acids as the prostate cancer markers described above, preferably one or more polynucleotide(s) selected from the nucleic acid probes or the primers described in the preceding Section 2, specifically, the polynucleotides described in the preceding Section 2, or variant(s) thereof, etc.

Specifically, the kit or the device of the present invention can comprise at least one or more polynucleotide(s) comprising (or consisting of) a nucleotide sequence represented by any of SEQ ID NOs: 1 to 135, 580 to 606 or a nucleotide sequence derived from the nucleotide sequence by the replacement of u with t, polynucleotide(s) comprising (or consisting of) a complementary sequence thereof, polynucleotide(s) hybridizing under stringent conditions to any of these polynucleotides, or variant(s) or fragment(s) comprising 15 or more consecutive nucleotides of any of these polynucleotide sequences.

The kit or the device of the present invention can further comprise one or more polynucleotide(s) comprising (or consisting of) a nucleotide sequence represented by any of SEQ ID NOs: 136 to 152, 607 to 611 or a nucleotide sequence derived from the nucleotide sequence by the replacement of u with t, polynucleotide(s) comprising (or consisting of) a complementary sequence thereof, polynucleotide(s) hybridizing under stringent conditions to any of these polynucleotides, variant(s) or fragment(s) comprising 15 or more consecutive nucleotides of any of these polynucleotide sequences.

The kit or the device of the present invention can further comprise one or more polynucleotide(s) comprising (or consisting of) a nucleotide sequence represented by any of SEQ ID NOs: 153 to 187 or a nucleotide sequence derived from the nucleotide sequence by the replacement of u with t, polynucleotide(s) comprising (or consisting of) a complementary sequence thereof, polynucleotide(s) hybridizing under stringent conditions to any of these polynucleotides, variant(s) or fragment(s) comprising 15 or more consecutive nucleotides of any of these polynucleotide sequences.

The fragment that can be contained in the kit or the device of the present invention is, for example, one or more, preferably two or more polynucleotides selected from the group consisting of the following polynucleotides (1) to (3):
(1) a polynucleotide comprising 15 or more consecutive nucleotides in a nucleotide sequence derived from a nucleotide sequence represented by any of SEQ ID NOs: 1 to 135, 580 to 606 by the replacement of u with t, or a complementary sequence thereof,
(2) a polynucleotide comprising 15 or more consecutive nucleotides in a nucleotide sequence derived from a nucleotide sequence represented by any of SEQ ID NOs: 136 to 152, 607 to 611 by the replacement of u with t, or a complementary sequence thereof, and
(3) a polynucleotide comprising 15 or more consecutive nucleotides in a nucleotide sequence derived from a nucleotide sequence represented by any of SEQ ID NOs: 153 to 187 by the replacement of u with t, or a complementary sequence thereof.

In a preferred embodiment, the polynucleotide is a polynucleotide consisting of a nucleotide sequence represented by any of SEQ ID NOs: 1 to 135, 580 to 606 or a nucleotide sequence derived from the nucleotide sequence by the replacement of u with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the polynucleotide is a polynucleotide consisting of a nucleotide sequence represented by any of SEQ ID NOs: 136 to 152, 607 to 611 or a nucleotide sequence derived from the nucleotide sequence by the replacement of u with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the polynucleotide is a polynucleotide consisting of a nucleotide sequence represented by any of SEQ ID NOs: 153 to 187 or a nucleotide sequence derived from the nucleotide sequence by the replacement of u with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the fragment can be a polynucleotide comprising 15 or more, preferably 17 or more, more preferably 19 or more consecutive nucleotides.

In the present invention, the size of the polynucleotide fragment is the number of bases in the range of, for example, 15 consecutive nucleotides to less than the total number of bases of the sequence, 17 consecutive nucleotides to less than the total number of bases of the sequence, or 19 consecutive nucleotides to less than the total number of bases of the sequence, in the nucleotide sequence of each polynucleotide.

Specific examples of the aforementioned polynucleotide combination constituting the kit or the device of the present invention can include combinations of 1, 2, 3, 4, 5, 6, 7, 8, 9, 10 or more of the polynucleotides consisting of nucleotide sequences represented by SEQ ID NOs as shown in Table 1 (SEQ ID NOs: 1 to 187 and 580 to 611 corresponding to the miRNA markers in the table). However, these are given merely for illustrative purposes, and various other possible combinations are included in the present invention.

The aforementioned combination constituting the kit or the device for discriminating a prostate cancer patient from a healthy subject according to the present invention is desirably, for example, a combination of two or more of the polynucleotides consisting of nucleotide sequences represented by SEQ ID NOs shown in Table 1. Usually, a combination of two of these polynucleotides can produce adequate performance.

The combination of two polynucleotides consisting of the nucleotide sequences or the complementary sequences thereof for specifically discriminating a prostate cancer patient from a healthy subject is preferably a combination comprising at least one or more of newly found polynucleotides consisting of the nucleotide sequences represented by SEQ ID NOs: 1 to 135, among the combinations constituted by two of the polynucleotides consisting of the nucleotide sequences represented by SEQ ID NOs: 1 to 187, and 580 to 611.

The combination of polynucleotides with cancer type specificity capable of discriminating a prostate cancer patient not only from a healthy subject but also from other cancer patients is preferably, for example, a combination of multiple polynucleotides comprising at least one polynucleotide selected from the group consisting of polynucleotides of SEQ ID NOs: 1, 3, 4, 5, 6, 7, 9, 10, 12, 14, 15, 16, 17, 18, 20, 24, 29, 35, 37, 42, 51, 55, 58, 61, 63, 64, 67, 70, 72, 79, 82, 89, 91, 97, 98, 101, 103, 104, 112, 113, 114, 116, 119, 126, 135, 136, 139, 140, 141, 145, 147, 154, 155, 156, 158, 169, 173, 175, 178, 182, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610 and 611 (hereinafter, this group is referred to as “cancer type-specific polynucleotide group 1”), with any of the polynucleotides of the other SEQ ID NOs.

The combination of polynucleotides with cancer type specificity capable of discriminating a prostate cancer patient not only from a healthy subject but also from other cancer patients is more preferably a combination of multiple polynucleotides selected from the cancer type-specific polynucleotide group 1.

The combination of polynucleotides with cancer type specificity capable of discriminating a prostate cancer patient not only from a healthy subject but also from other cancer patients is more preferably a combination comprising at least one or more polynucleotide(s) selected from the group consisting of polynucleotides of SEQ ID NOs: 1, 12, 16, 37, 42, 63, 119, 126, 139 173, 178, 599. 609, and 611 (hereinafter, this group is referred to as “cancer type-specific polynucleotide group 2”) included in the cancer type-specific polynucleotide group 1, among the combinations of multiple polynucleotides selected from the cancer type-specific polynucleotide group 1.

The number of the aforementioned polynucleotides with cancer type specificity used in the combination can be 1, 2, 3, 4, 5, 6, 7, 8, 9, 10 or more for the combination and is more preferably 4 or more for the combination. Usually, the combination of 4 of these polynucleotides can produce adequate performance.

Non-limiting examples of the combination of the polynucleotide consisting of the nucleotide sequence represented by SEQ ID NO: 1 or a complementary sequence thereof with polynucleotides consisting of nucleotide sequences represented by SEQ ID NOs of three polynucleotides selected from the cancer type-specific polynucleotide group 1 or complementary sequences thereof will be listed below.
(1) a combination of SEQ ID NOs: 1, 63, 139, and 600 (markers: hsa-miR-4443, hsa-miR-4707-3p, hsa-miR-760, and hsa-miR-6724-5p);
(2) a combination of SEQ ID NOs: 1, 12, 63, and 599 (markers: hsa-miR-4443, hsa-miR-4745-5p, hsa-miR-4707-3p, and hsa-miR-6717-5p);
(3) a combination of SEQ ID NOs: 1, 141, 173, and 599 (markers: hsa-miR-4443, hsa-miR-1203, hsa-miR-665, and hsa-miR-6717-5p);
(4) a combination of SEQ ID NOs: 1, 16, 139, and 178 (markers: hsa-miR-4443, hsa-miR-92a-2-5p, hsa-miR-760, and hsa-miR-614); and
(5) a combination of SEQ ID NOs: 1, 63, 173, and 599 (markers: hsa-miR-4443, hsa-miR-4707-3p, hsa-miR-665, and hsa-miR-6717-5p).

Non-limiting examples of the combination of the polynucleotide consisting of the nucleotide sequence represented by SEQ ID NO: 12 or a complementary sequence thereof with polynucleotides consisting of nucleotide sequences represented by SEQ ID NOs of three polynucleotides selected from the cancer type-specific polynucleotide group 1 or complementary sequences thereof will be further listed.
(1) a combination of SEQ ID NOs: 12, 42, 63, and 609 (markers: hsa-miR-4745-5p, hsa-miR-4525, hsa-miR-4707-3p, and hsa-miR-451a);
(2) a combination of SEQ ID NOs: 12, 16, 135, and 156 (markers: hsa-miR-4745-5p, hsa-miR-92a-2-5p, hsa-miR-6820-5p, and hsa-miR-125a-3p);
(3) a combination of SEQ ID NOs: 12, 16, 169, and 178 (markers: hsa-miR-4745-5p, hsa-miR-92a-2-5p, hsa-miR-4638-5p, and hsa-miR-614);
(4) a combination of SEQ ID NOs: 12, 16, 139, and 601 (markers: hsa-miR-4745-5p, hsa-miR-92a-2-5p, hsa-miR-760, and hsa-miR-6777-5p); and
(5) a combination of SEQ ID NOs: 12, 16, 42, and 607 (markers: hsa-miR-4745-5p, hsa-miR-92a-2-5p, hsa-miR-4525, and hsa-miR-16-5p).

Non-limiting examples of the combination of the polynucleotide consisting of the nucleotide sequence represented by SEQ ID NO: 16 or a complementary sequence thereof with polynucleotides consisting of nucleotide sequences represented by SEQ ID NOs of three polynucleotides selected from the cancer type-specific polynucleotide group 1 or complementary sequences thereof will be further listed.
(1) a combination of SEQ ID NOs: 16, 18, 139, and 178 (markers: hsa-miR-92a-2-5p, hsa-miR-4448, hsa-miR-760, and hsa-miR-614);
(2) a combination of SEQ ID NOs: 12, 16, 37, and 178 (markers: hsa-miR-4745-5p, hsa-miR-92a-2-5p, hsa-miR-3619-3p, and hsa-miR-614);
(3) a combination of SEQ ID NOs: 12, 16, 37, and 599 (markers: hsa-miR-4745-5p, hsa-miR-92a-2-5p, hsa-miR-3619-3p, and hsa-miR-6717-5p);
(4) a combination of SEQ ID NOs: 12, 16, 37, and 97 (markers: hsa-miR-4745-5p, hsa-miR-92a-2-5p, hsa-miR-3619-3p, and hsa-miR-6746-5p); and
(5) a combination of SEQ ID NOs: 12, 14, 16, and 599 (markers: hsa-miR-4745-5p, hsa-miR-3663-3p, hsa-miR-92a-2-5p, and hsa-miR-6717-5p).

Non-limiting examples of the combination of the polynucleotide consisting of the nucleotide sequence represented by SEQ ID NO: 37 or a complementary sequence thereof with polynucleotides consisting of nucleotide sequences represented by SEQ ID NOs of three polynucleotides selected from the cancer type-specific polynucleotide group 1 or complementary sequences thereof will be further listed.
(1) a combination of SEQ ID NOs: 37, 63, 139, and 611 (markers: hsa-miR-3619-3p, hsa-miR-4707-3p, hsa-miR-760, and hsa-miR-671-5p);
(2) a combination of SEQ ID NOs: 37, 42, 63, and 178 (markers: hsa-miR-3619-3p, hsa-miR-4525, hsa-miR-4707-3p, and hsa-miR-614);
(3) a combination of SEQ ID NOs: 37, 42, 63, and 599 (markers: hsa-miR-3619-3p, hsa-miR-4525, hsa-miR-4707-3p, and hsa-miR-6717-5p);
(4) a combination of SEQ ID NOs: 37, 42, 63, and 139 (markers: hsa-miR-3619-3p, hsa-miR-4525, hsa-miR-4707-3p, and hsa-miR-760); and
(5) a combination of SEQ ID NOs: 12, 16, 37, and 603 (markers: hsa-miR-4745-5p, hsa-miR-92a-2-5p, hsa-miR-3619-3p, and hsa-miR-6787-5p).

Non-limiting examples of the combination of the polynucleotide consisting of the nucleotide sequence represented by SEQ ID NO: 42 or a complementary sequence thereof with polynucleotides consisting of nucleotide sequences represented by SEQ ID NOs of three polynucleotides selected from the cancer type-specific polynucleotide group 1 or complementary sequences thereof will be further listed.
(1) a combination of SEQ ID NOs: 42, 63, 607, and 611 (markers: hsa-miR-4525, hsa-miR-4707-3p, hsa-miR-16-5p, and hsa-miR-671-5p);
(2) a combination of SEQ ID NOs: 42, 63, 609, and 611 (markers: hsa-miR-4525, hsa-miR-4707-3p, hsa-miR-451a, and hsa-miR-671-5p);
(3) a combination of SEQ ID NOs: 42, 63, 173, and 599 (markers: hsa-miR-4525, hsa-miR-4707-3p, hsa-miR-665, and hsa-miR-6717-5p);
(4) a combination of SEQ ID NOs: 12, 16, 42, and 609 (markers: hsa-miR-4745-5p, hsa-miR-92a-2-5p, hsa-miR-4525, and hsa-miR-451a); and
(5) a combination of SEQ ID NOs: 42, 63, 91, and 609 (markers: hsa-miR-4525, hsa-miR-4707-3p, hsa-miR-6784-5p, and hsa-miR-451a).

Non-limiting examples of the combination of the polynucleotide consisting of the nucleotide sequence represented by SEQ ID NO: 63 or a complementary sequence thereof with polynucleotides consisting of nucleotide sequences represented by SEQ ID NOs of three polynucleotides selected from the cancer type-specific polynucleotide group 1 or complementary sequences thereof will be further listed.
(1) a combination of SEQ ID NOs: 10, 42, 63, and 599 (markers: hsa-miR-642b-3p, hsa-miR-4525, hsa-miR-4707-3p, and hsa-miR-6717-5p);
(2) a combination of SEQ ID NOs: 42, 63, 599, and 609 (markers: hsa-miR-4525, hsa-miR-4707-3p, hsa-miR-6717-5p, and hsa-miR-451a);
(3) a combination of SEQ ID NOs: 42, 63, 583, and 609 (markers: hsa-miR-4525, hsa-miR-4707-3p, hsa-miR-1202, and hsa-miR-451a);
(4) a combination of SEQ ID NOs: 37, 42, 63, and 611 (markers: hsa-miR-3619-3p, hsa-miR-4525, hsa-miR-4707-3p, and hsa-miR-671-5p); and
(5) a combination of SEQ ID NOs: 12, 63, 70, and 599 (markers: hsa-miR-4745-5p, hsa-miR-4707-3p, hsa-miR-6756-5p, and hsa-miR-6717-5p).

Non-limiting examples of the combination of the polynucleotide consisting of the nucleotide sequence represented by SEQ ID NO: 119 or a complementary sequence thereof with polynucleotides consisting of nucleotide sequences represented by SEQ ID NOs of three polynucleotides selected from the cancer type-specific polynucleotide group 1 or complementary sequences thereof will be further listed.
(1) a combination of SEQ ID NOs: 12, 16, 37, and 119 (markers: hsa-miR-4745-5p, hsa-miR-92a-2-5p, hsa-miR-3619-3p, and hsa-miR-4492);
(2) a combination of SEQ ID NOs: 37, 63, 119, and 584 (markers: hsa-miR-3619-3p, hsa-miR-4707-3p, hsa-miR-4492, and hsa-miR-3162-5p);
(3) a combination of SEQ ID NOs: 63, 119, 173, and 178 (markers: hsa-miR-4707-3p, hsa-miR-4492, hsa-miR-665, and hsa-miR-614);
(4) a combination of SEQ ID NOs: 63, 119, 158, and 173 (markers: hsa-miR-4707-3p, hsa-miR-4492, hsa-miR-1228-5p, and hsa-miR-665); and
(5) a combination of SEQ ID NOs: 63, 119, 173, and 605 (markers: hsa-miR-4707-3p, hsa-miR-4492, hsa-miR-665, and hsa-miR-6845-5p).

Non-limiting examples of the combination of the polynucleotide consisting of the nucleotide sequence represented by SEQ ID NO: 126 or a complementary sequence thereof with polynucleotides consisting of nucleotide sequences represented by SEQ ID NOs of three polynucleotides selected from the cancer type-specific polynucleotide group 1 or complementary sequences thereof will be further listed.
(1) a combination of SEQ ID NOs: 16, 126, 597, and 599 (markers: hsa-miR-92a-2-5p, hsa-miR-3648, hsa-miR-5787, and hsa-miR-6717-5p);
(2) a combination of SEQ ID NOs: 16, 42, 126, and 599 (markers: hsa-miR-92a-2-5p, hsa-miR-4525, hsa-miR-3648, and hsa-miR-6717-5p);
(3) a combination of SEQ ID NOs: 16, 126, 139, and 601 (markers: hsa-miR-92a-2-5p, hsa-miR-3648, hsa-miR-760, and hsa-miR-6777-5p);
(4) a combination of SEQ ID NOs: 16, 126, 593, and 599 (markers: hsa-miR-92a-2-5p, hsa-miR-3648, hsa-miR-4725-3p, and hsa-miR-6717-5p); and
(5) a combination of SEQ ID NOs: 15, 16, 126, and 599 (markers: hsa-miR-3131, hsa-miR-92a-2-5p, hsa-miR-3648, and hsa-miR-6717-5p).

Non-limiting examples of the combination of the polynucleotide consisting of the nucleotide sequence represented by SEQ ID NO: 139 or a complementary sequence thereof with polynucleotides consisting of nucleotide sequences represented by SEQ ID NOs of three polynucleotides selected from the cancer type-specific polynucleotide group 1 or complementary sequences thereof will be further listed.
(1) a combination of SEQ ID NOs: 37, 63, 139, and 584 (markers: hsa-miR-3619-3p, hsa-miR-4707-3p, hsa-miR-760, and hsa-miR-3162-5p);
(2) a combination of SEQ ID NOs: 63, 139, 173, and 178 (markers: hsa-miR-4707-3p, hsa-miR-760, hsa-miR-665, and hsa-miR-614);
(3) a combination of SEQ ID NOs: 16, 63, 139, and 601 (markers: hsa-miR-92a-2-5p, hsa-miR-4707-3p, hsa-miR-760, and hsa-miR-6777-5p);
(4) a combination of SEQ ID NOs: 37, 63, 139, and 600 (markers: hsa-miR-3619-3p, hsa-miR-4707-3p, hsa-miR-760, and hsa-miR-6724-5p); and
(5) a combination of SEQ ID NOs: 16, 139, 178, and 586 (markers: hsa-miR-92a-2-5p, hsa-miR-760, hsa-miR-614, and hsa-miR-3622a-5p).

Non-limiting examples of the combination of the polynucleotide consisting of the nucleotide sequence represented by SEQ ID NO: 173 or a complementary sequence thereof with polynucleotides consisting of nucleotide sequences represented by SEQ ID NOs of three polynucleotides selected from the cancer type-specific polynucleotide group 1 or complementary sequences thereof will be further listed.
(1) a combination of SEQ ID NOs: 63, 139, 173, and 599 (markers: hsa-miR-4707-3p, hsa-miR-760, hsa-miR-665, and hsa-miR-6717-5p);
(2) a combination of SEQ ID NOs: 63, 119, 173, and 581 (markers: hsa-miR-4707-3p, hsa-miR-4492, hsa-miR-665, and hsa-miR-642a-3p);
(3) a combination of SEQ ID NOs: 63, 173, 582, and 599 (markers: hsa-miR-4707-3p, hsa-miR-665, hsa-miR-762, and hsa-miR-6717-5p);
(4) a combination of SEQ ID NOs: 63, 136, 173, and 599 (markers: hsa-miR-4707-3p, hsa-miR-615-5p, hsa-miR-665, and hsa-miR-6717-5p); and
(5) a combination of SEQ ID NOs: 29, 63, 173, and 178 (markers: hsa-miR-6726-5p, hsa-miR-4707-3p, hsa-miR-665, and hsa-miR-614).

Non-limiting examples of the combination of the polynucleotide consisting of the nucleotide sequence represented by SEQ ID NO: 178 or a complementary sequence thereof with polynucleotides consisting of nucleotide sequences represented by SEQ ID NOs of three polynucleotides selected from the cancer type-specific polynucleotide group 1 or complementary sequences thereof will be further listed.
(1) a combination of SEQ ID NOs: 16, 139, 178, and 601 (markers: hsa-miR-92a-2-5p, hsa-miR-760, hsa-miR-614, and hsa-miR-6777-5p);
(2) a combination of SEQ ID NOs: 16, 37, 139, and 178 (markers: hsa-miR-92a-2-5p, hsa-miR-3619-3p, hsa-miR-760, and hsa-miR-614);
(3) a combination of SEQ ID NOs: 1, 12, 16, and 178 (markers: hsa-miR-4443, hsa-miR-4745-5p, hsa-miR-92a-2-5p, and hsa-miR-614);
(4) a combination of SEQ ID NOs: 1, 63, 173, and 178 (markers: hsa-miR-4443, hsa-miR-4707-3p, hsa-miR-665, and hsa-miR-614); and
(5) a combination of SEQ ID NOs: 16, 139, 178, and 597 (markers: hsa-miR-92a-2-5p, hsa-miR-760, hsa-miR-614, and hsa-miR-5787).

Non-limiting examples of the combination of the polynucleotide consisting of the nucleotide sequence represented by SEQ ID NO: 599 or a complementary sequence thereof with polynucleotides consisting of nucleotide sequences represented by SEQ ID NOs of three polynucleotides selected from the cancer type-specific polynucleotide group 1 or complementary sequences thereof will be further listed.
(1) a combination of SEQ ID NOs: 12, 37, 63, and 599 (markers: hsa-miR-4745-5p, hsa-miR-3619-3p, hsa-miR-4707-3p, and hsa-miR-6717-5p);
(2) a combination of SEQ ID NOs: 42, 58, 63, and 599 (markers: hsa-miR-4525, hsa-miR-4530, hsa-miR-4707-3p, and hsa-miR-6717-5p);
(3) a combination of SEQ ID NOs: 1, 12, 16, and 599 (markers: hsa-miR-4443, hsa-miR-4745-5p, hsa-miR-92a-2-5p, and hsa-miR-6717-5p);
(4) a combination of SEQ ID NOs: 63, 119, 173, and 599 (markers: hsa-miR-4707-3p, hsa-miR-4492, hsa-miR-665, and hsa-miR-6717-5p); and
(5) a combination of SEQ ID NOs: 16, 18, 139, and 599 (markers: hsa-miR-92a-2-5p, hsa-miR-4448, hsa-miR-760, and hsa-miR-6717-5p).

Non-limiting examples of the combination of the polynucleotide consisting of the nucleotide sequence represented by SEQ ID NO: 609 or a complementary sequence thereof with polynucleotides consisting of nucleotide sequences represented by SEQ ID NOs of three polynucleotides selected from the cancer type-specific polynucleotide group 1 or complementary sequences thereof will be further listed.
(1) a combination of SEQ ID NOs: 42, 63, 585, and 609 (markers: hsa-miR-4525, hsa-miR-4707-3p, hsa-miR-3196, and hsa-miR-451a);
(2) a combination of SEQ ID NOs: 42, 63, 592, and 609 (markers: hsa-miR-4525, hsa-miR-4707-3p, hsa-miR-4723-5p, and hsa-miR-451a);
(3) a combination of SEQ ID NOs: 18, 42, 581, and 609 (markers: hsa-miR-4448, hsa-miR-4525, hsa-miR-642a-3p, and hsa-miR-451a);
(4) a combination of SEQ ID NOs: 12, 16, 599, and 609 (markers: hsa-miR-4745-5p, hsa-miR-92a-2-5p, hsa-miR-6717-5p, and hsa-miR-451a); and
(5) a combination of SEQ ID NOs: 16, 126, 599, and 609 (markers: hsa-miR-92a-2-5p, hsa-miR-3648, hsa-miR-6717-5p, and hsa-miR-451a).

Non-limiting examples of the combination of the polynucleotide consisting of the nucleotide sequence represented by SEQ ID NO: 611 or a complementary sequence thereof with polynucleotides consisting of nucleotide sequences represented by SEQ ID NOs of three polynucleotides selected from the cancer type-specific polynucleotide group 1 or complementary sequences thereof will be further listed.
(1) a combination of SEQ ID NOs: 12, 16, 37, and 611 (markers: hsa-miR-4745-5p, hsa-miR-92a-2-5p, hsa-miR-3619-3p, and hsa-miR-671-5p);
(2) a combination of SEQ ID NOs: 1, 63, 139, and 611 (markers: hsa-miR-4443, hsa-miR-4707-3p, hsa-miR-760, and hsa-miR-671-5p);
(3) a combination of SEQ ID NOs: 63, 158, 173, and 611 (markers: hsa-miR-4707-3p, hsa-miR-1228-5p, hsa-miR-665, and hsa-miR-671-5p);
(4) a combination of SEQ ID NOs: 16, 37, 139, and 611 (markers: hsa-miR-92a-2-5p, hsa-miR-3619-3p, hsa-miR-760, and hsa-miR-671-5p); and
(5) a combination of SEQ ID NOs: 16, 37, 595, and 611 (markers: hsa-miR-92a-2-5p, hsa-miR-3619-3p, hsa-miR-4739, and hsa-miR-671-5p).

The kit or the device of the present invention can also contain a polynucleotide that is already known or that will be found in future, to enable detection of prostate cancer, in addition to the polynucleotide(s) (which can include a variant, a fragment, and a derivative) according to the present invention described above.

The kit of the present invention can also contain an antibody for measuring a marker for prostate cancer examination known in the art, such as PSA, in addition to the polynucleotide(s) according to the present invention described above, and a variant thereof or a fragment thereof.

These polynucleotides and the variants thereof or the fragments thereof contained in the kit of the present invention can be packaged in different containers either individually or in any combination.

The kit of the present invention can contain a kit for extracting a nucleic acid (e.g., total RNA) from body fluids, cells, or tissues, a fluorescent material for labeling, an enzyme and a medium for nucleic acid amplification, an instruction manual, etc.

The device of the present invention is a device for cancer marker measurement in which nucleic acids such as the polynucleotides according to the present invention described above, variants thereof, derivatives thereof, or fragments thereof are bonded or attached to, for example, a solid phase. Examples of the material for the solid phase include plastics, paper, glass, and silicon. The material for the solid phase is preferably a plastic from the viewpoint of easy processability. The solid phase has any shape and is, for example, square, round, reed-shaped, or film-shaped. The device of the present invention includes, for example, a device for measurement by a hybridization technique. Specific examples thereof include blotting devices and nucleic acid arrays (e.g., microarrays, DNA chips, and RNA chips).

The nucleic acid array technique is a technique which involves bonding or attaching the nucleic acids one by one by use of a method [e.g., a method of spotting the nucleic acids using a high-density dispenser called spotter or arrayer onto the surface of the solid phase surface-treated, if necessary, by coating with L-lysine or the introduction of a functional group such as an amino group or a carboxyl group, a method of spraying the nucleic acids onto the solid phase using an inkjet which injects very small liquid droplets by a piezoelectric element or the like from a nozzle, or a method of sequentially synthesizing nucleotides on the solid phase] to prepare an array such as a chip and measuring a target nucleic acid(s) through the use of hybridization using this array.

The kit or the device of the present invention comprises nucleic acids capable of specifically binding to the polynucleotides of at least one or more, preferably at least two or more, more preferably at least three or more, most preferably at least five or more to all of the prostate cancer marker miRNAs, respectively, of the group 1 described above. The kit or the device of the present invention can optionally further comprise nucleic acids capable of specifically binding to the polynucleotides of at least one or more, preferably at least two or more, more preferably at least three or more, most preferably at least five or more to all of the prostate cancer marker miRNAs, respectively, of the group 2 described above. The kit or the device of the present invention can optionally further comprise nucleic acids capable of specifically binding to the polynucleotides of at least one or more, preferably at least two or more, more preferably at least three or more, most preferably at least five or more to all of the prostate cancer marker miRNAs, respectively, of the group 3 described above.

The kit or the device of the present invention can be used for detecting prostate cancer as described in the Section 4 below.

### 4. Method for Detecting Prostate Cancer

The present invention further provides a method for detecting prostate cancer, comprising using the kit or the device of the present invention (including the nucleic acid(s) that can be used in the present invention) described in the preceding Section 3 to measure an expression level(s) of one or more prostate cancer-derived gene(s) represented by an expression level(s) of prostate cancer-derived gene(s) selected from the following group: miR-4443, miR-1908-5p, miR-4257, miR-3197, miR-3188, miR-4649-5p, miR-1343-3p, miR-6861-5p, miR-1343-5p, miR-642b-3p, miR-6741-5p, miR-4745-5p, miR-6826-5p, miR-3663-3p, miR-3131, miR-92a-2-5p, miR-4258, miR-4448, miR-6125, miR-6880-5p, miR-6132, miR-4467, miR-6749-5p, miR-2392, miR-1273g-3p, miR-4746-3p, miR-1914-3p, miR-7845-5p, miR-6726-5p, miR-128-2-5p, miR-4651, miR-6765-3p, miR-3185, miR-4792, miR-6887-5p, miR-5572, miR-3619-3p, miR-6780b-5p, miR-4707-5p, miR-8063, miR-4454, miR-4525, miR-7975, miR-744-5p, miR-3135b, miR-4648, miR-6816-5p, miR-4741, miR-7150, miR-6791-5p, miR-1247-3p, miR-7977, miR-4497, miR-6090, miR-6781-5p, miR-6870-5p, miR-6729-5p, miR-4530, miR-7847-3p, miR-6825-5p, miR-4674, miR-3917, miR-4707-3p, miR-6885-5p, miR-6722-3p, miR-4516, miR-6757-5p, miR-6840-3p, miR-5195-3p, miR-6756-5p, miR-6800-5p, miR-6727-5p, miR-6126, miR-6872-3p, miR-4446-3p, miR-1268a, miR-1908-3p, miR-3679-5p, miR-4534, miR-4675, miR-7108-5p, miR-6799-5p, miR-4695-5p, miR-3178, miR-5090, miR-3180, miR-1237-5p, miR-4758-5p, miR-3184-5p, miR-4286, miR-6784-5p, miR-6768-5p, miR-6785-5p, miR-4706, miR-711, miR-1260a, miR-6746-5p, miR-6089, miR-6821-5p, miR-4667-5p, miR-8069, miR-4726-5p, miR-6124, miR-4532, miR-4486, miR-4728-5p, miR-4508, miR-128-1-5p, miR-4513, miR-6795-5p, miR-4689, miR-6763-5p, miR-8072, miR-6765-5p, miR-4419b, miR-7641, miR-3928-3p, miR-1227-5p, miR-4492, miR-296-3p, miR-6769a-5p, miR-6889-5p, miR-4632-5p, miR-4505, miR-3154, miR-3648, miR-4442, miR-3141, miR-7113-3p, miR-6819-5p, miR-3195, miR-1199-5p, miR-6738-5p, miR-4656, miR-6820-5p, miR-204-3p, miR-642a-3p, miR-762, miR-1202, miR-3162-5p, miR-3196, miR-3622a-5p, miR-3665, miR-3940-5p, miR-4294, miR-4466, miR-4476, miR-4723-5p, miR-4725-3p, miR-4730, miR-4739, miR-4787-5p, miR-5787, miR-6085, miR-6717-5p, miR-6724-5p, miR-6777-5p, miR-6778-5p, miR-6787-5p, miR-6789-5p, miR-6845-5p and miR-6893-5p, optionally an expression level of prostate cancer-derived gene(s) selected from the following group: miR-615-5p, miR-486-3p, miR-1225-3p, miR-760, miR-187-5p, miR-1203, miR-7110-5p, miR-371a-5p, miR-939-5p, miR-575, miR-92b-5p, miR-887-3p, miR-920, miR-1915-5p, miR-1231, miR-663b, miR-1225-5p, miR-16-5p, miR-423-5p, miR-451a, miR-564 and miR-671-5p, and optionally an expression level of prostate cancer-derived gene(s) selected from the following group: miR-4763-3p, miR-3656, miR-4488, miR-125a-3p, miR-1469, miR-1228-5p, miR-6798-5p, miR-1268b, miR-6732-5p, miR-1915-3p, miR-4433b-3p, miR-1207-5p, miR-4433-3p, miR-6879-5p, miR-4417, miR-30c-1-3p, miR-4638-5p, miR-6088, miR-4270, miR-6782-5p, miR-665, miR-486-5p, miR-4655-5p, miR-1275, miR-6806-5p, miR-614, miR-3937, miR-6752-5p, miR-6771-5p, miR-4450, miR-211-3p, miR-663a, miR-6842-5p, miR-7114-5p and miR-6779-5p in a sample in vitro, further comparing, for example, the expression level(s) of the gene(s) in the sample (e.g., blood, serum, or plasma) collected from a subject suspected of having prostate cancer with a control expression level in the sample collected from a healthy subject (including a non-prostate cancer patient), and evaluating the subject as having prostate cancer when the expression level of the target nucleic acid is statistically significantly different between the samples.

This method of the present invention permits limitedly invasive early diagnosis of cancer with high sensitivity and specificity and thereby brings about early treatment and improved prognosis. In addition, exacerbation of the disease or the effectiveness of surgical, radiotherapeutic, and chemotherapeutic treatments can be monitored.

The method for extracting the prostate cancer-derived gene from the sample such as blood, serum, or plasma according to the present invention is particularly preferably prepared by the addition of a reagent for RNA extraction in 3D-Gene™ RNA extraction reagent from liquid sample kit (Toray Industries, Inc.). A general acidic phenol method (acid guanidinium-phenol-chloroform (AGPC)) may be used, or Trizol® (Life Technologies Corp.) may be used. The prostate cancer-derived genes may be prepared by the addition of a reagent for RNA extraction containing acidic phenol, such as Trizol (Life Technologies Corp.) or Isogen (Nippon Gene Co., Ltd). Alternatively, a kit such as miRNeasy® Mini Kit (Qiagen N.V.) can be used, though the method is not limited thereto.

The present invention also provides use of the kit or the device of the present invention for detecting in vitro an expression product of a prostate cancer-derived miRNA gene(s) in a sample derived from a subject.

In the method of the present invention, a kit or a device comprising, each alone or in every possible composition, the polynucleotides that can be used in the present invention as described above is used as the kit or the device.

In the detection or (genetic) diagnosis of prostate cancer according to the present invention, each polynucleotide contained in the kit or the device of the present invention can be used as a probe or a primer. In the case of using the polynucleotide as a primer, TaqMan® MicroRNA Assays from Life Technologies Corp., miScript PCR System from Qiagen N.V., or the like can be used, though the method is not limited thereto.

The polynucleotide contained in the kit or the device of the present invention can be used as a primer or a probe according to a routine method in a method known in the art for specifically detecting the particular gene, for example, a hybridization technique such as Northern blot, Southern blot, in situ hybridization, Northern hybridization, or Southern hybridization, or a quantitative amplification technique such as quantitative RT-PCR. A body fluid such as blood, serum, plasma, or urine of the subject is collected as a sample to be assayed according to the type of the detection method used. Alternatively, total RNA prepared from such a body fluid by the method described above may be used, and various polynucleotides including cDNA prepared on the basis of the RNA may be used.

The kit or the device of the present invention is useful for the diagnosis of prostate cancer or the detection of the presence or absence of prostate cancer. Specifically, the detection of prostate cancer using the kit or the device can be performed by detecting in vitro an expression level(s) of a gene(s) using the nucleic acid probe(s) or the primer(s) contained in the kit or the device in a sample such as blood, serum, plasma, or urine from a subject suspected of having prostate cancer. The subject suspected of having prostate cancer can be evaluated as having prostate cancer when the expression level(s) of a target miRNA marker(s) measured using polynucleotide(s) (including any variant, any fragment, and any derivative thereof) consisting of a nucleotide sequence(s) represented by at least one or more of SEQ ID NOs: 1 to 135, 580 to 606, or a complementary sequence(s) thereof, optionally a nucleotide sequence(s) represented by one or more of SEQ ID NOs: 136 to 152, 607 to 611 or a complementary sequence(s) thereof, and optionally a nucleotide sequence(s) represented by one or more of SEQ ID NOs: 153 to 187 or a complementary sequence(s) thereof in the sample such as blood, serum, plasma, or urine of the subject is statistically significantly different from the expression level(s) thereof in the sample such as blood, serum, or plasma, or urine of a healthy subject.

The method of the present invention can be combined with rectal examination, transrectal ultrasonography of the prostate, or a diagnostic imaging method such as CT scan, MRI scan, or bone scintigraphy. The method of the present invention is capable of specifically detecting prostate cancer and can substantially discriminate prostate cancer from the other cancers.

The method for detecting the absence of an expression product of a prostate cancer-derived gene(s) or the presence of the expression product of a prostate cancer-derived gene(s) in a sample using the kit or the device of the present invention comprises collecting a body fluid such as blood, serum, plasma, or urine of a subject, and measuring the expression level(s) of the target gene(s) contained therein using one or more polynucleotide(s) (including a variant, a fragment, and a derivative) selected from the polynucleotide group of the present invention, to evaluate the presence or absence of prostate cancer or to detect prostate cancer. The method for detecting prostate cancer according to the present invention can also evaluate or diagnose, for example, the presence or absence of amelioration of the disease or the degree of amelioration thereof in a prostate cancer patient given a therapeutic drug for the amelioration of the disease.

The method of the present invention can comprise, for example, the following steps (a), (b), and (c):
(a) contacting a sample derived from a subject with a polynucleotide(s) in the kit or the device of the present invention in vitro;
(b) measuring an expression level(s) of the target nucleic acid(s) in the sample using the polynucleotide(s) as a nucleic acid probe(s) or a primer(s); and
(c) evaluating the presence or absence of prostate cancer (cells) in the subject on the basis of the step (b).

Specifically, the present invention provides a method for detecting prostate cancer, comprising measuring an expression level(s) of a target nucleic acid(s) in a sample of a subject using a nucleic acid(s) capable of specifically binding to at least one or more (preferably at least two or more) polynucleotide(s) selected from miR-4443, miR-1908-5p, miR-4257, miR-3197, miR-3188, miR-4649-5p, miR-1343-3p, miR-6861-5p, miR-1343-5p, miR-642b-3p, miR-6741-5p, miR-4745-5p, miR-6826-5p, miR-3663-3p, miR-3131, miR-92a-2-5p, miR-4258, miR-4448, miR-6125, miR-6880-5p, miR-6132, miR-4467, miR-6749-5p, miR-2392, miR-1273g-3p, miR-4746-3p, miR-1914-3p, miR-7845-5p, miR-6726-5p, miR-128-2-5p, miR-4651, miR-6765-3p, miR-3185, miR-4792, miR-6887-5p, miR-5572, miR-3619-3p, miR-6780b-5p, miR-4707-5p, miR-8063, miR-4454, miR-4525, miR-7975, miR-744-5p, miR-3135b, miR-4648, miR-6816-5p, miR-4741, miR-7150, miR-6791-5p, miR-1247-3p, miR-7977, miR-4497, miR-6090, miR-6781-5p, miR-6870-5p, miR-6729-5p, miR-4530, miR-7847-3p, miR-6825-5p, miR-4674, miR-3917, miR-4707-3p, miR-6885-5p, miR-6722-3p, miR-4516, miR-6757-5p, miR-6840-3p, miR-5195-3p, miR-6756-5p, miR-6800-5p, miR-6727-5p, miR-6126, miR-6872-3p, miR-4446-3p, miR-1268a, miR-1908-3p, miR-3679-5p, miR-4534, miR-4675, miR-7108-5p, miR-6799-5p, miR-4695-5p, miR-3178, miR-5090, miR-3180, miR-1237-5p, miR-4758-5p, miR-3184-5p, miR-4286, miR-6784-5p, miR-6768-5p, miR-6785-5p, miR-4706, miR-711, miR-1260a, miR-6746-5p, miR-6089, miR-6821-5p, miR-4667-5p, miR-8069, miR-4726-5p, miR-6124, miR-4532, miR-4486, miR-4728-5p, miR-4508, miR-128-1-5p, miR-4513, miR-6795-5p, miR-4689, miR-6763-5p, miR-8072, miR-6765-5p, miR-4419b, miR-7641, miR-3928-3p, miR-1227-5p, miR-4492, miR-296-3p, miR-6769a-5p, miR-6889-5p, miR-4632-5p, miR-4505, miR-3154, miR-3648, miR-4442, miR-3141, miR-7113-3p, miR-6819-5p, miR-3195, miR-1199-5p, miR-6738-5p, miR-4656, miR-6820-5p, miR-204-3p, miR-642a-3p, miR-762, miR-1202, miR-3162-5p, miR-3196, miR-3622a-5p, miR-3665, miR-3940-5p, miR-4294, miR-4466, miR-4476, miR-4723-5p, miR-4725-3p, miR-4730, miR-4739, miR-4787-5p, miR-5787, miR-6085, miR-6717-5p, miR-6724-5p, miR-6777-5p, miR-6778-5p, miR-6787-5p, miR-6789-5p, miR-6845-5p and miR-6893-5p and evaluating in vitro the presence or absence of prostate cancer in the subject using the measured expression level(s) and a control expression level(s) of a healthy subject measured in the same way as above.

In the present specification, the term “evaluation” is evaluation support based on results of in vitro examination, not physician's judgment.

As described above, in the method of the present invention, specifically, miR-4443 is hsa-miR-4443, miR-1908-5p is hsa-miR-1908-5p, miR-4257 is hsa-miR-4257, miR-3197 is hsa-miR-3197, miR-3188 is hsa-miR-3188, miR-4649-5p is hsa-miR-4649-5p, miR-1343-3p is hsa-miR-1343-3p, miR-6861-5p is hsa-miR-6861-5p, miR-1343-5p is hsa-miR-1343-5p, miR-642b-3p is hsa-miR-642b-3p, miR-6741-5p is hsa-miR-6741-5p, miR-4745-5p is hsa-miR-4745-5p, miR-6826-5p is hsa-miR-6826-5p, miR-3663-3p is hsa-miR-3663-3p, miR-3131 is hsa-miR-3131, miR-92a-2-5p is hsa-miR-92a-2-5p, miR-4258 is hsa-miR-4258, miR-4448 is hsa-miR-4448, miR-6125 is hsa-miR-6125, miR-6880-5p is hsa-miR-6880-5p, miR-6132 is hsa-miR-6132, miR-4467 is hsa-miR-4467, miR-6749-5p is hsa-miR-6749-5p, miR-2392 is hsa-miR-2392, miR-1273g-3p is hsa-miR-1273g-3p, miR-4746-3p is hsa-miR-4746-3p, miR-1914-3p is hsa-miR-1914-3p, miR-7845-5p is hsa-miR-7845-5p, miR-6726-5p is hsa-miR-6726-5p, miR-128-2-5p is hsa-miR-128-2-5p, miR-4651 is hsa-miR-4651, miR-6765-3p is hsa-miR-6765-3p, miR-3185 is hsa-miR-3185, miR-4792 is hsa-miR-4792, miR-6887-5p is hsa-miR-6887-5p, miR-5572 is hsa-miR-5572, miR-3619-3p is hsa-miR-3619-3p, miR-6780b-5p is hsa-miR-6780b-5p, miR-4707-5p is hsa-miR-4707-5p, miR-8063 is hsa-miR-8063, miR-4454 is hsa-miR-4454, miR-4525 is hsa-miR-4525, miR-7975 is hsa-miR-7975, miR-744-5p is hsa-miR-744-5p, miR-3135b is hsa-miR-3135b, miR-4648 is hsa-miR-4648, miR-6816-5p is hsa-miR-6816-5p, miR-4741 is hsa-miR-4741, miR-7150 is hsa-miR-7150, miR-6791-5p is hsa-miR-6791-5p, miR-1247-3p is hsa-miR-1247-3p, miR-7977 is hsa-miR-7977, miR-4497 is hsa-miR-4497, miR-6090 is hsa-miR-6090, miR-6781-5p is hsa-miR-6781-5p, miR-6870-5p is hsa-miR-6870-5p, miR-6729-5p is hsa-miR-6729-5p, miR-4530 is hsa-miR-4530, miR-7847-3p is hsa-miR-7847-3p, miR-6825-5p is hsa-miR-6825-5p, miR-4674 is hsa-miR-4674, miR-3917 is hsa-miR-3917, miR-4707-3p is hsa-miR-4707-3p, miR-6885-5p is hsa-miR-6885-5p, miR-6722-3p is hsa-miR-6722-3p, miR-4516 is hsa-miR-4516, miR-6757-5p is hsa-miR-6757-5p, miR-6840-3p is hsa-miR-6840-3p, miR-5195-3p is hsa-miR-5195-3p, miR-6756-5p is hsa-miR-6756-5p, miR-6800-5p is hsa-miR-6800-5p, miR-6727-5p is hsa-miR-6727-5p, miR-6126 is hsa-miR-6126, miR-6872-3p is hsa-miR-6872-3p, miR-4446-3p is hsa-miR-4446-3p, miR-1268a is hsa-miR-1268a, miR-1908-3p is hsa-miR-1908-3p, miR-3679-5p is hsa-miR-3679-5p, miR-4534 is hsa-miR-4534, miR-4675 is hsa-miR-4675, miR-7108-5p is hsa-miR-7108-5p, miR-6799-5p is hsa-miR-6799-5p, miR-4695-5p is hsa-miR-4695-5p, miR-3178 is hsa-miR-3178, miR-5090 is hsa-miR-5090, miR-3180 is hsa-miR-3180, miR-1237-5p is hsa-miR-1237-5p, miR-4758-5p is hsa-miR-4758-5p, miR-3184-5p is hsa-miR-3184-5p, miR-4286 is hsa-miR-4286, miR-6784-5p is hsa-miR-6784-5p, miR-6768-5p is hsa-miR-6768-5p, miR-6785-5p is hsa-miR-6785-5p, miR-4706 is hsa-miR-4706, miR-711 is hsa-miR-711, miR-1260a is hsa-miR-1260a, miR-6746-5p is hsa-miR-6746-5p, miR-6089 is hsa-miR-6089, miR-6821-5p is hsa-miR-6821-5p, miR-4667-5p is hsa-miR-4667-5p, miR-8069 is hsa-miR-8069, miR-4726-5p is hsa-miR-4726-5p, miR-6124 is hsa-miR-6124, miR-4532 is hsa-miR-4532, miR-4486 is hsa-miR-4486, miR-4728-5p is hsa-miR-4728-5p, miR-4508 is hsa-miR-4508, miR-128-1-5p is hsa-miR-128-1-5p, miR-4513 is hsa-miR-4513, miR-6795-5p is hsa-miR-6795-5p, miR-4689 is hsa-miR-4689, miR-6763-5p is hsa-miR-6763-5p, miR-8072 is hsa-miR-8072, miR-6765-5p is hsa-miR-6765-5p, miR-4419b is hsa-miR-4419b, miR-7641 is hsa-miR-7641, miR-3928-3p is hsa-miR-3928-3p, miR-1227-5p is hsa-miR-1227-5p, miR-4492 is hsa-miR-4492, miR-296-3p is hsa-miR-296-3p, miR-6769a-5p is hsa-miR-6769a-5p, miR-6889-5p is hsa-miR-6889-5p, miR-4632-5p is hsa-miR-4632-5p, miR-4505 is hsa-miR-4505, miR-3154 is hsa-miR-3154, miR-3648 is hsa-miR-3648, miR-4442 is hsa-miR-4442, miR-3141 is hsa-miR-3141, miR-7113-3p is hsa-miR-7113-3p, miR-6819-5p is hsa-miR-6819-5p, miR-3195 is hsa-miR-3195, miR-1199-5p is hsa-miR-1199-5p, miR-6738-5p is hsa-miR-6738-5p, miR-4656 is hsa-miR-4656, miR-6820-5p is hsa-miR-6820-5p, miR-204-3p is hsa-miR-204-3p, miR-642a-3p is hsa-miR-642a-3p, miR-762 is hsa-miR-762, miR-1202 is hsa-miR-1202, miR-3162-5p is hsa-miR-3162-5p, miR-3196 is hsa-miR-3196, miR-3622a-5p is hsa-miR-3622a-5p, miR-3665 is hsa-miR-3665, miR-3940-5p is hsa-miR-3940-5p, miR-4294 is hsa-miR-4294, miR-4466 is hsa-miR-4466, miR-4476 is hsa-miR-4476, miR-4723-5p is hsa-miR-4723-5p, miR-4725-3p is hsa-miR-4725-3p, miR-4730 is hsa-miR-4730, miR-4739 is hsa-miR-4739, miR-4787-5p is hsa-miR-4787-5p, miR-5787 is hsa-miR-5787, miR-6085 is hsa-miR-6085, miR-6717-5p is hsa-miR-6717-5p, miR-6724-5p is hsa-miR-6724-5p, miR-6777-5p is hsa-miR-6777-5p, miR-6778-5p is hsa-miR-6778-5p, miR-6787-5p is hsa-miR-6787-5p, miR-6789-5p is hsa-miR-6789-5p, miR-6845-5p is hsa-miR-6845-5p, and miR-6893-5p is hsa-miR-6893-5p.

In the method of the present invention, specifically, the nucleic acid(s) (specifically, probe(s) or primer(s)) is selected from the group consisting of the following polynucleotides (a) to (e):
(a) a polynucleotide consisting of a nucleotide sequence represented by any of SEQ ID NOs: 1 to 135 and 580 to 606 or a nucleotide sequence derived from the nucleotide sequence by the replacement of u with t, a variant thereof, a derivative thereof, or a fragment thereof comprising 15 or more consecutive nucleotides,
(b) a polynucleotide comprising a nucleotide sequence represented by any of SEQ ID NOs: 1 to 135 and 580 to 606,
(c) a polynucleotide consisting of a nucleotide sequence complementary to a nucleotide sequence represented by any of SEQ ID NOs: 1 to 135 and 580 to 606 or a nucleotide sequence derived from the nucleotide sequence by the replacement of u with t, a variant thereof, a derivative thereof, or a fragment thereof comprising 15 or more consecutive nucleotides,
(d) a polynucleotide comprising a nucleotide sequence complementary to a nucleotide sequence represented by any of SEQ ID NOs: 1 to 135 and 580 to 606 or a nucleotide sequence derived from the nucleotide sequence by the replacement of u with t, and
(e) a polynucleotide hybridizing under stringent conditions to any of the polynucleotides (a) to (d).

The method of the present invention can further use a nucleic acid capable of specifically binding to at least one or more polynucleotide(s) selected from miR-615-5p, miR-486-3p, miR-1225-3p, miR-760, miR-187-5p, miR-1203, miR-7110-5p, miR-371a-5p, miR-939-5p, miR-575, miR-92b-5p, miR-887-3p, miR-920, miR-1915-5p, miR-1231, miR-663b, miR-1225-5p, miR-16-5p, miR-423-5p, miR-451a, miR-564 and miR-671-5p.

Specifically, miR-615-5p is hsa-miR-615-5p, miR-486-3p is hsa-miR-486-3p, miR-1225-3p is hsa-miR-1225-3p, miR-760 is hsa-miR-760, miR-187-5p is hsa-miR-187-5p, miR-1203 is hsa-miR-1203, miR-7110-5p is hsa-miR-7110-5p, miR-371a-5p is hsa-miR-371a-5p, miR-939-5p is hsa-miR-939-5p, miR-575 is hsa-miR-575, miR-92b-5p is hsa-miR-92b-5p, miR-887-3p is hsa-miR-887-3p, miR-920 is hsa-miR-920, miR-1915-5p is hsa-miR-1915-5p, miR-1231 is hsa-miR-1231, miR-663b is hsa-miR-663b, miR-1225-5p is hsa-miR-1225-5p, miR-16-5p is hsa-miR-16-5p, miR-423-5p is hsa-miR-423-5p, miR-451a is hsa-miR-451a, miR-564 is hsa-miR-564, and miR-671-5p is hsa-miR-671-5p.

Specifically, the nucleic acid(s) is further selected from the group consisting of the following polynucleotides (f) to (j):
(f) a polynucleotide consisting of a nucleotide sequence represented by any of SEQ ID NOs: 136 to 152 and 607 to 611 or a nucleotide sequence derived from the nucleotide sequence by the replacement of u with t, a variant thereof, a derivative thereof, or a fragment thereof comprising 15 or more consecutive nucleotides,
(g) a polynucleotide comprising a nucleotide sequence represented by any of SEQ ID NOs: 136 to 152 and 607 to 611,
(h) a polynucleotide consisting of a nucleotide sequence complementary to a nucleotide sequence represented by any of SEQ ID NOs: 136 to 152 and 607 to 611 or a nucleotide sequence derived from the nucleotide sequence by the replacement of u with t, a variant thereof, a derivative thereof, or a fragment thereof comprising 15 or more consecutive nucleotides,
(i) a polynucleotide comprising a nucleotide sequence complementary to a nucleotide sequence represented by any of SEQ ID NOs: 136 to 152 and 607 to 611 or a nucleotide sequence derived from the nucleotide sequence by the replacement of u with t, and
(j) a polynucleotide hybridizing under stringent conditions to any of the polynucleotides (f) to (i).

The method of the present invention can further use a nucleic acid(s) capable of specifically binding to at least one or more polynucleotide(s) selected from miR-4763-3p, miR-3656, miR-4488, miR-125a-3p, miR-1469, miR-1228-5p, miR-6798-5p, miR-1268b, miR-6732-5p, miR-1915-3p, miR-4433b-3p, miR-1207-5p, miR-4433-3p, miR-6879-5p, miR-4417, miR-30c-1-3p, miR-4638-5p, miR-6088, miR-4270, miR-6782-5p, miR-665, miR-486-5p, miR-4655-5p, miR-1275, miR-6806-5p, miR-614, miR-3937, miR-6752-5p, miR-6771-5p, miR-4450, miR-211-3p, miR-663a, miR-6842-5p, miR-7114-5p and miR-6779-5p.

Specifically, miR-4763-3p is hsa-miR-4763-3p, miR-3656 is hsa-miR-3656, miR-4488 is hsa-miR-4488, miR-125a-3p is hsa-miR-125a-3p, miR-1469 is hsa-miR-1469, miR-1228-5p is hsa-miR-1228-5p, miR-6798-5p is hsa-miR-6798-5p, miR-1268b is hsa-miR-1268b, miR-6732-5p is hsa-miR-6732-5p, miR-1915-3p is hsa-miR-1915-3p, miR-4433b-3p is hsa-miR-4433b-3p, miR-1207-5p is hsa-miR-1207-5p, miR-4433-3p is hsa-miR-4433-3p, miR-6879-5p is hsa-miR-6879-5p, miR-4417 is hsa-miR-4417, miR-30c-1-3p is hsa-miR-30c-1-3p, miR-4638-5p is hsa-miR-4638-5p, miR-6088 is hsa-miR-6088, miR-4270 is hsa-miR-4270, miR-6782-5p is hsa-miR-6782-5p, miR-665 is hsa-miR-665, miR-486-5p is hsa-miR-486-5p, miR-4655-5p is hsa-miR-4655-5p, miR-1275 is hsa-miR-1275, miR-6806-5p is hsa-miR-6806-5p, miR-614 is hsa-miR-614, miR-3937 is hsa-miR-3937, miR-6752-5p is hsa-miR-6752-5p, miR-6771-5p is hsa-miR-6771-5p, miR-4450 is hsa-miR-4450, miR-211-3p is hsa-miR-211-3p, miR-663a is hsa-miR-663a, miR-6842-5p is hsa-miR-6842-5p, miR-7114-5p is hsa-miR-7114-5p, and miR-6779-5p is hsa-miR-6779-5p.

Specifically, the nucleic acid further used is a polynucleotide selected from the group consisting of the following polynucleotides (k) to (o):
(k) a polynucleotide consisting of a nucleotide sequence represented by any of SEQ ID NOs: 153 to 187 or a nucleotide sequence derived from the nucleotide sequence by the replacement of u with t, a variant thereof, a derivative thereof, or a fragment thereof comprising 15 or more consecutive nucleotides,
(l) a polynucleotide comprising a nucleotide sequence represented by any of SEQ ID NOs: 153 to 187,
(m) a polynucleotide consisting of a nucleotide sequence complementary to a nucleotide sequence represented by any of SEQ ID NOs: 153 to 187 or a nucleotide sequence derived from the nucleotide sequence by the replacement of u with t, a variant thereof, a derivative thereof, or a fragment thereof comprising 15 or more consecutive nucleotides,
(n) a polynucleotide comprising a nucleotide sequence complementary to a nucleotide sequence represented by any of SEQ ID NOs: 153 to 187 or a nucleotide sequence derived from the nucleotide sequence by the replacement of u with t, and
(o) a polynucleotide hybridizing under stringent conditions to any of the polynucleotides (k) to (n).

Examples of the sample used in the method of the present invention can include samples prepared from a living tissue (preferably a prostate tissue) or a body fluid such as blood, serum, plasma, or urine of the subject. The sample includes, specifically, for example, an RNA-containing sample prepared from the tissue, a polynucleotide-containing sample further prepared therefrom, a body fluid such as blood, serum, plasma, or urine, a portion or the whole of a living tissue collected from the subject by biopsy or the like, or a living tissue excised by surgery can be used, and the sample for measurement can be prepared therefrom.

In the present specification, the subject refers to a mammal, for example, a human, a monkey, a mouse or a rat without any limitation, and is preferably a human.

The steps of the method of the present invention can be changed according to the type of the sample to be assayed.

In the case of using RNA as an analyte, the detection of prostate cancer (cells) can comprise, for example, the following steps (a), (b), and (c):
(a) binding RNA prepared from the sample of the subject or a complementary polynucleotide (cDNA) transcribed therefrom to a polynucleotide in the kit or the device of the present invention;
(b) measuring the sample-derived RNA or the cDNA synthesized from the RNA, bound with the polynucleotide by hybridization using the polynucleotide as a nucleic acid probe or by quantitative RT-PCR using the polynucleotide as a primer; and
(c) evaluating the presence or absence of prostate cancer (or prostate cancer-derived gene expression) on the basis of the measurement results of the step (b).

For example, various hybridization methods can be used for detecting, examining, evaluating, or diagnosing prostate cancer (or prostate cancer-derived gene expression) in vitro according to the present invention. For example, Northern blot, Southern blot, RT-PCR, DNA chip analysis, in situ hybridization, Northern hybridization, or Southern hybridization can be used as such a hybridization method.

In the case of using the Northern blot, the presence or absence of expression of each gene or the expression level thereof in the RNA can be detected or measured by use of the nucleic acid probe that can be used in the present invention. Specific examples thereof can include a method which involves labeling the nucleic acid probe (or its complementary strand) with a radioisotope (³²P, ³³P, ³⁵S, etc.), a fluorescent material, or the like, hybridizing the labeled product with the living tissue-derived RNA of the subject transferred to a nylon membrane or the like according to a routine method, and then detecting and measuring a signal derived from the label (radioisotope or fluorescent material) on the formed DNA/RNA duplex using a radiation detector (examples thereof can include BAS-1800 II (Fujifilm Corp.)) or a fluorescence detector (examples thereof can include STORM 865 (GE Healthcare Japan Corp.)).

In the case of using the quantitative RT-PCR, the presence or absence of expression of each gene or the expression level thereof in the RNA can be detected or measured by use of the primer that can be used in the present invention. Specific examples thereof can include a method which involves preparing cDNA from the living tissue-derived RNA of the subject according to a routine method, hybridizing a pair of primers (consisting of a plus strand and a reverse strand binding to the cDNA) prepared from the polynucleotide for detection of the present invention with the cDNA such that the region of each target gene can be amplified with the cDNA as a template, and performing PCR according to a routine method to detect the obtained double-stranded DNA. The method for detecting the double-stranded DNA can include a method of performing the PCR using the primers labeled in advance with a radioisotope or a fluorescent material, a method of electrophoresing the PCR product on an agarose gel and staining the double-stranded DNA with ethidium bromide or the like for detection, and a method of transferring the produced double-stranded DNA to a nylon membrane or the like according to a routine method and hybridizing the double-stranded DNA to a labeled nucleic acid probe for detection.

In the case of using the nucleic acid array analysis, an RNA chip or a DNA chip in which the nucleic acid probes (single-stranded or double-stranded) of the present invention is attached to a substrate (solid phase) is used. Regions having the attached nucleic acid probes are referred to as probe spots, and regions having no attached nucleic acid probe are referred to as blank spots. Array in which a gene group immobilized on a solid-phase substrate is generally called a nucleic acid chip, a nucleic acid array, a microarray, or the like. The DNA or RNA array includes a DNA or RNA macroarray and a DNA or RNA microarray. In the present specification, the term “chip” includes all of these arrays. 3D-Gene® Human miRNA Oligo chip (Toray Industries, Inc.) can be used as the DNA chip, though the DNA chip is not limited thereto.

Examples of the measurement using the DNA chip can include, but are not limited to, a method of detecting and measuring a signal derived from the label on the nucleic acid probe using an image detector (examples thereof can include Typhoon 9410 (GE Healthcare Japan Corp.) and 3D-Gene® scanner (Toray Industries, Inc.)).

The “stringent conditions” used in the present specification are, as mentioned above, conditions under which a nucleic acid probe hybridizes to its target sequence to a larger extent (e.g., a measurement value equal to or larger than a mean of background measurement values+a standard deviation of the background measurement values×2) than that for other sequences.

The stringent conditions are defined by hybridization and subsequent conditions of washing. The hybridization conditions involves, for example, but not limited to, 30° C. to 60° C. for 1 to 24 hours in a solution containing SSC, a surfactant, formamide, dextran sulfate, a blocking agent, etc. In this context, 1×SSC is an aqueous solution (pH 7.0) containing 150 mM sodium chloride and 15 mM sodium citrate. The surfactant includes, for example, SDS (sodium dodecyl sulfate), Triton, or Tween. The hybridization conditions more preferably involve 3 to 10×SSC and 0.1 to 1% SDS. Examples of the washing conditions, following the hybridization, which is another condition to define the stringent conditions, can include conditions involving continuous washing at 30° C. in a solution containing 0.5×SSC and 0.1% SDS, at 30° C. in a solution containing 0.2×SSC and 0.1% SDS, and at 30° C. in a 0.05×SSC solution. It is desirable that the complementary strand should maintain its hybridized state with a target plus strand even by washing under such conditions. Specifically, examples of such a complementary strand can include a strand consisting of a nucleotide sequence in a completely complementary relationship with the nucleotide sequence of the target plus strand, and a strand consisting of a nucleotide sequence having at least 80%, preferably at least 85%, more preferably at least 90% or at least 95%, for example, at least 98% or at least 99% identity to the strand.

Other examples of the “stringent conditions” for the hybridization are described in, for example, Sambrook, J. & Russel, D., Molecular Cloning, A LABORATORY MANUAL, Cold Spring Harbor Laboratory Press, published on Jan. 15, 2001, Vol. 1, 7.42 to 7.45 and Vol. 2, 8.9 to 8.17, and can be used in the present invention.

Examples of the conditions for carrying out PCR using a polynucleotide fragment in the kit of the present invention as a primer include a treatment for approximately 15 seconds to 1 minute at 5 to 10° C. plus a Tm value calculated from the sequence of the primer, using a PCR buffer having composition such as 10 mM Tris-HCL (pH 8.3), 50 mM KCL, and 1 to 2 mM MgCl₂. Examples of the method for calculating such a Tm value include Tm value=2×(the number of adenine residues+the number of thymine residues)+4×(the number of guanine residues+the number of cytosine residues).

In the case of using the quantitative RT-PCR, a commercially available kit for measurement specially designed for quantitatively measuring miRNA, such as TaqMan® MicroRNA Assays (Life Technologies Corp.), LNA®-based MicroRNA PCR (Exiqon), or Ncode® miRNA qRT-PCT kit (Invitrogen Corp.) may be used.

For the calculation of gene expression levels, statistical analysis described in, for example, Statistical analysis of gene expression microarray data (Speed T., Chapman and Hall/CRC), and A beginner's guide Microarray gene expression data analysis (Causton H. C. et al., Blackwell publishing) can be used in the present invention, though the calculation method is not limited thereto. For example, twice, preferably 3 times, more preferably 6 times the standard deviation of the measurement values of the blank spots are added to the average measurement value of the blank spots on the DNA chip, and probe spots having a signal value equal to or larger than the resulting value can be regarded as detection spots. Alternatively, the average measurement value of the blank spots is regarded as a background and can be subtracted from the measurement values of the probe spots to determine gene expression levels. A missing value for a gene expression level can be excluded from the analyte, preferably replaced with the smallest value of the gene expression level in each DNA chip, or more preferably replaced with a value obtained by subtracting 0.1 from a logarithmic value of the smallest value of the gene expression level. In order to eliminate low-signal genes, only a gene having a gene expression level of 2⁶, preferably 2⁸, more preferably 2¹⁰ or larger in 20% or more, preferably 50%, more preferably 80% or more of the number of measured samples can be selected as the analyte. Examples of the normalization of the gene expression level include, but are not limited to, global normalization and quantile normalization (Bolstad, B. M. et al., 2003, Bioinformatics, Vol. 19, p. 185-193).

The present invention also provides a method comprising measuring a target gene or gene expression level in a sample derived from a subject using the polynucleotide, the kit, or the device (e.g., chip) for detection of the present invention, or a combination thereof, preparing a discriminant (discriminant function) with gene expression levels in a sample derived from a prostate cancer patient and a sample derived from a healthy subject as supervising samples, and determining or evaluating the presence and/or absence of the prostate cancer-derived gene in the sample.

Specifically, the present invention further provides the method comprising: a first step of measuring in vitro an expression level of a target gene in multiple samples known to be able to determine or evaluate the presence and/or absence of the prostate cancer-derived gene in the samples, using the polynucleotide, the kit, or the device (e.g., chip) for detection of the present invention, or a combination thereof, a second step of preparing a discriminant with the measurement values of the expression level of the target gene (target nucleic acid) obtained in the first step as supervising samples; a third step of measuring in vitro an expression level of the target gene in a sample derived from a subject in the same way as in the first step; and a fourth step of substituting the measurement value of the expression level of the target gene obtained in the third step into the discriminant obtained in the second step, and determining or evaluating the presence and/or absence of the prostate cancer-derived gene in the sample on the basis of the results obtained from the discriminant, wherein the target gene can be detected using the polynucleotide or using a polynucleotide for detection, a variant thereof, or a fragment thereof contained in the kit or the device (e.g., chip). In this context, the discriminant can be prepared by use of Fisher's linear discriminant analysis, nonlinear discriminant analysis based on Mahalanobis' distance, neural network, Support Vector Machine (SVM), or the like, though the method is not limited thereto.

When a clustering boundary is a straight line or a hyperplane, the linear discriminant analysis is a method for determining the association of a cluster using Formula 1 as a discriminant. In this context, x represents an explanatory variable, w represents a coefficient of the explanatory variable, and w0 represents a constant term.

$$f ( x ) = {w}_{0} + \text{?} {w}_{i} {x}_{i} \qquad Formula 1$$

$$\text{?} \text{indicates text missing or illegible when filed}$$

Values obtained from the discriminant are referred to as discriminant scores. The measurement values of a newly offered data set can be substituted as explanatory variables into the discriminant to determine clusters on the basis of the signs of the discriminant scores.

The Fisher's linear discriminant analysis, one type of linear discriminant analysis, is a dimension reduction method for selecting a dimension suitable for classification, and constructs a synthetic variable with high discriminant performance by focusing on the variance of the synthetic variables and minimizing the variance of data having the same label (Venables, W. N. et al., Modern Applied Statistics with S. Fourth edition. Springer., 2002). In the Fisher's linear discriminant analysis, direction w of projection is determined so as to maximize Formula 2. In this context, μ represents an average input, ng represents the number of data associated to class g, and μg represents an average input of the data associated to class g. The numerator and the denominator are intra-class variance and inter-class variance, respectively, when each data is projected in the direction of the vector w. Discriminant coefficient wi is determined by maximizing this ratio (Takafumi Kanamori et al., “Pattern Recognition”, Kyoritsu Shuppan Co., Ltd., (2009); and Richard O. et al., Pattern Classification Second Edition., Wiley-Interscience, 2000).

$$J ( w ) = \frac{\text{?} {n}_{g} ( {w}^{T} \text{?} - {w}^{T} μ ) {( {w}^{T} \text{?} - {w}^{T} μ )}^{T}}{\text{?} {n}_{g} ( {w}^{T} \text{?} - {w}^{T} {μ}_{g} ) ( {w}^{T} {x}_{i} - \text{?} )} \qquad Formula 2$$

$$subjectto μ = \text{?} \frac{{x}_{i}}{n} , \text{?} = \text{?} \frac{{x}_{i}}{{n}_{g}}$$

$$\text{?} \text{indicates text missing or illegible when filed}$$

The Mahalanobis' distance is calculated according to Formula 3 in consideration of data correlation and can be used as nonlinear discriminant analysis for determining, an associated cluster which has a closer Mahalanobis' distance from each cluster. In this context, μ represents a central vector of each cluster, and S-1 represents an inverse matrix of the variance-covariance matrix of the cluster. The central vector is calculated from explanatory variable x, and an average vector, a median value vector, or the like can be used.

$$D ( x , μ ) = {\{ ( x - μ \text{?} {S}^{- 1} ( x - μ ) \}}^{\frac{1}{2}} \qquad Formula 3$$

$$\text{?} \text{indicates text missing or illegible when filed}$$

SVM is a discriminant analysis method devised by V. Vapnik (The Nature of Statistical Leaning Theory, Springer, 1995). Particular data points of a data set that has known classes are defined as explanatory variables, and classes are defined as objective variables. A boundary plane called hyperplane for correctly classifying the data set into the known classes is determined, and a discriminant for data classification is determined using the boundary plane. Then, the measurement values of a newly offered data set can be substituted as explanatory variables into the discriminant to determine classes. In this respect, the results of the discriminant analysis may be classes, may be a probability of being classified into correct classes, or may be the distance from the hyperplane. In SVM, a method of nonlinearly converting a feature vector to a high dimension and performing linear discriminant analysis in the space is known as a method for tackling nonlinear problems. An expression in which an inner product of two factors in a nonlinearly mapped space is expressed only by inputs in their original spaces is called kernel. Examples of the kernel can include a linear kernel, a RBF (radial basis function) kernel, and a Gaussian kernel. While highly dimensional mapping is performed according to the kernel, the optimum discriminant, i.e., a discriminant, can be actually constructed by mere calculation according to the kernel, which avoids calculating features in the mapped space (e.g., Hideki Aso et al., Frontier of Statistical Science 6 “Statistics of pattern recognition and learning—New concepts and approaches”, Iwanami Shoten, Publishers (2004); Nello Cristianini et al., Introduction to SVM, Kyoritsu Shuppan Co., Ltd. (2008)).

C-support vector classification (C-SVC), one type of SVM, involves preparing a hyperplane by supervising with the explanatory variables of two groups and classifying an unknown data set into either of the groups (C. Cortes et al., 1995, Machine Learning, Vol. 20, p. 273-297).

Exemplary calculation of a C-SVC discriminant that can be used in the method of the present invention will be given below. First, all subjects are divided into two groups, i.e., a prostate cancer patient group and a healthy subject group. For example, prostate tissue examination can be used for a reference under which each subject is confirmed as a prostate cancer patient or a healthy subject.

Next, a data set consisting of comprehensive gene expression levels of serum-derived samples of the two divided groups (hereinafter, this data set is referred to as a training cohort) is prepared, and a C-SVC discriminant is determined by using genes found to differ clearly in their gene expression levels between the two groups as explanatory variables, and this grouping as objective variables (e.g., −1 and +1). An optimizing objective function is represented by Formula 4 wherein e represents all input vectors, y represents an objective variable, a represents a Lagrange's undetermined multiplier vector, Q represents a positive definite matrix, and C represents a parameter for adjusting constrained conditions.

$$m \text{?} n \frac{1}{1} {a}^{T} Q a - {e}^{T} a \qquad Formula 4$$

$$subject to {y}^{T} a = 0 , 0 \leq {a}_{i} \leq C , i = 1 , … , l ,$$

$$\text{?} \text{indicates text missing or illegible when filed}$$

Formula 5 is a finally obtained discriminant, and an associated group can be determined on the basis of the sign of a value obtained according to the discriminant. In this context, x represents a support vector, y represents a label indicating the association with a group, a represents the corresponding coefficient, b represents a constant term, and K represents a kernel function.

$$f ( x ) = sgn ( \text{?} {a}_{i} K ( \text{?} , x ) + b ) \qquad Formula 5$$

$$\text{?} \text{indicates text missing or illegible when filed}$$

For example, a RBF kernel defined by Formula 6 can be used as the kernel function. In this context, x represents a support vector, and y represents a kernel parameter for adjusting the complexity of the hyperplane.

$$K ( {x}_{i} , {x}_{j} ) = exp ( - r {‖ {x}_{i} - {x}_{j} ‖}^{2} ) , r < 0 \qquad Formula 6$$

In addition, an approach such as neural network, k-nearest neighbor algorithms, decision trees, or logistic regression analysis can be selected as a method for determining or evaluating the presence and/or absence of expression of a prostate cancer-derived target gene in a sample derived from a subject, or for evaluating the expression level thereof by comparison with a control derived from a healthy subject.

The method of the present invention can comprise, for example, the following steps (a), (b), and (c):
(a) measuring an expression level of a target gene in tissues containing prostate cancer-derived genes derived from prostate cancer patients and/or samples already known to be tissues containing no prostate cancer-derived gene derived from healthy subjects, using the polynucleotide, the kit, or the device (e.g., DNA chip) for detection according to the present invention;
(b) preparing the discriminants of Formulae 1 to 3, 5, and 6 described above from the measurement values of the expression level measured in the step (a); and
(c) measuring an expression level of the target gene in a sample derived from a subject using the polynucleotide, the kit, or the device (e.g., DNA chip) for detection according to the present invention, substituting the obtained measurement value into the discriminants prepared in the step (b), and determining or evaluating the presence and/or absence of the prostate cancer-derived target gene in the sample, or evaluating the expression level thereof by comparison with a healthy subject-derived control, on the basis of the obtained results.

In this context, in the discriminants of Formulae 1 to 3, 5, and 6, x represents an explanatory variable and includes a value obtained by measuring a polynucleotide selected from the polynucleotides described above in the Section 2, or any fragment thereof. Specifically, the explanatory variable for discriminating a prostate cancer patient from a healthy subject according to the present invention is a gene expression level selected from, for example, the following expression levels (1) to (3):
(1) a gene expression level in the serum of a prostate cancer patient or a healthy subject measured by any DNA comprising 15 or more consecutive nucleotides in a nucleotide sequence represented by any of SEQ ID NOs: 1 to 135, 580 to 606 or a complementary sequence thereof,
(2) a gene expression level in the serum of a prostate cancer patient or a healthy subject measured by any DNA comprising 15 or more consecutive nucleotides in a nucleotide sequence represented by any of SEQ ID NOs: 136 to 152, 607 to 611 or a complementary sequence thereof, and
(3) a gene expression level in the serum of a prostate cancer patient or a healthy subject measured by any DNA comprising 15 or more consecutive nucleotides in a nucleotide sequence represented by any of SEQ ID NOs: 153 to 187 or a complementary sequence thereof.

As described above, for the method for determining or evaluating the presence and/or absence of a prostate cancer-derived gene in a sample derived from a subject, a discriminant prepared from a training cohort is required. For enhancing the discriminant accuracy of the discriminant, it is necessary for the discriminant to use genes that show clear difference between two groups in the training cohort.

Each gene that is used for an explanatory variable in a discriminant is preferably determined as follows. First, comprehensive gene expression levels of a prostate cancer patient group and comprehensive gene expression levels of a healthy subject group in a training cohort are used as a data set, the degree of difference in the expression level of each gene between the two groups is determined through the use of, for example, the P value of t test, which is parametric analysis, or the P value of Mann-Whitney's U test or Wilcoxon test, which is nonparametric analysis.

The gene can be regarded as being statistically significant when the critical rate (significance level) of the P value obtained by the test is smaller than, for example, 5%, 1%, or 0.01%.

In order to correct an increased probability of type I error attributed to the repetition of an analytical test, a method known in the art, for example, Bonferroni or Holm method, can be used for the correction (e.g., Yasushi Nagata et al., “Basics of statistical multiple comparison methods”, Scientist Press Co., Ltd. (2007)). As an example of the Bonferroni correction, for example, the P value obtained by an analytical test is multiplied by the number of repetitions of the test, i.e., the number of genes used in the analysis, and the obtained value can be compared with a desired significance level to suppress a probability of causing type I error in the whole test.

Instead of the test, the absolute value (fold change) of an expression ratio of a median value of each gene expression level between gene expression levels of a prostate cancer patient group and gene expression levels of a healthy subject group may be calculated to select a gene that is used for an explanatory variable for a discriminant. Alternatively, ROC curves based on the gene expression levels of a prostate cancer patient group and a healthy subject group may be used, and a gene that is used for an explanatory variable in a discriminant can be selected on the basis of an AUROC value.

Next, a discriminant that can be calculated by various methods described above is prepared using any number of genes having large difference in their gene expression levels determined here. Examples of the method for constructing a discriminant that produces the largest discriminant accuracy include a method of constructing a discriminant in every combination of genes that satisfy the significance level of a P value, and a method of constructing a discriminant by repetitively evaluating the genes for use while adding the genes one by one in a descending order of the gene expression difference (Furey T S. et al., 2000, Bioinformatics., Vol. 16, p. 906-14). A gene expression level of another independent prostate cancer patient or healthy subject is substituted as an explanatory variable into this discriminant to calculate a result of the discriminant analysis that indicates the group to which this independent prostate cancer patient or healthy subject associated. Specifically, the found gene set for diagnosis and the discriminant constructed using the gene set for diagnosis can be evaluated in an independent sample group to find a more universal gene set for diagnosis capable of detecting prostate cancer and a more universal method for discriminating prostate cancer.

Split-sample method is preferably used for evaluating the discriminant performance (generality) of the discriminant. Specifically, a data set is divided into a training cohort and a validation cohort, and gene selection by a statistical test and construction of a discriminant are performed in the training cohort. Accuracy, sensitivity, and specificity are calculated using results of discriminant analysis in a validation cohort according to the discriminant and a true group to which the validation cohort associated, to evaluate the discriminant performance. On the other hand, instead of dividing a data set, gene selection by a statistical test and construction of a discriminant may be performed using all of samples, and accuracy, sensitivity, and specificity can be calculated by the discriminant of newly prepared samples according to the discriminant to evaluate the discriminant performance.

The present invention provides a polynucleotide for detection or for disease diagnosis useful in the diagnosis and treatment of prostate cancer, a method for detecting prostate cancer using the polynucleotide, and a kit and a device for the detection of prostate cancer, comprising the polynucleotide. Particularly, in order to select a gene for diagnosis and prepare a discriminant so as to exhibit accuracy beyond a prostate cancer diagnosis method using existing tumor markers PSA, a gene set for diagnosis and a discriminant for the method of the present invention can be constructed, which exhibit accuracy beyond PSA, for example, by comparing genes expressed in serum derived from a patient who is confirmed to be negative using PSA but finally found to have prostate cancer by detailed examination such as computed tomography using a contrast medium, with genes expressed in serum derived from a patient who has no prostate cancer.

For example, the gene set for diagnosis is set to any combination selected from one or two or more of the polynucleotides based on a nucleotide sequence represented by any of SEQ ID NOs: 1 to 135, 580 to 606, or a complementary sequence thereof as described above, optionally one or two or more of the polynucleotides based on a nucleotide sequence represented by any of SEQ ID NOs: 136 to 152, 607 to 611, or a complementary sequence thereof, and optionally one or two or more of the polynucleotides based on a nucleotide sequence represented by any of SEQ ID NOs: 153 to 187, or a complementary sequence thereof. Further, a discriminant is constructed using expression levels of the gene set for diagnosis in samples derived from class I prostate cancer patients and samples derived from class II healthy subjects as a result of tissue diagnosis. As a result, the presence or absence of prostate cancer-derived genes in an unknown sample can be determined with 100% accuracy at the maximum by measuring expression levels of the gene set for diagnosis in the unknown sample.

### EXAMPLES

Hereinafter, the present invention will be described further specifically with reference to Examples below. However, the scope of the present invention is not intended to be limited by these Examples.

### Reference Example 1

<Collection of Samples from Prostate Cancer Patient and Healthy Subject>

Serum was collected after obtainment of informed consent, using VENOJECT II vacuum blood collecting tube VP-AS109K60 (Terumo Corp.) from each of 94 healthy male subjects, and 35 prostate cancer patients (30 cases with stage II, 1 case with stage III, and 4 cases with stage IV) (Table 2-1) who were confirmed to have no cancer in organs other than the prostate, and used as a training cohort. Likewise, serum was collected after obtainment of informed consent, using VENOJECT II vacuum blood collecting tube VP-AS109K60 (Terumo Corp.) from each of 47 healthy male subjects, and 17 prostate cancer patients (15 cases with stage II and 2 cases with stage III) (Table 2-2) who were confirmed to have no cancer in organs other than the prostate, and used as a validation cohort.

### <Extraction of Total RNA>

Total RNA was obtained from 300 μL of the serum sample obtained from each of 193 persons in total of 141 healthy male subjects and 52 prostate cancer patients in the training cohort and the validation cohort, using a reagent for RNA extraction in 3D-Gene® RNA extraction reagent from liquid sample kit (Toray Industries, Inc.) according to the protocol provided by the manufacturer.

### <Measurement of Gene Expression Level>

miRNAs in the total RNA obtained from the serum sample of each of 193 persons in total of 141 healthy male subjects and 52 prostate cancer patients in the aforementioned training cohort and the validation cohort were fluorescently labeled using 3D-Gene® miRNA Labeling kit (Toray Industries, Inc.) according to the protocol (ver 2.20) provided by the manufacturer. The oligo DNA chip used was 3D-Gene® Human miRNA Oligo chip (Toray Industries, Inc.) with mounted probes having sequences complementary to 2,555 miRNAs among the miRNAs registered in miRBase Release 20. Hybridization of the miRNAs in the total RNA with the probes on the DNA chip under stringent conditions and washing following the hybridization were performed according to the protocol provided by the manufacturer. The DNA chip was scanned using 3D-Gene® scanner (Toray Industries, Inc.) to obtain images. Fluorescence intensity was digitized using 3D-Gene® Extraction (Toray Industries, Inc.). The digitized fluorescence intensity was converted to a logarithmic value having a base of 2 and used as a gene expression level, from which a blank value was subtracted. A missing value was replaced with a value obtained by subtracting 0.1 from a logarithmic value of the smallest value of the gene expression level in each DNA chip. As a result, the comprehensive gene expression levels of the miRNAs in the serum were obtained for the 52 prostate cancer patients and the 141 healthy male subjects. Calculation and statistical analysis using the digitized gene expression levels of the miRNAs were carried out using R language 3.0.2 (R Development Core Team (2013). R: A language and environment for statistical computing. R Foundation for Statistical Computing, URL http://www.R-project.org/.) and MASS package 7.3-30 (Venables, W. N. & Ripley, B. D. (2002) Modern Applied Statistics with S. Fourth Edition. Springer, New York. ISBN 0-387-95457-0).

### Reference Example 2

<Collection of Sample from Patients with Cancer Other than Prostate Cancer>

Serum was collected using VENOJECT II vacuum blood collecting tube VP-AS109K60 (Terumo Corp.) from each of 63 breast cancer patients who were confirmed to have no cancer in other organs after obtainment of informed consent, and used as a training cohort together with the samples of 35 prostate cancer patients and 99 healthy male subjects of Reference Example 1. Likewise, serum was collected using VENOJECT II vacuum blood collecting tube VP-AS109K60 (Terumo Corp.) from each of 30 breast cancer patients who were confirmed to have no cancer in other organs after obtainment of informed consent, and used as a validation cohort together with the samples of 17 prostate cancer patients who were confirmed to have no cancer in organs other than the prostate and 51 healthy male subjects of Reference Example 1. Subsequent operations were conducted in the same way as in Reference Example 1.

### Example 1

<Selection of Gene Marker Using Samples in the Training Cohort, and Method for Evaluating Prostate Cancer Discriminant Performance with the Single Gene Marker Using Samples in the Validation Cohort>

In this Example, a gene marker for discriminating a prostate cancer patient from a healthy subject was selected from the training cohort and studied in samples of the validation cohort independent of the training cohort.

Specifically, first, the miRNA expression levels of the training cohort and the validation cohort obtained in the preceding Reference Examples 1 were combined and normalized by quantile normalization.

Next, genes for diagnosis were selected in the training cohort. Here, in order to acquire diagnostic markers with higher reliability, only genes that showed gene expression levels of 2⁶ or higher in 50% or more of the samples in either of the prostate cancer patient group in the training cohort or the healthy subject group in the training cohort were selected. In order to further acquire statistically significant genes for discriminating a prostate cancer patient group from a healthy subject group, the P value obtained by two-tailed t-test assuming equal variance as to each gene expression level was corrected by the Bonferroni method, and genes that satisfied p<0.01 were acquired as gene markers for use in explanatory variables of a discriminant. The obtained genes are described in Table 2.

In this way, hsa-miR-4443, hsa-miR-1908-5p, hsa-miR-4257, hsa-miR-3197, hsa-miR-3188, hsa-miR-4649-5p, hsa-miR-1343-3p, hsa-miR-6861-5p, hsa-miR-1343-5p, hsa-miR-642b-3p, hsa-miR-6741-5p, hsa-miR-4745-5p, hsa-miR-6826-5p, hsa-miR-3663-3p, hsa-miR-3131, hsa-miR-92a-2-5p, hsa-miR-4258, hsa-miR-4448, hsa-miR-6125, hsa-miR-6880-5p, hsa-miR-6132, hsa-miR-4467, hsa-miR-6749-5p, hsa-miR-2392, hsa-miR-1273g-3p, hsa-miR-4746-3p, hsa-miR-1914-3p, hsa-miR-7845-5p, hsa-miR-6726-5p, hsa-miR-128-2-5p, hsa-miR-4651, hsa-miR-6765-3p, hsa-miR-3185, hsa-miR-4792, hsa-miR-6887-5p, hsa-miR-5572, hsa-miR-3619-3p, hsa-miR-6780b-5p, hsa-miR-4707-5p, hsa-miR-8063, hsa-miR-4454, hsa-miR-4525, hsa-miR-7975, hsa-miR-744-5p, hsa-miR-3135b, hsa-miR-4648, hsa-miR-6816-5p, hsa-miR-4741, hsa-miR-7150, hsa-miR-6791-5p, hsa-miR-1247-3p, hsa-miR-7977, hsa-miR-4497, hsa-miR-6090, hsa-miR-6781-5p, hsa-miR-6870-5p, hsa-miR-6729-5p, hsa-miR-4530, hsa-miR-7847-3p, hsa-miR-6825-5p, hsa-miR-4674, hsa-miR-3917, hsa-miR-4707-3p, hsa-miR-6885-5p, hsa-miR-6722-3p, hsa-miR-4516, hsa-miR-6757-5p, hsa-miR-6840-3p, hsa-miR-5195-3p, hsa-miR-6756-5p, hsa-miR-6800-5p, hsa-miR-6727-5p, hsa-miR-6126, hsa-miR-6872-3p, hsa-miR-4446-3p, hsa-miR-1268a, hsa-miR-1908-3p, hsa-miR-3679-5p, hsa-miR-4534, hsa-miR-4675, hsa-miR-7108-5p, hsa-miR-6799-5p, hsa-miR-4695-5p, hsa-miR-3178, hsa-miR-5090, hsa-miR-3180, hsa-miR-1237-5p, hsa-miR-4758-5p, hsa-miR-3184-5p, hsa-miR-4286, hsa-miR-6784-5p, hsa-miR-6768-5p, hsa-miR-6785-5p, hsa-miR-4706, hsa-miR-711, hsa-miR-1260a, hsa-miR-6746-5p, hsa-miR-6089, hsa-miR-6821-5p, hsa-miR-4667-5p, hsa-miR-8069, hsa-miR-4726-5p, hsa-miR-6124, hsa-miR-4532, hsa-miR-4486, hsa-miR-4728-5p, hsa-miR-4508, hsa-miR-128-1-5p, hsa-miR-4513, hsa-miR-6795-5p, hsa-miR-4689, hsa-miR-6763-5p, hsa-miR-8072, hsa-miR-6765-5p, hsa-miR-4419b, hsa-miR-7641, hsa-miR-3928-3p, hsa-miR-1227-5p, hsa-miR-4492, hsa-miR-296-3p, hsa-miR-6769a-5p, hsa-miR-6889-5p, hsa-miR-4632-5p, hsa-miR-4505, hsa-miR-3154, hsa-miR-3648, hsa-miR-4442, hsa-miR-3141, hsa-miR-7113-3p, hsa-miR-6819-5p, hsa-miR-3195, hsa-miR-1199-5p, hsa-miR-6738-5p, hsa-miR-4656, hsa-miR-6820-5p, hsa-miR-615-5p, hsa-miR-486-3p, hsa-miR-1225-3p, hsa-miR-760, hsa-miR-187-5p, hsa-miR-1203, hsa-miR-7110-5p, hsa-miR-371a-5p, hsa-miR-939-5p, hsa-miR-575, hsa-miR-92b-5p, hsa-miR-887-3p, hsa-miR-920, hsa-miR-1915-5p, hsa-miR-1231, hsa-miR-663 and hsa-miR-1225-5p genes, and the nucleotide sequences of SEQ ID NOs: 1 to 152 related thereto were found.

A discriminant for determining the presence or absence of prostate cancer was further prepared by Fisher's linear discriminant analysis with the expression levels of these genes as an index. Specifically, any newly found polynucleotide consisting of a nucleotide sequence represented by any of SEQ ID NOs: 1 to 135 among the 152 genes selected in the training cohort was applied to Formula 2 to construct a discriminant. Calculated accuracy, sensitivity, and specificity are shown in Table 4. In this respect, a discriminant coefficient and a constant term are shown in Table 5.

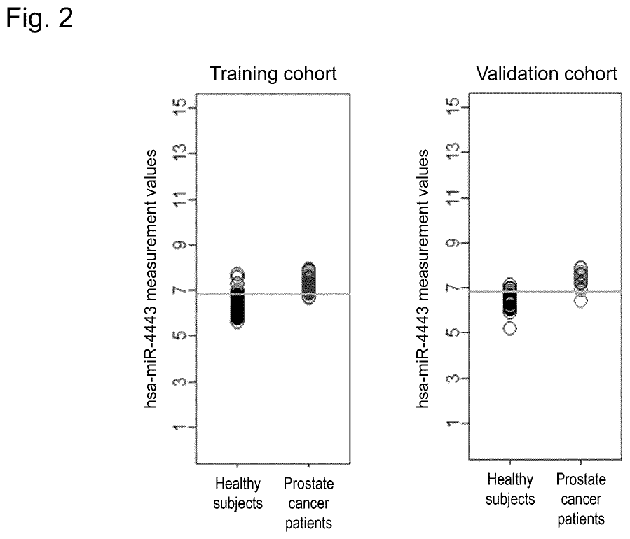

Next, accuracy, sensitivity, and specificity in the validation cohort were calculated using the discriminant thus prepared, and the discriminant performance of the selected polynucleotides was validated using the independent samples (Table 4). For example, the expression level measurement value of the nucleotide sequence represented by SEQ ID NO: 1 was compared between the healthy subjects (47 persons) and the prostate cancer patients (17 persons) in the validation cohort. The results showing that the gene expression level measurement values in the training cohort were significantly lower in the prostate cancer patient group than in the healthy subject group (see the left diagram of FIG. 2), were also reproducible in the validation cohort (see the right diagram of FIG. 2). Likewise, the results obtained about the other polynucleotides shown in SEQ ID NOs: 2 to 152 showed that the gene expression level measurement values were significantly lower (−) or higher (+) in the prostate cancer patient group than in the healthy subject group. These results were able to be validated in the validation cohort. For example, as for this nucleotide sequence represented by SEQ ID NO: 1, the number of samples that were correctly identified in the detection of prostate cancer was calculated using the threshold (6.84) that was set in the training cohort and discriminated between the two groups. As a result, 15 true positives, 44 true negatives, 3 false positive, and 2 false negatives were obtained. From these values, 92.2% accuracy, 88.2% sensitivity, and 93.6% specificity were obtained as detection performance. In this way, the detection performance was calculated as to all of the polynucleotides shown in SEQ ID NOs: 1 to 152, and described in Table 4.

Among the polynucleotides consisting of the nucleotide sequences represented by SEQ ID NOs: 1 to 152 shown in Table 3, for example, 141 polynucleotides consisting of the nucleotide sequences represented by SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 92, 93, 94, 95, 96, 97, 98, 99, 100, 101, 103, 104, 105, 106, 107, 108, 109, 110, 111, 112, 113, 114, 116, 119, 120, 121, 123, 124, 126, 127, 128, 131, 132, 135, 136, 137, 138, 139, 140, 141, 142, 143, 144, 145, 146, 147, 148, 149, 150, 151 and 152 exhibited sensitivity of 88.2%, 94.1%, 76.5%, 88.2%, 88.2%, 94.1%, 76.5%, 64.7%, 88.2%, 76.5%, 64.7%, 82.4%, 70.6%, 88.2%, 52.9%, 47.1%, 70.6%, 94.1%, 70.6%, 76.5%, 76.5%, 70.6%, 70.6%, 29.4%, 58.8%, 88.2%, 58.8%, 76.5%, 64.7%, 76.5%, 64.7%, 47.1%, 76.5%, 82.4%, 70.6%, 47.1%, 64.7%, 58.8%, 52.9%, 82.4%, 64.7%, 70.6%, 64.7%, 70.6%, 70.6%, 76.5%, 58.8%, 58.8%, 52.9%, 64.7%, 47.1%, 41.2%, 70.6%, 52.9%, 29.4%, 35.3%, 41.2%, 58.8%, 52.9%, 41.2%, 70.6%, 52.9%, 35.3%, 64.7%, 29.4%, 70.6%, 70.6%, 76.5%, 58.8%, 70.6%, 35.3%, 58.8%, 58.8%, 47.1%, 70.6%, 76.5%, 58.8%, 82.4%, 23.5%, 52.9%, 41.2%, 47.1%, 64.7%, 41.2%, 41.2%, 35.3%, 47.1%, 47.1%, 41.2%, 29.4%, 41.2%, 64.7%, 35.3%, 70.6%, 29.4%, 47.1%, 29.4%, 52.9%, 64.7%, 47.1%, 23.5%, 35.3%, 47.1%, 35.3%, 35.3%, 52.9%, 23.5%, 35.3%, 47.1%, 52.9%, 23.5%, 23.5%, 29.4%, 52.9%, 41.2%, 23.5%, 23.5%, 41.2%, 47.1%, 29.4%, 58.8%, 29.4%, 23.5%, 29.4%, 58.8%, 88.2%, 76.5%, 58.8%, 52.9%, 47.1%, 35.3%, 52.9%, 29.4%, 47.1%, 76.5%, 58.8%, 29.4%, 29.4%, 29.4%, 41.2% and 23.5% respectively, in the validation cohort (Table 4). Non-Patent Literature 3 has reported that the existing prostate cancer marker PSA has general sensitivity of 20.5%. These results were able to demonstrate that, for example, the 141 polynucleotides consisting of the nucleotide sequences represented by SEQ ID Nos: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 92, 93, 94, 95, 96, 97, 98, 99, 100, 101, 103, 104, 105, 106, 107, 108, 109, 110, 111, 112, 113, 114, 116, 119, 120, 121, 123, 124, 126, 127, 128, 131, 132, 135, 136, 137, 138, 139, 140, 141, 142, 143, 144, 145, 146, 147, 148, 149, 150, 151 and 152 can discriminate, each alone, prostate cancer in the validation cohort with sensitivity beyond PSA.

### Example 2

<Method for Evaluating Prostate Cancer Discriminant Performance with Combination of Multiple Gene Markers Using Samples in the Validation Cohort>

In this Example, a method for evaluating prostate cancer discriminant performance with combination of the gene markers selected in Example 1 was studied.

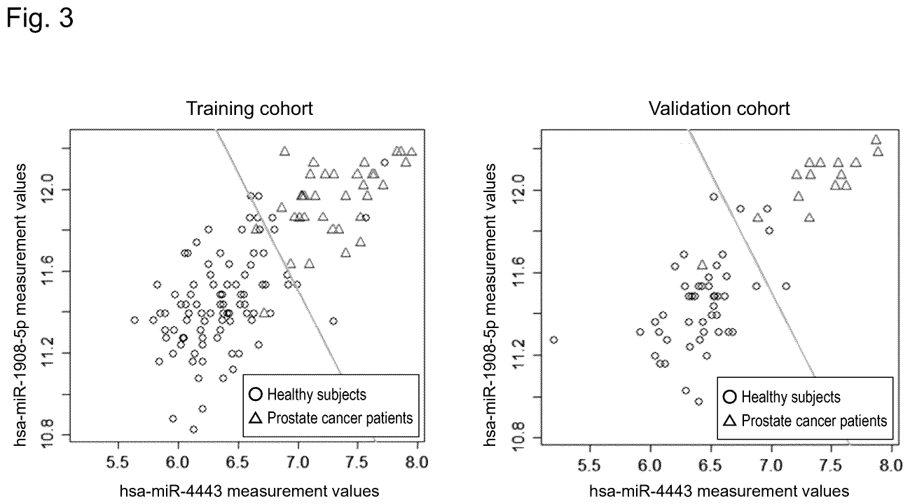

Specifically, Fisher's linear discriminant analysis was conducted as to 11,340 combinations of two expression level measurement values comprising at least one or more of the expression level measurement values of the newly found polynucleotides consisting of the nucleotide sequences represented by SEQ ID NOs: 1 to 135 among the polynucleotides consisting of the nucleotide sequences represented by SEQ ID NOs: 1 to 152 selected in Example 1, to construct a discriminant for determining the presence or absence of prostate cancer. Next, accuracy, sensitivity, and specificity in the validation cohort were calculated using the discriminant thus prepared, and the discriminant performance of the selected polynucleotides was validated using the independent samples. For example, the expression level measurement values of the nucleotide sequences represented by SEQ ID NO: 1 and SEQ ID NO: 2 were compared between the healthy subjects (47 persons) and the prostate cancer patients (17 persons) in the validation cohort. As a result, a scatter diagram that significantly separated the gene expression level measurement values of the prostate cancer patient group from those of the healthy subject group was obtained in the training cohort (see the left diagram of FIG. 3). These results were also reproducible for the validation cohort (see the right diagram of FIG. 3). Likewise, a scatter diagram that significantly separated the gene expression level measurement values of the prostate cancer patient group from those of the healthy subject group was also obtained as to the other combinations of two expression level measurement values comprising at least one or more of the expression level measurement values of the newly found polynucleotides consisting of the nucleotide sequences represented by SEQ ID NOs: 1 to 135 among the polynucleotides consisting of the nucleotide sequences represented by SEQ ID NOs: 1 to 152. These results were able to be validated in the validation cohort. For example, as for these nucleotide sequences represented by SEQ ID NO: 1 and SEQ ID NO: 2, the number of samples correctly identified in the detection of prostate cancer was calculated using the threshold (0=1.15x+y+19.53) that was set in the training cohort and discriminated between the two groups. As a result, 16 true positives, 45 true negatives, 2 false positive, and 1 false negatives were obtained. From these values, 95.3% accuracy, 94.1% sensitivity, and 95.7% specificity were obtained as detection performance.

In this way, the detection performance was calculated as to all combinations (11,340 combinations) of two expression level measurement values comprising at least one or more of the expression level measurement values of the newly found polynucleotides consisting of the nucleotide sequences represented by SEQ ID NOs: 1 to 135 among the polynucleotides consisting of the nucleotide sequences represented by SEQ ID NOs: 1 to 152. Among them, 151 combinations comprising the expression level measurement value of the polynucleotide consisting of the nucleotide sequence represented by SEQ ID NO: 1 and their detection performance are described in Table 6 as an example. For example, the combinations of the expression level measurement values of the polynucleotides consisting of the nucleotide sequences represented by SEQ ID NOs: 1 and 2, SEQ ID NOs: 1 and 3, SEQ ID NOs: 1 and 4, and SEQ ID NOs: 1 and 5 exhibited sensitivity of 94.1%, 88.2%, 88.2%, and 94.1%, respectively, in the validation cohort (Table 6). In this way, 11,326 combinations of two expression level measurement values of the polynucleotides having sensitivity beyond the existing prostate cancer marker PSA (general sensitivity: 20.5%) were obtained in the validation cohort. All of the polynucleotides represented by the nucleotide sequences 1 to 152 described in Table 3 obtained in Example 1 were employed at least once in these combinations. These results were able to demonstrate that the combinations of two expression level measurement values comprising at least one or more of the expression level measurement values of the newly found polynucleotides consisting of the nucleotide sequences represented by SEQ ID NOs: 1 to 135 among the polynucleotides consisting of the nucleotide sequences represented by SEQ ID NOs: 1 to 152 has the performance of detecting prostate cancer with sensitivity beyond PSA.

Thus, markers capable of detecting prostate cancer with excellent sensitivity are obtained even if 3, 4, 5, 6, 7, 8, 9, 10 or more of the expression level measurement values of the polynucleotides consisting of the nucleotide sequences represented by SEQ ID NOs: 1 to 152 are combined. For example, the polynucleotides consisting of the nucleotide sequences represented by SEQ ID NOs: 1 to 135 newly found in Example 1 were ranked in the descending order of their P values which indicates statistical significance, and prostate cancer detection sensitivity was evaluated using combinations of one or more polynucleotides to which the polynucleotides (miRNAs) were added one by one from the top to the bottom of the rank accordingly. In short, the order to combine the polynucleotides (miRNAs) in this evaluation is in reverse in terms of SEQ ID NOs, such as SEQ ID NO: 135 to SEQ ID NOs: 134, 133, . . . , shown in Table 3. As a result, the sensitivity in the validation cohort was 29.4% for 1 polynucleotide, 47.1% for 2 polynucleotides, 76.5% for 3 polynucleotides, 82.4% for 5 polynucleotides, 82.4% for 10 polynucleotides, 88.2% for 20 polynucleotides, 100% for 50 polynucleotides, and 100% for 100 polynucleotides. These values of the sensitivity were higher than the general sensitivity (20.5%) of the existing prostate cancer marker PSA, demonstrating that even combinations of multiple (i.e., two or more) miRNAs can serve as excellent markers for the detection of prostate cancer. In this context, the combinations of multiple miRNAs are not limited to the combinations of the miRNAs added in the order of the statistically significant difference as described above, and any combination of multiple polynucleotides (miRNAs) can be used in the detection of prostate cancer.

From these results, it can be concluded that all of the polynucleotides consisting of the nucleotide sequences represented by SEQ ID NOs: 1 to 152 serve as excellent diagnostic markers.

TABLE 2-1
Training cohort

| Sample name | Cancer stage |
|---|---|
| PR04 | II |
| PR06 | IV |
| PR08 | II |
| PR09 | II |
| PR12 | II |
| PR19 | II |
| PR21 | II |
| PR22 | II |
| PR23 | II |
| PR29 | II |
| PR30 | II |
| PR32 | III |
| PR46 | II |
| PR48 | II |
| PR51 | II |
| PR52 | II |
| PR53 | II |
| PR64 | II |
| PR65 | II |
| PR66 | II |
| PR69 | IV |
| PR73 | II |
| PR75 | II |
| PR80 | IV |
| PR81 | II |
| PR83 | II |
| PR84 | II |
| PR85 | II |
| PR87 | II |
| PR90 | II |
| PR93 | II |
| PR94 | II |
| PR97 | II |
| PR99 | IV |
| PR101 | II |

TABLE 2-2
Validation cohort

| Sample name | Cancer stage |
|---|---|
| PR01 | II |
| PR17 | II |
| PR26 | III |
| PR27 | II |
| PR28 | III |
| PR33 | II |
| PR40 | II |
| PR45 | II |
| PR59 | II |
| PR62 | II |
| PR67 | II |
| PR71 | II |
| PR82 | II |
| PR91 | II |
| PR92 | II |
| PR96 | II |
| PR100 | II |

TABLE 3

| | | | Expression |
|---|---|---|---|
| | | | level in |
| | | | prostate cancer |
| | | | patient |
| | | P value after | relative to |
| SEQ ID NO: | Gene name | Bonferroni correction | healthy subject |
| 1 | hsa-miR-4443 | 2.10E−23 | + |
| 2 | hsa-miR-1908-5p | 7.83E−18 | + |
| 3 | hsa-miR-4257 | 2.21E−17 | − |
| 4 | hsa-miR-3197 | 5.78E−17 | + |
| 5 | hsa-miR-3188 | 5.96.E−17 | + |
| 6 | hsa-miR-4649-5p | 6.34.E−17 | − |
| 7 | hsa-miR-1343-3p | 2.48.E−16 | − |
| 8 | hsa-miR-6861-5p | 1.15.E−15 | − |
| 9 | hsa-miR-1343-5p | 3.73.E−15 | + |
| 10 | hsa-miR-642b-3p | 3.94.E−15 | − |
| 11 | hsa-miR-6741-5p | 3.03.E−14 | − |
| 12 | hsa-miR-4745-5p | 4.76.E−14 | − |
| 13 | hsa-miR-6826-5p | 1.27.E−13 | − |
| 14 | hsa-miR-3663-3p | 1.61.E−13 | − |
| 15 | hsa-miR-3131 | 5.67.E−13 | − |
| 16 | hsa-miR-92a-2-5p | 1.04.E−12 | + |
| 17 | hsa-miR-4258 | 1.59.E−12 | − |
| 18 | hsa-miR-4448 | 2.54.E−12 | + |
| 19 | hsa-miR-6125 | 4.39.E−12 | + |
| 20 | hsa-miR-6880-5p | 6.24.E−12 | + |
| 21 | hsa-miR-6132 | 8.70.E−12 | + |
| 22 | hsa-miR-4467 | 1.45.E−11 | + |
| 23 | hsa-miR-6749-5p | 1.46.E−11 | − |
| 24 | hsa-miR-2392 | 1.68.E−11 | + |
| 25 | hsa-miR-1273g-3p | 2.09.E−11 | − |
| 26 | hsa-miR-4746-3p | 2.43.E−11 | + |
| 27 | hsa-miR-1914-3p | 2.94.E−11 | − |
| 28 | hsa-miR-7845-5p | 3.03.E−11 | + |
| 29 | hsa-miR-6726-5p | 5.00.E−11 | − |
| 30 | hsa-miR-128-2-5p | 5.60.E−11 | − |
| 31 | hsa-miR-4651 | 6.14.E−11 | − |
| 32 | hsa-miR-6765-3p | 6.43.E−11 | − |
| 33 | hsa-miR-3185 | 7.07.E−11 | + |
| 34 | hsa-miR-4792 | 7.39.E−11 | + |
| 35 | hsa-miR-6887-5p | 9.57.E−11 | − |
| 36 | hsa-miR-5572 | 1.01.E−10 | + |
| 37 | hsa-miR-3619-3p | 1.89.E−10 | − |
| 38 | hsa-miR-6780b-5p | 2.55.E−10 | + |
| 39 | hsa-miR-4707-5p | 2.83.E−10 | + |
| 40 | hsa-miR-8063 | 2.93.E−10 | − |
| 41 | hsa-miR-4454 | 3.34.E−10 | − |
| 42 | hsa-miR-4525 | 3.73.E−10 | − |
| 43 | hsa-miR-7975 | 3.87.E−10 | − |
| 44 | hsa-miR-744-5p | 4.00.E−10 | + |
| 45 | hsa-miR-3135b | 4.73.E−10 | − |
| 46 | hsa-miR-4648 | 5.10.E−10 | + |
| 47 | hsa-miR-6816-5p | 6.76.E−10 | + |
| 48 | hsa-miR-4741 | 9.16.E−10 | + |
| 49 | hsa-miR-7150 | 1.34.E−09 | + |
| 50 | hsa-miR-6791-5p | 2.31.E−09 | + |
| 51 | hsa-miR-1247-3p | 3.07.E−09 | + |
| 52 | hsa-miR-7977 | 3.35.E−09 | − |
| 53 | hsa-miR-4497 | 4.19.E−09 | − |
| 54 | hsa-miR-6090 | 5.36.E−09 | + |
| 55 | hsa-miR-6781-5p | 8.00.E−09 | + |
| 56 | hsa-miR-6870-5p | 1.48.E−08 | + |
| 57 | hsa-miR-6729-5p | 1.56.E−08 | + |
| 58 | hsa-miR-4530 | 2.60.E−08 | + |
| 59 | hsa-miR-7847-3p | 3.09.E−08 | − |
| 60 | hsa-miR-6825-5p | 3.86.E−08 | + |
| 61 | hsa-miR-4674 | 3.88.E−08 | − |
| 62 | hsa-miR-3917 | 4.11.E−08 | − |
| 63 | hsa-miR-4707-3p | 4.52.E−08 | + |
| 64 | hsa-miR-6885-5p | 5.06.E−08 | − |
| 65 | hsa-miR-6722-3p | 5.76.E−08 | + |
| 66 | hsa-miR-4516 | 6.32.E−08 | − |
| 67 | hsa-miR-6757-5p | 6.81.E−08 | − |
| 68 | hsa-miR-6840-3p | 1.30.E−07 | − |
| 69 | hsa-miR-5195-3p | 1.45.E−07 | − |
| 70 | hsa-miR-6756-5p | 1.48.E−07 | − |
| 71 | hsa-miR-6800-5p | 1.61.E−07 | + |
| 72 | hsa-miR-6727-5p | 1.65.E−07 | − |
| 73 | hsa-miR-6126 | 1.87.E−07 | + |
| 74 | hsa-miR-6872-3p | 2.21.E−07 | − |
| 75 | hsa-miR-4446-3p | 3.28.E−07 | − |
| 76 | hsa-miR-1268a | 4.54.E−07 | + |
| 77 | hsa-miR-1908-3p | 5.41.E−07 | − |
| 78 | hsa-miR-3679-5p | 5.53.E−07 | + |
| 79 | hsa-miR-4534 | 7.45.E−07 | + |
| 80 | hsa-miR-4675 | 7.91.E−07 | − |
| 81 | hsa-miR-7108-5p | 1.01.E−06 | + |
| 82 | hsa-miR-6799-5p | 1.57.E−06 | + |
| 83 | hsa-miR-4695-5p | 3.59.E−06 | + |
| 84 | hsa-miR-3178 | 4.54.E−06 | + |
| 85 | hsa-miR-5090 | 4.93.E−06 | − |
| 86 | hsa-miR-3180 | 6.40.E−06 | + |
| 87 | hsa-miR-1237-5p | 9.54.E−06 | + |
| 88 | hsa-miR-4758-5p | 1.50.E−05 | − |
| 89 | hsa-miR-3184-5p | 1.60.E−05 | + |
| 90 | hsa-miR-4286 | 1.96.E−05 | − |
| 91 | hsa-miR-6784-5p | 2.81.E−05 | + |
| 92 | hsa-miR-6768-5p | 3.47.E−05 | + |
| 93 | hsa-miR-6785-5p | 3.51.E−05 | − |
| 94 | hsa-miR-4706 | 3.72.E−05 | − |
| 95 | hsa-miR-711 | 4.59.E−05 | + |
| 96 | hsa-miR-1260a | 5.06.E−05 | − |
| 97 | hsa-miR-6746-5p | 5.35.E−05 | − |
| 98 | hsa-miR-6089 | 7.26.E−05 | + |
| 99 | hsa-miR-6821-5p | 7.94.E−05 | + |
| 100 | hsa-miR-4667-5p | 8.38.E−05 | + |
| 101 | hsa-miR-8069 | 9.70.E−05 | + |
| 102 | hsa-miR-4726-5p | 1.11.E−04 | − |
| 103 | hsa-miR-6124 | 1.59.E−04 | + |
| 104 | hsa-miR-4532 | 1.87.E−04 | − |
| 105 | hsa-miR-4486 | 1.92.E−04 | + |
| 106 | hsa-miR-4728-5p | 1.96.E−04 | − |
| 107 | hsa-miR-4508 | 2.20.E−04 | + |
| 108 | hsa-miR-128-1-5p | 3.56.E−04 | + |
| 109 | hsa-miR-4513 | 3.75.E−04 | − |
| 110 | hsa-miR-6795-5p | 5.28.E−04 | − |
| 111 | hsa-miR-4689 | 5.85.E−04 | − |
| 112 | hsa-miR-6763-5p | 6.01.E−04 | + |
| 113 | hsa-miR-8072 | 6.56.E−04 | + |
| 114 | hsa-miR-6765-5p | 6.67.E−04 | + |
| 115 | hsa-miR-4419b | 7.40.E−04 | − |
| 116 | hsa-miR-7641 | 8.72.E−04 | − |
| 117 | hsa-miR-3928-3p | 9.57.E−04 | + |
| 118 | hsa-miR-1227-5p | 9.66.E−04 | + |
| 119 | hsa-miR-4492 | 1.12.E−03 | − |
| 120 | hsa-miR-296-3p | 1.39.E−03 | − |
| 121 | hsa-miR-6769a-5p | 1.42.E−03 | − |
| 122 | hsa-miR-6889-5p | 1.46.E−03 | + |
| 123 | hsa-miR-4632-5p | 1.74.E−03 | + |
| 124 | hsa-miR-4505 | 1.94.E−03 | + |
| 125 | hsa-miR-3154 | 1.97.E−03 | + |
| 126 | hsa-miR-3648 | 2.03.E−03 | − |
| 127 | hsa-miR-4442 | 2.15.E−03 | − |
| 128 | hsa-miR-3141 | 3.29.E−03 | + |
| 129 | hsa-miR-7113-3p | 3.29.E−03 | + |
| 130 | hsa-miR-6819-5p | 5.95.E−03 | − |
| 131 | hsa-miR-3195 | 6.78.E−03 | + |
| 132 | hsa-miR-1199-5p | 7.39.E−03 | − |
| 133 | hsa-miR-6738-5p | 8.00.E−03 | − |
| 134 | hsa-miR-4656 | 8.53.E−03 | − |
| 135 | hsa-miR-6820-5p | 9.18.E−03 | + |
| 136 | hsa-miR-615-5p | 1.89.E−11 | − |
| 137 | hsa-miR-486-3p | 4.76.E−11 | − |
| 138 | hsa-miR-1225-3p | 8.87.E−11 | + |
| 139 | hsa-miR-760 | 1.05.E−10 | − |
| 140 | hsa-miR-187-5p | 9.50.E−09 | − |
| 141 | hsa-miR-1203 | 6.86.E−08 | + |
| 142 | hsa-miR-7110-5p | 2.08.E−07 | + |
| 143 | hsa-miR-371a-5p | 4.75.E−07 | − |
| 144 | hsa-miR-939-5p | 9.56.E−07 | + |
| 145 | hsa-miR-575 | 2.41.E−06 | + |
| 146 | hsa-miR-92b-5p | 2.89.E−06 | + |
| 147 | hsa-miR-887-3p | 1.35.E−05 | + |
| 148 | hsa-miR-920 | 3.39.E−05 | − |
| 149 | hsa-miR-1915-5p | 2.55.E−04 | − |
| 150 | hsa-miR-1231 | 3.11.E−04 | + |
| 151 | hsa-miR-663b | 1.18.E−03 | − |
| 152 | hsa-miR-1225-5p | 8.49.E−03 | + |

TABLE 4

| SEQ | Training cohort | | | Validation cohort | | |
|---|---|---|---|---|---|---|
| ID NO: | Accuracy (%) | Sensitivity (%) | Specificity (%) | Accuracy (%) | Sensitivity (%) | Specificity (%) |
| 1 | 93 | 85.7 | 95.7 | 92.2 | 88.2 | 93.6 |
| 2 | 87.6 | 85.7 | 88.3 | 92.2 | 94.1 | 91.5 |
| 3 | 89.9 | 77.1 | 94.7 | 89.1 | 76.5 | 93.6 |
| 4 | 85.3 | 71.4 | 90.4 | 95.3 | 88.2 | 97.9 |
| 5 | 89.1 | 77.1 | 93.6 | 92.2 | 88.2 | 93.6 |
| 6 | 88.4 | 77.1 | 92.6 | 92.2 | 94.1 | 91.5 |
| 7 | 86 | 74.3 | 90.4 | 82.8 | 76.5 | 85.1 |
| 8 | 86.8 | 74.3 | 91.5 | 84.4 | 64.7 | 91.5 |
| 9 | 83.7 | 74.3 | 87.2 | 92.2 | 88.2 | 93.6 |
| 10 | 86.8 | 68.6 | 93.6 | 92.2 | 76.5 | 97.9 |
| 11 | 86 | 68.6 | 92.6 | 85.9 | 64.7 | 93.6 |
| 12 | 86 | 74.3 | 90.4 | 90.6 | 82.4 | 93.6 |
| 13 | 89.1 | 77.1 | 93.6 | 89.1 | 70.6 | 95.7 |
| 14 | 79.8 | 42.9 | 93.6 | 95.3 | 88.2 | 97.9 |
| 15 | 83.7 | 68.6 | 89.4 | 76.6 | 52.9 | 85.1 |
| 16 | 87.6 | 77.1 | 91.5 | 73.4 | 47.1 | 83 |
| 17 | 82.9 | 57.1 | 92.6 | 81.2 | 70.6 | 85.1 |
| 18 | 88.4 | 65.7 | 96.8 | 93.8 | 94.1 | 93.6 |
| 19 | 88.4 | 82.9 | 90.4 | 84.4 | 70.6 | 89.4 |
| 20 | 82.9 | 57.1 | 92.6 | 92.2 | 76.5 | 97.9 |
| 21 | 87.6 | 62.9 | 96.8 | 92.2 | 76.5 | 97.9 |
| 22 | 82.2 | 60 | 90.4 | 89.1 | 70.6 | 95.7 |
| 23 | 82.2 | 60 | 90.4 | 89.1 | 70.6 | 95.7 |
| 24 | 84.5 | 57.1 | 94.7 | 71.9 | 29.4 | 87.2 |
| 25 | 87.6 | 62.9 | 96.8 | 82.8 | 58.8 | 91.5 |
| 26 | 84.5 | 65.7 | 91.5 | 93.8 | 88.2 | 95.7 |
| 27 | 82.2 | 65.7 | 88.3 | 76.6 | 58.8 | 83 |
| 28 | 81.4 | 57.1 | 90.4 | 89.1 | 76.5 | 93.6 |
| 29 | 87.6 | 68.6 | 94.7 | 85.9 | 64.7 | 93.6 |
| 30 | 85.3 | 60 | 94.7 | 87.5 | 76.5 | 91.5 |
| 31 | 82.2 | 57.1 | 91.5 | 82.8 | 64.7 | 89.4 |
| 32 | 84.5 | 60 | 93.6 | 79.7 | 47.1 | 91.5 |
| 33 | 83.7 | 65.7 | 90.4 | 90.6 | 76.5 | 95.7 |
| 34 | 89.9 | 74.3 | 95.7 | 87.5 | 82.4 | 89.4 |
| 35 | 81.4 | 57.1 | 90.4 | 85.9 | 70.6 | 91.5 |
| 36 | 79.8 | 57.1 | 88.3 | 78.1 | 47.1 | 89.4 |
| 37 | 84.5 | 60 | 93.6 | 87.5 | 64.7 | 95.7 |
| 38 | 81.4 | 54.3 | 91.5 | 82.8 | 58.8 | 91.5 |
| 39 | 79.1 | 54.3 | 88.3 | 87.5 | 52.9 | 100 |
| 40 | 83.7 | 74.3 | 87.2 | 90.6 | 82.4 | 93.6 |
| 41 | 85.3 | 60 | 94.7 | 79.7 | 64.7 | 85.1 |
| 42 | 79.1 | 48.6 | 90.4 | 89.1 | 70.6 | 95.7 |
| 43 | 81.4 | 54.3 | 91.5 | 85.9 | 64.7 | 93.6 |
| 44 | 85.3 | 54.3 | 96.8 | 90.6 | 70.6 | 97.9 |
| 45 | 89.9 | 74.3 | 95.7 | 89.1 | 70.6 | 95.7 |
| 46 | 82.9 | 60 | 91.5 | 84.4 | 76.5 | 87.2 |
| 47 | 83.7 | 60 | 92.6 | 79.7 | 58.8 | 87.2 |
| 48 | 82.9 | 54.3 | 93.6 | 82.8 | 58.8 | 91.5 |
| 49 | 84.5 | 57.1 | 94.7 | 79.7 | 52.9 | 89.4 |
| 50 | 78.3 | 48.6 | 89.4 | 87.5 | 64.7 | 95.7 |
| 51 | 82.2 | 48.6 | 94.7 | 75 | 47.1 | 85.1 |
| 52 | 80.6 | 51.4 | 91.5 | 79.7 | 41.2 | 93.6 |
| 53 | 79.8 | 60 | 87.2 | 85.9 | 70.6 | 91.5 |
| 54 | 80.6 | 42.9 | 94.7 | 87.5 | 52.9 | 100 |
| 55 | 82.9 | 57.1 | 92.6 | 76.6 | 29.4 | 93.6 |
| 56 | 80.6 | 54.3 | 90.4 | 76.6 | 35.3 | 91.5 |
| 57 | 85.3 | 51.4 | 97.9 | 81.2 | 41.2 | 95.7 |
| 58 | 80.6 | 54.3 | 90.4 | 81.2 | 58.8 | 89.4 |
| 59 | 79.1 | 51.4 | 89.4 | 81.2 | 52.9 | 91.5 |
| 60 | 78.3 | 51.4 | 88.3 | 76.6 | 41.2 | 89.4 |
| 61 | 81.4 | 48.6 | 93.6 | 85.9 | 70.6 | 91.5 |
| 62 | 82.9 | 54.3 | 93.6 | 82.8 | 52.9 | 93.6 |
| 63 | 82.9 | 60 | 91.5 | 78.1 | 35.3 | 93.6 |
| 64 | 80.6 | 42.9 | 94.7 | 89.1 | 64.7 | 97.9 |
| 65 | 78.3 | 40 | 92.6 | 79.7 | 29.4 | 97.9 |
| 66 | 80.6 | 45.7 | 93.6 | 84.4 | 70.6 | 89.4 |
| 67 | 80.6 | 57.1 | 89.4 | 84.4 | 70.6 | 89.4 |
| 68 | 79.1 | 42.9 | 92.6 | 85.9 | 76.5 | 89.4 |
| 69 | 82.2 | 48.6 | 94.7 | 81.2 | 58.8 | 89.4 |
| 70 | 79.8 | 51.4 | 90.4 | 92.2 | 70.6 | 100 |
| 71 | 79.8 | 45.7 | 92.6 | 79.7 | 35.3 | 95.7 |
| 72 | 79.8 | 51.4 | 90.4 | 78.1 | 58.8 | 85.1 |
| 73 | 77.5 | 42.9 | 90.4 | 81.2 | 58.8 | 89.4 |
| 74 | 81.4 | 51.4 | 92.6 | 73.4 | 47.1 | 83 |
| 75 | 79.1 | 54.3 | 88.3 | 82.8 | 70.6 | 87.2 |
| 76 | 76 | 42.9 | 88.3 | 84.4 | 76.5 | 87.2 |
| 77 | 78.3 | 51.4 | 88.3 | 79.7 | 58.8 | 87.2 |
| 78 | 80.6 | 51.4 | 91.5 | 92.2 | 82.4 | 95.7 |
| 79 | 78.3 | 42.9 | 91.5 | 68.8 | 23.5 | 85.1 |
| 80 | 79.1 | 40 | 93.6 | 84.4 | 52.9 | 95.7 |
| 81 | 79.1 | 45.7 | 91.5 | 81.2 | 41.2 | 95.7 |
| 82 | 79.1 | 45.7 | 91.5 | 78.1 | 47.1 | 89.4 |
| 83 | 76.7 | 42.9 | 89.4 | 89.1 | 64.7 | 97.9 |
| 84 | 80.6 | 45.7 | 93.6 | 78.1 | 41.2 | 91.5 |
| 85 | 79.8 | 37.1 | 95.7 | 84.4 | 41.2 | 100 |
| 86 | 79.8 | 45.7 | 92.6 | 75 | 35.3 | 89.4 |
| 87 | 78.1 | 32.4 | 94.7 | 85.9 | 47.1 | 100 |
| 88 | 79.1 | 31.4 | 96.8 | 84.4 | 47.1 | 97.9 |
| 89 | 75.2 | 34.3 | 90.4 | 76.6 | 41.2 | 89.4 |
| 90 | 76.7 | 37.1 | 91.5 | 76.6 | 29.4 | 93.6 |
| 91 | 74.4 | 34.3 | 89.4 | 70.3 | 17.6 | 89.4 |
| 92 | 83.7 | 51.4 | 95.7 | 79.7 | 41.2 | 93.6 |
| 93 | 77.5 | 42.9 | 90.4 | 84.4 | 64.7 | 91.5 |
| 94 | 79.8 | 42.9 | 93.6 | 76.6 | 35.3 | 91.5 |
| 95 | 82.2 | 48.6 | 94.7 | 89.1 | 70.6 | 95.7 |
| 96 | 78.3 | 45.7 | 90.4 | 73.4 | 29.4 | 89.4 |
| 97 | 76 | 34.3 | 91.5 | 75 | 47.1 | 85.1 |
| 98 | 74.4 | 25.7 | 92.6 | 76.6 | 29.4 | 93.6 |
| 99 | 78.3 | 42.9 | 91.5 | 85.9 | 52.9 | 97.9 |
| 100 | 73.6 | 22.9 | 92.6 | 87.5 | 64.7 | 95.7 |
| 101 | 79.8 | 45.7 | 92.6 | 84.4 | 47.1 | 97.9 |
| 102 | 76 | 37.1 | 90.4 | 67.2 | 17.6 | 85.1 |
| 103 | 79.1 | 31.4 | 96.8 | 76.6 | 23.5 | 95.7 |
| 104 | 77.5 | 28.6 | 95.7 | 81.2 | 35.3 | 97.9 |
| 105 | 77.5 | 34.3 | 93.6 | 81.2 | 47.1 | 93.6 |
| 106 | 73.6 | 31.4 | 89.4 | 79.7 | 35.3 | 95.7 |
| 107 | 77.5 | 25.7 | 96.8 | 75 | 35.3 | 89.4 |
| 108 | 76 | 34.3 | 91.5 | 84.4 | 52.9 | 95.7 |
| 109 | 76.7 | 34.3 | 92.6 | 75 | 23.5 | 93.6 |
| 110 | 76 | 22.9 | 95.7 | 78.1 | 35.3 | 93.6 |
| 111 | 75.2 | 14.3 | 97.9 | 84.4 | 47.1 | 97.9 |
| 112 | 72.9 | 20 | 92.6 | 85.9 | 52.9 | 97.9 |
| 113 | 75.2 | 22.9 | 94.7 | 78.1 | 23.5 | 97.9 |
| 114 | 73.6 | 17.1 | 94.7 | 76.6 | 23.5 | 95.7 |
| 115 | 76.7 | 28.6 | 94.7 | 73.4 | 17.6 | 93.6 |
| 116 | 73.6 | 28.6 | 90.4 | 75 | 29.4 | 91.5 |
| 117 | 79.1 | 34.3 | 95.7 | 75 | 17.6 | 95.7 |
| 118 | 74.4 | 22.9 | 93.6 | 71.9 | 17.6 | 91.5 |
| 119 | 73.6 | 22.9 | 92.6 | 85.9 | 52.9 | 97.9 |
| 120 | 73.6 | 25.7 | 91.5 | 79.7 | 41.2 | 93.6 |
| 121 | 77.5 | 34.3 | 93.6 | 75 | 23.5 | 93.6 |
| 122 | 74.4 | 25.7 | 92.6 | 70.3 | 11.8 | 91.5 |
| 123 | 76.7 | 25.7 | 95.7 | 78.1 | 23.5 | 97.9 |
| 124 | 81.4 | 40 | 96.8 | 84.4 | 41.2 | 100 |
| 125 | 74.4 | 20 | 94.7 | 71.9 | 17.6 | 91.5 |
| 126 | 76.7 | 28.6 | 94.7 | 82.8 | 47.1 | 95.7 |
| 127 | 75.2 | 31.4 | 91.5 | 76.6 | 29.4 | 93.6 |
| 128 | 76.7 | 22.9 | 96.8 | 87.5 | 58.8 | 97.9 |
| 129 | 69.8 | 22.9 | 87.2 | 73.4 | 11.8 | 95.7 |
| 130 | 74.4 | 22.9 | 93.6 | 75 | 11.8 | 97.9 |
| 131 | 74.4 | 28.6 | 91.5 | 81.2 | 29.4 | 100 |
| 132 | 74.4 | 22.9 | 93.6 | 75 | 23.5 | 93.6 |
| 133 | 68.2 | 11.4 | 89.4 | 75 | 11.8 | 97.9 |
| 134 | 75.2 | 20 | 95.7 | 76.6 | 17.6 | 97.9 |
| 135 | 73.6 | 22.9 | 92.6 | 78.1 | 29.4 | 95.7 |
| 136 | 86.8 | 65.7 | 94.7 | 75 | 58.8 | 80.9 |
| 137 | 86 | 68.6 | 92.6 | 93.8 | 88.2 | 95.7 |
| 138 | 86.8 | 68.6 | 93.6 | 85.9 | 76.5 | 89.4 |
| 139 | 79.8 | 54.3 | 89.4 | 81.2 | 58.8 | 89.4 |
| 140 | 80.6 | 51.4 | 91.5 | 73.4 | 52.9 | 80.9 |
| 141 | 80.6 | 45.7 | 93.6 | 84.4 | 47.1 | 97.9 |
| 142 | 76 | 48.6 | 86.2 | 78.1 | 35.3 | 93.6 |
| 143 | 79.8 | 42.9 | 93.6 | 79.7 | 52.9 | 89.4 |
| 144 | 72.9 | 42.9 | 84 | 76.6 | 29.4 | 93.6 |
| 145 | 79.8 | 48.6 | 91.5 | 82.8 | 47.1 | 95.7 |
| 146 | 79.1 | 48.6 | 90.4 | 92.2 | 76.5 | 97.9 |
| 147 | 74.4 | 34.3 | 89.4 | 87.5 | 58.8 | 97.9 |
| 148 | 75.2 | 34.3 | 90.4 | 67.2 | 29.4 | 80.9 |
| 149 | 76 | 28.6 | 93.6 | 78.1 | 29.4 | 95.7 |
| 150 | 76 | 28.6 | 93.6 | 78.1 | 29.4 | 95.7 |
| 151 | 79.8 | 34.3 | 96.8 | 81.2 | 41.2 | 95.7 |
| 152 | 72.9 | 14.3 | 94.7 | 78.1 | 23.5 | 97.9 |

TABLE 5

| | Discriminant | |
|---|---|---|
| SEQ ID NO: | coefficient | Constant term |
| 1 | 2.841 | 19.421 |
| 2 | 4.435 | 51.871 |
| 3 | 3.207 | 21.845 |
| 4 | 3.253 | 31.499 |
| 5 | 4.075 | 25.795 |
| 6 | 2.840 | 29.134 |
| 7 | 2.539 | 19.203 |
| 8 | 4.359 | 31.785 |
| 9 | 4.100 | 43.343 |
| 10 | 2.475 | 23.187 |
| 11 | 4.875 | 33.924 |
| 12 | 2.662 | 31.800 |
| 13 | 2.576 | 15.891 |
| 14 | 3.758 | 45.427 |
| 15 | 3.007 | 21.322 |
| 16 | 2.086 | 20.103 |
| 17 | 2.415 | 21.597 |
| 18 | 1.386 | 8.309 |
| 19 | 5.265 | 63.510 |
| 20 | 2.601 | 20.485 |
| 21 | 3.480 | 28.174 |
| 22 | 2.098 | 21.131 |
| 23 | 5.034 | 50.773 |
| 24 | 4.361 | 26.275 |
| 25 | 2.837 | 21.020 |
| 26 | 3.180 | 21.510 |
| 27 | 4.832 | 36.754 |
| 28 | 3.240 | 22.334 |
| 29 | 3.297 | 32.746 |
| 30 | 2.751 | 29.763 |
| 31 | 5.736 | 63.070 |
| 32 | 1.809 | 15.805 |
| 33 | 2.566 | 18.600 |
| 34 | 1.963 | 13.501 |
| 35 | 3.448 | 22.503 |
| 36 | 2.577 | 17.708 |
| 37 | 2.326 | 19.136 |
| 38 | 3.057 | 27.631 |
| 39 | 4.748 | 35.803 |
| 40 | 2.880 | 23.980 |
| 41 | 2.262 | 26.203 |
| 42 | 2.961 | 20.754 |
| 43 | 2.220 | 21.988 |
| 44 | 2.353 | 16.969 |
| 45 | 3.102 | 24.441 |
| 46 | 1.594 | 9.958 |
| 47 | 4.468 | 45.625 |
| 48 | 3.732 | 37.591 |
| 49 | 4.378 | 34.624 |
| 50 | 4.896 | 45.653 |
| 51 | 4.268 | 27.572 |
| 52 | 2.192 | 21.441 |
| 53 | 3.013 | 38.151 |
| 54 | 6.888 | 90.453 |
| 55 | 5.516 | 58.347 |
| 56 | 3.641 | 27.465 |
| 57 | 7.874 | 99.518 |
| 58 | 2.492 | 24.657 |
| 59 | 4.058 | 26.380 |
| 60 | 2.350 | 15.623 |
| 61 | 3.450 | 35.983 |
| 62 | 3.384 | 20.446 |
| 63 | 3.330 | 22.289 |
| 64 | 2.906 | 32.309 |
| 65 | 6.296 | 54.722 |
| 66 | 4.911 | 64.684 |
| 67 | 3.206 | 23.658 |
| 68 | 3.285 | 29.269 |
| 69 | 3.237 | 22.571 |
| 70 | 5.038 | 42.229 |
| 71 | 4.159 | 36.268 |
| 72 | 6.806 | 87.077 |
| 73 | 3.063 | 33.575 |
| 74 | 2.552 | 15.751 |
| 75 | 2.791 | 20.526 |
| 76 | 3.285 | 37.356 |
| 77 | 3.362 | 22.864 |
| 78 | 2.811 | 19.633 |
| 79 | 3.759 | 26.864 |
| 80 | 2.982 | 22.991 |
| 81 | 3.997 | 37.078 |
| 82 | 4.484 | 37.972 |
| 83 | 4.600 | 35.223 |
| 84 | 6.026 | 73.901 |
| 85 | 4.239 | 33.902 |
| 86 | 5.314 | 47.015 |
| 87 | 4.798 | 61.512 |
| 88 | 6.806 | 59.152 |
| 89 | 2.706 | 22.080 |
| 90 | 2.498 | 18.719 |
| 91 | 3.833 | 48.285 |
| 92 | 3.325 | 32.674 |
| 93 | 2.793 | 25.551 |
| 94 | 3.860 | 30.344 |
| 95 | 3.878 | 32.579 |
| 96 | 2.688 | 18.916 |
| 97 | 4.301 | 28.806 |
| 98 | 6.386 | 86.216 |
| 99 | 3.660 | 32.730 |
| 100 | 4.747 | 30.458 |
| 101 | 5.928 | 76.530 |
| 102 | 4.003 | 27.083 |
| 103 | 2.947 | 21.339 |
| 104 | 3.195 | 38.076 |
| 105 | 3.103 | 22.617 |
| 106 | 5.105 | 36.656 |
| 107 | 8.087 | 105.473 |
| 108 | 2.927 | 22.240 |
| 109 | 4.111 | 25.157 |
| 110 | 4.803 | 30.149 |
| 111 | 3.332 | 31.704 |
| 112 | 3.855 | 27.615 |
| 113 | 4.606 | 57.067 |
| 114 | 4.801 | 51.079 |
| 115 | 3.144 | 19.952 |
| 116 | 1.519 | 11.331 |
| 117 | 3.217 | 19.269 |
| 118 | 6.074 | 58.552 |
| 119 | 5.508 | 57.411 |
| 120 | 2.408 | 14.813 |
| 121 | 4.332 | 28.554 |
| 122 | 3.286 | 24.338 |
| 123 | 4.276 | 34.402 |
| 124 | 3.879 | 33.369 |
| 125 | 4.935 | 30.296 |
| 126 | 2.311 | 30.293 |
| 127 | 3.246 | 31.192 |
| 128 | 4.684 | 33.975 |
| 129 | 3.468 | 20.714 |
| 130 | 6.033 | 46.013 |
| 131 | 3.614 | 30.304 |
| 132 | 2.869 | 19.654 |
| 133 | 4.117 | 30.189 |
| 134 | 3.842 | 27.896 |
| 135 | 3.012 | 23.016 |
| 136 | 2.496 | 16.713 |
| 137 | 3.062 | 24.479 |
| 138 | 3.805 | 22.035 |
| 139 | 3.410 | 30.192 |
| 140 | 2.159 | 21.828 |
| 141 | 2.667 | 17.063 |
| 142 | 1.850 | 14.572 |
| 143 | 3.628 | 27.064 |
| 144 | 2.613 | 20.101 |
| 145 | 1.927 | 12.938 |
| 146 | 3.654 | 29.801 |
| 147 | 2.419 | 17.967 |
| 148 | 2.581 | 15.080 |
| 149 | 1.552 | 10.112 |
| 150 | 3.511 | 23.568 |
| 151 | 3.078 | 27.364 |
| 152 | 3.739 | 27.780 |

TABLE 6

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 1_2 | 96.1 | 91.4 | 97.9 | 95.3 | 94.1 | 95.7 |
| 1_3 | 94.6 | 94.3 | 94.7 | 96.9 | 88.2 | 100 |
| 1_4 | 94.6 | 91.4 | 95.7 | 92.2 | 88.2 | 93.6 |
| 1_5 | 93.8 | 88.6 | 95.7 | 95.3 | 94.1 | 95.7 |
| 1_6 | 93 | 88.6 | 94.7 | 92.2 | 88.2 | 93.6 |
| 1_7 | 96.1 | 94.3 | 96.8 | 93.8 | 88.2 | 95.7 |
| 1_8 | 94.6 | 91.4 | 95.7 | 93.8 | 88.2 | 95.7 |
| 1_9 | 93.8 | 88.6 | 95.7 | 93.8 | 88.2 | 95.7 |
| 1_10 | 93.8 | 88.6 | 95.7 | 95.3 | 94.1 | 95.7 |
| 1_11 | 96.1 | 97.1 | 95.7 | 95.3 | 94.1 | 95.7 |
| 1_12 | 94.6 | 94.3 | 94.7 | 96.9 | 94.1 | 97.9 |
| 1_13 | 96.1 | 91.4 | 97.9 | 95.3 | 88.2 | 97.9 |
| 1_14 | 93.8 | 85.7 | 96.8 | 92.2 | 88.2 | 93.6 |
| 1_15 | 94.6 | 91.4 | 95.7 | 95.3 | 88.2 | 97.9 |
| 1_16 | 96.9 | 94.3 | 97.9 | 92.2 | 88.2 | 93.6 |
| 1_17 | 93 | 85.7 | 95.7 | 92.2 | 88.2 | 93.6 |
| 1_18 | 93.8 | 88.6 | 95.7 | 95.3 | 88.2 | 97.9 |
| 1_19 | 93.8 | 88.6 | 95.7 | 92.2 | 88.2 | 93.6 |
| 1_20 | 93 | 85.7 | 95.7 | 95.3 | 94.1 | 95.7 |
| 1_21 | 91.5 | 77.1 | 96.8 | 96.9 | 88.2 | 100 |
| 1_22 | 93 | 85.7 | 95.7 | 93.8 | 94.1 | 93.6 |
| 1_23 | 91.5 | 82.9 | 94.7 | 92.2 | 88.2 | 93.6 |
| 1_24 | 93.8 | 88.6 | 95.7 | 92.2 | 88.2 | 93.6 |
| 1_25 | 93.8 | 88.6 | 95.7 | 95.3 | 94.1 | 95.7 |
| 1_26 | 93.8 | 88.6 | 95.7 | 95.3 | 94.1 | 95.7 |
| 1_27 | 92.2 | 82.9 | 95.7 | 93.8 | 94.1 | 93.6 |
| 1_28 | 93.8 | 88.6 | 95.7 | 93.8 | 88.2 | 95.7 |
| 1_29 | 93 | 88.6 | 94.7 | 95.3 | 94.1 | 95.7 |
| 1_30 | 93 | 85.7 | 95.7 | 93.8 | 88.2 | 95.7 |
| 1_31 | 94.6 | 91.4 | 95.7 | 93.8 | 94.1 | 93.6 |
| 1_32 | 93.8 | 91.4 | 94.7 | 90.6 | 88.2 | 91.5 |
| 1_33 | 94.6 | 91.4 | 95.7 | 95.3 | 94.1 | 95.7 |
| 1_34 | 96.1 | 94.3 | 96.8 | 93.8 | 88.2 | 95.7 |
| 1_35 | 94.6 | 91.4 | 95.7 | 95.3 | 88.2 | 97.9 |
| 1_36 | 93 | 85.7 | 95.7 | 90.6 | 88.2 | 91.5 |
| 1_37 | 93 | 88.6 | 94.7 | 95.3 | 88.2 | 97.9 |
| 1_38 | 93 | 82.9 | 96.8 | 93.8 | 88.2 | 95.7 |
| 1_39 | 92.2 | 82.9 | 95.7 | 95.3 | 94.1 | 95.7 |
| 1_40 | 93 | 85.7 | 95.7 | 92.2 | 88.2 | 93.6 |
| 1_41 | 94.6 | 88.6 | 96.8 | 92.2 | 88.2 | 93.6 |
| 1_42 | 95.3 | 91.4 | 96.8 | 98.4 | 94.1 | 100 |
| 1_43 | 94.6 | 91.4 | 95.7 | 92.2 | 88.2 | 93.6 |
| 1_44 | 93 | 85.7 | 95.7 | 93.8 | 88.2 | 95.7 |
| 1_45 | 94.6 | 88.6 | 96.8 | 92.2 | 88.2 | 93.6 |
| 1_46 | 93 | 85.7 | 95.7 | 93.8 | 88.2 | 95.7 |
| 1_47 | 93 | 85.7 | 95.7 | 92.2 | 88.2 | 93.6 |
| 1_48 | 94.6 | 91.4 | 95.7 | 93.8 | 88.2 | 95.7 |
| 1_49 | 93.8 | 85.7 | 96.8 | 93.8 | 88.2 | 95.7 |
| 1_50 | 95.3 | 91.4 | 96.8 | 93.8 | 94.1 | 93.6 |
| 1_51 | 93.8 | 85.7 | 96.8 | 95.3 | 88.2 | 97.9 |
| 1_52 | 93.8 | 88.6 | 95.7 | 92.2 | 88.2 | 93.6 |
| 1_53 | 92.2 | 85.7 | 94.7 | 93.8 | 94.1 | 93.6 |
| 1_54 | 92.2 | 82.9 | 95.7 | 96.9 | 94.1 | 97.9 |
| 1_55 | 92.2 | 82.9 | 95.7 | 92.2 | 88.2 | 93.6 |
| 1_56 | 92.2 | 85.7 | 94.7 | 92.2 | 88.2 | 93.6 |
| 1_57 | 93 | 85.7 | 95.7 | 96.9 | 94.1 | 97.9 |
| 1_58 | 93 | 85.7 | 95.7 | 93.8 | 88.2 | 95.7 |
| 1_59 | 92.2 | 85.7 | 94.7 | 93.8 | 88.2 | 95.7 |
| 1_60 | 92.2 | 85.7 | 94.7 | 90.6 | 88.2 | 91.5 |
| 1_61 | 93 | 85.7 | 95.7 | 93.8 | 88.2 | 95.7 |
| 1_62 | 93 | 85.7 | 95.7 | 93.8 | 88.2 | 95.7 |
| 1_63 | 93.8 | 91.4 | 94.7 | 93.8 | 88.2 | 95.7 |
| 1_64 | 93.8 | 88.6 | 95.7 | 92.2 | 88.2 | 93.6 |
| 1_65 | 93 | 85.7 | 95.7 | 92.2 | 88.2 | 93.6 |
| 1_66 | 91.5 | 82.9 | 94.7 | 95.3 | 88.2 | 97.9 |
| 1_67 | 92.2 | 85.7 | 94.7 | 93.8 | 88.2 | 95.7 |
| 1_68 | 93 | 85.7 | 95.7 | 92.2 | 88.2 | 93.6 |
| 1_69 | 93 | 82.9 | 96.8 | 93.8 | 88.2 | 95.7 |
| 1_70 | 93.8 | 94.3 | 93.6 | 95.3 | 94.1 | 95.7 |
| 1_71 | 93 | 85.7 | 95.7 | 92.2 | 88.2 | 93.6 |
| 1_72 | 91.5 | 85.7 | 93.6 | 90.6 | 88.2 | 91.5 |
| 1_73 | 93 | 85.7 | 95.7 | 92.2 | 88.2 | 93.6 |
| 1_74 | 94.6 | 88.6 | 96.8 | 92.2 | 88.2 | 93.6 |
| 1_75 | 92.2 | 85.7 | 94.7 | 92.2 | 88.2 | 93.6 |
| 1_76 | 92.2 | 82.9 | 95.7 | 93.8 | 88.2 | 95.7 |
| 1_77 | 93.8 | 88.6 | 95.7 | 92.2 | 88.2 | 93.6 |
| 1_78 | 94.6 | 91.4 | 95.7 | 93.8 | 94.1 | 93.6 |
| 1_79 | 95.3 | 91.4 | 96.8 | 96.9 | 94.1 | 97.9 |
| 1_80 | 93 | 85.7 | 95.7 | 92.2 | 88.2 | 93.6 |
| 1_81 | 93 | 85.7 | 95.7 | 92.2 | 88.2 | 93.6 |
| 1_82 | 93 | 85.7 | 95.7 | 92.2 | 88.2 | 93.6 |
| 1_83 | 93 | 85.7 | 95.7 | 92.2 | 88.2 | 93.6 |
| 1_84 | 94.6 | 91.4 | 95.7 | 95.3 | 94.1 | 95.7 |
| 1_85 | 94.6 | 91.4 | 95.7 | 95.3 | 88.2 | 97.9 |
| 1_86 | 93 | 85.7 | 95.7 | 92.2 | 88.2 | 93.6 |
| 1_87 | 93 | 85.3 | 95.7 | 92.2 | 88.2 | 93.6 |
| 1_88 | 91.5 | 80 | 95.7 | 96.9 | 94.1 | 97.9 |
| 1_89 | 93 | 85.7 | 95.7 | 92.2 | 94.1 | 91.5 |
| 1_90 | 94.6 | 91.4 | 95.7 | 92.2 | 88.2 | 93.6 |
| 1_91 | 92.2 | 82.9 | 95.7 | 93.8 | 88.2 | 95.7 |
| 1_92 | 92.2 | 82.9 | 95.7 | 92.2 | 88.2 | 93.6 |
| 1_93 | 93 | 85.7 | 95.7 | 92.2 | 88.2 | 93.6 |
| 1_94 | 92.2 | 85.7 | 94.7 | 93.8 | 88.2 | 95.7 |
| 1_95 | 95.3 | 91.4 | 96.8 | 93.8 | 88.2 | 95.7 |
| 1_96 | 94.6 | 88.6 | 96.8 | 90.6 | 88.2 | 91.5 |
| 1_97 | 94.6 | 91.4 | 95.7 | 92.2 | 88.2 | 93.6 |
| 1_98 | 92.2 | 82.9 | 95.7 | 93.8 | 88.2 | 95.7 |
| 1_99 | 93.8 | 91.4 | 94.7 | 95.3 | 94.1 | 95.7 |
| 1_100 | 94.6 | 88.6 | 96.8 | 90.6 | 88.2 | 91.5 |
| 1_101 | 93 | 85.7 | 95.7 | 92.2 | 88.2 | 93.6 |
| 1_102 | 94.6 | 88.6 | 96.8 | 95.3 | 94.1 | 95.7 |
| 1_103 | 93 | 85.7 | 95.7 | 93.8 | 88.2 | 95.7 |
| 1_104 | 93 | 82.9 | 96.8 | 95.3 | 88.2 | 97.9 |
| 1_105 | 92.2 | 85.7 | 94.7 | 92.2 | 88.2 | 93.6 |
| 1_106 | 93 | 88.6 | 94.7 | 95.3 | 88.2 | 97.9 |
| 1_107 | 92.2 | 85.7 | 94.7 | 92.2 | 94.1 | 91.5 |
| 1_108 | 93.8 | 88.6 | 95.7 | 92.2 | 88.2 | 93.6 |
| 1_109 | 93.8 | 85.7 | 96.8 | 92.2 | 88.2 | 93.6 |
| 1_110 | 93.8 | 88.6 | 95.7 | 95.3 | 94.1 | 95.7 |
| 1_111 | 93 | 85.7 | 95.7 | 93.8 | 88.2 | 95.7 |
| 1_112 | 91.5 | 82.9 | 94.7 | 92.2 | 88.2 | 93.6 |
| 1_113 | 93 | 85.7 | 95.7 | 92.2 | 88.2 | 93.6 |
| 1_114 | 93 | 85.7 | 95.7 | 92.2 | 88.2 | 93.6 |
| 1_115 | 92.2 | 85.7 | 94.7 | 95.3 | 88.2 | 97.9 |
| 1_116 | 93 | 88.6 | 94.7 | 93.8 | 94.1 | 93.6 |
| 1_117 | 93 | 88.6 | 94.7 | 93.8 | 88.2 | 95.7 |
| 1_118 | 93 | 85.7 | 95.7 | 93.8 | 88.2 | 95.7 |
| 1_119 | 93.8 | 85.7 | 96.8 | 93.8 | 94.1 | 93.6 |
| 1_120 | 92.2 | 82.9 | 95.7 | 95.3 | 88.2 | 97.9 |
| 1_121 | 93 | 85.7 | 95.7 | 93.8 | 88.2 | 95.7 |
| 1_122 | 93 | 85.7 | 95.7 | 92.2 | 88.2 | 93.6 |
| 1_123 | 93 | 85.7 | 95.7 | 93.8 | 88.2 | 95.7 |
| 1_124 | 91.5 | 80 | 95.7 | 92.2 | 88.2 | 93.6 |
| 1_125 | 94.6 | 88.6 | 96.8 | 93.8 | 94.1 | 93.6 |
| 1_126 | 93.8 | 85.7 | 96.8 | 92.2 | 88.2 | 93.6 |
| 1_127 | 93.8 | 88.6 | 95.7 | 93.8 | 88.2 | 95.7 |
| 1_128 | 93.8 | 91.4 | 94.7 | 92.2 | 88.2 | 93.6 |
| 1_129 | 93 | 85.7 | 95.7 | 92.2 | 88.2 | 93.6 |
| 1_130 | 93 | 82.9 | 96.8 | 95.3 | 88.2 | 97.9 |
| 1_131 | 90.7 | 82.9 | 93.6 | 93.8 | 88.2 | 95.7 |
| 1_132 | 93 | 85.7 | 95.7 | 92.2 | 88.2 | 93.6 |
| 1_133 | 94.6 | 88.6 | 96.8 | 95.3 | 88.2 | 97.9 |
| 1_134 | 93.8 | 88.6 | 95.7 | 95.3 | 94.1 | 95.7 |
| 1_135 | 93 | 85.7 | 95.7 | 93.8 | 88.2 | 95.7 |
| 1_136 | 96.9 | 97.1 | 96.8 | 93.8 | 94.1 | 93.6 |
| 1_137 | 91.5 | 80 | 95.7 | 92.2 | 88.2 | 93.6 |
| 1_138 | 93.8 | 88.6 | 95.7 | 93.8 | 94.1 | 93.6 |
| 1_139 | 92.2 | 85.7 | 94.7 | 96.9 | 94.1 | 97.9 |
| 1_140 | 94.6 | 88.6 | 96.8 | 92.2 | 88.2 | 93.6 |
| 1_141 | 95.3 | 91.4 | 96.8 | 93.8 | 88.2 | 95.7 |
| 1_142 | 93 | 85.7 | 95.7 | 92.2 | 88.2 | 93.6 |
| 1_143 | 92.2 | 82.9 | 95.7 | 92.2 | 88.2 | 93.6 |
| 1_144 | 93 | 85.7 | 95.7 | 92.2 | 88.2 | 93.6 |
| 1_145 | 93 | 85.7 | 95.7 | 92.2 | 88.2 | 93.6 |
| 1_146 | 93 | 85.7 | 95.7 | 93.8 | 88.2 | 95.7 |
| 1_147 | 93 | 85.7 | 95.7 | 92.2 | 88.2 | 93.6 |
| 1_148 | 93 | 85.7 | 95.7 | 93.8 | 88.2 | 95.7 |
| 1_149 | 92.2 | 82.9 | 95.7 | 93.8 | 88.2 | 95.7 |
| 1_150 | 93.8 | 88.6 | 95.7 | 96.9 | 94.1 | 97.9 |
| 1_151 | 93 | 85.7 | 95.7 | 93.8 | 88.2 | 95.7 |
| 1_152 | 92.2 | 85.7 | 94.7 | 92.2 | 88.2 | 93.6 |

### Example 3

<Selection of Gene Marker Using all Samples and Method for Evaluating Prostate Cancer Discriminant Performance with Acquired Gene Marker>

In this Example, the samples in the training cohort and the validation cohort used in Examples 1 and 2 were integrated, and selection of a gene marker and evaluation of its prostate cancer discriminant performance were conducted using all of the samples.

Specifically, the miRNA expression levels in the serum of the 52 prostate cancer patients and the 141 healthy male subjects obtained in the preceding Reference Examples were normalized by quantile normalization. In order to acquire diagnostic markers with higher reliability, only genes that showed gene expression levels of 2⁶ or higher in 50% or more of the samples in either of the prostate cancer patient group or the healthy subject group were selected in the gene marker selection. In order to further acquire statistical significance for discriminating a prostate cancer patient group from a healthy subject group, the P value obtained by two-tailed t-test assuming equal variance as to each gene expression level was corrected by the Bonferroni method, and genes that satisfied p<0.01 were selected as gene markers for use in explanatory variables of a discriminant. The obtained genes are described in Table 7. In this way, hsa-miR-4763-3p, hsa-miR-3656, hsa-miR-4488, hsa-miR-125a-3p, hsa-miR-1469, hsa-miR-1228-5p, hsa-miR-6798-5p, hsa-miR-1268b, hsa-miR-6732-5p, hsa-miR-1915-3p, hsa-miR-4433b-3p, hsa-miR-1207-5p, hsa-miR-4433-3p, hsa-miR-6879-5p, hsa-miR-4417, hsa-miR-30c-1-3p, hsa-miR-4638-5p, hsa-miR-6088, hsa-miR-4270, hsa-miR-6782-5p, hsa-miR-665, hsa-miR-486-5p, hsa-miR-4655-5p, hsa-miR-1275, hsa-miR-6806-5p, hsa-miR-614, hsa-miR-3937, hsa-miR-6752-5p, hsa-miR-6771-5p, hsa-miR-4450, hsa-miR-211-3p, hsa-miR-663a, hsa-miR-6842-5p, hsa-miR-7114-5p and hsa-miR-6779-5p genes, and the nucleotide sequences of SEQ ID NOs: 153 to 187 related thereto were found in addition to the genes described in Table 3. As with the nucleotide sequences of SEQ ID NOs: 1 to 152, the results obtained about the polynucleotides shown in the nucleotide sequences of SEQ ID NOs: 153 to 187 also showed that the measurement values were significantly lower (−) or higher (+) in the prostate cancer patient group than in the healthy subject group (Table 7). These results were able to be validated in the validation cohort. The presence or absence of prostate cancer in the newly obtained samples can be determined by the methods described in Examples 1 and 2 by using the gene expression level measurement values described in Table 7 either alone or in combination with the gene expression level measurement values described in Table 3.

TABLE 7

| | | | Expression level in |
|---|---|---|---|
| | | P value after | prostate cancer |
| | | Bonferroni | patient with relative |
| SEQ ID NO: | Gene name | correction | to healthy subject |
| 1 | hsa-miR-4443 | 1.11E−37 | + |
| 2 | hsa-miR-1908-5p | 1.13E−31 | + |
| 3 | hsa-miR-4257 | 6.48E−24 | − |
| 4 | hsa-miR-3197 | 1.28E−30 | + |
| 5 | hsa-miR-3188 | 5.67E−27 | + |
| 6 | hsa-miR-4649-5p | 3.70E−27 | − |
| 7 | hsa-miR-1343-3p | 7.09E−23 | − |
| 8 | hsa-miR-6861-5p | 1.80E−24 | − |
| 9 | hsa-miR-1343-5p | 2.82E−24 | + |
| 10 | hsa-miR-642b-3p | 1.07E−27 | − |
| 11 | hsa-miR-6741-5p | 6.82E−22 | − |
| 12 | hsa-miR-4745-5p | 1.13E−23 | − |
| 13 | hsa-miR-6826-5p | 3.61E−19 | − |
| 14 | hsa-miR-3663-3p | 3.08E−23 | − |
| 15 | hsa-miR-3131 | 3.50E−15 | − |
| 16 | hsa-miR-92a-2-5p | 4.74E−16 | + |
| 17 | hsa-miR-4258 | 5.92E−20 | − |
| 18 | hsa-miR-4448 | 7.18E−20 | + |
| 19 | hsa-miR-6125 | 2.60E−19 | + |
| 20 | hsa-miR-6880-5p | 4.86E−19 | + |
| 21 | hsa-miR-6132 | 2.01E−19 | + |
| 22 | hsa-miR-4467 | 7.91E−20 | + |
| 23 | hsa-miR-6749-5p | 1.81E−19 | − |
| 24 | hsa-miR-2392 | 2.70E−11 | + |
| 25 | hsa-miR-1273g-3p | 3.27E−19 | − |
| 26 | hsa-miR-4746-3p | 4.55E−21 | + |
| 27 | hsa-miR-1914-3p | 8.27E−15 | − |
| 28 | hsa-miR-7845-5p | 5.79E−19 | + |
| 29 | hsa-miR-6726-5p | 7.72E−19 | − |
| 30 | hsa-miR-128-2-5p | 5.33E−19 | − |
| 31 | hsa-miR-4651 | 5.90E−18 | − |
| 32 | hsa-miR-6765-3p | 8.39E−16 | − |
| 33 | hsa-miR-3185 | 1.60E−19 | + |
| 34 | hsa-miR-4792 | 1.45E−17 | + |
| 35 | hsa-miR-6887-5p | 1.16E−14 | − |
| 36 | hsa-miR-5572 | 4.90E−16 | + |
| 37 | hsa-miR-3619-3p | 2.51E−16 | − |
| 38 | hsa-miR-6780b-5p | 1.37E−16 | + |
| 39 | hsa-miR-4707-5p | 1.51E−17 | + |
| 40 | hsa-miR-8063 | 5.05E−20 | − |
| 41 | hsa-miR-4454 | 6.07E−14 | − |
| 42 | hsa-miR-4525 | 6.00E−19 | − |
| 43 | hsa-miR-7975 | 6.13E−15 | − |
| 44 | hsa-miR-744-5p | 5.25E−18 | + |
| 45 | hsa-miR-3135b | 1.17E−09 | − |
| 46 | hsa-miR-4648 | 9.53E−17 | + |
| 47 | hsa-miR-6816-5p | 2.60E−15 | + |
| 48 | hsa-miR-4741 | 5.52E−16 | + |
| 49 | hsa-miR-7150 | 2.35E−13 | + |
| 50 | hsa-miR-6791-5p | 6.63E−17 | + |
| 51 | hsa-miR-1247-3p | 6.77E−13 | + |
| 52 | hsa-miR-7977 | 2.22E−14 | − |
| 53 | hsa-miR-4497 | 4.39E−16 | − |
| 54 | hsa-miR-6090 | 4.58E−17 | + |
| 55 | hsa-miR-6781-5p | 1.08E−11 | + |
| 56 | hsa-miR-6870-5p | 4.41E−09 | + |
| 57 | hsa-miR-6729-5p | 6.57E−14 | + |
| 58 | hsa-miR-4530 | 1.48E−10 | + |
| 59 | hsa-miR-7847-3p | 6.31E−12 | − |
| 60 | hsa-miR-6825-5p | 3.31E−12 | + |
| 61 | hsa-miR-4674 | 7.19E−14 | − |
| 62 | hsa-miR-3917 | 1.78E−12 | − |
| 63 | hsa-miR-4707-3p | 6.32E−12 | + |
| 64 | hsa-miR-6885-5p | 1.69E−14 | − |
| 65 | hsa-miR-6722-3p | 1.09E−10 | + |
| 66 | hsa-miR-4516 | 9.57E−15 | − |
| 67 | hsa-miR-6757-5p | 1.02E−11 | − |
| 68 | hsa-miR-6840-3p | 6.73E−14 | − |
| 69 | hsa-miR-5195-3p | 1.21E−11 | − |
| 70 | hsa-miR-6756-5p | 1.46E−15 | − |
| 71 | hsa-miR-6800-5p | 3.18E−11 | + |
| 72 | hsa-miR-6727-5p | 2.88E−09 | − |
| 73 | hsa-miR-6126 | 4.50E−12 | + |
| 74 | hsa-miR-6872-3p | 4.58E−09 | − |
| 75 | hsa-miR-4446-3p | 1.90E−12 | − |
| 76 | hsa-miR-1268a | 1.09E−13 | + |
| 77 | hsa-miR-1908-3p | 2.75E−10 | − |
| 78 | hsa-miR-3679-5p | 4.14E−15 | + |
| 79 | hsa-miR-4534 | 1.65E−06 | + |
| 80 | hsa-miR-4675 | 8.56E−11 | − |
| 81 | hsa-miR-7108-5p | 5.97E−11 | + |
| 82 | hsa-miR-6799-5p | 1.21E−10 | + |
| 83 | hsa-miR-4695-5p | 2.08E−13 | + |
| 84 | hsa-miR-3178 | 1.33E−10 | + |
| 85 | hsa-miR-5090 | 6.85E−11 | − |
| 86 | hsa-miR-3180 | 1.01E−09 | + |
| 87 | hsa-miR-1237-5p | 7.78E−13 | + |
| 88 | hsa-miR-4758-5p | 1.97E−09 | − |
| 89 | hsa-miR-3184-5p | 4.70E−10 | + |
| 90 | hsa-miR-4286 | 2.39E−08 | − |
| 91 | hsa-miR-6784-5p | 1.24E−07 | + |
| 92 | hsa-miR-6768-5p | 2.85E−07 | + |
| 93 | hsa-miR-6785-5p | 2.78E−10 | − |
| 94 | hsa-miR-4706 | 3.20E−06 | − |
| 95 | hsa-miR-711 | 7.50E−11 | + |
| 96 | hsa-miR-1260a | 3.06E−07 | − |
| 97 | hsa-miR-6746-5p | 6.04E−06 | − |
| 98 | hsa-miR-6089 | 1.19E−08 | + |
| 99 | hsa-miR-6821-5p | 4.27E−10 | + |
| 100 | hsa-miR-4667-5p | 9.12E−07 | + |
| 101 | hsa-miR-8069 | 1.81E−09 | + |
| 102 | hsa-miR-4726-5p | 2.71E−05 | − |
| 103 | hsa-miR-6124 | 9.11E−05 | + |
| 104 | hsa-miR-4532 | 2.46E−09 | − |
| 105 | hsa-miR-4486 | 6.30E−09 | + |
| 106 | hsa-miR-4728-5p | 8.48E−09 | − |
| 107 | hsa-miR-4508 | 1.66E−06 | + |
| 108 | hsa-miR-128-1-5p | 2.04E−08 | + |
| 109 | hsa-miR-4513 | 1.44E−06 | − |
| 110 | hsa-miR-6795-5p | 1.12E−06 | − |
| 111 | hsa-miR-4689 | 8.95E−09 | − |
| 112 | hsa-miR-6763-5p | 2.59E−09 | + |
| 113 | hsa-miR-8072 | 1.32E−07 | + |
| 114 | hsa-miR-6765-5p | 4.48E−05 | + |
| 115 | hsa-miR-4419b | 1.22E−04 | − |
| 116 | hsa-miR-7641 | 3.99E−08 | − |
| 117 | hsa-miR-3928-3p | 7.30E−06 | + |
| 118 | hsa-miR-1227-5p | 6.47E−06 | + |
| 119 | hsa-miR-4492 | 3.11E−10 | − |
| 120 | hsa-miR-296-3p | 1.31E−06 | − |
| 121 | hsa-miR-6769a-5p | 2.26E−05 | − |
| 122 | hsa-miR-6889-5p | 5.29E−04 | + |
| 123 | hsa-miR-4632-5p | 3.39E−05 | + |
| 124 | hsa-miR-4505 | 6.21E−06 | + |
| 125 | hsa-miR-3154 | 1.41E−05 | + |
| 126 | hsa-miR-3648 | 2.83E−06 | − |
| 127 | hsa-miR-4442 | 2.03E−07 | − |
| 128 | hsa-miR-3141 | 3.73E−07 | + |
| 129 | hsa-miR-7113-3p | 4.11E−05 | + |
| 130 | hsa-miR-6819-5p | 5.08E−03 | − |
| 131 | hsa-miR-3195 | 1.18E−04 | + |
| 132 | hsa-miR-1199-5p | 8.59E−05 | − |
| 133 | hsa-miR-6738-5p | 2.49E−05 | − |
| 134 | hsa-miR-4656 | 1.45E−05 | − |
| 135 | hsa-miR-6820-5p | 3.40E−04 | + |
| 136 | hsa-miR-615-5p | 1.98E−14 | − |
| 137 | hsa-miR-486-3p | 9.28E−17 | − |
| 138 | hsa-miR-1225-3p | 3.41E−16 | + |
| 139 | hsa-miR-760 | 4.58E−15 | − |
| 140 | hsa-miR-187-5p | 7.21E−11 | − |
| 141 | hsa-miR-1203 | 8.06E−14 | + |
| 142 | hsa-miR-7110-5p | 7.39E−11 | + |
| 143 | hsa-miR-371a-5p | 3.27E−12 | − |
| 144 | hsa-miR-939-5p | 2.77E−11 | + |
| 145 | hsa-miR-575 | 1.85E−10 | + |
| 146 | hsa-miR-92b-5p | 7.45E−16 | + |
| 147 | hsa-miR-887-3p | 3.99E−12 | + |
| 148 | hsa-miR-920 | 1.63E−05 | − |
| 149 | hsa-miR-1915-5p | 1.24E−07 | − |
| 150 | hsa-miR-1231 | 1.35E−07 | + |
| 151 | hsa-miR-663b | 6.03E−07 | − |
| 152 | hsa-miR-1225-5p | 2.89E−06 | + |
| 153 | hsa-miR-4763-3p | 1.50E−07 | + |
| 154 | hsa-miR-3656 | 2.20E−06 | + |
| 155 | hsa-miR-4488 | 3.80E−06 | + |
| 156 | hsa-miR-125a-3p | 8.47E−06 | − |
| 157 | hsa-miR-1469 | 8.73E−06 | + |
| 158 | hsa-miR-1228-5p | 1.34E−05 | + |
| 159 | hsa-miR-6798-5p | 1.73E−05 | + |
| 160 | hsa-miR-1268b | 1.93E−05 | + |
| 161 | hsa-miR-6732-5p | 2.42E−05 | + |
| 162 | hsa-miR-1915-3p | 3.96E−05 | + |
| 163 | hsa-miR-4433b-3p | 4.24E−05 | + |
| 164 | hsa-miR-1207-5p | 4.14E−05 | + |
| 165 | hsa-miR-4433-3p | 4.84E−05 | + |
| 166 | hsa-miR-6879-5p | 5.79E−05 | + |
| 167 | hsa-miR-4417 | 8.44E−05 | + |
| 168 | hsa-miR-30c-1-3p | 8.49E−05 | + |
| 169 | hsa-miR-4638-5p | 7.97E−05 | + |
| 170 | hsa-miR-6088 | 2.07E−04 | − |
| 171 | hsa-miR-4270 | 2.44E−04 | − |
| 172 | hsa-miR-6782-5p | 6.53E−04 | + |
| 173 | hsa-miR-665 | 7.52E−04 | − |
| 174 | hsa-miR-486-5p | 9.25E−04 | + |
| 175 | hsa-miR-4655-5p | 1.04E−03 | + |
| 176 | hsa-miR-1275 | 1.11E−03 | + |
| 177 | hsa-miR-6806-5p | 1.78E−03 | − |
| 178 | hsa-miR-614 | 1.92E−03 | − |
| 179 | hsa-miR-3937 | 2.41E−03 | + |
| 180 | hsa-miR-6752-5p | 2.47E−03 | + |
| 181 | hsa-miR-6771-5p | 3.30E−03 | − |
| 182 | hsa-miR-4450 | 3.79E−03 | + |
| 183 | hsa-miR-211-3p | 6.22E−03 | − |
| 184 | hsa-miR-663a | 5.44E−03 | + |
| 185 | hsa-miR-6842-5p | 8.58E−03 | + |
| 186 | hsa-miR-7114-5p | 8.30E−03 | − |
| 187 | hsa-miR-6779-5p | 8.35E−03 | − |

### Example 4

<Method for Evaluating Prostate Cancer-Specific Discriminant Performance with Combination of Multiple Gene Markers Using Samples in the Validation Cohort>

In this Example, gene expression levels of miRNAs in serum were compared between prostate cancer patients and a control group that consists of healthy subjects and breast cancer patients, in the same way as the method described in Example 1 in the training cohort obtained in Reference Example 2 to select a statistically significant gene for diagnosis. Polynucleotides consisting of the nucleotide sequences represented by SEQ ID NOs: 580 to 611 thus newly selected were each further combined with the gene markers selected in Example 1 to study a method for evaluating prostate cancer-specific discriminant performance.

Specifically, first, the miRNA expression levels in the training cohort and the validation cohort obtained in Reference Example 2 mentioned above were combined and normalized by quantile normalization. Next, Fisher's linear discriminant analysis was conducted as to combinations of 1 to 4 expression level measurement values comprising at least one or more of the expression level measurement values of the polynucleotides consisting of the nucleotide sequences represented by SEQ ID NOs: 1 to 187, 580 to 611, to construct a discriminant for determining the presence or absence of prostate cancer. Next, accuracy, sensitivity, and specificity in the validation cohort obtained in Reference Example 2 were calculated using the discriminant thus prepared, with the prostate cancer patient group as a positive sample group, and the healthy subject group and the breast cancer patient group as a negative sample group. The discriminant performance of the selected polynucleotides was validated using the independent samples.

Most of polynucleotides consisting of the nucleotide sequences represented by these SEQ ID NOs (SEQ ID NOs: 1 to 187, and 580 to 611 corresponding to the miRNA markers of Table 1) or complementary sequences thereof mentioned above were able to provide relatively high accuracy, sensitivity, and specificity in the determination of the presence or absence of prostate cancer, and furthermore, were able to specifically discriminate prostate cancer from the other cancers. For example, among the combinations of multiple polynucleotides selected from the group consisting of polynucleotides consisting of the nucleotide sequences represented by SEQ ID NOs: 1, 3, 4, 5, 6, 7, 9, 10, 12, 14, 15, 16, 17, 18, 20, 24, 29, 35, 37, 42, 51, 55, 58, 61, 63, 64, 67, 70, 72, 79, 82, 89, 91, 97, 98, 101, 103, 104, 112, 113, 114, 116, 119, 126, 135, 136, 139, 140, 141, 145, 147, 154, 155, 156, 158, 169, 173, 175, 178, 182, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610 and 611, or complementary sequences thereof (the cancer type-specific polynucleotide group 1) as polynucleotides capable of specifically binding to target markers, combinations comprising at least one or more polynucleotide(s) selected from the group consisting of polynucleotides of SEQ ID NOs: 1, 12, 16, 37, 42, 63, 119, 126, 139, 173, 178, 599, 609 and 611 (the cancer type-specific polynucleotide group 2) that were included in the cancer type-specific polynucleotide group 1, were able to specifically discriminate prostate cancer from the other cancers with high accuracy.

The number of the aforementioned polynucleotides with cancer type specificity in the combination can be 1, 2, 3, 4, 5, 6, 7, 8, 9, 10 or more for the combination. The combinations of 4 or more of these polynucleotides were able to exhibit discriminant accuracy of 85% or higher.

Specifically, the discriminant accuracy of the measurement using the polynucleotide consisting of the nucleotide sequence represented by SEQ ID NO: 1 or a complementary sequence thereof is shown in Table 8-1. The measurement using the combination of one polynucleotide comprising at least one polynucleotide consisting of the nucleotide sequence represented by SEQ ID NO: 1 or a complementary sequence thereof exhibited accuracy of 94.4% in the training cohort and accuracy of 91.8% in the validation cohort. Also, for example, the measurement using the combination of two polynucleotides comprising at least one polynucleotide consisting of the nucleotide sequence represented by SEQ ID NO: 1 or a complementary sequence thereof exhibited the highest accuracy of 96.4% in the training cohort and the highest accuracy of 90.8% in the validation cohort. Furthermore, for example, the measurement using the combinations of three polynucleotides comprising at least one polynucleotide consisting of the nucleotide sequence represented by SEQ ID NO: 1 or a complementary sequence thereof exhibited the highest accuracy of 98.5% in the training cohort and the highest accuracy of 92.9% in the validation cohort. Furthermore, for example, the measurement using the combinations of four polynucleotides comprising at least one polynucleotide consisting of the nucleotide sequence represented by SEQ ID NO: 1 or a complementary sequence thereof exhibited the highest accuracy of 95.4% in the training cohort and the highest accuracy of 92.9% in the validation cohort.

The discriminant accuracy of the measurement using the polynucleotide consisting of the nucleotide sequence represented by SEQ ID NO: 12 or a complementary sequence thereof is shown in Table 8-2. The measurement using the combination of one polynucleotide comprising at least one polynucleotide consisting of the nucleotide sequence represented by SEQ ID NO: 12 or a complementary sequence thereof exhibited accuracy of 65.5% in the training cohort and accuracy of 56.1% in the validation cohort. Also, for example, the measurement using the combination of two polynucleotides comprising at least one polynucleotide consisting of the nucleotide sequence represented by SEQ ID NO: 12 or a complementary sequence thereof exhibited the highest accuracy of 94.9% in the training cohort and the highest accuracy of 91.8% in the validation cohort. Furthermore, for example, the measurement using the combinations of three polynucleotides comprising at least one polynucleotide consisting of the nucleotide sequence represented by SEQ ID NO: 12 or a complementary sequence thereof exhibited the highest accuracy of 98.0% in the training cohort and the highest accuracy of 93.9% in the validation cohort. Furthermore, for example, the measurement using the combinations of four polynucleotides comprising at least one polynucleotide consisting of the nucleotide sequence represented by SEQ ID NO: 12 or a complementary sequence thereof exhibited the highest accuracy of 98.5% in the training cohort and the highest accuracy of 94.9% in the validation cohort.

The discriminant accuracy of the measurement using the polynucleotide consisting of the nucleotide sequence represented by SEQ ID NO: 16 or a complementary sequence thereof is shown in Table 8-3. The measurement using the combination of one polynucleotide comprising at least one polynucleotide consisting of the nucleotide sequence represented by SEQ ID NO: 16 or a complementary sequence thereof exhibited accuracy of 71.6% in the training cohort and accuracy of 74.5% in the validation cohort. Also, for example, the measurement using the combination of two polynucleotides comprising at least one polynucleotide consisting of the nucleotide sequence represented by SEQ ID NO: 16 or a complementary sequence thereof exhibited the highest accuracy of 95.4% in the training cohort and the highest accuracy of 93.9% in the validation cohort. Furthermore, for example, the measurement using the combinations of three polynucleotides comprising at least one polynucleotide consisting of the nucleotide sequence represented by SEQ ID NO: 16 or a complementary sequence thereof exhibited the highest accuracy of 97.5% in the training cohort and the highest accuracy of 94.9% in the validation cohort. Furthermore, for example, the measurement using the combinations of four polynucleotides comprising at least one polynucleotide consisting of the nucleotide sequence represented by SEQ ID NO: 16 or a complementary sequence thereof exhibited the highest accuracy of 98.0% in the training cohort and the highest accuracy of 88.8% in the validation cohort.

The discriminant accuracy of the measurement using the polynucleotide consisting of the nucleotide sequence represented by SEQ ID NO: 37 or a complementary sequence thereof is shown in Table 8-4. The measurement using the combination of one polynucleotide comprising at least one polynucleotide consisting of the nucleotide sequence represented by SEQ ID NO: 37 or a complementary sequence thereof exhibited accuracy of 73.6% in the training cohort and accuracy of 72.4% in the validation cohort. Also, for example, the measurement using the combination of two polynucleotides comprising at least one polynucleotide consisting of the nucleotide sequence represented by SEQ ID NO: 37 or a complementary sequence thereof exhibited the highest accuracy of 95.9% in the training cohort and the highest accuracy of 92.9% in the validation cohort. Furthermore, for example, the measurement using the combinations of three polynucleotides comprising at least one polynucleotide consisting of the nucleotide sequence represented by SEQ ID NO: 37 or a complementary sequence thereof exhibited the highest accuracy of 97.0% in the training cohort and the highest accuracy of 92.9% in the validation cohort. Furthermore, for example, the measurement using the combinations of four polynucleotides comprising at least one polynucleotide consisting of the nucleotide sequence represented by SEQ ID NO: 37 or a complementary sequence thereof exhibited the highest accuracy of 97.0% in the training cohort and the highest accuracy of 89.8% in the validation cohort.

The discriminant accuracy of the measurement using the polynucleotide consisting of the nucleotide sequence represented by SEQ ID NO: 42 or a complementary sequence thereof is shown in Table 8-5. The measurement using the combination of one polynucleotide comprising at least one polynucleotide consisting of the nucleotide sequence represented by SEQ ID NO: 42 or a complementary sequence thereof exhibited accuracy of 57.4% in the training cohort and accuracy of 59.2% in the validation cohort. Also, for example, the measurement using the combination of two polynucleotides comprising at least one polynucleotide consisting of the nucleotide sequence represented by SEQ ID NO: 42 or a complementary sequence thereof exhibited the highest accuracy of 95.4% in the training cohort and the highest accuracy of 93.9% in the validation cohort. Furthermore, for example, the measurement using the combinations of three polynucleotides comprising at least one polynucleotide consisting of the nucleotide sequence represented by SEQ ID NO: 42 or a complementary sequence thereof exhibited the highest accuracy of 97.5% in the training cohort and the highest accuracy of 95.9% in the validation cohort. Furthermore, for example, the measurement using the combinations of four polynucleotides comprising at least one polynucleotide consisting of the nucleotide sequence represented by SEQ ID NO: 42 or a complementary sequence thereof exhibited the highest accuracy of 96.9% in the training cohort and the highest accuracy of 94.9% in the validation cohort.

The discriminant accuracy of the measurement using the polynucleotide consisting of the nucleotide sequence represented by SEQ ID NO: 63 or a complementary sequence thereof is shown in Table 8-6. The measurement using the combination of one polynucleotide comprising at least one polynucleotide consisting of the nucleotide sequence represented by SEQ ID NO: 63 or a complementary sequence thereof exhibited accuracy of 72.6% in the training cohort and accuracy of 73.5% in the validation cohort. Also, for example, the measurement using the combination of two polynucleotides comprising at least one polynucleotide consisting of the nucleotide sequence represented by SEQ ID NO: 63 or a complementary sequence thereof exhibited the highest accuracy of 94.9% in the training cohort and the highest accuracy of 92.9% in the validation cohort. Furthermore, for example, the measurement using the combinations of three polynucleotides comprising at least one polynucleotide consisting of the nucleotide sequence represented by SEQ ID NO: 63 or a complementary sequence thereof exhibited the highest accuracy of 95.9% in the training cohort and the highest accuracy of 95.9% in the validation cohort. Furthermore, for example, the measurement using the combinations of four polynucleotides comprising at least one polynucleotide consisting of the nucleotide sequence represented by SEQ ID NO: 63 or a complementary sequence thereof exhibited the highest accuracy of 94.4% in the training cohort and the highest accuracy of 89.8% in the validation cohort.

The discriminant accuracy of the measurement using the polynucleotide consisting of the nucleotide sequence represented by SEQ ID NO: 119 or a complementary sequence thereof is shown in Table 8-7. The measurement using the combination of one polynucleotide comprising at least one polynucleotide consisting of the nucleotide sequence represented by SEQ ID NO: 119 or a complementary sequence thereof exhibited accuracy of 46.9% in the training cohort and accuracy of 48.0% in the validation cohort. Also, for example, the measurement using the combination of two polynucleotides comprising at least one polynucleotide consisting of the nucleotide sequence represented by SEQ ID NO: 119 or a complementary sequence thereof exhibited the highest accuracy of 94.9% in the training cohort and the highest accuracy of 91.8% in the validation cohort. Furthermore, for example, the measurement using the combinations of three polynucleotides comprising at least one polynucleotide consisting of the nucleotide sequence represented by SEQ ID NO: 119 or a complementary sequence thereof exhibited the highest accuracy of 97.4% in the training cohort and the highest accuracy of 91.8% in the validation cohort. Furthermore, for example, the measurement using the combinations of four polynucleotides comprising at least one polynucleotide consisting of the nucleotide sequence represented by SEQ ID NO: 119 or a complementary sequence thereof exhibited the highest accuracy of 96.4% in the training cohort and the highest accuracy of 89.8% in the validation cohort.

The discriminant accuracy of the measurement using the polynucleotide consisting of the nucleotide sequence represented by SEQ ID NO: 126 or a complementary sequence thereof is shown in Table 8-8. The measurement using the combination of one polynucleotide comprising at least one polynucleotide consisting of the nucleotide sequence represented by SEQ ID NO: 126 or a complementary sequence thereof exhibited accuracy of 66.0% in the training cohort and accuracy of 53.1% in the validation cohort. Also, for example, the measurement using the combination of two polynucleotides comprising at least one polynucleotide consisting of the nucleotide sequence represented by SEQ ID NO: 126 or a complementary sequence thereof exhibited the highest accuracy of 94.4% in the training cohort and the highest accuracy of 91.8% in the validation cohort. Furthermore, for example, the measurement using the combinations of three polynucleotides comprising at least one polynucleotide consisting of the nucleotide sequence represented by SEQ ID NO: 126 or a complementary sequence thereof exhibited the highest accuracy of 96.4% in the training cohort and the highest accuracy of 90.8% in the validation cohort. Furthermore, for example, the measurement using the combinations of four polynucleotides comprising at least one polynucleotide consisting of the nucleotide sequence represented by SEQ ID NO: 126 or a complementary sequence thereof exhibited the highest accuracy of 93.9% in the training cohort and the highest accuracy of 91.8% in the validation cohort.

The discriminant accuracy of the measurement using the polynucleotide consisting of the nucleotide sequence represented by SEQ ID NO: 139 or a complementary sequence thereof is shown in Table 8-9. The measurement using the combination of one polynucleotide comprising at least one polynucleotide consisting of the nucleotide sequence represented by SEQ ID NO: 139 or a complementary sequence thereof exhibited accuracy of 43.7% in the training cohort and accuracy of 40.8% in the validation cohort. Also, for example, the measurement using the combination of two polynucleotides comprising at least one polynucleotide consisting of the nucleotide sequence represented by SEQ ID NO: 139 or a complementary sequence thereof exhibited the highest accuracy of 94.4% in the training cohort and the highest accuracy of 92.9% in the validation cohort. Furthermore, for example, the measurement using the combinations of three polynucleotides comprising at least one polynucleotide consisting of the nucleotide sequence represented by SEQ ID NO: 139 or a complementary sequence thereof exhibited the highest accuracy of 96.4% in the training cohort and the highest accuracy of 94.9% in the validation cohort. Furthermore, for example, the measurement using the combinations of four polynucleotides comprising at least one polynucleotide consisting of the nucleotide sequence represented by SEQ ID NO: 139 or a complementary sequence thereof exhibited the highest accuracy of 92.4% in the training cohort and the highest accuracy of 91.8% in the validation cohort.

The discriminant accuracy of the measurement using the polynucleotide consisting of the nucleotide sequence represented by SEQ ID NO: 173 or a complementary sequence thereof is shown in Table 8-10. The measurement using the combination of one polynucleotide comprising at least one polynucleotide consisting of the nucleotide sequence represented by SEQ ID NO: 173 or a complementary sequence thereof exhibited accuracy of 43.7% in the training cohort and accuracy of 55.1% in the validation cohort. Also, for example, the measurement using the combination of two polynucleotides comprising at least one polynucleotide consisting of the nucleotide sequence represented by SEQ ID NO: 173 or a complementary sequence thereof exhibited the highest accuracy of 94.9% in the training cohort and the highest accuracy of 91.8% in the validation cohort. Furthermore, for example, the measurement using the combinations of three polynucleotides comprising at least one polynucleotide consisting of the nucleotide sequence represented by SEQ ID NO: 173 or a complementary sequence thereof exhibited the highest accuracy of 97.0% in the training cohort and the highest accuracy of 91.8% in the validation cohort. Furthermore, for example, the measurement using the combinations of four polynucleotides comprising at least one polynucleotide consisting of the nucleotide sequence represented by SEQ ID NO: 173 or a complementary sequence thereof exhibited the highest accuracy of 92.4% in the training cohort and the highest accuracy of 95.9% in the validation cohort.

The discriminant accuracy of the measurement using the polynucleotide consisting of the nucleotide sequence represented by SEQ ID NO: 178 or a complementary sequence thereof is shown in Table 8-11. The measurement using the combination of one polynucleotide comprising at least one polynucleotide consisting of the nucleotide sequence represented by SEQ ID NO: 178 or a complementary sequence thereof exhibited accuracy of 68.0% in the training cohort and the highest accuracy of 72.4% in the validation cohort. Also, for example, the measurement using the combination of two polynucleotides comprising at least one polynucleotide consisting of the nucleotide sequence represented by SEQ ID NO: 178 or a complementary sequence thereof exhibited the highest accuracy of 94.4% in the training cohort and the highest accuracy of 91.8% in the validation cohort. Furthermore, for example, the measurement using the combinations of three polynucleotides comprising at least one polynucleotide consisting of the nucleotide sequence represented by SEQ ID NO: 178 or a complementary sequence thereof exhibited the highest accuracy of 96.4% in the training cohort and the highest accuracy of 94.9% in the validation cohort. Furthermore, for example, the measurement using the combinations of four polynucleotides comprising at least one polynucleotide consisting of the nucleotide sequence represented by SEQ ID NO: 178 or a complementary sequence thereof exhibited the highest accuracy of 96.4% in the training cohort and the highest accuracy of 93.9% in the validation cohort.

The discriminant accuracy of the measurement using the polynucleotide consisting of the nucleotide sequence represented by SEQ ID NO: 599 or a complementary sequence thereof is shown in Table 8-12. The measurement using the combination of one polynucleotide comprising at least one polynucleotide consisting of the nucleotide sequence represented by SEQ ID NO: 599 or a complementary sequence thereof exhibited accuracy of 61.4% in the training cohort and the highest accuracy of 65.3% in the validation cohort. Also, for example, the measurement using the combination of two polynucleotides comprising at least one polynucleotide consisting of the nucleotide sequence represented by SEQ ID NO: 599 or a complementary sequence thereof exhibited the highest accuracy of 94.4% in the training cohort and the highest accuracy of 91.8% in the validation cohort. Furthermore, for example, the measurement using the combinations of three polynucleotides comprising at least one polynucleotide consisting of the nucleotide sequence represented by SEQ ID NO: 599 or a complementary sequence thereof exhibited the highest accuracy of 97.5% in the training cohort and the highest accuracy of 92.9% in the validation cohort. Furthermore, for example, the measurement using the combinations of four polynucleotides comprising at least one polynucleotide consisting of the nucleotide sequence represented by SEQ ID NO: 599 or a complementary sequence thereof exhibited the highest accuracy of 96.4% in the training cohort and the highest accuracy of 94.9% in the validation cohort.

The discriminant accuracy of the measurement using the polynucleotide consisting of the nucleotide sequence represented by SEQ ID NO: 609 or a complementary sequence thereof is shown in Table 8-13. The measurement using the combination of one polynucleotide comprising at least one polynucleotide consisting of the nucleotide sequence represented by SEQ ID NO: 609 or a complementary sequence thereof exhibited accuracy of 59.7% in the training cohort and accuracy of 65.3% in the validation cohort. Also, for example, the measurement using the combination of two polynucleotides comprising at least one polynucleotide consisting of the nucleotide sequence represented by SEQ ID NO: 609 or a complementary sequence thereof exhibited the highest accuracy of 95.4% in the training cohort and the highest accuracy of 91.8% in the validation cohort. Furthermore, for example, the measurement using the combinations of three polynucleotides comprising at least one polynucleotide consisting of the nucleotide sequence represented by SEQ ID NO: 609 or a complementary sequence thereof exhibited the highest accuracy of 96.4% in the training cohort and the highest accuracy of 91.8% in the validation cohort. Furthermore, for example, the measurement using the combinations of four polynucleotides comprising at least one polynucleotide consisting of the nucleotide sequence represented by SEQ ID NO: 609 or a complementary sequence thereof exhibited the highest accuracy of 96.4% in the training cohort and the highest accuracy of 88.8% in the validation cohort.

The discriminant accuracy of the measurement using the polynucleotide consisting of the nucleotide sequence represented by SEQ ID NO: 611 or a complementary sequence thereof is shown in Table 8-14. The measurement using the combination of one polynucleotide comprising at least one polynucleotide consisting of the nucleotide sequence represented by SEQ ID NO: 611 or a complementary sequence thereof exhibited accuracy of 55.8% in the training cohort and accuracy of 62.2% in the validation cohort. Also, for example, the measurement using the combination of two polynucleotides comprising at least one polynucleotide consisting of the nucleotide sequence represented by SEQ ID NO: 611 or a complementary sequence thereof exhibited the highest accuracy of 94.9% in the training cohort and the highest accuracy of 91.8% in the validation cohort. Furthermore, for example, the measurement using the combinations of three polynucleotides comprising at least one polynucleotide consisting of the nucleotide sequence represented by SEQ ID NO: 611 or a complementary sequence thereof exhibited the highest accuracy of 98.0% in the training cohort and the highest accuracy of 90.8% in the validation cohort. Furthermore, for example, the measurement using the combinations of four polynucleotides comprising at least one polynucleotide consisting of the nucleotide sequence represented by SEQ ID NO: 611 or a complementary sequence thereof exhibited the highest accuracy of 96.4% in the training cohort and the highest accuracy of 90.80 in the validation cohort.

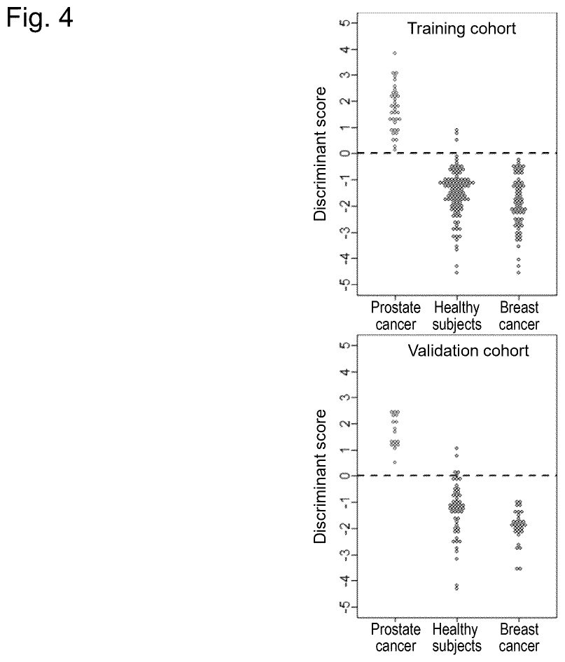

The expression level measurement values of the nucleotide sequences represented by SEQ ID NOs: 12, 16, 135, and 156 were compared among 35 prostate cancer patients, 99 healthy subjects, and 63 breast cancer patients in the training cohort. As a result, a scatter diagram that significantly separated the discriminant score of the prostate cancer patient group from the discriminant scores of the other groups was obtained in the training cohort (see the upper diagram of FIG. 4). These results were also reproducible in the validation cohort (see the lower diagram of FIG. 4).

TABLE 8-1

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 1 | 94.4 | 91.4 | 95.1 | 91.8 | 94.1 | 91.4 |
| 1_597 | 96.4 | 97.1 | 96.3 | 90.8 | 88.2 | 91.4 |
| 1_7_29 | 98.5 | 100 | 98.1 | 92.9 | 94.1 | 92.6 |
| 1_63_139_600 | 94.9 | 91.4 | 95.7 | 91.8 | 88.2 | 92.6 |
| 1_12_63_599 | 95.4 | 100 | 94.4 | 91.8 | 94.1 | 91.4 |
| 1_141_173_599 | 95.4 | 97.1 | 95.1 | 91.8 | 94.1 | 91.4 |
| 1_16_139_178 | 95.4 | 100 | 94.4 | 92.9 | 94.1 | 92.6 |
| 1_63_173_599 | 93.9 | 94.3 | 93.8 | 90.8 | 94.1 | 90.1 |

TABLE 8-2

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 12 | 65.5 | 74.3 | 63.6 | 56.1 | 70.6 | 53.1 |
| 1_12 | 94.9 | 94.3 | 95.1 | 91.8 | 94.1 | 91.4 |
| 1_7_12 | 98 | 100 | 97.5 | 93.9 | 94.1 | 93.8 |
| 12_42_63_609 | 92.3 | 97.1 | 91.3 | 89.8 | 100 | 87.7 |
| 12_16_135_156 | 98.5 | 100 | 98.1 | 94.9 | 100 | 93.8 |
| 12_16_169_178 | 94.9 | 100 | 93.8 | 88.8 | 100 | 86.4 |
| 12_16_139_601 | 94.9 | 100 | 93.8 | 91.8 | 100 | 90.1 |
| 12_16_42_607 | 97 | 100 | 96.3 | 93.9 | 100 | 92.6 |

TABLE 8-3

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 16 | 71.6 | 97.1 | 66 | 74.5 | 100 | 69.1 |
| 1_16 | 95.4 | 94.3 | 95.7 | 93.9 | 94.1 | 93.8 |
| 1_16_42 | 97.5 | 97.1 | 97.5 | 94.9 | 94.1 | 95.1 |
| 16_18_139_178 | 94.4 | 97.1 | 93.8 | 92.9 | 94.1 | 92.6 |
| 12_16_37_178 | 98 | 100 | 97.5 | 88.8 | 100 | 86.4 |
| 12_16_37_599 | 97.5 | 100 | 96.9 | 89.8 | 100 | 87.7 |
| 12_16_37_97 | 96.4 | 100 | 95.7 | 89.8 | 100 | 87.7 |
| 12_14_16_599 | 95.4 | 100 | 94.4 | 87.8 | 94.1 | 86.4 |

TABLE 8-4

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 37 | 73.6 | 77.1 | 72.8 | 72.4 | 82.4 | 70.4 |
| 1_37 | 95.9 | 97.1 | 95.7 | 92.9 | 88.2 | 93.8 |
| 1_37_135 | 97 | 97.1 | 96.9 | 92.9 | 88.2 | 93.8 |
| 37_63_139_611 | 93.4 | 88.6 | 94.4 | 88.8 | 94.1 | 87.7 |
| 37_42_63_178 | 91.4 | 94.3 | 90.7 | 90.8 | 94.1 | 90.1 |
| 37_42_63_599 | 91.4 | 91.4 | 91.4 | 91.8 | 94.1 | 91.4 |
| 37_42_63_139 | 91.9 | 91.4 | 92 | 91.8 | 94.1 | 91.4 |
| 12_16_37_603 | 97 | 100 | 96.3 | 89.8 | 100 | 87.7 |

TABLE 8-5

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 42 | 57.4 | 48.6 | 59.3 | 59.2 | 52.9 | 60.5 |
| 1_42 | 95.4 | 94.3 | 95.7 | 93.9 | 94.1 | 93.8 |
| 1_3_42 | 97.5 | 94.3 | 98.1 | 95.9 | 94.1 | 96.3 |
| 42_63_607_611 | 90.4 | 88.6 | 90.7 | 90.8 | 100 | 88.9 |
| 42_63_609_611 | 90.8 | 88.6 | 91.3 | 91.8 | 100 | 90.1 |
| 42_63_173_599 | 89.3 | 91.4 | 88.9 | 90.8 | 100 | 88.9 |
| 12_16_42_609 | 96.9 | 100 | 96.3 | 94.9 | 100 | 93.8 |
| 42_63_91_609 | 88.3 | 91.4 | 87.6 | 90.8 | 100 | 88.9 |

TABLE 8-6

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 63 | 72.6 | 88.6 | 69.1 | 73.5 | 88.2 | 70.4 |
| 1_63 | 94.9 | 91.4 | 95.7 | 92.9 | 94.1 | 92.6 |
| 1_42_63 | 95.9 | 94.3 | 96.3 | 95.9 | 94.1 | 96.3 |
| 10_42_63_599 | 92.9 | 97.1 | 92 | 91.8 | 100 | 90.1 |
| 42_63_599_609 | 88.8 | 91.4 | 88.2 | 91.8 | 100 | 90.1 |
| 42_63_583_609 | 94.4 | 91.4 | 95 | 89.8 | 100 | 87.7 |
| 37_42_63_611 | 93.9 | 91.4 | 94.4 | 94.9 | 100 | 93.8 |
| 12_63_70_599 | 90.9 | 100 | 88.9 | 89.8 | 94.1 | 88.9 |

TABLE 8-7

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 119 | 46.9 | 62.9 | 43.5 | 48 | 58.8 | 45.7 |
| 1_119 | 94.9 | 91.4 | 95.7 | 91.8 | 94.1 | 91.4 |
| 1_16_119 | 97.4 | 100 | 96.9 | 91.8 | 88.2 | 92.6 |
| 12_16_37_119 | 96.4 | 100 | 95.7 | 89.8 | 100 | 87.7 |
| 37_63_119_584 | 93.4 | 88.6 | 94.4 | 87.8 | 94.1 | 86.4 |
| 63_119_173_178 | 87.2 | 88.6 | 87 | 82.7 | 94.1 | 80.2 |
| 63_119_158_173 | 85.7 | 88.6 | 85.1 | 84.7 | 88.2 | 84 |
| 63_119_173_605 | 87.2 | 88.6 | 87 | 82.7 | 88.2 | 81.5 |

TABLE 8-8

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 126 | 66 | 71.4 | 64.8 | 53.1 | 64.7 | 50.6 |
| 1_126 | 94.4 | 94.3 | 94.4 | 91.8 | 94.1 | 91.4 |
| 1_126_597 | 96.4 | 97.1 | 96.3 | 90.8 | 88.2 | 91.4 |
| 16_126_597_599 | 90.9 | 100 | 88.9 | 81.6 | 88.2 | 80.2 |
| 16_42_126_599 | 92.9 | 94.3 | 92.6 | 92.9 | 100 | 91.4 |
| 16_126_139_601 | 93.9 | 100 | 92.6 | 91.8 | 100 | 90.1 |
| 16_126_593_599 | 89.8 | 97.1 | 88.3 | 85.7 | 94.1 | 84 |
| 15_16_126_599 | 91.4 | 97.1 | 90.1 | 81.6 | 94.1 | 79 |

TABLE 8-9

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 139 | 43.7 | 62.9 | 39.5 | 40.8 | 64.7 | 35.8 |
| 1_139 | 94.4 | 91.4 | 95.1 | 92.9 | 94.1 | 92.6 |
| 1_139_141 | 96.4 | 97.1 | 96.3 | 94.9 | 94.1 | 95.1 |
| 37_63_139_584 | 92.4 | 91.4 | 92.6 | 90.8 | 94.1 | 90.1 |
| 63_139_173_178 | 85.3 | 91.4 | 84 | 89.8 | 94.1 | 88.9 |
| 16_63_139_601 | 92.4 | 97.1 | 91.4 | 91.8 | 94.1 | 91.4 |
| 37_63_139_600 | 89.8 | 91.4 | 89.5 | 88.8 | 94.1 | 87.7 |
| 16_139_178_586 | 91.4 | 100 | 89.5 | 92.9 | 100 | 91.4 |

TABLE 8-10

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 173 | 43.7 | 51.4 | 42 | 55.1 | 58.8 | 54.3 |
| 1_173 | 94.9 | 94.3 | 95.1 | 91.8 | 94.1 | 91.4 |
| 1_15_173 | 97 | 97.1 | 96.9 | 91.8 | 94.1 | 91.4 |
| 63_139_173_599 | 84.8 | 88.6 | 84 | 89.8 | 94.1 | 88.9 |
| 63_119_173_581 | 90.3 | 91.4 | 90.1 | 89.8 | 94.1 | 88.9 |
| 63_173_582_599 | 88.3 | 91.4 | 87.7 | 84.5 | 88.2 | 83.8 |
| 63_136_173_599 | 92.4 | 94.3 | 92 | 95.9 | 94.1 | 96.3 |
| 29_63_173_178 | 87.8 | 91.4 | 87 | 88.8 | 88.2 | 88.9 |

TABLE 8-11

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 178 | 68 | 68.6 | 67.9 | 72.4 | 82.4 | 70.4 |
| 1_178 | 94.4 | 91.4 | 95.1 | 91.8 | 94.1 | 91.4 |
| 1_15_178 | 96.4 | 97.1 | 96.3 | 94.9 | 94.1 | 95.1 |
| 16_139_178_601 | 93.4 | 100 | 92 | 90.8 | 100 | 88.9 |
| 16_37_139_178 | 93.4 | 94.3 | 93.2 | 91.8 | 94.1 | 91.4 |
| 1_12_16_178 | 96.4 | 100 | 95.7 | 93.9 | 100 | 92.6 |
| 1_63_173_178 | 94.9 | 94.3 | 95.1 | 91.8 | 94.1 | 91.4 |
| 16_139_178_597 | 93.9 | 100 | 92.6 | 89.8 | 100 | 87.7 |

TABLE 8-12

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 599 | 61.4 | 74.3 | 58.6 | 65.3 | 82.4 | 61.7 |
| 1_599 | 94.4 | 91.4 | 95.1 | 91.8 | 94.1 | 91.4 |
| 3_112_599 | 97.5 | 97.1 | 97.5 | 92.9 | 94.1 | 92.6 |
| 12_37_63_599 | 91.9 | 97.1 | 90.7 | 88.8 | 94.1 | 87.7 |
| 42_58_63_599 | 90.9 | 94.3 | 90.1 | 87.8 | 94.1 | 86.4 |
| 1_12_16_599 | 96.4 | 100 | 95.7 | 94.9 | 100 | 93.8 |
| 63_119_173_599 | 87.2 | 88.6 | 87 | 80.6 | 88.2 | 79 |
| 16_18_139_599 | 94.9 | 97.1 | 94.4 | 92.9 | 94.1 | 92.6 |

TABLE 8-13

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 609 | 59.7 | 77.1 | 55.9 | 65.3 | 82.4 | 61.7 |
| 1_609 | 95.4 | 94.3 | 95.7 | 91.8 | 94.1 | 91.4 |
| 1_10_609 | 96.4 | 94.3 | 96.9 | 91.8 | 94.1 | 91.4 |
| 42_63_585_609 | 89.8 | 91.4 | 89.4 | 91.8 | 100 | 90.1 |
| 42_63_592_609 | 88.8 | 88.6 | 88.8 | 89.8 | 100 | 87.7 |
| 18_42_581_609 | 93.4 | 94.3 | 93.2 | 90.8 | 94.1 | 90.1 |
| 12_16_599_609 | 96.4 | 100 | 95.7 | 88.8 | 100 | 86.4 |
| 16_126_599_609 | 87.2 | 97.1 | 85.1 | 84.7 | 88.2 | 84.0 |

TABLE 8-14

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 611 | 55.8 | 54.3 | 56.2 | 62.2 | 58.8 | 63 |
| 1_611 | 94.9 | 94.3 | 95.1 | 91.8 | 94.1 | 91.4 |
| 10_15_611 | 98 | 100 | 97.5 | 90.8 | 100 | 88.9 |
| 12_16_37_611 | 96.4 | 100 | 95.7 | 90.8 | 100 | 88.9 |
| 1_63_139_611 | 94.4 | 88.6 | 95.7 | 91.8 | 88.2 | 92.6 |
| 63_158_173_611 | 87.8 | 88.6 | 87.7 | 83.7 | 88.2 | 82.7 |
| 16_37_139_611 | 93.9 | 97.1 | 93.2 | 90.8 | 100 | 88.9 |
| 16_37_595_611 | 91.9 | 97.1 | 90.7 | 84.7 | 82.4 | 85.2 |

As shown in these Examples, the kit, device and the method of the present invention can detect prostate cancer more sensitively than the existing tumor markers and therefore permit early decision to carry out the surgical resection of the cancer site. As a result, improvement in 5-year survival rate and reduction in the rate of recurrence can be achieved.

### INDUSTRIAL APPLICABILITY

According to the present invention, prostate cancer can be effectively detected by a simple and inexpensive method. This permits early detection, diagnosis and treatment of prostate cancer. The method of the present invention can detect prostate cancer with limited invasiveness using the blood of a patient and therefore allows prostate cancer to be detected conveniently and rapidly.

All publications, patents, and patent applications cited herein are incorporated herein by reference in their entirety.

1. A method for detecting prostate cancer in a human subject, comprising:
measuring an expression level of hsa-miR-3197 in a blood, serum, or plasma sample from the subject;
comparing the measured expression level of hsa-miR-3197 to a control expression level in a sample from a healthy subject;
detecting an increased level of hsa-miR-3197 in the sample from the subject as compared to the control expression level from the sample from the healthy subject;
wherein the increased level of hsa-miR-3197 indicates that the subject has prostate cancer; and
wherein the method further comprises treating the subject for the prostate cancer or performing a diagnostic procedure on the subject with the prostate cancer;
wherein the treating comprises surgery, radiotherapy, chemotherapy or the combination thereof; and
wherein the diagnostic procedure comprises rectal examination, transrectal ultrasonography of the prostate, or imaging of prostate tissue.

2. The method according to claim 1, wherein the expression level of hsa-miR-3197 in the sample is measured by using a kit comprising a nucleic acid(s) that specifically binds to hsa-miR-3197.

3. The method according to claim 2, wherein the kit further comprises at least one nucleic acid capable of specifically binding to at least one polynucleotide selected from the group consisting of other prostate cancer markers: miR-1908-5p, miR-4257, miR-3188, miR-4649-5p, miR-1343-3p, miR-6861-5p, miR-1343-5p, miR-642b-3p, miR-4745-5p, miR-6826-5p, miR-3663-3p, miR-3131, miR-92a-2-5p, miR-4258, miR-4448, miR-6125, miR-6880-5p, miR-6132, miR-4467, miR-6749-5p, miR-2392, miR-1273g-3p, miR-4746-3p, miR-1914-3p, miR-7845-5p, miR-6726-5p, miR-128-2-5p, miR-4651, miR-6765-3p, miR-3185, miR-4792, miR-6887-5p, miR-5572, miR-3619-3p, miR-6780b-5p, miR-4707-5p, miR-8063, miR-4454, miR-4525, miR-7975, miR-744-5p, miR-3135b, miR-4648, miR-6816-5p, miR-7150, miR-6791-5p, miR-1247-3p, miR-7977, miR-4497, miR-6090, miR-6781-5p, miR-6870-5p, miR-6729-5p, miR-4530, miR-7847-3p, miR-6825-5p, miR-4674, miR-3917, miR-4707-3p, miR-6885-5p, miR-6722-3p, miR-4516, miR-6757-5p, miR-6840-3p, miR-5195-3p, miR-6756-5p, miR-6800-5p, miR-6727-5p, miR-6126, miR-6872-3p, miR-4446-3p, miR-1268a, miR-1908-3p, miR-3679-5p, miR-4534, miR-4675, miR-7108-5p, miR-6799-5p, miR-4695-5p, miR-3178, miR-5090, miR-3180, miR-1237-5p, miR-4758-5p, miR-3184-5p, miR-4286, miR-6784-5p, miR-6768-5p, miR-6785-5p, miR-4706, miR-711, miR-1260a, miR-6746-5p, miR-6089, miR-6821-5p, miR-4667-5p, miR-8069, miR-4726-5p, miR-6124, miR-4532, miR-4486, miR-4728-5p, miR-4508, miR-128-1-5p, miR-4513, miR-6795-5p, miR-4689, miR-6763-5p, miR-8072, miR-6765-5p, miR-4419b, miR-7641, miR-1227-5p, miR-4492, miR-296-3p, miR-6769a-5p, miR-6889-5p, miR-4632-5p, miR-4505, miR-3154, miR-3648, miR-4442, miR-3141, miR-7113-3p, miR-6819-5p, miR-3195, miR-1199-5p, miR-6738-5p, miR-4656, miR-6820-5p, miR-204-3p, miR-642a-3p, miR-762, miR-1202, miR-3162-5p, miR-3196, miR-3622a-5p, miR-3665, miR-3940-5p, miR-4294, miR-4466, miR-4476, miR-4723-5p, miR-4725-3p, miR-4730, miR-4739, miR-4787-5p, miR-5787, miR-6085, miR-6717-5p, miR-6724-5p, miR-6777-5p, miR-6778-5p, miR-6787-5p, miR-6789-5p, miR-6845-5p miR-6893-5p, and/or miR-615-5p, miR-486-3p, miR-1225-3p, miR-760, miR-187-5p, miR-1203, miR-7110-5p, miR-371a-5p, miR-939-5p, miR-575, miR-92b-5p, miR-887-3p, miR-920, miR-1915-5p, miR-1231, miR-663b, miR-1225-5p, miR-16-5p, miR-423-5p, miR-451a, miR-564, miR-671-5p, miR-4763-3p, miR-3656, miR-4488, miR-125a-3p, miR-1469, miR-1228-5p, miR-6798-5p, miR-1268b, miR-6732-5p, miR-1915-3p, miR-4433b-3p, miR-1207-5p, miR-4433-3p, miR-6879-5p, miR-4417, miR-30c-1-3p, miR-4638-5p, miR-6088, miR-4270, miR-6782-5p, miR-665, miR-486-5p, miR-4655-5p, miR-1275, miR-6806-5p, miR-614, miR-3937, miR-6752-5p, miR-6771-5p, miR-4450, miR-211-3p, miR-663a, miR-6842-5p, miR-7114-5p and miR-6779-5p.

4. The method according to claim 3, comprising performing the diagnostic procedure on the subject.

5. The method according to claim 1, wherein the expression level of hsa-miR-3197 in the sample is measured by using a device comprising a nucleic acid(s) that specifically binds to hsa-miR-3197.

6. The method according to claim 5, wherein the device further comprises at least one nucleic acid capable of specifically binding to at least one polynucleotide selected from the group consisting of other prostate cancer markers: miR-1908-5p, miR-4257, miR-3188, miR-4649-5p, miR-1343-3p, miR-6861-5p, miR-1343-5p, miR-642b-3p, miR-4745-5p, miR-6826-5p, miR-3663-3p, miR-3131, miR-92a-2-5p, miR-4258, miR-4448, miR-6125, miR-6880-5p, miR-6132, miR-4467, miR-6749-5p, miR-2392, miR-1273g-3p, miR-4746-3p, miR-1914-3p, miR-7845-5p, miR-6726-5p, miR-128-2-5p, miR-4651, miR-6765-3p, miR-3185, miR-4792, miR-6887-5p, miR-5572, miR-3619-3p, miR-6780b-5p, miR-4707-5p, miR-8063, miR-4454, miR-4525, miR-7975, miR-744-5p, miR-3135b, miR-4648, miR-6816-5p, miR-7150, miR-6791-5p, miR-1247-3p, miR-7977, miR-4497, miR-6090, miR-6781-5p, miR-6870-5p, miR-6729-5p, miR-4530, miR-7847-3p, miR-6825-5p, miR-4674, miR-3917, miR-4707-3p, miR-6885-5p, miR-6722-3p, miR-4516, miR-6757-5p, miR-6840-3p, miR-5195-3p, miR-6756-5p, miR-6800-5p, miR-6727-5p, miR-6126, miR-6872-3p, miR-4446-3p, miR-1268a, miR-1908-3p, miR-3679-5p, miR-4534, miR-4675, miR-7108-5p, miR-6799-5p, miR-4695-5p, miR-3178, miR-5090, miR-3180, miR-1237-5p, miR-4758-5p, miR-3184-5p, miR-4286, miR-6784-5p, miR-6768-5p, miR-6785-5p, miR-4706, miR-711, miR-1260a, miR-6746-5p, miR-6089, miR-6821-5p, miR-4667-5p, miR-8069, miR-4726-5p, miR-6124, miR-4532, miR-4486, miR-4728-5p, miR-4508, miR-128-1-5p, miR-4513, miR-6795-5p, miR-4689, miR-6763-5p, miR-8072, miR-6765-5p, miR-4419b, miR-7641, miR-1227-5p, miR-4492, miR-296-3p, miR-6769a-5p, miR-6889-5p, miR-4632-5p, miR-4505, miR-3154, miR-3648, miR-4442, miR-3141, miR-7113-3p, miR-6819-5p, miR-3195, miR-1199-5p, miR-6738-5p, miR-4656, miR-6820-5p, miR-204-3p, miR-642a-3p, miR-762, miR-1202, miR-3162-5p, miR-3196, miR-3622a-5p, miR-3665, miR-3940-5p, miR-4294, miR-4466, miR-4476, miR-4723-5p, miR-4725-3p, miR-4730, miR-4739, miR-4787-5p, miR-5787, miR-6085, miR-6717-5p, miR-6724-5p, miR-6777-5p, miR-6778-5p, miR-6787-5p, miR-6789-5p, miR-6845-5p miR-6893-5p, and/or miR-615-5p, miR-486-3p, miR-1225-3p, miR-760, miR-187-5p, miR-1203, miR-7110-5p, miR-371a-5p, miR-939-5p, miR-575, miR-92b-5p, miR-887-3p, miR-920, miR-1915-5p, miR-1231, miR-663b, miR-1225-5p, miR-16-5p, miR-423-5p, miR-451a, miR-564, miR-671-5p, miR-4763-3p, miR-3656, miR-4488, miR-125a-3p, miR-1469, miR-1228-5p, miR-6798-5p, miR-1268b, miR-6732-5p, miR-1915-3p, miR-4433b-3p, miR-1207-5p, miR-4433-3p, miR-6879-5p, miR-4417, miR-30c-1-3p, miR-4638-5p, miR-6088, miR-4270, miR-6782-5p, miR-665, miR-486-5p, miR-4655-5p, miR-1275, miR-6806-5p, miR-614, miR-3937, miR-6752-5p, miR-6771-5p, miR-4450, miR-211-3p, miR-663a, miR-6842-5p, miR-7114-5p and miR-6779-5p.
